(19) United States
(12) Patent Application Publication
(10) Pub. No.: US 2005/0192347 A1
(43) Pub. Date: Sep. 1, 2005

(54) Urea and thiourea compounds and compositions for cholesterol management and related uses

(76) Inventors: Jean-Louis Henri Dasseux, Toulouse (FR); Carmen Daniela Oniciu, Gainesville, FL (US)

Correspondence Address: JONES DAY, 51 Louisiana Aveue, N.W, WASHINGTON, DC, 20001-2113, US

(21) Appl. No.: 11/021,777

(22) Filed: Dec. 23, 2004

Related U.S. Application Data
(60) Provisional application No. 60/608,928, filed on Dec. 23, 2003.

(51) Int. Cl. A61K 31/277; A61K 31/17; C07C 335/20; C07C 275/30

(52) U.S. Cl. 514/521; 514/585; 514/595; 558/410; 564/27; 564/47

(57) ABSTRACT

The present invention relates to novel urea and thiourea compounds, compositions comprising urea or thiourea compounds, and methods useful for treating and preventing aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. In certain embodiments, the compounds, compositions, and methods of the invention are useful in combination therapy with other therapeutics, such as hypocholesterolemic and hypoglycemic agents.

This application claims the benefit of U.S. provisional application No. 60/608,928, filed Dec. 23, 2003, which is incorporated by reference herein in its entirety.

### 1. FIELD OF THE INVENTION

The invention encompasses urea and thiourea compounds and pharmaceutically acceptable salts, hydrates, solvates, and mixtures thereof; compositions comprising urea and thiourea compounds and pharmaceutically acceptable salts, hydrates, solvates, and mixtures thereof; and methods for treating or preventing a disease or disorder such as, but not limited to, aging, Alzheimer's Disease, cancer, cardiovascular disease, diabetic nephropathy, diabetic retinopathy, a disorder of glucose metabolism, dyslipidemia, dyslipoproteinemia, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, or enhancing bile production, or enhancing reverse lipid transport, which method comprise administering a urea or thiourea compound or composition of the invention to a patient in need thereof. The compounds of the invention can also treat or prevent inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism.

### 2. BACKGROUND OF THE INVENTION

Obesity, hyperlipidemia, and diabetes have been shown to play a causal role in atherosclerotic cardiovascular diseases, which currently account for a considerable proportion of morbidity in Western society. Further, one human disease, termed “Syndrome X” or “Metabolic Syndrome”, is manifested by defective glucose metabolism (insulin resistance), elevated blood pressure (hypertension), and a blood lipid imbalance (dyslipidemia). See e.g. Reaven, 1993, Annu. Rev. Med. 44:121-131.

The evidence linking elevated serum cholesterol to coronary heart disease is overwhelming. Circulating cholesterol is carried by plasma lipoproteins, which are particles of complex lipid and protein composition that transport lipids in the blood. Low density lipoprotein (LDL) and high density lipoprotein (HDL) are the major cholesterol-carrier proteins. LDL is believed to be responsible for the delivery of cholesterol from the liver, where it is synthesized or obtained from dietary sources, to extrahepatic tissues in the body. The term “reverse cholesterol transport” describes the transport of cholesterol from extrahepatic tissues to the liver, where it is catabolized and eliminated. It is believed that plasma HDL particles play a major role in the reverse transport process, acting as scavengers of tissue cholesterol. HDL is also responsible for the removal of non-cholesterol lipid, oxidized cholesterol and other oxidized products from the bloodstream.

Atherosclerosis, for example, is a slowly progressive disease characterized by the accumulation of cholesterol within the arterial wall. Compelling evidence supports the belief that lipids deposited in atherosclerotic lesions are derived primarily from plasma apolipoprotein B (apo B)-containing lipoproteins, which include chylomicrons, CLDL, intermediate-density lipoproteins (IDL), and LDL. The apo B-containing lipoprotein, and in particular LDL, has popularly become known as the “bad” cholesterol. In contrast, HDL serum levels correlate inversely with coronary heart disease. Indeed, high serum levels of HDL are regarded as a negative risk factor. It is hypothesized that high levels of plasma HDL are not only protective against coronary artery disease, but may actually induce regression of atherosclerotic plaque (e.g., see Badimon et al., 1992, Circulation 86:(Suppl. III)86-94; Dansky and Fisher, 1999, Circulation 100:1762 3.). Thus, HDL has popularly become known as the “good” cholesterol.

### 2.1 Cholesterol Transport

The fat-transport system can be divided into two pathways: an exogenous one for cholesterol and triglycerides absorbed from the intestine and an endogenous one for cholesterol and triglycerides entering the bloodstream from the liver and other non-hepatic tissue.

In the exogenous pathway, dietary fats are packaged into lipoprotein particles called chylomicrons, which enter the bloodstream and deliver their triglycerides to adipose tissue for storage and to muscle for oxidation to supply energy. The remnant of the chylomicron, which contains cholesteryl esters, is removed from the circulation by a specific receptor found only on liver cells. This cholesterol then becomes available again for cellular metabolism or for recycling to extrahepatic tissues as plasma lipoproteins.

In the endogenous pathway, the liver secretes a large, very-low-density lipoprotein particle (VLDL) into the blood stream. The core of VLDL consists mostly of triglycerides synthesized in the liver, with a smaller amount of cholesteryl esters either synthesized in the liver or recycled from chylomicrons. Two predominant proteins are displayed on the surface of VLDL, apolipoprotein B-100 (apo B-100) and apolipoprotein E (apo E), although other apolipoproteins are present, such as apolipoprotein CIII (apo CIII) and apolipoprotein CII (apo CII). When VLDL reaches the capillaries of adipose tissue or of muscle, its triglyceride is extracted. This results in the formation of a new kind of particle called intermediate-density lipoprotein (IDL) or VLDL remnant, decreased in size and enriched in cholesteryl esters relative to a VLDL, but retaining its two apoproteins.

In human beings, about half of the IDL particles are removed from the circulation quickly, generally within two to six hours of their formation. This is because IDL particles bind tightly to liver cells, which extract IDL cholesterol to make new VLDL and bile acids. The IDL not taken up by the liver is catabolized by the hepatic lipase, an enzyme bound to the proteoglycan on liver cells. Apo E dissociates from IDL as it is transformed to LDL. Apo B-100 is the sole protein of LDL.

Primarily, the liver takes up and degrades circulating cholesterol to bile acids, which are the end products of cholesterol metabolism. The uptake of cholesterol-containing particles is mediated by LDL receptors, which are present in high concentrations on hepatocytes. The LDL receptor binds both apo E and apo B-100 and is responsible for binding and removing both IDL and LDL from the circulation. In addition, remnant receptors are responsible for clearing chylomicrons and VLDL remnants (i.e., IDL). However, the affinity of apo E for the LDL receptor is greater than that of apo B-100. As a result, the LDL particles have a much longer circulating life span than IDL particles; LDL circulates for an average of two and a half days before binding to the LDL receptors in the liver and other tissues. High serum levels of LDL, the “bad” cholesterol, are positively associated with coronary heart disease. For example, in atherosclerosis, cholesterol derived from circulating LDL accumulates in the walls of arteries. This accumulation forms bulky plaques that inhibit the flow of blood until a clot eventually forms, obstructing an artery and causing a heart attack or stroke.

Ultimately, the amount of intracellular cholesterol liberated from the LDL controls cellular cholesterol metabolism. The accumulation of cellular cholesterol derived from VLDL and LDL controls three processes. First, it reduces the ability of the cell to make its own cholesterol by turning off the synthesis of HMGCoA reductase, a key enzyme in the cholesterol biosynthetic pathway. Second, the incoming LDL-derived cholesterol promotes storage of cholesterol by the action of cholesterol acyltransferase (“ACAT”), the cellular enzyme that converts cholesterol into cholesteryl esters that are deposited in storage droplets. Third, the accumulation of cholesterol within the cell drives a feedback mechanism that inhibits cellular synthesis of new LDL receptors. Cells, therefore, adjust their complement of LDL receptors so that enough cholesterol is brought in to meet their metabolic needs, without overloading (for a review, see Brown & Goldstein, in The Pharmacological Basis Of Therapeutics, 8th Ed., Goodman & Gilman, Pergamon Press, New York, 1990, Ch. 36, pp. 874-896).

High levels of apo B-containing lipoproteins can be trapped in the subendothelial space of an artery and undergo oxidation. The oxidized lipoprotein is recognized by scavenger receptors on macrophages. Binding of oxidized lipoprotein to the scavenger receptors can enrich the macrophages with cholesterol and cholesteryl esters independently of the LDL receptor. Macrophages can also produce cholesteryl esters by the action of ACAT. LDL can also be complexed to a high molecular weight glycoprotein called apolipoprotein(a), also known as apo(a), through a disulfide bridge. The LDL-apo(a) complex is known as Lipoprotein(a) or Lp(a). Elevated levels of Lp(a) are detrimental, having been associated with atherosclerosis, coronary heart disease, myocardial infarction, stroke, cerebral infarction, and restenosis following angioplasty.

### 2.2 Reverse Cholesterol Transport

Peripheral (non-hepatic) cells predominantly obtain their cholesterol from a combination of local synthesis and uptake of preformed sterol from VLDL and LDL. Cells expressing scavenger receptors, such as macrophages and smooth muscle cells, can also obtain cholesterol from oxidized apo B-containing lipoproteins. In contrast, reverse cholesterol transport (RCT) is the pathway, by which peripheral cell cholesterol can be returned to the liver for recycling to extrahepatic tissues, hepatic storage, or excretion into the intestine in bile. The RCT pathway represents the only means of eliminating cholesterol from most extrahepatic tissues and is crucial to the maintenance of the structure and function of most cells in the body.

The enzyme in blood involved in the RCT pathway, lecithin:cholesterol acyltransferase (LCAT), converts cell-derived cholesterol to cholesteryl esters, which are sequestered in HDL destined for removal. LCAT is produced mainly in the liver and circulates in plasma associated with the HDL fraction. Cholesterol ester transfer protein (CETP) and another lipid transfer protein, phospholipid transfer protein (PLTP), contribute to further remodeling the circulating HDL population (see for example Bruce et al., 1998, Annu. Rev. Nutr. 18:297 330). PLTP supplies lecithin to HDL, and CETP can move cholesteryl esters made by LCAT to other lipoproteins, particularly apoB-containing lipoproteins, such as VLDL. HDL triglycerides can be catabolized by the extracellular hepatic triglyceride lipase, and lipoprotein cholesterol is removed by the liver via several mechanisms.

Each HDL particle contains at least one molecule, and usually two to four molecules, of apolipoprotein A I (apo A I). Apo A I is synthesized by the liver and small intestine as preproapolipoprotein, which is secreted as a proprotein that is rapidly cleaved to generate a mature polypeptide having 243 amino acid residues. Apo A I consists mainly of a 22 amino acid repeating segment, spaced with helix-breaking proline residues. Apo A I forms three types of stable structures with lipids: small, lipid-poor complexes referred to as pre-beta-1 HDL; flattened discoidal particles, referred to as pre-beta-2 HDL, which contain only polar lipids (e.g., phospholipid and cholesterol); and spherical particles containing both polar and nonpolar lipids, referred to as spherical or mature HDL (HDL3 and HDL2). Most HDL in the circulating population contains both apo A I and apo A II, a second major HDL protein. This apo A I- and apo A II-containing fraction is referred to herein as the AI/AII-HDL fraction of HDL. But the fraction of HDL containing only apo A I, referred to herein as the AI HDL fraction, appears to be more effective in RCT. Certain epidemiologic studies support the hypothesis that the AI-HDL fraction is antiartherogenic (Parra et al., 1992, Arterioscler. Thromb. 12:701-707; Decossin et al., 1997, Eur. J. Clin. Invest. 27:299-307).

Although the mechanism for cholesterol transfer from the cell surface is unknown, it is believed that the lipid-poor complex, pre-beta-1 HDL, is the preferred acceptor for cholesterol transferred from peripheral tissue involved in RCT. Cholesterol newly transferred to pre-beta-1 HDL from the cell surface rapidly appears in the discoidal pre-beta-2 HDL. PLTP may increase the rate of disc formation (Lagrost et al., 1996, J. Biol. Chem. 271:19058-19065), but data indicating a role for PLTP in RCT is lacking. LCAT reacts preferentially with discoidal and spherical HDL, transferring the 2-acyl group of lecithin or phosphatidylethanolamine to the free hydroxyl residue of fatty alcohols, particularly cholesterol, to generate cholesteryl esters (retained in the HDL) and lysolecithin. The LCAT reaction requires an apolipoprotein such as apo A I or apo A-IV as an activator. ApoA-I is one of the natural cofactors for LCAT. The conversion of cholesterol to its HDL-sequestered ester prevents re-entry of cholesterol into the cell, resulting in the ultimate removal of cellular cholesterol. Cholesteryl esters in the mature HDL particles of the AI-HDL fraction are removed by the liver and processed into bile more effectively than those derived from the AI/AII-HDL fraction. This may be due, in part, to the more effective binding of AI-HDL to the hepatocyte membrane. Several HDL receptors have been identified, the most well characterized of which is the scavenger receptor class B, type I (SR BI) (Acton et al., 1996, Science 271:518-520). The SR-BI is expressed most abundantly in steroidogenic tissues (e.g., the adrenals), and in the liver (Landshulz et al., 1996, J. Clin. Invest. 98:984-995; Rigotti et al., 1996, J. Biol. Chem. 271:33545-33549). Other proposed HDL receptors include HB1 and HB2 (Hidaka and Fidge, 1992, Biochem J. 15:161 7; Kurata et al., 1998, J. Atherosclerosis and Thrombosis 4:112 7).

While there is a consensus that CETP is involved in the metabolism of VLDL- and LDL-derived lipids, its role in RCT remains controversial. However, changes in CETP activity or its acceptors, VLDL and LDL, play a role in “remodeling” the HDL population. For example, in the absence of CETP, the HDL becomes enlarged particles that are poorly removed from the circulation (for reviews on RCT and HDL, See Fielding & Fielding, 1995, J. Lipid Res. 36:211-228; Barrans et al., 1996, Biochem. Biophys. Acta. 1300:73-85; Hirano et al., 1997, Arterioscler. Thromb. Vasc. Biol. 17:1053-1059).

### 2.3 Reverse Transport of Other Lipids

HDL is not only involved in the reverse transport of cholesterol, but also plays a role in the reverse transport of other lipids, i.e., the transport of lipids from cells, organs, and tissues to the liver for catabolism and excretion. Such lipids include sphingomyelin, oxidized lipids, and lysophophatidylcholine. For example, Robins and Fasulo (1997, J. Clin. Invest. 99:380 384) have shown that HDL stimulates the transport of plant sterol by the liver into bile secretions.

### 2.4 Peroxisome Proliferator Activated Receptor Pathway

Peroxisome proliferators are a structurally diverse group of compounds that, when administered to rodents, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, 1985, Ann. Rev. Cell Biol. 1:489 530; Vamecq and Draye, 1989, Essays Biochem. 24:1115 225; and Nelali et al., 1988, Cancer Res. 48:5316 5324). Chemicals included in this group are the fibrate class of hypolipidemic drugs, herbicides, and phthalate plasticizers (Reddy and Lalwani, 1983, Crit. Rev. Toxicol. 12:1 58). Peroxisome proliferation can also be elicited by dietary or physiological factors, such as a high fat diet and cold acclimatization.

Insight into the mechanism whereby peroxisome proliferators exert their pleiotropic effects was provided by the identification of a member of the nuclear hormone receptor superfamily activated by these chemicals (Isseman and Green, 1990, Nature 347:645 650). This receptor, termed peroxisome proliferator activated receptor α (PPARα), was subsequently shown to be activated by a variety of medium and long chain fatty acids. PPARα activates transcription by binding to DNA sequence elements, termed peroxisome proliferator response elements (PPRE), in the form of a heterodimer with the retinoid X receptor (RXR). RXR is activated by 9-cis retinoic acid (see Kliewer et al., 1992, Nature 358:771 774; Gearing et al., 1993, Proc. Natl. Acad. Sci. USA 90:1440 1444, Keller et al., 1993, Proc. Natl. Acad. Sci. USA 90:2160 2164; Heyman et al., 1992, Cell 68:397 406, and Levin et al., 1992, Nature 355:359 361). Since the discovery of PPARα, additional isoforms of PPAR have been identified, e.g., PPARβ, PPARγ and PPARδ, which have similar functions and are similarly regulated.

PPARs have been identified in the enhancers of a number of gene-encoding proteins that regulate lipid metabolism. These proteins include the three enzymes required for peroxisomal β-oxidation of fatty acids; apolipoprotein A-I; medium chain acyl-CoA dehydrogenase, a key enzyme in mitochondrial β-oxidation; and aP2, a lipid binding protein expressed exclusively in adipocytes (reviewed in Keller and Whali, 1993, TEM, 4:291 296; see also Staels and Auwerx, 1998, Atherosclerosis 137 Sulpl:S19 23). The nature of the PPAR target genes coupled with the activation of PPARs by fatty acids and hypolipidemic drugs suggests a physiological role for the PPARs in lipid homeostasis.

Pioglitazone, an antidiabetic compound of the thiazolidinedione class, was reported to stimulate expression of a chimeric gene containing the enhancer/promoter of the lipid binding protein aP2 upstream of the chloroamphenicol acetyl transferase reporter gene (Harris and Kletzien, 1994, Mol. Pharmacol. 45:439 445). Deletion analysis led to the identification of an approximately 30 bp region accounting for pioglitazone responsiveness. In an independent study, this 30 bp fragment was shown to contain a PPRE (Tontonoz et al., 1994, Nucleic Acids Res. 22:5628 5634). Taken together, these studies suggested the possibility that the thiazolidinediones modulate gene expression at the transcriptional level through interactions with a PPAR and reinforce the concept of the interrelatedness of glucose and lipid metabolism.

### 2.5 Current Cholesterol Management Therapies

In the past two decades or so, the segregation of cholesterolemic compounds into HDL and LDL regulators and recognition of the desirability of decreasing blood levels of the latter has led to the development of a number of drugs. However, many of these drugs have undesirable side effects and/or are contraindicated in certain patients, particularly when administered in combination with other drugs.

Bile-acid-binding resins are a class of drugs that interrupt the recycling of bile acids from the intestine to the liver. Examples of bile-acid-binding resins are cholestyramine (QUESTRAN LIGHT, Bristol-Myers Squibb), and colestipol hydrochloride (COLESTID, Pharmacia & Upjohn Company). When taken orally, these positively charged resins bind to negatively charged bile acids in the intestine. Because the resins cannot be absorbed from the intestine, they are excreted, carrying the bile acids with them. The use of such resins, however, at best only lowers serum cholesterol levels by about 20%. Moreover, their use is associated with gastrointestinal side-effects, including constipation and certain vitamin deficiencies. Moreover, since the resins bind to drugs, other oral medications must be taken at least one hour before or four to six hours subsequent to ingestion of the resin, complicating heart patients' drug regimens.

The statins are inhibitors of cholesterol synthesis. Sometimes, the statins are used in combination therapy with bile-acid-binding resins. Lovastatin (MEVACOR, Merck & Co., Inc.), a natural product derived from a strain of Aspergillus; pravastatin (PRAVACHOL, Bristol-Myers Squibb Co.); and atorvastatin (LIPITOR, Warner Lambert) block cholesterol synthesis by inhibiting HMGCoA reductase, the key enzyme involved in the cholesterol biosynthetic pathway. Lovastatin significantly reduces serum cholesterol and LDL-serum levels. However, serum HDL levels are only slightly increased following lovastatin administration. The mechanism of the LDL-lowering effect may involve both reduction of VLDL concentration and induction of cellular expression of LDL-receptor, leading to reduced production and/or increased catabolism of LDL. Side effects, including liver and kidney dysfunction are associated with the use of these drugs.

Nicotinic acid, also known as niacin, is a water-soluble vitamin B-complex used as a dietary supplement and antihyperlipidemic agent. Niacin diminishes the production of VLDL and is effective at lowering LDL. It is used in combination with bile-acid-binding resins. Niacin can increase HDL when administered at therapeutically effective doses; however, its usefulness is limited by serious side effects.

Fibrates are a class of lipid-lowering drugs used to treat various forms of hyperlipidemia, elevated serum triglycerides, which may also be associated with hypercholesterolemia. Fibrates appear to reduce the VLDL fraction and modestly increase HDL; however, the effects of these drugs on serum cholesterol is variable. In the United States, fibrates have been approved for use as antilipidemic drugs, but have not received approval as hypercholesterolemia agents. For example, clofibrate (ATROMID-S, Wyeth-Ayerst Laboratories) is an antilipidemic agent that acts to lower serum triglycerides by reducing the VLDL fraction. Although ATROMID-S may reduce serum cholesterol levels in certain patient subpopulations, the biochemical response to the drug is variable, and is not always possible to predict which patients will obtain favorable results. ATROMID-S has not been shown to be effective for prevention of coronary heart disease. The chemically and pharmacologically related drug, gemfibrozil (LOPID, Parke-Davis), is a lipid regulating agent which moderately decreases serum triglycerides and VLDL cholesterol. LOPID also increases HDL cholesterol, particularly the HDL2 and HDL3 subfractions, as well as both the AI/AII-HDL fractions. However, the lipid response to LOPID is heterogeneous, especially among different patient populations. Moreover, while prevention of coronary heart disease was observed in male patients between the ages of 40 and 55 without history or symptoms of existing coronary heart disease, it is not clear to what extent these findings can be extrapolated to other patient populations (e.g., women, older and younger males). Indeed, no efficacy was observed in patients with established coronary heart disease. Serious side-effects are associated with the use of fibrates, including toxicity; malignancy, particularly malignancy of gastrointestinal cancer; gallbladder disease; and an increased incidence in non-coronary mortality. These drugs are not indicated for the treatment of patients with high LDL or low HDL as their only lipid abnormality.

Oral estrogen replacement therapy may be considered for moderate hypercholesterolemia in post-menopausal women. However, increases in HDL may be accompanied with an increase in triglycerides. Estrogen treatment is, of course, limited to a specific patient population, postmenopausal women, and is associated with serious side effects, including induction of malignant neoplasms; gall bladder disease; thromboembolic disease; hepatic adenoma; elevated blood pressure; glucose intolerance; and hypercalcemia.

Long chain carboxylic acids, particularly long chain α,ω-dicarboxylic acids with distinctive substitution patterns, and their simple derivatives and salts, have been disclosed for treating atherosclerosis, obesity, and diabetes (See, e.g., Bisgaier et al., 1998, J. Lipid Res. 39:17-30, and references cited therein; International Patent Publication WO 98/30530; U.S. Pat. No. 4,689,344; International Patent Publication WO 99/00116; and U.S. Pat. No. 5,756,344). However, some of these compounds, for example the α,ω-dicarboxylic acids substituted at their α,α′-carbons (U.S. Pat. No. 3,773,946), while having serum triglyceride and serum cholesterol-lowering activities, have no value for treatment of obesity and hypercholesterolemia (U.S. Pat. No. 4,689,344).

U.S. Pat. No. 4,689,344 discloses β,β,β′,β′-tetrasubstituted-α,ω-alkanedioic acids that are optionally substituted at their α,α,α′,α′-positions, and alleges that they are useful for treating obesity, hyperlipidemia, and diabetes. According to this reference, both triglycerides and cholesterol are lowered significantly by compounds such as 3,3,14,14-tetramethylhexadecane-1,16-dioic acid. U.S. Pat. No. 4,689,344 further discloses that the β,β,β′,β′-tetramethyl-alkanediols of U.S. Pat. No. 3,930,024 also are not useful for treating hypercholesterolemia or obesity.

Other compounds are disclosed in U.S. Pat. No. 4,711,896. In U.S. Pat. No. 5,756,544, α,ω-dicarboxylic acid-terminated dialkane ethers are disclosed to have activity in lowering certain plasma lipids, including Lp(a), triglycerides, VLDL-cholesterol, and LDL-cholesterol, in animals, and elevating others, such as HDL-cholesterol. The compounds are also stated to increase insulin sensitivity. In U.S. Pat. No. 4,613,593, phosphates of dolichol, a polyprenol isolated from swine liver, are stated to be useful in regenerating liver tissue, and in treating hyperuricuria, hyperlipemia, diabetes, and hepatic diseases in general.

U.S. Pat. No. 4,287,200 discloses azolidinedione derivatives with anti-diabetic, hypolipidemic, and anti-hypertensive properties. However, the administration of these compounds to patients can produce side effects such as bone marrow depression, and both liver and cardiac cytotoxicity. Further, the compounds disclosed by U.S. Pat. No. 4,287,200 stimulate weight gain in obese patients.

It is clear that none of the commercially available cholesterol management drugs has a general utility in regulating lipid, lipoprotein, insulin and glucose levels in the blood. Thus, compounds that have one or more of these utilities are clearly needed. Further, there is a clear need to develop safer drugs that are efficacious at lowering serum cholesterol, increasing HDL serum levels, preventing coronary heart disease, and/or treating existing disease such as atherosclerosis, obesity, diabetes, and other diseases that are affected by lipid metabolism and/or lipid levels. There is also a clear need to develop drugs that may be used with other lipid-altering treatment regimens in a synergistic manner. There is still a further need to provide useful therapeutic agents whose solubility and Hydrophile/Lipophile Balance (HLB) can be readily varied.

The recitation of any reference in Section 2 of this application is not an admission that the reference is available as prior art to this application.

### 3. SUMMARY OF THE INVENTION

In one embodiment, the invention encompasses compounds of formula I: 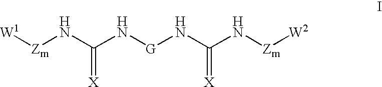
or a pharmaceutically acceptable salt, hydrate, solvate, or a mixture thereof wherein:
(a) X is O or S;
(b) each occurrence of Z is independently CH₂, CH═CH, or phenyl, wherein each occurrence of m is independently an integer ranging from 1 to 9, but when Z is phenyl then its associated m is 1;
(c) G is (CH₂)ᵣ, CH₂CH═CHCH₂, CH═CH, CH₂-phenyl-CH₂, or phenyl, wherein r is 2,3, or 4;
(d) W¹ and W² are independently L, V, C(R¹)(R²)—(CH₂)_{c}—C(R³)(R⁴)—(CH₂)ₙ—Y, or C(R¹)(R²)—(CH₂)_{c}—V, wherein c is 1 or 2 and n is an independent integer ranging from 0 to 4;
(e) each occurrence of R¹ and R² is independently (C₁-C₆)alkyl, (C₂-C₆)alkenyl, (C₂-C₆)alkynyl, phenyl, or benzyl or when W¹ or W² is C(R¹)(R²)—(CH₂)_{c}—C(R³)(R⁴)—Y, then R¹ and R² can both be H; or R¹ and R² and the carbon to which they are both attached are taken together to form a (C₃-C₇)cycloakyl group;
(f) R³ is H, (C₁-C₆)alkyl, (C₂-C₆)alkenyl, (C₂-C₆)alkynyl, (C₁-C₆)alkoxy, phenyl, benzyl, Cl, Br, CN, NO₂, or CF₃;
(g) R⁴ is OH, (C₁-C₆)alkyl, (C₂-C₆)alkenyl, (C₂-C₆)alkynyl, (C₁-C₆)alkoxy, phenyl, benzyl, Cl, Br, CN, NO₂, or CF₃;
(h) L is C(R¹)(R²)—(CH₂)ₙ—Y; where n is an independent integer ranging from 0 to 4;
(i) V is 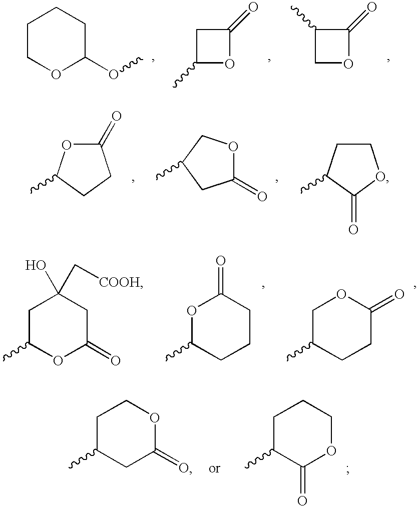
(j) each occurrence of Y is independently (C₁-C₆)alkyl, OH, COOH, CHO, COOR⁵, SO₃H, 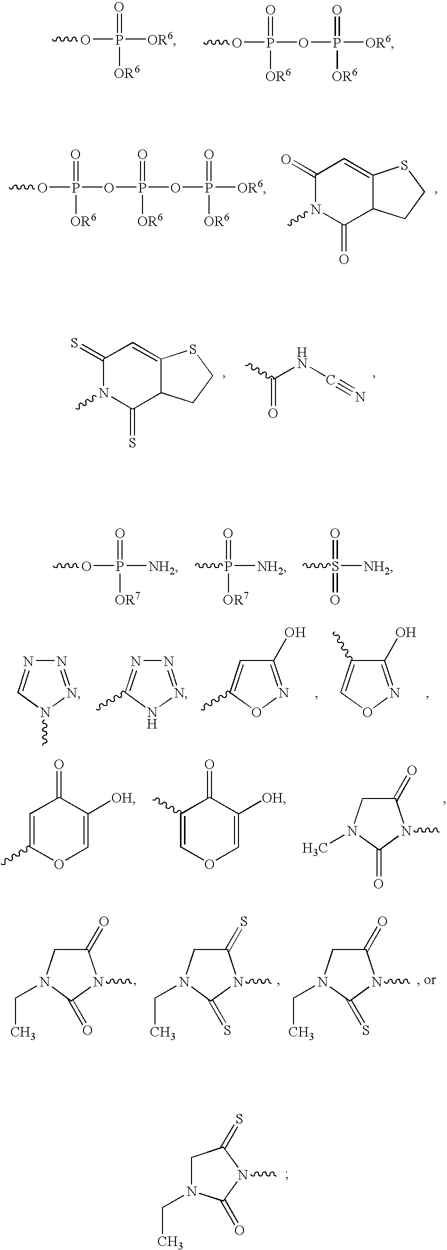
   where:
      (i) R⁵ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
      (ii) each occurrence of R⁶ is independently H, (C₁-C₆)alkyl, (C₂-C₆)alkenyl, or (C₂-C₆)alkynyl and is unsubstituted or substituted with one or two halo, OH, C₁-C₆ alkoxy, or phenyl groups; and
      (iii) each occurrence of R⁷ is independently H, (C₁-C₆)alkyl, (C₂-C₆)alkenyl, or (C₂-C₆)alkynyl;

Preferred compounds of formula I are those wherein:
(a) X is O;
(b) W¹ and W² are independently L, V, or C(R¹)(R²)—(CH₂)_{c}—V, where c is 1 or 2; and
(c) R¹ and R² are independently (C₁-C₆)alkyl, (C₂-C₆)alkenyl, (C₂-C₆)alkynyl, phenyl, or benzyl.

Other preferred compounds of formula I are those wherein W¹ is L.

Other preferred compounds of formula I are those wherein W¹ is V.

Other preferred compounds of formula I are those wherein W¹ is C(R¹)(R²)—(CH₂)_{c}—C(R³)(R⁴)—(CH₂)ₙ—Y.

Other preferred compounds of formula I are those wherein W¹ is C(R¹)(R²)—(CH₂)_{c}—V.

Other preferred compounds of formula I are those wherein W¹ and W² are independent L groups.

Other preferred compounds of formula I are those wherein each occurrence of Y is independently OH, COOR⁵, or COOH.

In another embodiment, the invention encompasses compounds of formula Ia: 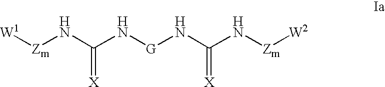
or a pharmaceutically acceptable salt, hydrate, solvate, or a mixture thereof wherein:
(a) X is O or S;
(b) each occurrence of Z is independently CH₂ or CH═CH, wherein each occurrence of m is independently an integer ranging from 1 to 9;
(b) G is (CH₂)ᵣ, CH₂CH═CHCH₂, or CH═CH, where r is 2, 3, or 4;
(c) W¹ and W² are independently L, V, or C(R¹)(R²)—(CH₂)_{c}—V, where c is 1 or 2;
(d) each occurrence of R¹ and R² is independently (C₁-C₆)alkyl, (C₂-C₆)alkenyl, (C₂-C₆)alkynyl, phenyl, or benzyl;
(e) L is C(R¹)(R²)—(CH₂)ₙ—Y, where n is an independent integer ranging from 0 to 4;
(f) V is 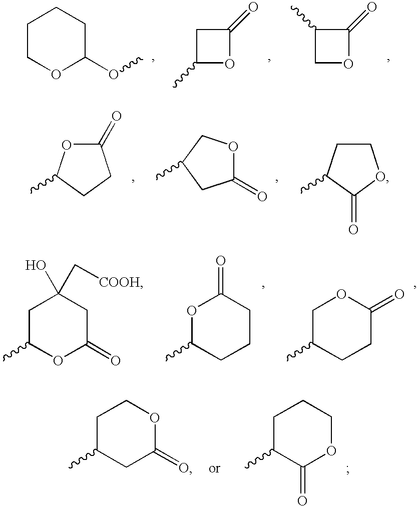
(g) each occurrence of Y is independently (C₁-C₆)alkyl, OH, COOH, CHO, COOR⁵, SO₃H, 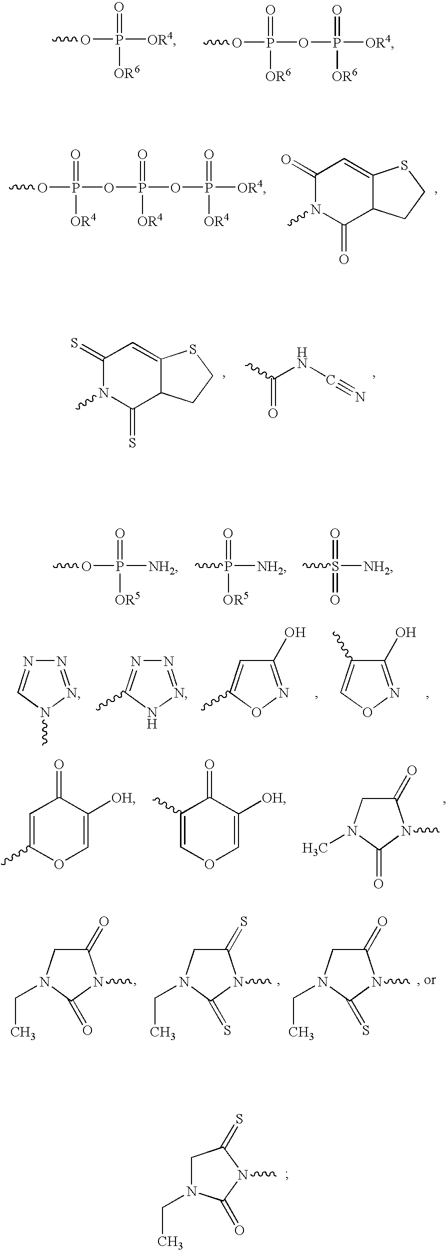
   where:
   (i) R³ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
   (ii) each occurrence of R⁴ is independently H, (C₁-C₆)alkyl, (C₂-C₆)alkenyl, or (C₂-C₆)alkynyl and is unsubstituted or substituted with one or two halo, OH, C₁-C₆alkoxy, or phenyl groups; and
   (iii) each occurrence of R⁵ is independently H, (C₁-C₆)alkyl, (C₂-C₆)alkenyl, or (C₂-C₆)alkynyl.

Preferably, in formula Ia each occurrence of Y is independently OH, COOR³, or COOH.

In yet another embodiment, the invention encompassses compounds of formula Ib: 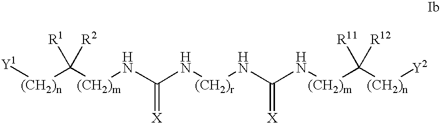
or a pharmaceutically acceptable salt, hydrate, solvate, or a mixture thereof wherein:
(a) X is O or S;
(b) each occurrence of m is independently an integer ranging from 1 to 9;
(c) r is 2, 3, or 4;
(d) each occurrence of n is an independent integer ranging from 0 to 4;
(e) each occurrence of R¹ and R² is independently (C₁-C₆)alkyl, (C₂-C₆)alkenyl, (C₂-C₆)alkynyl, phenyl, benzyl, or R¹, R¹, and the carbon to which they are both attached are taken together to form a (C₃-C₇)cycloalkyl group;
(f) each occurrence of R¹¹ and R¹² is independently H, (C₁-C₆)alkyl, (C₂-C₆)alkenyl, (C₂-C₆)alkynyl, phenyl, benzyl or R¹¹, R¹², and the carbon to which they are both attached are taken together to form a (C₃-C₇)cycloalkyl group; and
(g) each occurrence of Y is independently (C₁-C₆)alkyl, OH, COOH, CHO, COOR⁵, SO₃H, 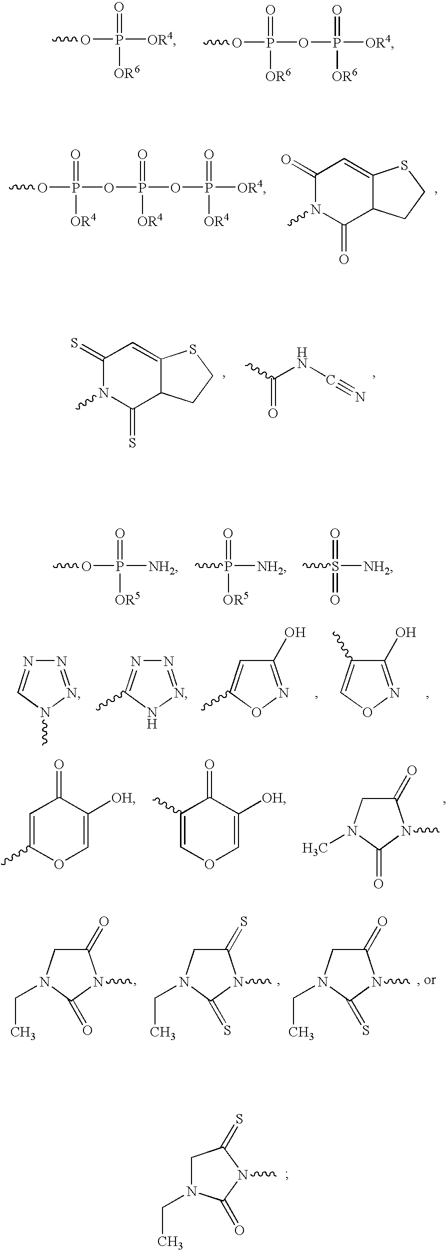
   where:
   (i) R³ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
   (ii) each occurrence of R⁴ is independently H, (C₁-C₆)alkyl, (C₂-C₆)alkenyl, or (C₂-C₆)alkynyl and is unsubstituted or substituted with one or two halo, OH, C₁-C₆ alkoxy, or phenyl groups; and
   (iii) each occurrence of R⁵ is independently H, (C₁-C₆)alkyl, (C₂-C₆)alkenyl, or (C₂-C₆)alkynyl.

Preferably in formula Ib, each occurrence of Y is independently OH, COOR³, or COOH.

In still another embodiment, the invention encompasses compounds of formula Ic: 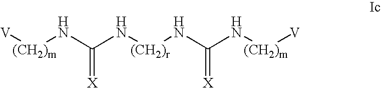
or a pharmaceutically acceptable salt, hydrate, solvate, or a mixture thereof wherein:
(a) X is O or S;
(b) each occurrence of m is an independent integer ranging from 1 to 9;
(c) r is 2, 3, or 4;
(d) V is

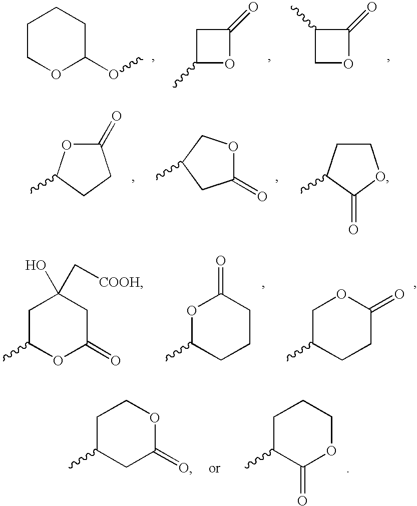

In another embodiment, the invention encompasses compounds of formula II: 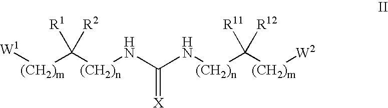
or a pharmaceutically acceptable salt, hydrate, solvate, or a mixture thereof wherein:
(a) X is O or S;
(b) R¹ and R² are independently (C₁-C₆)alkyl, (C₂-C₆)alkenyl, (C₂-C₆)alkynyl, phenyl, or benzyl; or R¹, R², and the carbon to which they are both attached are taken together to form a (C₃-C₇)cycloalkyl group;
(c) R¹¹ and R¹² are independently H, (C₁-C₆)alkyl, (C₂-C₆)alkenyl, (C₂-C₆)alkynyl, phenyl, benzyl; or R¹¹, R¹², and the carbon to which they are both attached are taken together to form a (C₃-C₇)cycloalkyl group;
(c) each occurrence of n is independently an integer ranging from 1 to 7;
(d) each occurrence of m is independently an integer ranging from 0 to 4;
(e) each occurrence of W¹ and W² is independently (C₁-C₆)alkyl, CH₂OH, COOH, CHO, OC(O)R³, C(O)OR³, SO₃H, 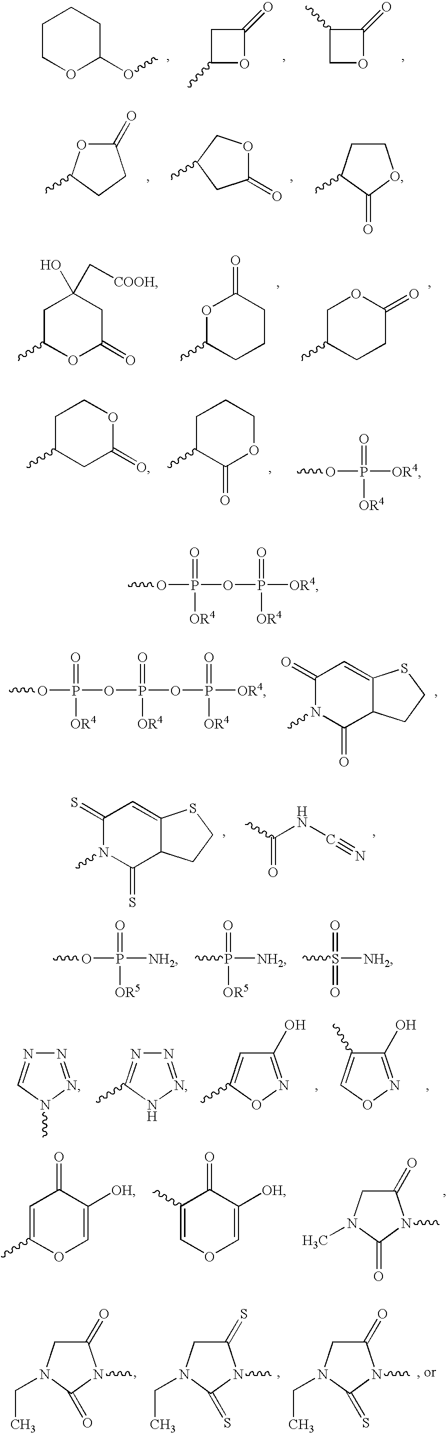 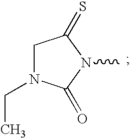
   where:
   (i) R³ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
   (ii) each occurrence of R⁴ is independently H, (C₁-C₆)alkyl, (C₂-C₆)alkenyl, or (C₂-C₆)alkynyl and is unsubstituted or substituted with one or two halo, OH, C₁-C₆ alkoxy, or phenyl groups;
   (iii) each occurrence of R⁵ is independently H, (C₁-C₆)alkyl, (C₂-C₆)alkenyl, or (C₂-C₆)alkynyl; and
   (iv) each occurrence of n is independently an integer ranging from 0 to 4.

Preferred compounds of formula II are those wherein each occurrence of W is independently OH, COOR³, COOH,

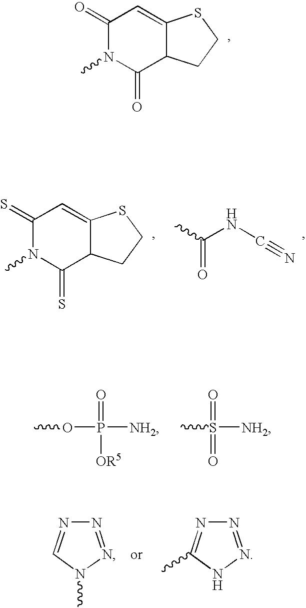

Other preferred compounds of formula II are those wherein R¹ and R² are independent (C₁-C₆)alkyl groups.

Other preferred compounds of formula II are those wherein m is 0.

Other preferred compounds of formula II are those wherein m is 1.

Other preferred compounds of formula II are those wherein R¹ and R² are each independently (C₁-C₆)alkyl.

Other preferred compounds of formula II are those wherein R¹ and R² are each methyl.

Other preferred compounds of formula II are those wherein W¹ and/or W² is COOH or CH₂OH.

In another embodiment, the invention relates to a compound of the formula IIa: 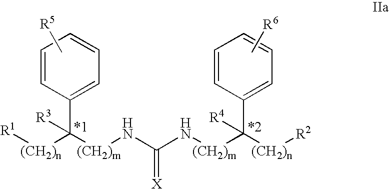
or a pharmaceutically acceptable salt, hydrate, solvate, or a mixture thereof wherein:
(a) X is O or S;
(b) R¹ and R² are (C₁-C₆)alkyl, CH₂OH, COOH, CHO, OC(O)R³, C(O)OR³, SO₃H, 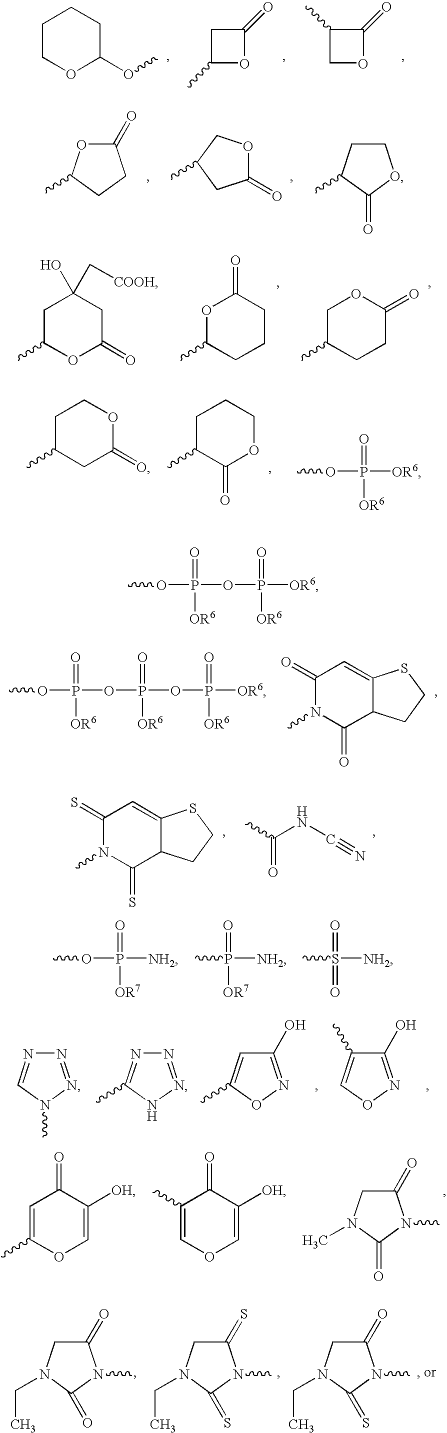 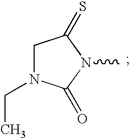
   where:
   (i) R⁷ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
   (ii) each occurrence of R⁸ is independently H, (C₁-C₆)alkyl, (C₂-C₆)alkenyl, or (C₂-C₆)alkynyl and is unsubstituted or substituted with one or two halo, OH, C₁-C₆ alkoxy, or phenyl groups, and
   (iii) each occurrence of R⁹ is independently H, (C₁-C₆)alkyl, (C₂-C₆)alkenyl, or (C₂—C₆)alkynyl;
(c) R³ and R⁴ are (C₁-C₆)alkyl, (C₂-C₆)alkenyl, (C₂-C₆)alkynyl, phenyl, or benzyl;
(d) R⁵ and R⁶ are hydrogen, halogen, (C₁-C₄)alkyl, (C₁-C₄)alkoxy, (C₆)aryloxy, CN, or NO₂, N(R⁵)₂ where R⁵ is H, (C₁-C₄)alkyl, phenyl, or benzyl;
(e) each occurrence of m is independently an integer ranging from 1 to 5;
(f) each occurrence of n is independently an integer ranging from 0 to 4; and
(g) C*¹ and C*² represent independent chiral-carbon centers, wherein each center may independently be R or S.

Preferred compounds of formula IIa are those wherein each occurrence of R¹ and R² is independently OH, COOR⁷, or COOH.

Other preferred compounds of formula IIa are those wherein m is 0.

Other preferred compounds of formula IIa are those wherein m is 1.

Other preferred compounds of formula IIa are those wherein R¹ and/or R² is COOH or CH₂OH.

Other preferred compounds of formula IIa are those wherein R³ and R⁴ are each independently (C₁-C₆)alkyl.

Other preferred compounds of formula IIa are those wherein R³ and R⁴ are each methyl.

Other preferred compounds of formula IIa are those wherein C*¹ is of the stereochemical configuration R or substantially R.

Other preferred compounds of formula IIa are those wherein C*¹ is of the stereochemical configuration S or substantially S.

Other preferred compounds of formula IIa are those wherein C*² is of the stereochemical configuration R or substantially R.

Other preferred compounds of formula IIa are those wherein C*² is of the stereochemical configuration S or substantially S.

In a particular embodiment, compounds of formula IIa are those wherein C*¹C*² are of the stereochemical configuration (S¹,S²) or substantially (S¹,S²).

In another particular embodiment, compounds of formula IIa are those wherein C*¹ C*² are of the stereochemical configuration (S¹,R²) or substantially (S¹,R²).

In another particular embodiment, compounds of formula IIa are those wherein C*¹ C*² are of the stereochemical configuration (R¹,R²) or substantially (R¹,R²).

In another particular embodiment, compounds of formula IIa are those wherein C*¹ C*² are of the stereochemical configuration (R¹,S²) or substantially (R¹,S²).

The compounds of the invention are useful in medical applications for treating or preventing aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. As used herein, the phrase “compounds of the invention” means, collectively, the compounds of formulas I, II and pharmaceutically acceptable salts, hydrates, solvates, clathrates, enantiomers, diastereomers, racemates, or mixures of stereoisomers thereof. Compounds of formula I encompass subgroup formulas Ia, Ib and Ic. Compounds of formula II encompass subgroup formula IIa. Thus, “compound of the invention” collectively means compound of formulas I, Ia, Ib, Ic, II and IIa are pharmaceutically acceptable salts, hydrates, solvates, clathrates, enantiomers, diastereomers, racemates, or mixures of stereoisomers thereof. The compounds of the invention are identified herein by their chemical structure and/or chemical name. Where a compound is referred to by both a chemical structure and a chemical name, and the chemical structure and chemical name conflict, the chemical structure is determinative of the compound's identity.

The present invention may be understood more fully by reference to the following detailed description and illustrative examples, which are intended to exemplify non-limiting embodiments of the invention.

### 4. DETAILED DESCRIPTION OF THE INVENTION

The present invention provides novel compounds useful for treating or preventing a disease or disorder such as, but not limited to, aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), and a thrombotic disorder, which method comprise administering a urea or thiourea compound or composition of the invention. The compounds of the invention can also treat or prevent inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism.

In this regard, the compounds of the invention are particularly useful when incorporated in a pharmaceutical composition having a carrier, excipient, diluent, or a mixture thereof. A composition of the invention need not contain additional ingredients, such as an excipient, other than a compound of the invention. Accordingly, in one embodiment, the compositions of the invention can omit pharmaceutically acceptable excipients and diluents and can be delivered in a gel cap or drug delivery device. Accordingly, the present invention provides methods for treating or preventing aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism, comprising administering to a patient in need thereof a therapeutically effective amount of a compound or composition of the invention.

In certain embodiments of the invention, a compound of the invention is administered in combination with another therapeutic agent. The other therapeutic agent provides additive or synergistic value relative to the administration of a compound of the invention alone. The therapeutic agent can be a lovastatin; a thiazolidinedione or fibrate; a bile-acid-binding-resin; a niacin; an anti-obesity drug; a hormone; a tyrophostine; a sulfonylurea-based drug; a biguanide; a phosphodiesterase-5-inhibitor; an α-glucosidase inhibitor; an apolipoprotein A-I agonist; apolipoprotein E; a cardiovascular drug; an HDL-raising drug; an HDL enhancer; or a regulator of the apolipoprotein A-I, apolipoprotein A-IV and/or apolipoprotein genes.

Some illustrative compounds of the invention are listed in Table 1 below:

TABLE 1

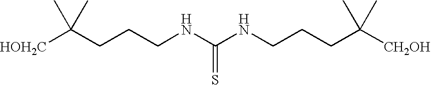

1) 1,3-Bis-(5-hydroxy-4,4-dimethylpentyl)thiourea

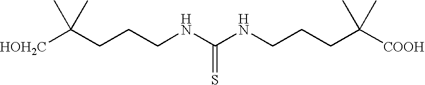

2) 5-[3-(5-Hydroxy-4,4-dimethylpentyl)thioureido]-2,2-dimethyl pentanoic acid

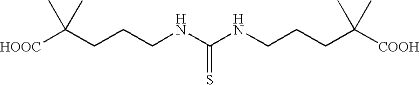

3) 5-[3-(4-Carboxy-4-methylpentyl)thioureido]-2,2-dimethyl pentanoic acid

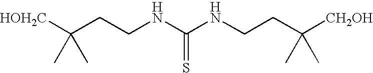

4) 1,3-Bis-(4-hydroxy-3,3-dimethylbutyl)thiourea

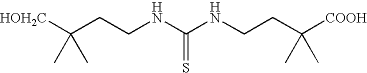

5) 4- [3-(4-Hydroxy-3,3-dimethylbutyl)thioureido]-2,2-dimethyl butyric acid

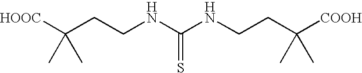

6) 4- [3-(3 -Carboxy-3-methylbutyl)thioureido]-2,2-dimethyl butyric acid

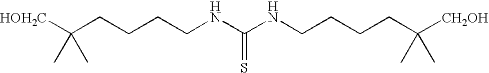

7) 1,3-Bis-(6-hydroxy-5,5-dimethylhexyl)thiourea

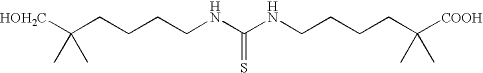

8) 6-[3-(6-Hydroxy-5,5-dimethylhexyl)thioureido]-2,2-dimethyl hexanoic acid

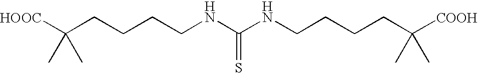

9) 6-[3-(5-Carboxy-5-methylhexyl)thioureido]-2,2-dimethyl hexanoic acid

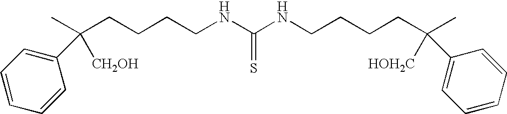

10) 1,3-Bis-(6-hydroxy-5-methyl-5-phenyl-hexyl)-thiourea

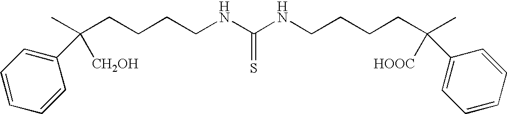

11) 6-[3-(6-Hydroxy-5-methyl-5-phenylhexyl)thioureido]-2-methyl-2-phenyl hexanoic

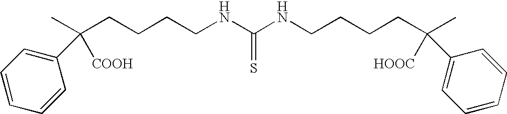

12) 6-[3-(5-Carboxy-5-phenylhexyl)thioureido]-2-methyl-2-phenyl hexanoic acid

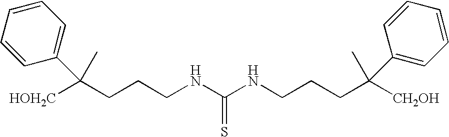

13) 1,3-Bis-(5-hydroxy-4-methyl-4-phenylpentyl)thiourea

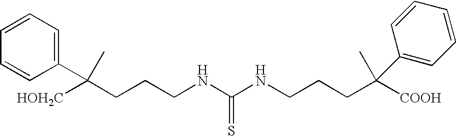

14) 5-[3-(5-Hydroxy-4-methyl-4-phenylpentyl)thioureido]-2-methyl-2-phenyl pentanoic

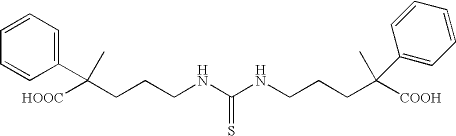

15) 5-[3-(4-Carboxy-4-phenylpentyl)thioureido]-2-methyl-2-phenyl pentanoic acid

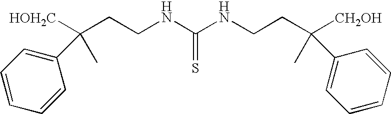

16) 1,3-Bis-(4-hydroxy-3-methyl-3-phenylbutyl)thiourea

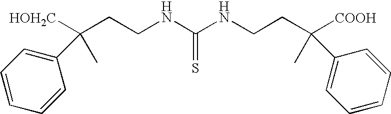

17) 4-[3-(4-Hydroxy-3-methyl-3-phenylbutyl)thioureido]-2-methyl-2-phenyl butyric acid

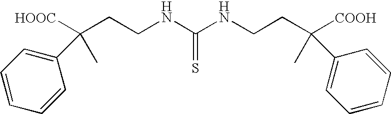

18) 4- [3-(3-Carboxy-3-phenylbutyl)thioureido]-2-methyl-2-phenyl butyric acid

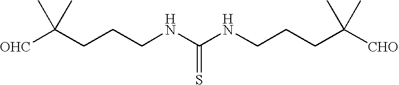

19) 1,3-Bis-(4,4-dimethyl-5-oxo-pentyl)thiourea

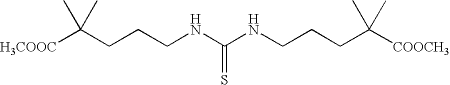

20) 5-[3-(4-Methoxycarbonyl-4-methylpentyl)thioureido]-2,2-dimethyl pentanoic acid
methyl ester

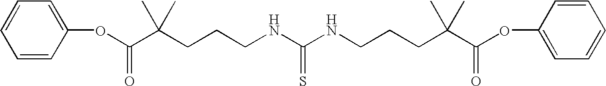

21) 2,2-Dimethyl-5-[3-(4-methyl-4-phenoxycarbonylpentyl)thioureido]pentanoic acid
phenyl ester

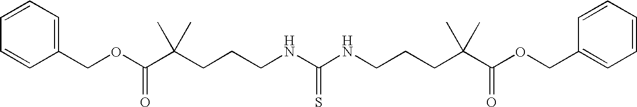

22) 5-[3-(4-Benzyloxycarbonyl-4-methylpentyl)thioureido]-2,2-dimethyl pentanoic acid
benzyl ester

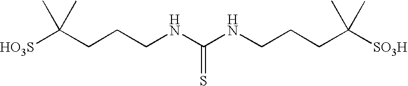

23) 2-Methyl-5-[3-(4-methyl-4-sulfopentyl)thioureido]pentane-2-sulfonic acid

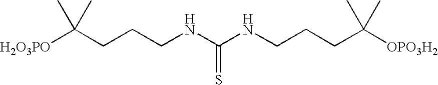

24) Phosphoric acid mono-{1,1-dimethyl-4-[3-(4-methyl-4-
phosphonooxypentyl)thioureido]butyl}ester

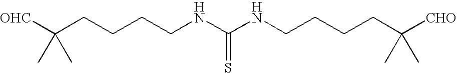

25) 1,3-Bis-(5,5-dimethyl-6-oxohexyl)thiourea

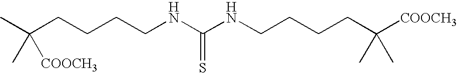

26) 6-[3-(5-Methoxycarbonyl-5-methylhexyl)thioureido]-2,2-dimethyl hexanoic acid
methyl ester

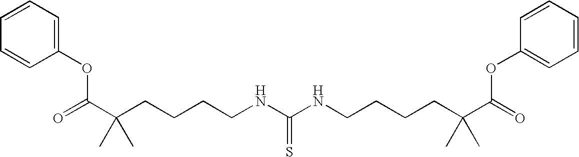

27) 2,2-Dimethyl-6-[3-(5-methyl-5-phenoxycarbonylhexyl)thioureidol hexanoic acid
phenyl ester

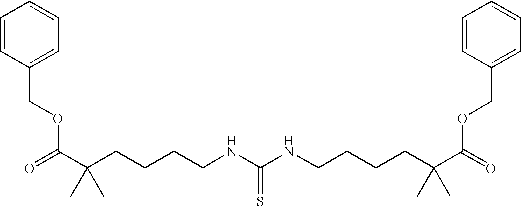

28) 6-[3-(5-Benzyloxycarbonyl-5-methylhexyl)thioureido]-2,2-dimethyl hexanoic acid
benzyl ester

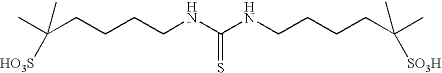

29) 2-Methyl-6-[3-(5-methyl-5-sulfohexyl)thioureido]hexane-2-sulfonic acid

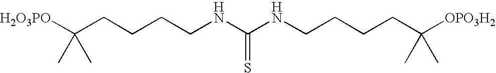

30) Phosphoric acid mono-(1,1 -dimethyl-5-[3-(5-methyl-5-
phosphonooxyhexyl)thioureido]pentyl}ester

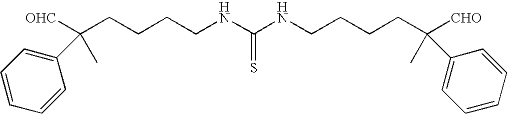

31) 1-(6-Hydroxy-5-methyl-5-phenylhexyl)-3-(5-methyl-6-oxo-5-phenylhexyl)thiourea

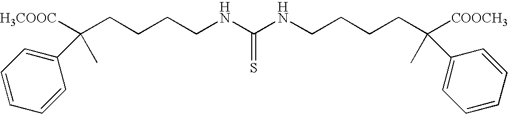

32) 2-Methyl-6-[3-(5-methyl-6-methylperoxy-5-phenylhexyl)thioureido]-2-
phenylhexanoic acid methyl ester

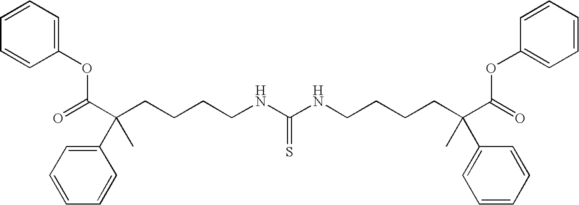

33) 2-Methyl-6-[3-(5-phenoxycarbonyl-5-phenylhexyl)thioureido]-2-phenyl hexanoic acid
phenyl ester

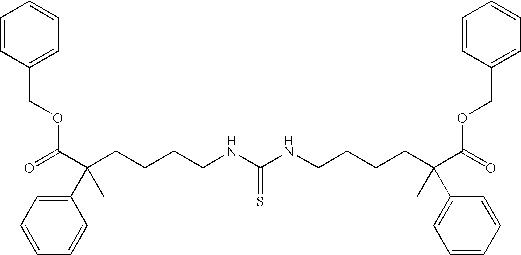

34) 6-[3-(5-Benzyloxycarbonyl-5-phenylhexyl)thioureido]-2-methyl-2-phenyl hexanoic
acid benzyl ester

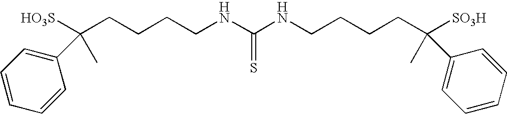

35) 6-[3-(5-Methyl-5-phenyl-hexyl)-thioureido]-2-phenyl-hexane-2-sulfonic acid

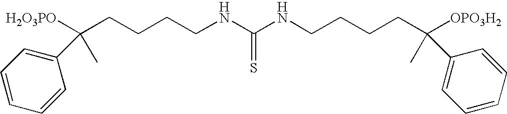

36) Phosphoric acid mono-{1-methyl-1-phenyl-5-]3-(5-phenyl-5-
phosphonooxyhexyl)thioureido]pentyl}ester

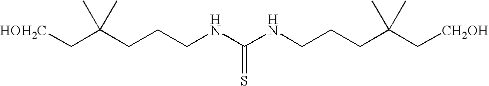

37) 1,3-Bis-(6-hydroxy-4,4-dimethylhexyl)thiourea

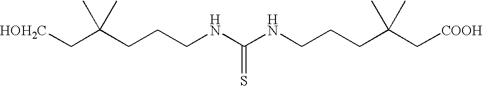

38) 6-[3-(6-Hydroxy-4,4-dimethylhexyl)thioureido]-3,3-dimethyl hexanoic acid

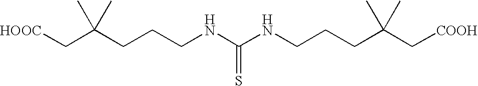

39) 6-[3-(6-Hydroperoxy-4,4-dimethylhexyl)thioureido]-3,3-dimethyl hexanoic acid

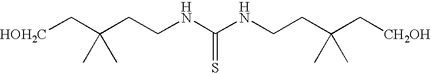

40) 1,3-Bis-(5-hydroxy-3,3-dimethylpentyl)thiourea

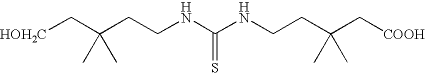

41) 5-[3-(5-Hydroxy-3,3-dimethylpentyl)thioureido]-3,3-dimethyl pentanoic acid

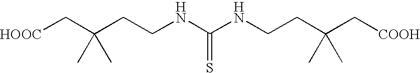

42) 5-[3-(4-Carboxy-3,3-dimethylbutyl)thioureido]-3,3-dimethyl pentanoic acid

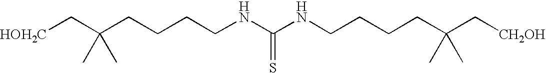

43) 1,3-Bis-(7-hydroxy-5,5-dimethylheptyl)thiourea

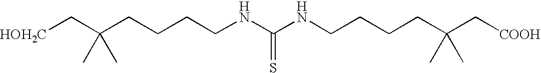

44) 7-[3-(7-Hydroxy-5,5-dimethylheptyl)thioureido]-3,3-dimethyl heptanoic acid

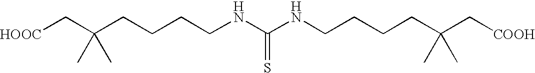

45) 7-[3-(6-Carboxy-5,5-dimethylhexyl)thioureido]-3,3-dimethyl heptanoic acid

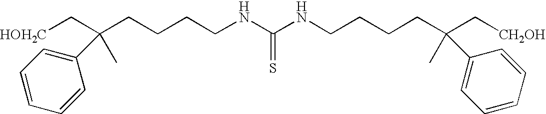

46) 1,3-Bis-(7-hydroxy-5-methyl-5-phenylheptyl)thiourea

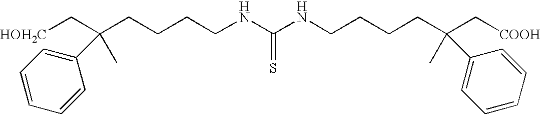

47) 7-[3-(7-Hydroxy-5-methyl-5-phenylheptyl)thioureido]-3-methyl-3-phenyl heptanoic

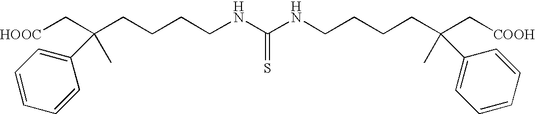

48) 7-[3-(6-Carboxy-5-methyl-5-phenylhexyl)thioureido]-3-methyl-3-phenyl heptanoic
acid

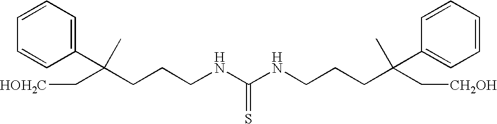

49) 1,3-Bis-(6-hydroxy-4-methyl-4-phenylhexyl)thiourea

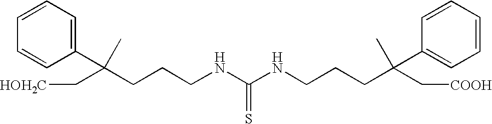

50) 6-[3-(6-Hydroxy-4-methyl-4-phenylhexyl)thioureido]-3-methyl-3-phenyl hexanoic
acid

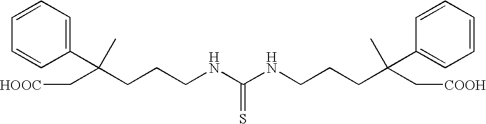

51) 6-[3-(5-Carboxy-4-methyl-4-phenylpentyl)thioureido]-3-methyl-3-phenyl hexanoic
acid

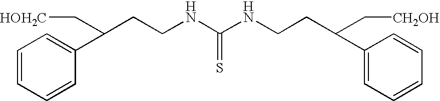

52) 1,3-Bis-(5-hydroxy-3-methyl-3-phenylpentyl)thiourea

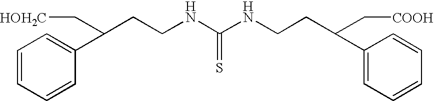

53) 5-[3-(5-Hydroxy-3-methyl-3-phenylpentyl)thioureido]-3-methyl-3-phenylpentanoic
acid

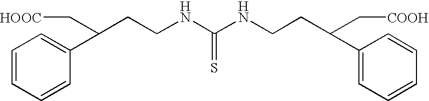

54) 5-[3-(4-Carboxy-3-methyl-3-phenylbutyl)thioureido]-3-methyl-3-phenyl pentanoic
acid

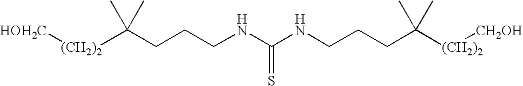

55) 1,3-Bis-(7-hydroxy-4,4-dimethylheptyl)thiourea

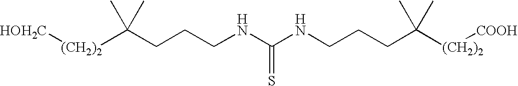

56) 7-[3-(7-Hydroxy-4,4-dimethylheptyl)thioureido]-4,4-dimethyl heptanoic acid

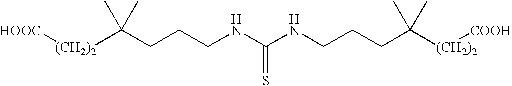

57) 7-[3-(6-Carboxy-4,4-dimethylhexyl)thioureido]-4,4-dimethyl heptanoic acid

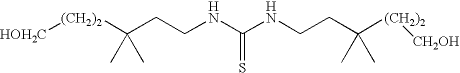

58) 1,3-Bis-(6-hydroxy-3,3-dimethylhexyl)thiourea

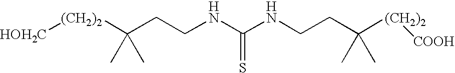

59) 6-[3-(6-Hydroxy-3,3-dimethylhexyl)thioureido]-4,4-dimethyl hexanoic acid

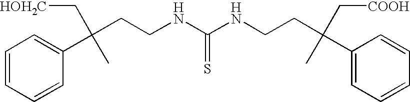

60) 5-[3-(5-Hydroxy-3-methyl-3-phenylpentyl)thioureido]-3-methyl-3-phenyl pentanoic
acid

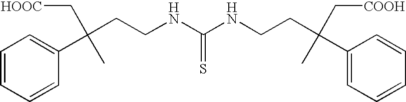

61) 5-[3-(5-Hydroperoxy-3-methyl-3-phenylpentyl)thioureido]-3-methyl-3-phenyl
pentanoic acid

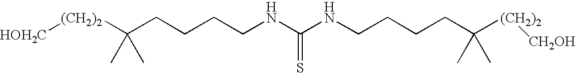

62) 1,3-Bis-(8-hydroxy-5,5-dimethyloctyl)thiourea

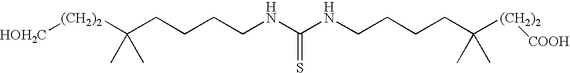

63) 2-{5-[3-(8-Hydroxy-5,5-dimethyloctyl)thioureido]-1,1-dimethylpentyl}acrylic acid

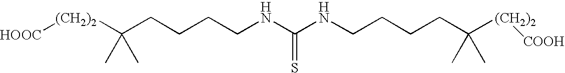

64) 2-{5-[3-(8-Hydroperoxy-5,5-dimethyloctyl)thioureido]-1,1-dimethylpentyl}acrylic
acid

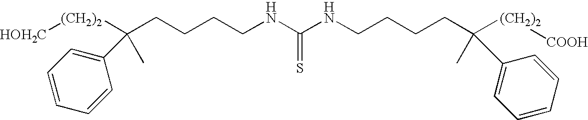

65) 1-(6-Hydroxymethyl-5-methyl-5-phenyl-hept-6-enyl)-3-(8-hydroxy-5-methyl-5-
phenyloctyl)thiourea

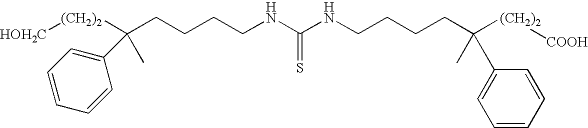

66) 8-[3-(8-Hydroxy-5-methyl-5-phenyloctyl)thioureido]-4-methyl-4-phenyl octanoic acid

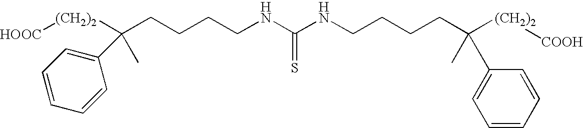

67) 2-{5-[3-(8-Hydroperoxy-5-methyl-5-phenyloctyl)thioureido]-1-methyl-1-
phenylpentyl}acrylic acid

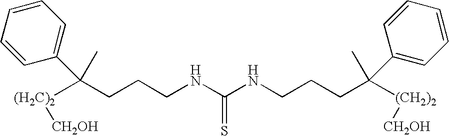

68) 1,3-Bis-(7-hydroxy-4-methyl-4-phenylheptyl)thiourea

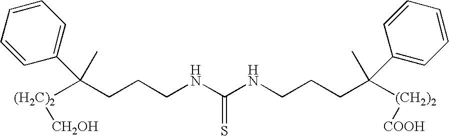

69) 7-[3-(7-Hydroxy-4-methyl-4-phenylheptyl)thioureido]-4-methyl-4-phenyl heptanoic
acid

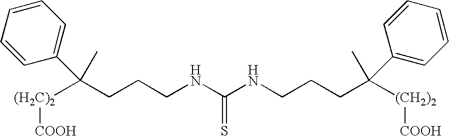

70) 7-[3-(6-Carboxy-4-methyl-4-phenylhexyl)thioureido]-4-methyl-4-phenyl heptanoic
acid

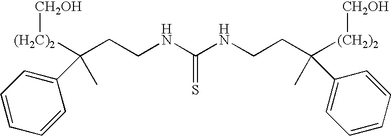

71) 1,3-Bis-(6-hydroxy-3-methyl-3-phenylhexyl)thiourea

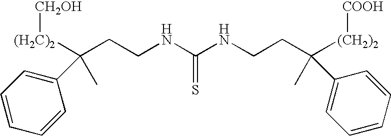

72) 6-[3-(6-Hydroxy-3-methyl-3-phenylhexyl)thioureido]-4-methyl-4-phenyl hexanoic
acid

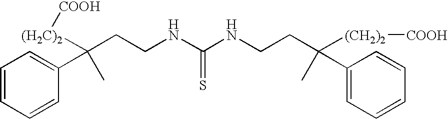

73) 6-[3-(5-Carboxy-3-methyl-3-phenylpentyl)thioureido]-4-methyl-4-phenyl hexanoic
acid

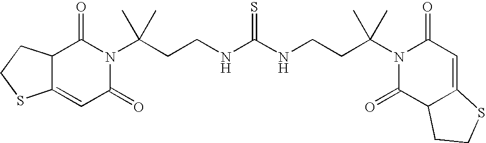

74) 1,3-Bis-[3-(4,6-dioxo-2,3,3a,6-tetrahydro-4H-thieno[3,2-c]pyridin-5-yl)-3-
methylbutyl]thiourea

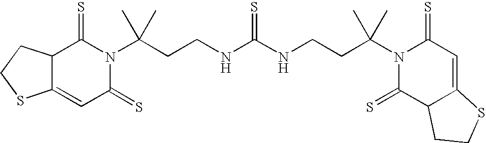

75) 1,3-Bis-[3-(4,6-dithioxo-2,3,3a,6-tetrahydro-4H-thieno[3,2-c]pyridin-5-yl)-3-
methylbutyl]thiourea

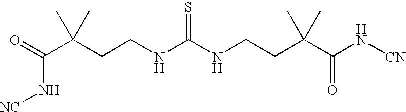

76) N,N′-Bis-[4-(N″-cyano-2,2-dimethylbutamide)]thiourea

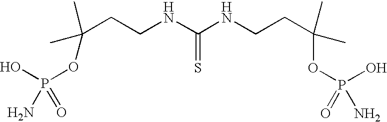

77) Phosphoramidic acid mono-(3-{3-[3-(amino-hydroxy-phosphoryloxy)-3-
methylbutyl]thioureido}-1,1-dimethyipropyl) ester

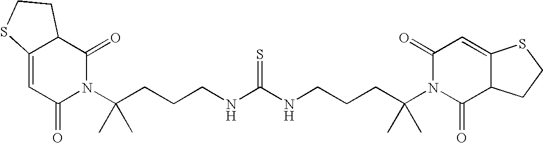

78) 1,3-Bis-[4-(4,6-dioxo-2,3,3a,6-tetrahydro-4H-thieno[3,2-c]pyridin-5-yl)-4-
methylpentyl]thiourea

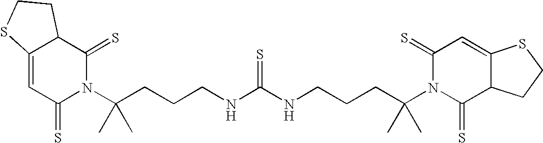

79) 1,3-Bis-[4-(4,6-dithioxo-2,3,3a,6-tetrahydro-4H-thieno[3,2-c]pyridin-5-yl)-4-
methylpentyl]-thiourea

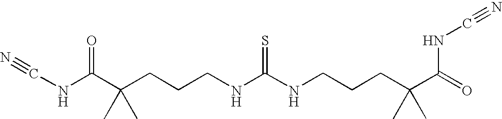

80) N,N′-Bis-[5-(N″-cyano-2,2-dimethylpentamide)]thiourea

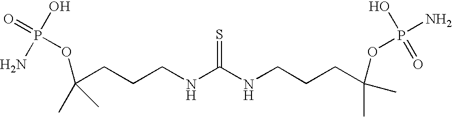

81) Phosphoramidic acid mono-(4-{3-[4-(amino-hydroxy-phosphoryloxy)-4-
methylpentyl]thioureido}-1,1-dimethylbutyl) ester

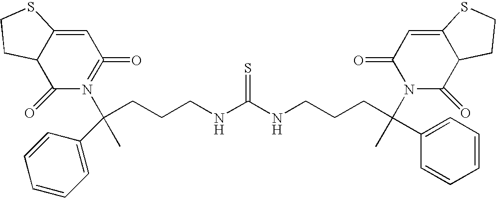

82) 1,3-Bis-[4-(4,6-dioxo-2,3,3a,6-tetrahydro-4H-thieno[3,2-c]pyridin-5-yl)-4-
phenylpentyl]thiourea

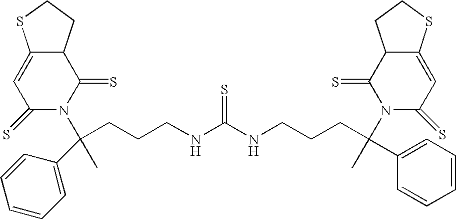

83) 1,3-Bis-[4-(4,6-dithioxo-2,3,3a,6-tetrahydro-4H-thieno[3,2-c]pyridin-5-yl)-4-
phenylpentyl]thiourea

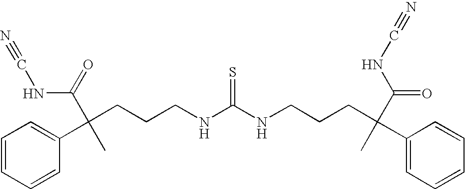

84) N,N′-Bis-[5-(N″-cyano-2-methyl-2-phenylpentamide)]thiourea

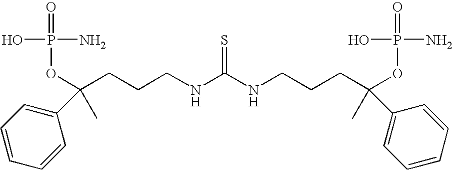

85) Phosphoramidic acid mono-(4-{3-[4-(amino-hydroxy-phosphoryloxy)-4-
phenylpentyl]thioureido}-1-methyl-1-phenylbutyl) ester

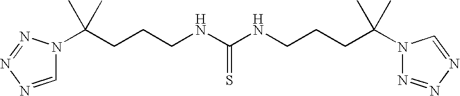

86) 1,3-Bis-(4-methyl-4-tetrazol-1-yl-pentyl)thiourea

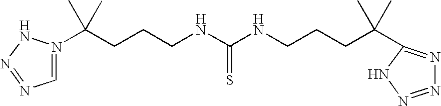

87) 1,3-Bis-[4-methyl-4-(1H-tetrazol-5-yl)pentyl]thiourea

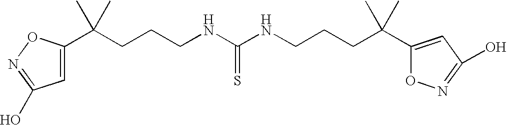

88) 1,3-Bis-[4-(3-hydroxy-isoxazol-5-yl)-4-methylpentyl]thiourea

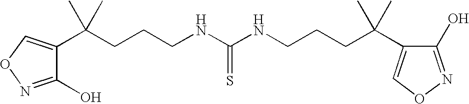

89) 1,3-Bis-[4-(3-hydroxy-isoxazol-4-yl)-4-methylpentyl]thiourea

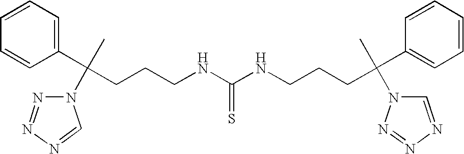

90) 1,3-Bis-[4-phenyl-4-(tetrazol-1-yl)penty]thiourea

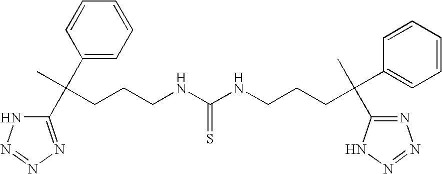

91) 1,3-Bis-[4-phenyl-4-(1H-tetrazol-5-yl)pentyl]thiourea

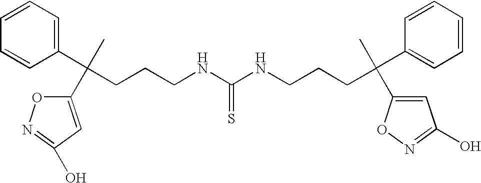

92) 1,3-Bis-[4-(3-hydroxy-isoxazol-5-yl)-4-phenylpentyl]thiourea

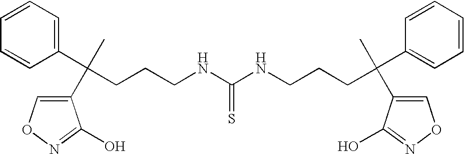

93) 1,3-Bis-[4-(3-hydroxy-isoxazol-4-yl)-4-phenylpentyl]thiourea

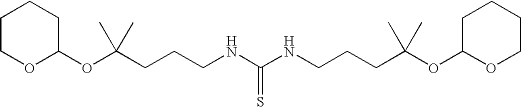

94) 1,3-Bis-[4-methyl-4-(tetrahydro-pyran-2-yloxy)pentyl]thiourea

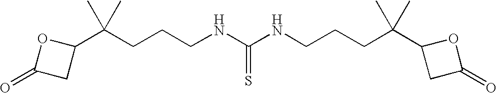

95) 1,3-Bis-[4-methyl-4-(4-oxo-oxetan-2-yl)pentyl]thiourea

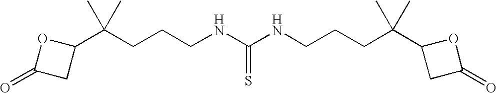

96) 1,3-Bis-[4-methyl-4-(4-oxo-oxetan-2-yl)pentyl]thiourea

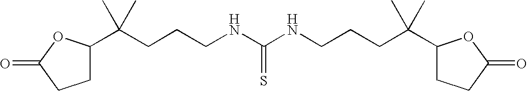

97) 1,3-Bis-[4-methyl-4-(5-oxo-tetrahydro-furan-2-yl)pentyl]thiourea

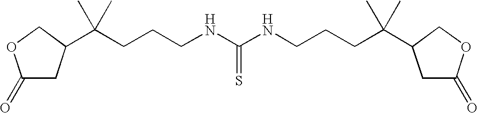

98) 1,3-Bis-[4-methyl-4-(5-oxo-tetrahydro-furan-3-yl)pentyl]thiourea

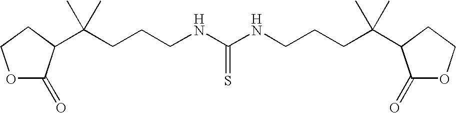

99) 1,3-Bis-[4-methyl-4-(2-oxo-tetrahydro-furan-3-yl)pentyl]thiourea

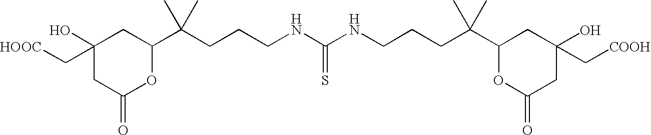

100) [2-(4-{3-[4-(4-Carboxymethyl-4-hydroxy-6-oxo-tetrahydro-pyran-2-yl)-4-
methylpentyl]thioureido}-1,1-dimethylbutyl)-4-hydroxy-6-oxo-tetrahydro-pyran-4-yl]
acetic acid

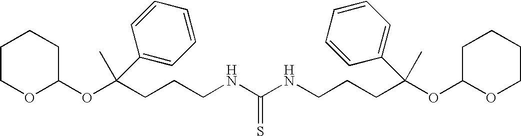

101) 1,3-Bis-[4-phenyl-4-(tetrahydro-pyran-2-yloxy)pentyl]thiourea

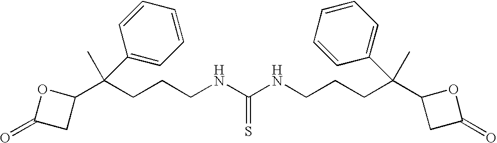

102) 1,3-Bis-[4-(4-oxo-oxetan-2-yl)-4-phenylpentyl]thiourea

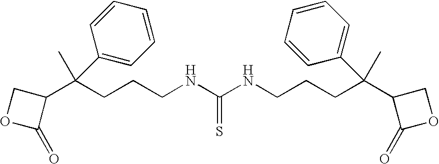

103) 1,3-Bis-[4-(2-oxo-oxetan-3-yl)-4-phenylpentyl]thiourea

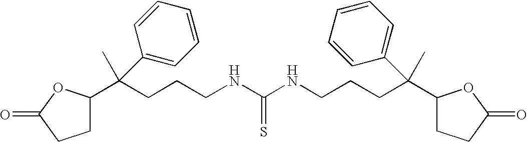

104) 1,3-Bis-[4-(5-oxo-tetrahydro-furan-2-yl)-4-phenylpentyl]thiourea

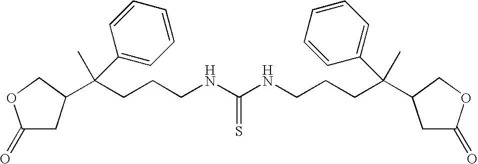

105) 1,3-Bis-[4-(5-oxo-tetrahydro-furan-3-yl)-4-phenylpentyl]thiourea

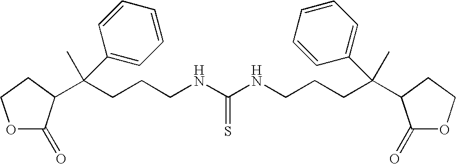

106) 1,3-Bis-[4-(2-oxo-tetrahydro-furan-3-yl)-4-phenylpentyl]thiourea

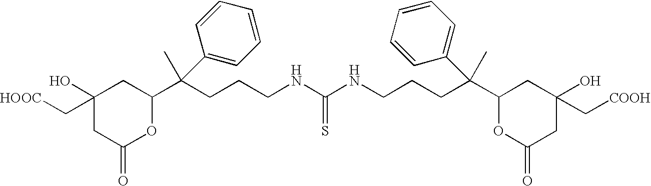

107) [2-(4-{3-[4-(4-Carboxymethyl-4-hydroxy-6-oxo-tetrahydro-pyran-2-yl)-4-
phenylpentyl]thioureido}-1-methyl-1-phenylbutyl)-4-hydroxy-6-oxo-tetrahydro-pyran-4-
yl] acetic acid

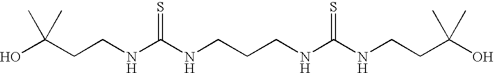

108) 1-(3-Hydroxy-3-methylbutyl)-3-{3-[3-(3-hydroxy-3-
methylbutyl)thioureido]propyl}thiourea

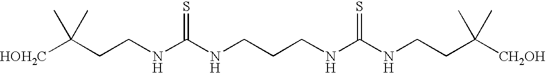

109) 1-(4-Hydroxy-3,3-dimethylbutyl)-3-{3-[3-(4-hydroxy-3,3-
dimethylbutyl)thioureido]propyl}thiourea

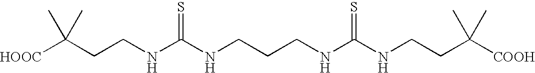

110) 4-(3-{3-[3-(3-Carboxy-3-methylbutyl)thioureido]propyl}thioureido)-2,2-dimethyl
butyric acid

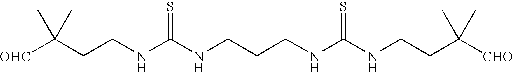

111) 1-(3,3-Dimethyl-4-oxobutyl)-3-{3-[3-(3,3-dimethyl-4-
oxobutyl)thioureido]propyl}thiourea

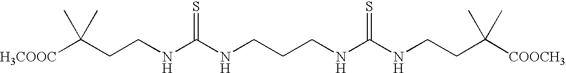

112) 4-(3-{3-[3-(3-Methoxycarbonyl-3-methylbutyl)thioureido]propyl}thioureido)-2,2-
dimethyl butyric acid methyl ester

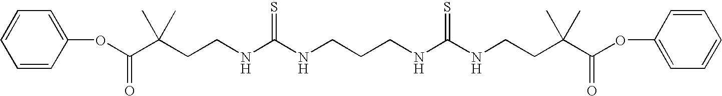

113) 2,2-Dimethyl-4-(3-{3-[3-(3-methyl-3-
phenoxycarbonylbutyl)thioureido]propyl}thioureido) butyric acid phenyl ester

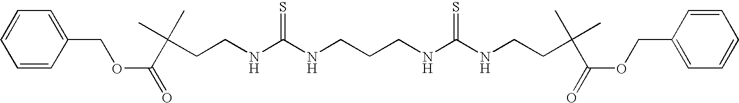

114) 4-(3-{3-[3-(3-Benzyloxycarbonyl-3-methylbutyl)thioureido]propyl}thioureido)-2,2-
dimethyl butyric acid benzyl ester

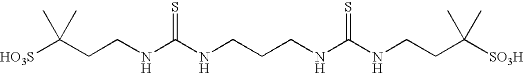

115) 2-Methyl-4-(3-{3-[3-(3-methyl-3-sulfobutyl)thioureido]propyl}thioureido)butane-2-
sulfonic acid

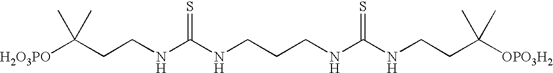

116) Phosphoric acid mono-[1,1-dimethyl-3-(3-{3-[3-(3-methyl-3-
phosphonooxybutyl)thioureido]propyl}thioureido)propyl] ester

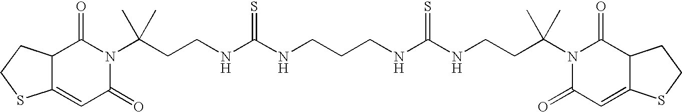

117) 1-[3 -(4,6-Dioxo-2,3,3a,6-tetrahydro-4H-thieno[3,2-c]pyridin-5-yl)-3 -methylbutyl]
3-(3-{3-[3-(4,6-dioxo-2,3,3a,6-tetrahydro-4H-thieno[3,2-c]pyridin-5-yl)-3-
methylbutyl]thioureido}propyl)thiourea

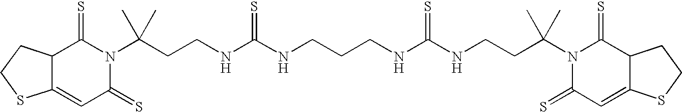

118) 1-[3-(4,6-Dithioxo-2,3,3a,6-tetrahydro-4H-thieno[3,2-c]pyridin-5-yl)-3-
methylbutyl]-3-(3-{3-[3-(4,6-dithioxo-2,3,3a,6-tetrahydro-4H-thieno[3,2-c]pyridin-5-yl)-
3-methylbutyl]thioureido}propyl)thiourea

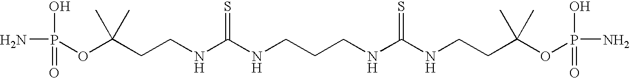

119) Phosphoramidic acid mono-[1,1-dimethyl-3-(3-{3-[3-(3-methyl-3-
phosphonooxybutyl)thioureido]propyl}thioureido)propyl] ester

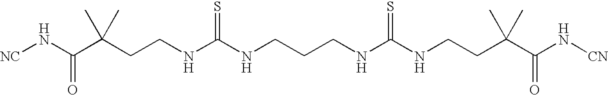

120) N-cyano-4-[3-(3-{3-(N-cyano-carbamoyl)-3-
methylbutyl]thioureido}propylthioureido]-2,2-dimethylbutyramide

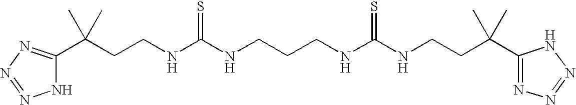

121) 1-[3-Methyl-3-(1H-tetrazol-5-yl)-butyl]-3-(3-{3-[3-methyl-3-(1H-tetrazol-5-
yl)butyl]
thioureido}propyl)thiourea

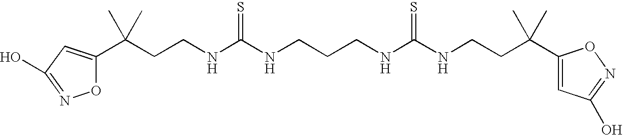

122) 1-[3-(3-Hydroxy-isoxazol-5-yl)-3-methylbutyl]-3-(3-{3-[3-(3-hydroxy-isoxazol-5-
yl)-3-methylbutyl]thioureido}propyl)thiourea

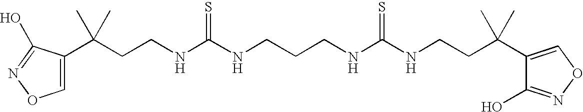

123) 1-[3-(3-Hydroxy-isoxazol-4-yl)-3-methylbutyl]-3-(3-{3-[3-(3-hydroxy-isoxazol-4-
yl)-3-methylbutyl]thioureido}propyl)thiourea

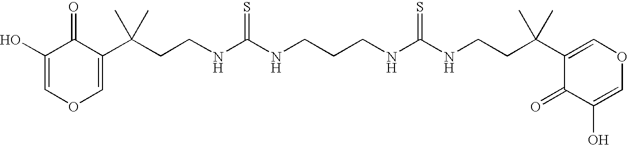

124) 1-[3-(5-Hydroxy-4-oxo-4H-pyran-3-yl)-3-methylbutyl]-3-(3-{3-[3-(5-hydroxy-4-
oxo-4H-pyran-3-yl)-
3-methylbutyl]thioureido}propyl)thiourea

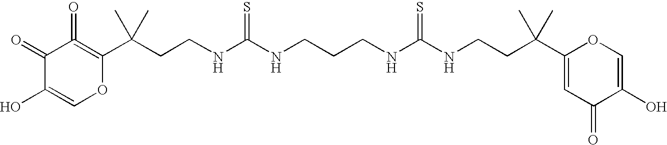

125) 1-[3-(5-Hydroxy-4-oxo-4H-pyran-2-yl)-3-methylbutyl]-3-(3-{3-[3-(5-hydroxy-4-
oxo-4H-pyran-2-yl)-3-methylbutyl]thioureido}propyl)thiourea

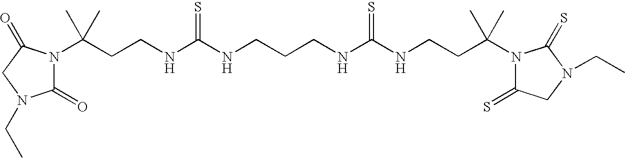

126) 1-(3-{3-[3-(3-Ethyl-2,5-dioxo-imidazolidin-1-yl)-3-
methylbutyl]thioureido}propyl)-3-[3-(3-ethyl-2,5-dithioxo-imidazolidin-1-yl)-3-
methylbutyl]thiourea

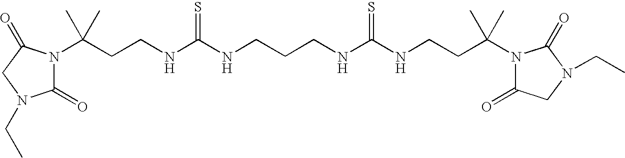

127) 1-[3-(3-Ethyl-2,5-dioxo-imidazolidin-1-yl)-3-methylbutyl]-3-(3-{3-[3-(3-ethyl-2,5-
dioxo-imidazolidin-1-yl)-3-methylbutyl]thioureido}propyl)thiourea

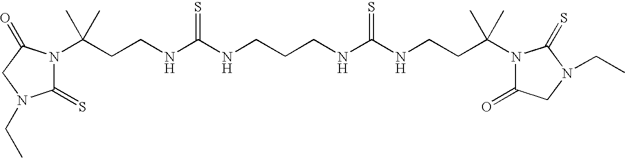

128) 1-[3-(3-Ethyl-5-oxo-2-thioxo-imidazolidin-1-yl)-3-methylbutyl]-3-(3-{3-[3-(3-
ethyl-5-oxo-2-thioxo-imidazolidin-1-yl)-3-methylbutyl]thioureido}propyl)thiourea

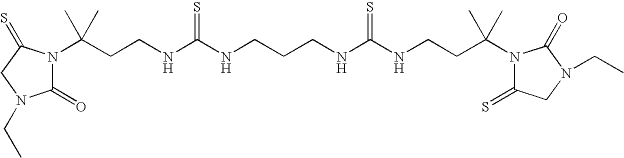

129) 1-[3-(3-Ethyl-2-oxo-5-thioxo-imidazolidin-1-yl)-3-methylbutyl]-3-(3-{3-[3-(3-
ethyl-2-oxo-5-thioxo-imidazolidin-1-yl)-3-methylbutyl]thioureido}propyl)thiourea

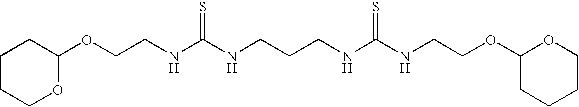

130) 1-[2-(Tetrahydro-pyran-2-yloxy)-ethyl]-3-(3-{3-[2-(tetrahydro-pyran-2-yloxy)ethyl]
thioureido}propyl)thiourea

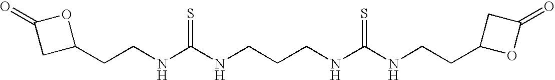

131) 1-[2-(4-Oxo-oxetan-2-yl)ethyl]-3-(3-{3-[2-(4-oxo-oxetan-2-
yl)ethyl]thioureido}propyl)thiourea

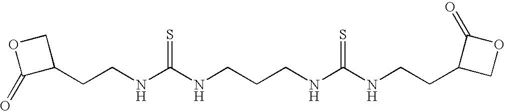

132) 1-[2-(2-Oxo-oxetan-3-yl)ethyl]-3-(3-{3-[2-(2-oxo-oxetan-3-yl)ethyl]thioureido
propyl)thiourea

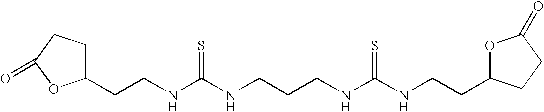

133) 1-[2-(5-Oxo-tetrahydro-furan-2-yl)ethyl]-3-(3-{3-[2-(5-oxo-tetrahydro-furan-2-yl)
ethyl]thioureido}propyl)thiourea

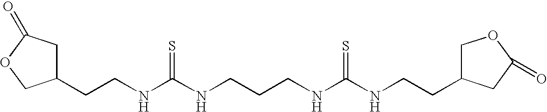

134) 1-[2-(5-Oxo-tetrahydro-furan-3-yl)ethyl]-3-(3-{3-[2-(5-oxo-tetrahydro-furan-3-yl)
ethyl]thioureido}propyl)thiourea

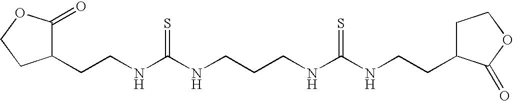

135) 1-[2-(2-Oxo-tetrahydro-furan-3-yl)ethyl]-3-(3-{3-[2-(2-oxo-tetrahydro-furan-3-yl)
ethyl]thioureido}propyl)thiourea

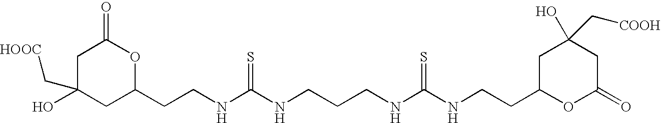

136) (2-{2-[3-(3-{3-[2-(4-Carboxymethyl-4-hydroxy-6-oxo-tetrahydro-pyran-2-yl)ethyl]
thioureido}propyl)thioureido]ethyl}-4-hydroxy-6-oxo-tetrahydro-pyran-4-yl) acetic acid

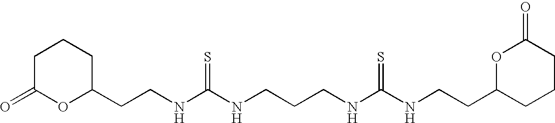

137) 1-[2-(6-Oxo-tetrahydro-pyran-2-yl)ethyl]-3-(3-{3-[2-(6-oxo-tetrahydro-pyran-2-yl)
ethyl]thioureido}propyl)thiourea

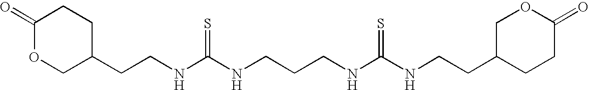

138) 1-[2-(6-Oxo-tetrahydro-pyran-3-yl)ethyl]-3-(3-{3-[2-(6-oxo-tetrahydro-pyran-3-yl)
ethyl]thioureido}propyl)thiourea

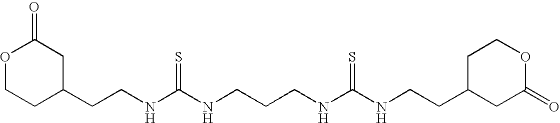

139) 1-[2-(2-Oxo-tetrahydro-pyran-4-yl)ethyl]-3-(3-{3-[2-(2-oxo-tetrahydro-pyran-4-yl)
ethyl]thioureido}propyl)thiourea

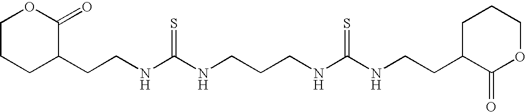

140) 1-[2-(2-Oxo-tetrahydro-pyran-3-yl)ethyl]-3-(3-{3-[2-(2-oxo-tetrahydro-pyran-3-yl)
ethyl]thioureido}propyl)thiourea

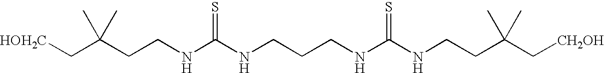

141) 1-(5-Hydroxy-3,3-dimethylpentyl)-3-{3-[3-(5-hydroxy-3,3-
dimethylpentyl)thioureido]propyl}thiourea

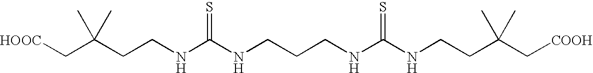

142) 5-(3-{3-[3-(4-Carboxy-3,3-dimethylbutyl)thioureido]propyl}thioureido)-3,3-
dimethyl pentanoic acid

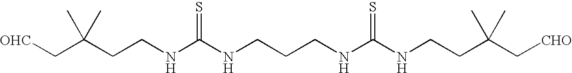

143) 1-(3,3-Dimethyl-5-oxo-pentyl)-3-{3-[3-(3,3-dimethyl-5-oxo-
pentyl)thioureidol]propylthiourea

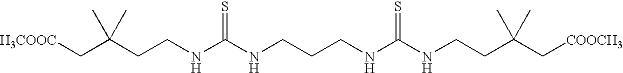

144) 5-(3-{3-[3-(4-Methoxycarbonyl-3,3-dimethylbutyl)thioureido]propyl}thioureido)
3,3-dimethyl pentanoic acid methyl ester

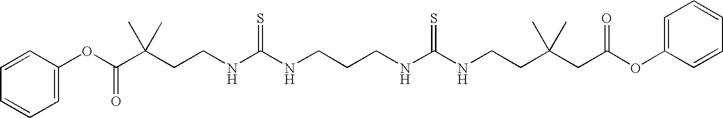

145) 3 ,3-Dimethyl-5-(3-{3-[3-(3-methyl-3-phenoxycarbonylbutyl)thioureido]propyl}
thioureido) pentanoic acid phenyl ester

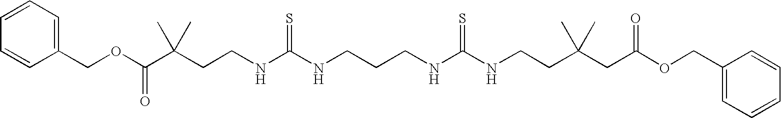

146) 5-(3-{3-[3-(3-Benzyloxycarbonyl-3-methylbutyl)thioureido]propyl}thioureido)-3,3-
dimethyl pentanoic acid benzyl ester

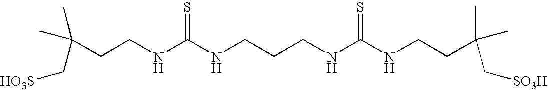

147) 4-(3-{3-[3-(3,3-Dimethyl-4-sulfobutyl)thioureido]propyl}thioureido)-2,2-
dimethylbutane-1-sulfonic acid

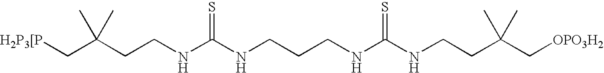

148) Phosphoricacidmono-[4-(3-{3-[3-(3,3-dimethyl-4-
phosphonooxybutyl)thioureido]propyl}thioureido)-2,2-dimethylbutyl] ester

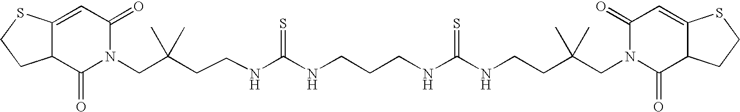

149) 1-[4-(4,6-Dioxo-2,3,3a,6-tetrahydro-4H-thieno[3,2-c]pyridin-5-yl)-3,3-
dimethylbutyl]-3-(3 -{3-[4-(4,6-dioxo-2,3,3a,6-tetrahydro-4H-thieno[3,2-c]pyridin-5-yl)-
3 ,3-dimethylbutyl]thioureido}propyl)thiourea

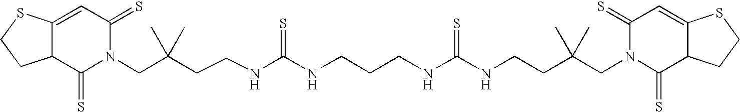

150) 1-[4-(4,6-Dithioxo-2,3,3a,6-tetrahydro-4H-thieno[3,2-c pyridin-5-yl)-3,3-
dimethylbutyl]-3-(3-{3-[4-(4,6-dithioxo-2,3,3a,6-tetrahydro-4H-thieno[3,2-c]pyridin-5-
yl)-3,3-dimethylbutyl]thioureido}propyl)thiourea

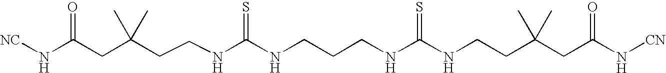

151) 5-[3-(3-{3-[4-(N-cyano-carbamoyl)-3,3-
dimethylbutyl]thioureido}propyl)thioureido]-3,3-dimethyl pentanoic acid cyanamide

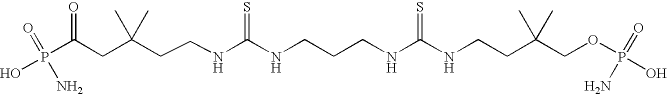

152) Phosphoramidicacidmono-{4-[3-(3-{3-[4-(amino-hydroxy-phosphoryloxy)-3,3-
dimethylbutyl]thioureido}propyl)thioureido]-2,2-dimethylbutyl} ester

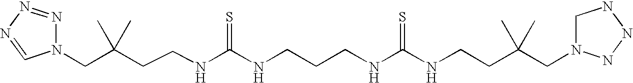

153) 1-(3,3-Dimethyl-4-tetrazol-1-yl-butyl)-3-{3-[3-(3,3-dimethyl-4-tetrazol-1-yl
butyl)thioureido]propyl}thiourea

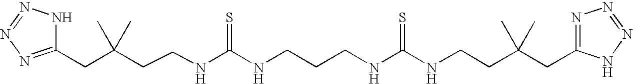

154) 1-[3,3-Dimethyl-4-(1H-tetrazol-5-yl)butyl]-3-(3-{3-[3,3-dimethyl-4-(1H-tetrazol-5-
yl)butyl]thioureido}propyl)thiourea

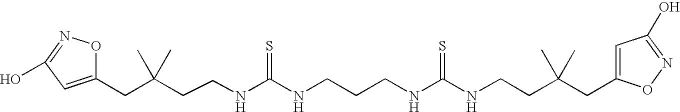

155) 1-[4-(3-Hydroxy-isoxazol-5-yl)-3,3-dimethylbutyl]-3-(3-{3-[4-(3-hydroxy-isoxazol-
5-yl)-3,3-dimethylbutyl]thioureido}propyl)thiourea

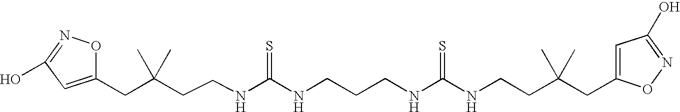

156) 1-[4-(3-Hydroxy-isoxazol-4-yl)-3,3-dimethylbutyl]-3-(3-{3-[4-(3 -hydroxy-isoxazol
4-yl)-3,3-dimethylbutyl]thioureido}propyl)thiourea

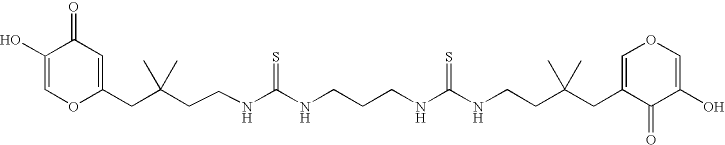

157) 1-[4-(5-Hydroxy-4-oxo-4H-pyran-3-yl)-3,3-dimethylbutyl]-3-(3-{3-[4-(5-hydroxy-
4-oxo-4H-pyran-2-yl)-3,3-dimethylbutyl]thioureido}propyl)thiourea

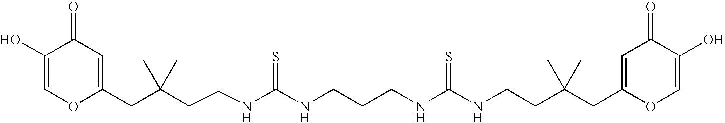

158) 1-[4-(5-Hydroxy-4-oxo-4H-pyran-2-yl)-3,3-dimethylbutyl]-3-(3-{3-[4-(5-hydroxy-
4-oxo-4H-pyran-2-yl)-3,3-dimethylbutyl]thioureido}propyl)thiourea

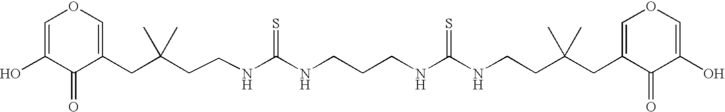

159) 1-[4-(5-Hydroxy-4-oxo-4H-pyran-3-yl)-3,3-dimethylbutyl]-3-(3-{3-[4-(5-hydroxy-
4-oxo-4H-pyran-3-yl)-3,3-dimethylbutyl]thioureido}propyl)thiourea

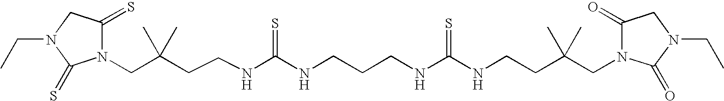

160) 1-[4-(3-Ethyl-2,5-dioxo-imidazolidin-1-yl)-3,3-dimethylbutyl]-3-(3-{3-[4-(3-ethyl-
2,5-dithioxo-imidazolidin-1-yl)-3,3-dimethylbutyl]thioureido}propyl)thiourea

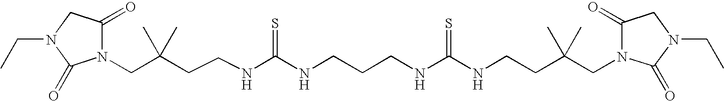

161) 1-[4-(3-Ethyl-2,5-dioxo-imidazolidin-1-yl)-3,3-dimethylbutyl]-3-(3-{3-[4-(3-ethyl-
2,5-dioxo-imidazolidin-1-yl)-3,3-dimethylbutyl]thioureido}propyl)thiourea

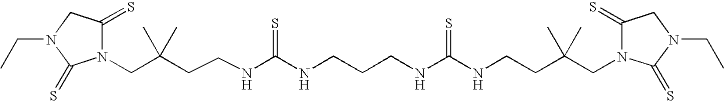

162) 1-[4-(3-Ethyl-2,5-dithioxo-imidazolidin-1-yl)-3,3-dimethylbutyl]-3-(3-{3-[4-(3-
ethyl-2,5-dithioxo-imidazolidin-1-yl)-3,3-dimethylbutyl]thioureido}propyl)thiourea

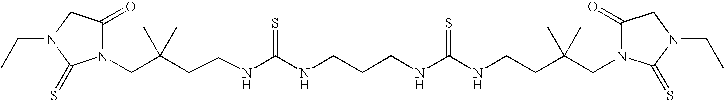

163) 1-[4-(3-Ethyl-5-oxo-2-thioxo-imidazolidin-1-yl)-3,3-dimethylbutyl]-3-(3-{3-[4-(3-
ethyl-5-oxo-2-thioxo-imidazolidin-1-yl)-3,3-dimethylbutyl]thioureido}propyl)thiourea

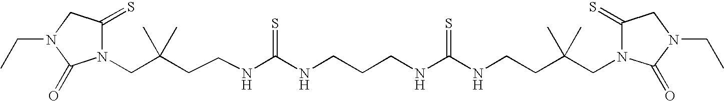

164) 1-[4-(3-Ethyl-2-oxo-5-thioxo-imidazolidin-1-yl)-3,3-dimethylbutyl]-3-(3-{3-[4-(3-
ethyl-2-oxo-5-thioxo-imidazolidin-1-yl)-3,3-dimethylbutyl]thioureido}propyl)thiourea

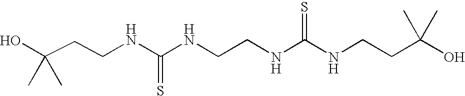

165) 1-(3-Hydroxy-3-methylbutyl)-3-{2-[3-(3-hydroxy-3-
methylbutyl)thioureido]ethyl}thiourea

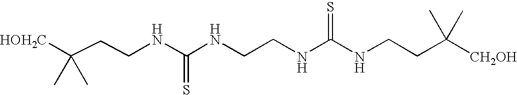

166) 1-(4-Hydroxy-3,3-dimethylbutyl)-3-{2-[3-(4-hydroxy-3,3-
dimethylbutyl)thioureido]ethyl}thiourea

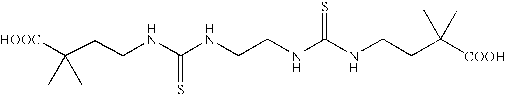

167) 4-(3-{2-[3-(3-Carboxy-3-methylbutyl)thioureido]ethyl}thioureido)-2,2-dimethyl
butyric acid

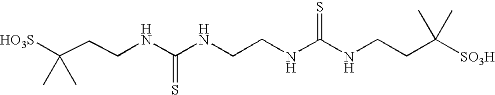

168) 2-Methyl-4-(3-{2-[3-(3-methyl-3-sulfobutyl)thioureido]ethyl}thioureido)butane-2-
sulfonic acid

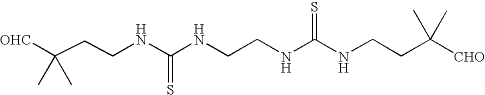

169) 1-(3,3-Dimethyl-4-oxobutyl)-3-{2-[3-(3,3-dimethyl-4-
oxobutyl)thioureido]ethyl}thiourea

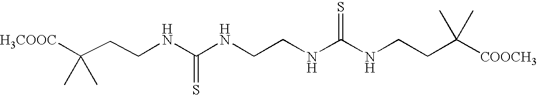

170) 4-(3-{2-[3-(3-Methoxycarbonyl-3-methylbutyl)thioureido]ethyl}thioureido)-2,2-
dimethyl butyric acid methyl ester

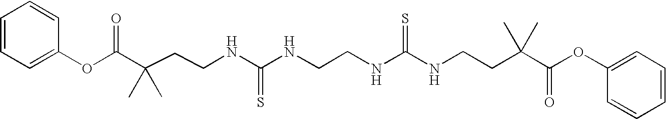

171) 2,2-Dimethyl-4-(3-{2-[3-(3-methyl-3-
phenoxycarbonylbutyl)thioureido]ethyl}thioureido) butyric acid phenyl ester

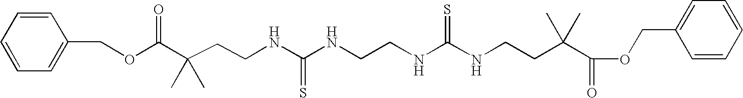

172) 4-(3-{2-[3-(3-Benzyloxycarbonyl-3-methylbutyl)thioureido]ethyl}thioureido)-2,2-
dimethyl butyric acid benzyl ester

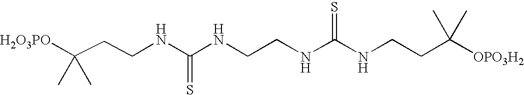

173) Phosphoricacidmono-[1,1-dimethyl-3-(3-{2-[3-(3-methyl-3-
phosphonooxybutyl)thioureido]ethyl}thioureido)propyl] ester

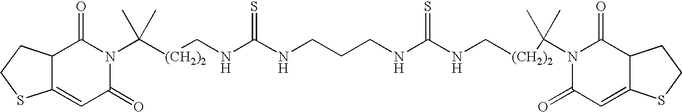

174) 1-[4-(4,6-Dioxo-2,3,3a,6-tetrahydro-4H-thieno[3,2-c]pyridin-5-yl)-4-methylpentyl]-
3-(3-{3-[4-(4,6-dioxo-2,3,3a,6-tetrahydro-4H-thieno[3,2-c]pyridin-5-yl)-4-
methylpentyl]thioureido}propyl)thiourea

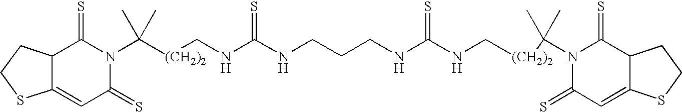

175) 1-[4-(4,6-Dithioxo-2,3,3a,6-tetrahydro-4H-thieno[3,2-c]pyridin-5-yl)-4-
methylpentyl]-3-(3-{3-[4-(4,6-dithioxo-2,3,3a,6-tetrahydro-4H-thieno[3,2-c]pyridin-5-
yl)-4-methylpentyl]thioureido}propyl)thiourea

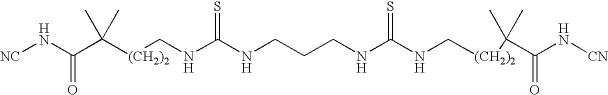

176) 5-[3-(3-{3-[4-(N-cyano-carbamoyl)-4-methylpentyl]thioureido}propyl)thioureido]-
2,2-dimethyl pentanoic acid cyanamide

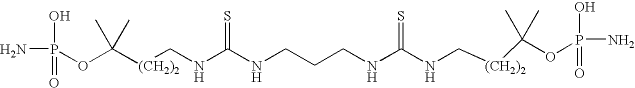

177) Phosphoramidicacidmono-{4-[3-(3-{3-[4-(amino-hydroxy-phosphoryloxy)-4-
methylpentyl]thioureido}propyl)thioureidol-1,1-dimethylbutyl}ester

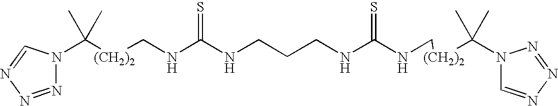

178) 1-(4-Methyl-4-tetrazol-1-yl-pentyl)-3-{3-[3-(4-methyl-4-tetrazol-1-yl-
pentyl)thioureido]propyl}thiourea

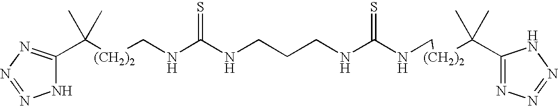

179) 1-[4-Methyl-4-(1H-tetrazol-5-yl)pentyl]-3-(3-{3-[4-methyl-4-(1H-tetrazol-5-
yl)pentyl]thioureido}propyl)thiourea

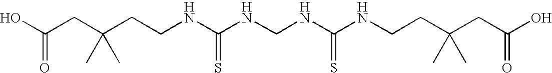

180) Bis-[1-carboxyl-3,3-dimethylpentylthioureido]methane

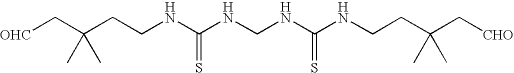

181) Bis-[1-aldo-3,3-dimethylpentylthioureido]methane

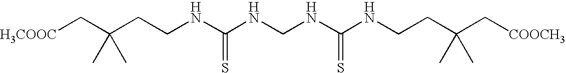

182) Bis-[1-methoxycarbonyl-3,3-dimethylpentylthioureido]methane

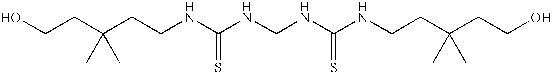

183) Bis-[1-hydroxy-3,3-dimethylpentylthioureido]methane

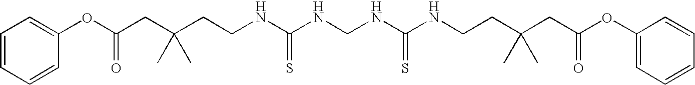

184) Bis-[1-phenoxycarbonyl-3,3-dimethylpentylthioureido]methane

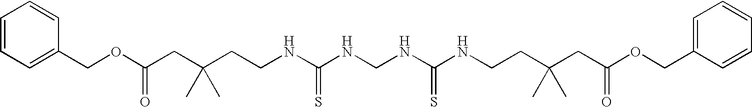

185) Bis-[benzyloxycarbonyl-3,3-dimethylpentylthioureido]methane

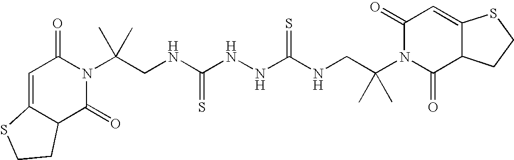

186) 1,6-Bis-[2′-(4″,6″-dioxo-2″,3″,3a″,6″-tetrahydro-4′H-thieno[3′′′,2′′′-c]pyridin-5″-yl)-
2′-methylpropyl]bithiourea

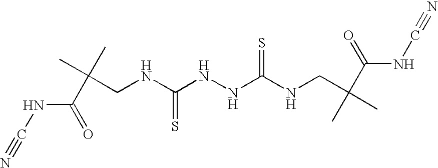

187) 1,6-Bis-[1′-(N-cyanamido)-2′,2′-dimethylpropyl]bithiourea

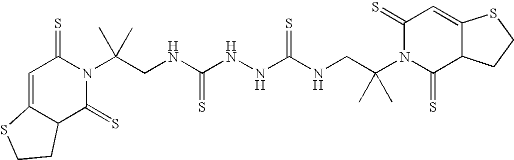

188) 1,6-Bis-[2′-(4″,6″-dithioxo-2″,3″,3a″,6″-tetrahydro-4″H-thieno[3′′′,2′′′-c]pyridin-5
yl)-2′-methylpropyl]bithiourea

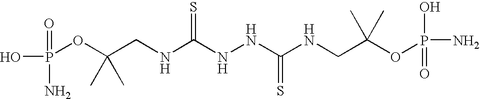

189) 1,6-Bis-[2′-(aminohydroxyphosphoryloxy)-2′-methylpropyl]bithiourea

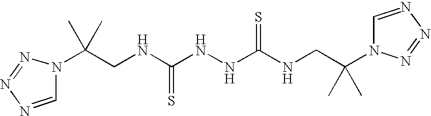

190) 1,6-Bis-[2′-methyl-2′-(tetrazol-1″-yl)propyl]bithiourea

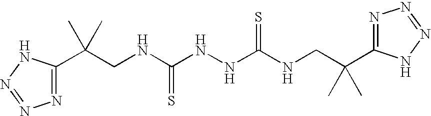

191) 1,6-Bis-[2′-methyl-2′-(1″H-tetrazol-5″-yl)propyl]bithiourea

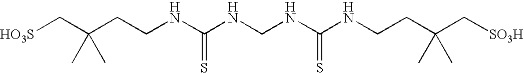

192) Bis-[1-sulfo-2,2-dimethylbutylthioureido]methane

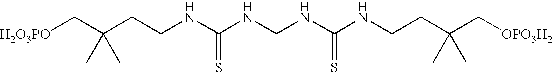

193) Bis-‘-phosphonooxy-2,2-dimethylbutylthioureido]methane

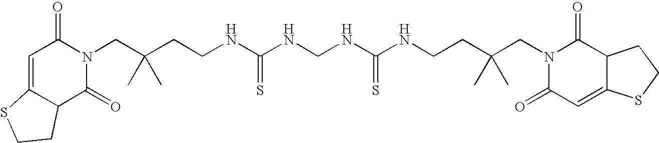

194) Bis-‘-(4′,6′-dioxo-2′,3′,3a′,6′-tetrahydro-4′H-thieno[3″,2″-c]pyridin-5′-yl)-2,2-
dimethylbutylthioureido]methane

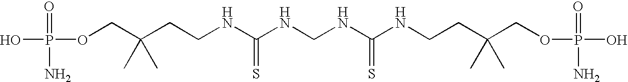

195) Bis-‘-(aminohydroxyphosphoryloxy)-2,2-dimethylbutylthioureido]methane

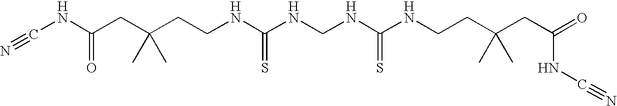

196) Bis-‘-(N-cyanoamido)-3,3-dimethylpentylthioureido]methane

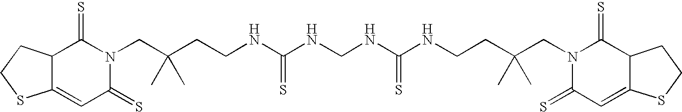

197) Bis-‘-(4′,6′-dithioxo-2′,3′,3a′,6′-tetrahydro-4′H-thieno[3′,2′-c]pyridin-5′-yl)-2,2-
dimethylbutylthioureido]methane

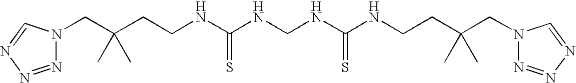

198) Bis-‘-(tetrazol-1″-yl)-2,2-dimethylbutylthioureido]methane

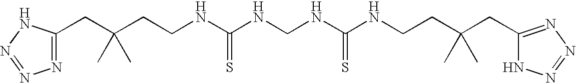

199) Bis-‘-(1″H-tetrazol-5″-yl)-2,2-dimethylbutylthioureido]methane

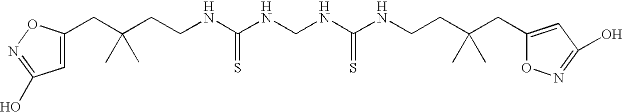

200) Bis-‘-(3′-hydroxy-isoazol-5′-yl)-2,2-dimethylbutylthioureido]methane

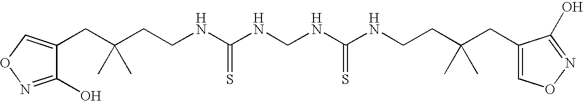

201) Bis-‘-(3′-hydorxy-isoxazol-4′-yl)-2,2-dimethylbutylthioureido]methane

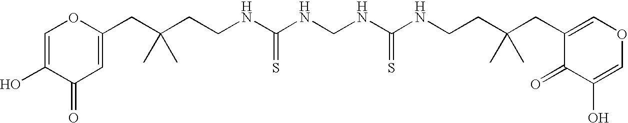

202) Bis-‘-(5′-hydroxy-4′-oxo-4′H-pyran-2′-yl)-2,2-dimethybutylthioureido]methane

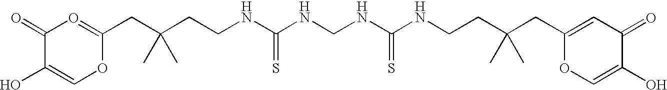

203) Bis-‘-(5′-hydroxy-4′-oxo-4′H-pyran-3′-yl)-2,2-dimethybutylthioureido]methane

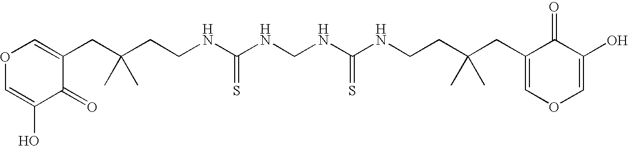

204) Bis-‘-(5′-hydroxy-4′-oxo-4′H-pyran-3′-yl)-2,2-dimethybutylthioureido]methane

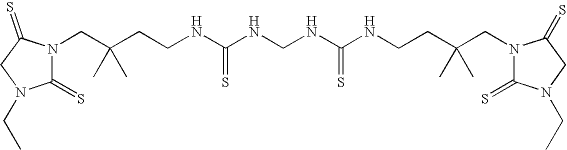

205) Bis-‘-(3′-ethyl-2′,5′-dithioxo-imidazolidin-1′-yl)-2,2-
dimethylbutylthioureidol]methane

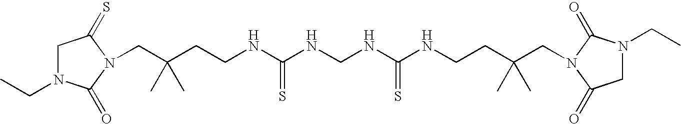

206) [{4-(3″-ethyl-2″,5″-dithioxo-imidazolidin-1′-yl)-3,3-dimethylbutylthioureido}-{4′-
(3′′′-ethyl-2′′′,5′′′-dioxo-imidazolidin-1′′′-yl)-3′,3′-dimethylbutylthioureido}]methane

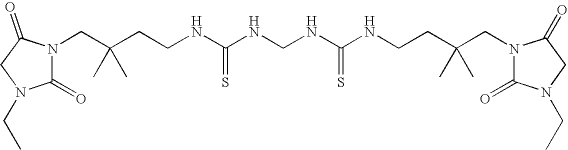

207) Bis- ‘-(3′-ethyl-2′,5′-dioxo-imidazolidin-1′-yl)-2,2-dimethylbutylthioureido]methane

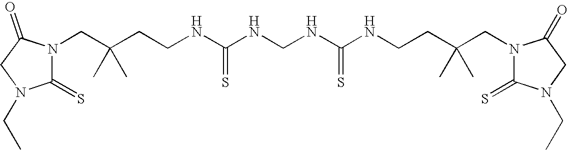

208) Bis-‘-(3′-ethyl-5′-oxo-2′-thioxo-imidazolidin-1′-yl)-2,2-
dimethylbutylthioureido]methane

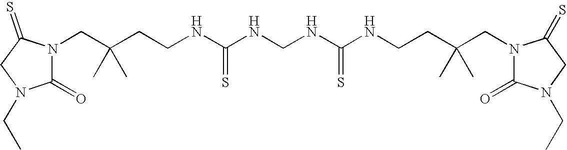

209) Bis-‘-(3′-ethyl-2′-oxo-5′-thioxo-imidazolidin-1′-yl)-2,2-
dimethylbutylthioureido]methane

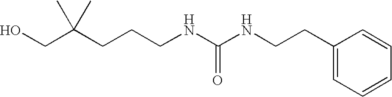

210) 1-(5-Hydroxy-4,4-dimethylpentyl)-3-phenethylurea

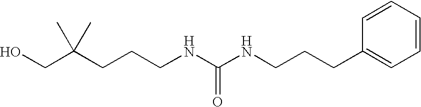

211) 1-(5-Hydroxy-4,4-dimethylpentyl)-3-(3-phenylpropyl)urea

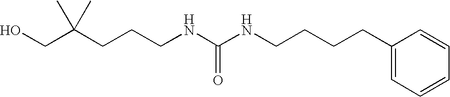

212) 1-(5-Hydroxy-4,4-dimethylpentyl)-3-(4-phenylbutyl)urea

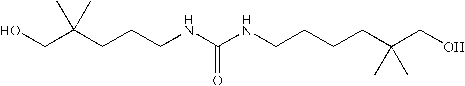

213) 1-(5-Hydroxy-4,4-dimethylpentyl)-3-(6-hydroxy-5,5-dimethylhexyl)urea

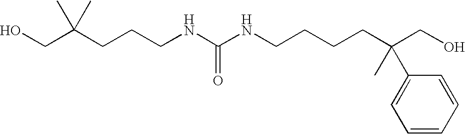

214) 1-(5-Hydroxy-4,4-dimethylpentyl)-3-(6-hydroxy-5-methyl-5-phenylhexyl)urea

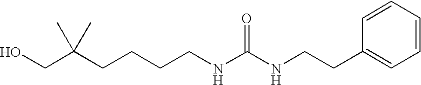

215) 1-(6-Hydroxy-5,5-dimethylhexyl)-3-phenethylurea

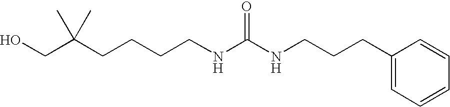

216) 1-(6-Hydroxy-5,5-dimethylhexyl)-3-(3-phenylpropyl)urea

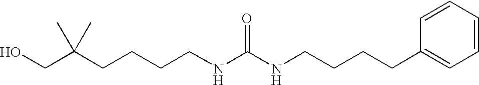

217) 1-(6-Hydroxy-5,5-dimethylhexyl)-3-(4-phenylbutyl)urea

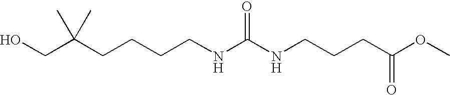

218) methyl 4-(3-(6-hydroxy-5,5-dimethylhexyl)ureido)butanoate

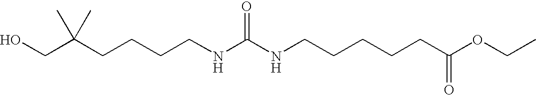

219) Ethyl 6-(3-(6-hydroxy-5,5-dimethylhexyl)ureido)hexanoate

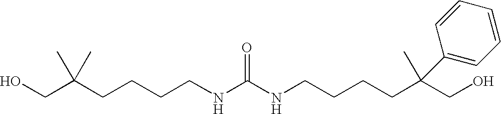

220) 1-(6-Hydroxy-5,5-dimethylhexyl)-3-(6-hydroxy-5-methyl-5-phenylhexyl)urea

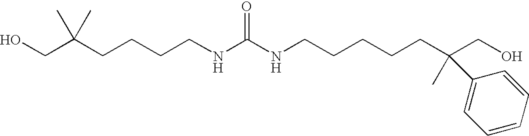

221) 1-(6-Hydroxy-5,5-dimethylhexyl)-3-(7-hydroxy-6-methyl-6-phenylheptyl)urea

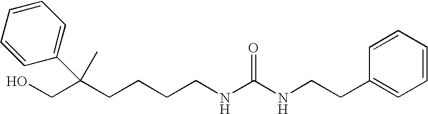

222) 1-(6-Hydroxy-5-methyl-5-phenylhexyl)-3-phenethylurea

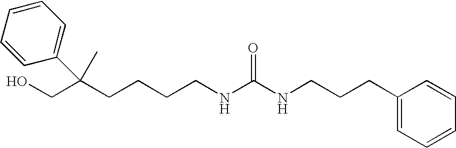

223) 1-(6-Hydroxy-5-methyl-5-phenylhexyl)-3-(3-phenylpropyl)urea

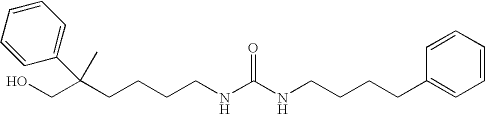

224) 1-(6-Hydroxy-5-methyl-5-phenylhexyl)-3-(4-phenylbutyl)urea

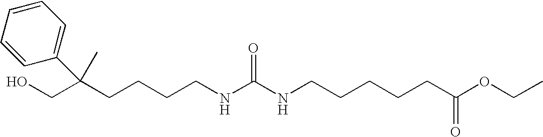

225) Ethyl 6-(3 -(6-hydroxy-5-methyl-5-phenylhexyl)ureido)hexanoate

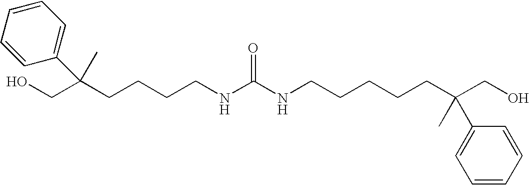

226) 1-(6-Hydroxy-5-methyl-5-phenylhexyl)-3-(7-hydroxy-6-methyl-6-phenylheptyl)urea

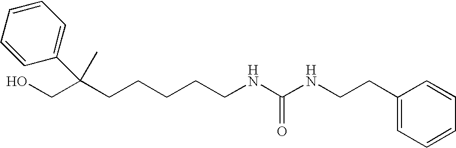

227) 1-(7-Hydroxy-6-methyl-6-phenylheptyl)-3-phenethylurea

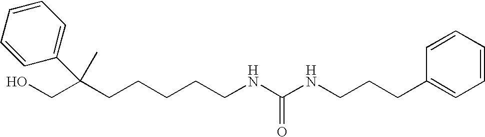

228) 1-(7-hydroxy-6-methyl-6-phenylheptyl)-3-(3-phenylpropyl)urea

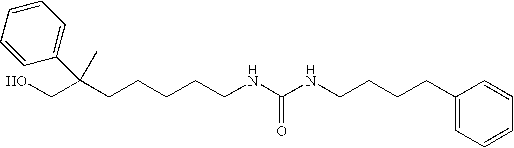

229) 1-(7-Hydroxy-6-methyl-6-phenylheptyl)-3-(4-phenylbutyl)urea

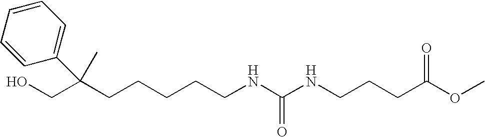

230) Ethyl 7-(3-(7-hydroxy-6-methyl-6-phenylhexyl)ureido)butanoate

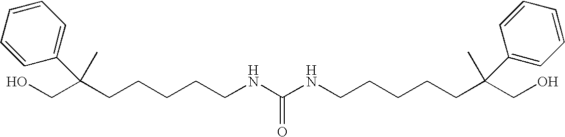

231) 1,3-Bis(7-hydroxy-6-methyl-6-phenylheptyl)urea

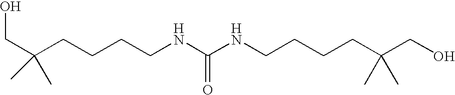

232) 1,3-Bis(6-hydroxy-5,5-dimethylhexyl)urea

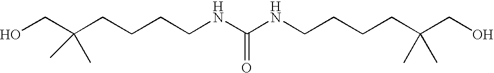

233) 1,3-Bis(5-hydroxy-4,4-dimethylpentyl)urea

### 4.1 Definitions and Abbreviations

The following abbreviations are used herein and have the indicated definitions: Apo(a) is apolipoprotein(a); Apo A-I is apolipoprotein A-I; Apo B is apolipoprotein B; Apo E is apolipoprotein E; FH is Familial hypercholesterolemia; FCH is Familial combined hyperlipidemia; GDM is Gestational diabetes mellitus; HDL is High density lipoprotein; IDL is Intermediate density lipoprotein; IDDM is Insulin dependent diabetes mellitus; LDH is Lactate dehdyrogenase; LDL is Low density lipoprotein; Lp(a) is Lipoprotein (a); MODY is Maturity onset diabetes of the young; NIDDM is Non-insulin dependent diabetes mellitus; PPAR is Peroxisome proliferator activated receptor; RXR is Retinoid X receptor; and VLDL is Very low density lipoprotein.

The terms “compound A” and “compound B” refer to the compounds 1,3-bis-(6-hydroxy-5,5-dimethyl-hexyl)-urea and 1,3-bis-(5-hydroxy-4,4,-dimethyl-hexyl)-urea, having the respective structures:

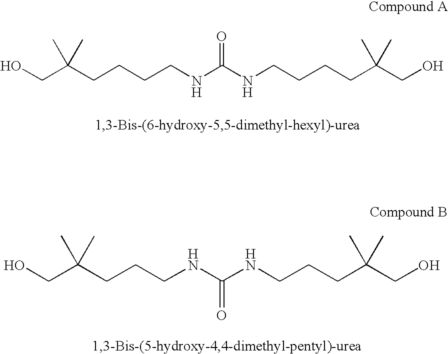

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compound's enantiomers and stereoisomers, that is, both the stereomerically pure form (e.g., geometrically pure, enantiomerically pure, or diastereomerically pure) and enantiomeric and stereoisomeric mixtures.

A compound of the invention is considered optically active or enantiomerically pure (i.e., substantially the R-form or substantially the S-form) with respect to a chiral center when the compound is about 90% ee (enantiomeric excess) or greater, preferably, equal to or greater than 95% ee with respect to a particular chiral center. A compound of the invention is considered to be in enantiomerically-enriched form when the compound has an enantiomeric excess of greater than about 80% ee with respect to a particular chiral center. A compound of the invention is considered diastereomerically pure with respect to multiple chiral centers when the compound is about 90% de (diastereomeric excess) or greater, preferably, equal to or greater than 95% de with respect to a particular chiral center. A compound of the invention is considered to be in diastereomerically-enriched form when the compound has an diastereomeric excess of greater than about 80% de with respect to a particular chiral center. As used herein, a racemic mixture means about 50% of one enantiomer and about 50% of is corresponding enantiomer relative to all chiral centers in the molecule. Thus, the invention encompasses all enantiomerically-pure, enantiomerically-enriched, diastereomerically pure, diastereomerically enriched, and racemic mixtures of compounds of Formulas I through IIa.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds of the invention are defined herein by their chemical structures and/or chemical names. Where a compound is referred to by both a chemical structure and a chemical name, and the chemical structure and chemical name conflict, the chemical structure is determinative of the compound's identity.

When administered to a patient, e.g., to an animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least 95%, preferably at least 98%, of a single ether compound of the invention by weight of the isolate.

The phrase “pharmaceutically acceptable salt(s),” as used herein includes, but are not limited to, salts of acidic or basic groups that may be present in the compounds of the invention. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds of the invention that include an amino moiety also can form pharmaceutically acceptable salts with various amino acids, in addition to the acids mentioned above. Compounds of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein, the term “solvate” means a compound of the invention or a salt thereof, that further includes a stoichiometric or non-stoichiometric amount of a solvent bound by non-covalent intermolecular forces. Preferred solvents are volatile, non-toxic, and/or acceptable for administration to humans in trace amounts.

As used herein, the term “hydrate” means a compound of the invention or a salt thereof, that further includes a stoichiometric or non-stoichiometric amount of water bound by non-covalent intermolecular forces.

As used herein, the term “clathrate” means a compound of the invention or a salt thereof in the form of a crystal lattice that contains spaces (e.g., channels) that have a guest molecule (e.g., a solvent or water) trapped within.

“Altering lipid metabolism” indicates an observable (measurable) change in at least one aspect of lipid metabolism, including but not limited to total blood lipid content, blood HDL cholesterol, blood LDL cholesterol, blood VLDL cholesterol, blood triglyceride, blood Lp(a), blood apo A-I, blood apo E and blood non-esterified fatty acids.

“Altering glucose metabolism” indicates an observable (measurable) change in at least one aspect of glucose metabolism, including but not limited to total blood glucose content, blood insulin, the blood insulin to blood glucose ratio, insulin sensitivity, and oxygen consumption.

As used herein, the term “alkyl group” means a saturated, monovalent unbranched or branched hydrocarbon chain. Examples of alkyl groups include, but are not limited to, (C₁-C₆)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or substituted with one or two suitable substituents.

An “alkenyl group” means a monovalent unbranched or branched hydrocarbon chain having one or more double bonds therein. The double bond of an alkenyl group can be unconjugated or conjugated to another unsaturated group. Suitable alkenyl groups include, but are not limited to (C₂-C₆)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

An “alkynyl group” means monovalent unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted with one or two suitable substituents.

An “aryl group” means a monocyclic or polycyclic-aromatic radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or two suitable substituents. Preferably, the aryl group is a monocyclic ring, wherein the ring comprises 6 carbon atoms, referred to herein as “(C₆)aryl”.

A “heteroaryl group” means a monocyclic- or polycyclic aromatic ring comprising carbon atoms, hydrogen atoms, and one or more heteroatoms, preferably 1 to 3 heteroatoms, independently selected from nitrogen, oxygen, and sulfur. Illustrative examples of heteroaryl groups include, but are not limited to, pyridinyl, pyridazinyl, pyrimidinyl, pyrazyl, triazinyl, pyrrolyl, pyrazolyl, imidazolyl, (1,2,3)- and (1,2,4)-triazolyl, pyrazinyl, pyrimidinyl, tetrazolyl, furyl, thiophenyl, isoxazolyl, thiazolyl, furyl, phenyl, isoxazolyl, and oxazolyl. A heteroaryl group can be unsubstituted or substituted with one or two suitable substituents. Preferably, a heteroaryl group is a monocyclic ring, wherein the ring comprises 2 to 5 carbon atoms and 1 to 3 heteroatoms, referred to herein as “(C₂-C₅)heteroaryl”.

A “cycloalkyl group” means a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted by one or two suitable substituents. Preferably, the cycloalkyl group is a monocyclic ring or bicyclic ring.

A “heterocycloalkyl group” means a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as (C₁-C₆)heterocycloalkyl.

As used herein a “heterocyclic radical” or “heterocyclic ring” means a heterocycloalkyl group or a heteroaryl group.

The term “alkoxy group” means an —O-alkyl group, wherein alkyl is as defined above. An alkoxy group can be unsubstituted or substituted with one or two suitable substituents. Preferably, the alkyl chain of an alkyloxy group is from 1 to 6 carbon atoms in length, referred to herein as “(C₁-C₆)alkoxy”.

The term “aryloxy group” means an —O-aryl group, wherein aryl is as defined above. An aryloxy group can be unsubstituted or substituted with one or two suitable substituents. Preferably, the aryl ring of an aryloxy group is a monocyclic ring, wherein the ring comprises 6 carbon atoms, referred to herein as “(C₆)aryloxy”.

The term “benzyl” means —CH₂-phenyl.

The term “phenyl” means —C₆H₅. A phenyl group can be unsubstituted or substituted with one or two suitable substituents.

A “hydrocarbyl” group means a monovalent group selected from (C₁-C₈)alkyl, (C₂-C₈)alkenyl, and (C₂-C₈)alkynyl, optionally substituted with one or two suitable substituents. Preferably, the hydrocarbon chain of a hydrocarbyl group is from 1 to 6 carbon atoms in length, referred to herein as “(C₁-C₆)hydrocarbyl”.

A “carbonyl” group is a divalent group of the formula —C(O)—.

An “alkoxycarbonyl” group means a monovalent group of the formula —C(O)— alkoxy. Preferably, the hydrocarbon chain of an alkoxycarbonyl group is from 1 to 8 carbon atoms in length, referred to herein as a “lower alkoxycarbonyl” group.

A “carbamoyl” group means the radical —C(O)N(R′)₂, wherein R′ is chosen from the group consisting of hydrogen, alkyl, and aryl.

As used herein, “halogen” means fluorine, chlorine, bromine, or iodine. Correspondingly, the meaning of the terms “halo” and “Hal” encompass fluoro, chloro, bromo, and iodo.

As used herein, a “suitable substituent” means a group that does not nullify the synthetic or pharmaceutical utility of the compounds of the invention or the intermediates useful for preparing them. Examples of suitable substituents include, but are not limited to: (C₁-C₈)alkyl; (C₁-C₈)alkenyl; (C₁-C₈)alkynyl; (C₆)aryl; (C₂-C₅)heteroaryl; (C₃-C₇)cycloalkyl; (C₁-C₈)alkoxy; (C₆)aryloxy; —CN; —OH; oxo; halo, —CO₂H; —NH₂; —NH((C₁-C₈)alkyl); —N((C₁-C₈)alkyl)₂; —NH((C₆)aryl); —N((C₆)aryl)₂; —CHO; —CO((C₁-C₈)alkyl); —CO((C₆)aryl); —CO₂((C₁-C₈)alkyl); and —CO₂((C₆)aryl). One of skill in the art can readily choose a suitable substituent based on the stability and pharmacological and synthetic activity of the compound of the invention.

### 4.2 Synthesis of the Compounds of the Invention

The compounds of the invention can be obtained via the synthetic methodology illustrated in Schemes 1-14. Starting materials useful for preparing the compounds of the invention and intermediates thereof, are commercially available or can be prepared from commercially available materials using known synthetic methods and reagents.

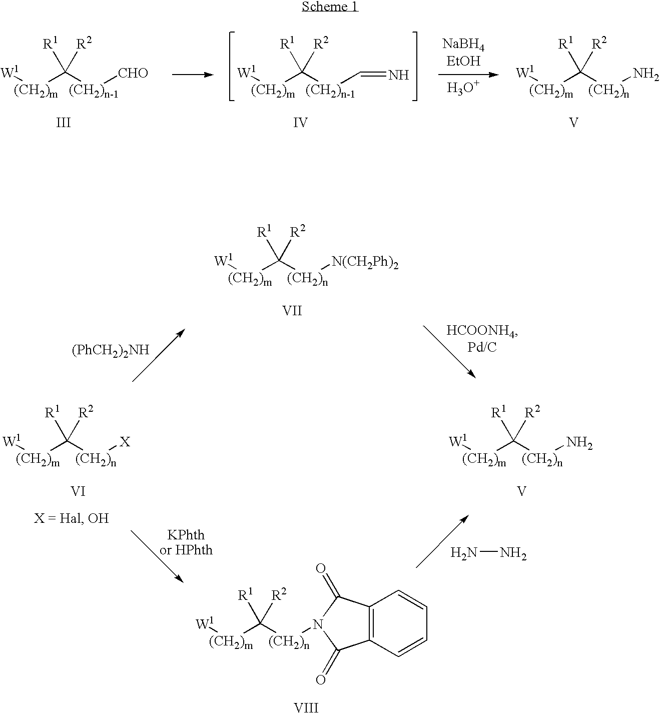

Amines V are commercially available (e.g., Aldrich Chemical Co., Milwaukee, Wis.) or can be prepared by well-known synthetic methods. Scheme 1 illustrates the synthesis of amines V from aldehydes III via the imine IV (see Wang et al. J. Org. Chem. 1995, 60, 7364, Tanaka et al. J. Med. Chem. 1998, 41, 2390, Smith and March, ADVANCED ORGANIC CHEMISTRY: REACTIONS, MECHANISMS AND STRUCTURES, 5^{th} Ed.; Wiley: New York, 2001; p 1203, and references cited herein, and methods referenced in Larock, COMPREHENSIVE ORGANIC TRANSFORMATIONS, 2^{nd} Ed., Wiley: New York 1999, p. 835). In a typical procedure, a mixture of aldehyde and ammonium formate or ammonium oxalate is heated at temperatures higher than 120° C., preferably at 140° C., until no more water is distilled off. Then the temperature of the reaction mixture is raised to over 150° C., preferably 180-200° C., for 2 to 10 hours. The reaction mixture is cooled at room temperature, treated with concentrated HCl at 0° C. or higher for 2 to 6 hours, and the organic impurities extracted with an organic solvent such as diethyl ether, t-butyl methyl ether, benzene, toluene, hexane, preferably toluene. Afterwards, the aqueous layer is made alkaline with an aqueous sodium hydroxide solution and the amine is extracted in an organic solvent and purified by methods commonly used in the field. Amines V are also prepared from a halide VI (X=Hal) and dibenzylamine. In a typical procedure, halide VI is treated with dibenzylamine neat at temperatures in the range of 100 to 150° C., preferably 130° C., or in diglyme in the presence of potassium carbonate at temperatures in the range of 120 to 180° C., preferably at 140° C., until no more change in the starting material is observed by an analytical method such as but not limited to HPLC or tlc. When the reaction is complete, the amine is converted into a hydrochloride salt and is precipitated as a hydrochloride salt in a dry solvent such as 2-propanol. The dibenzylamine derivative VII is treated with 10% Pd/C and ammonium formate in methanol at reflux for 2 to 24 hours, then filtered through Celite and concentrated in vacuo to yield the crude amine V, which is purified by usual methods. For a representative example see Purchase et al. J. Org. Chem. 1991, 56, 457-459.

Scheme 1 also illustrates the preparation of amines of formula V by Gabriel synthesis starting from halo-derivatives VI (for general references see Gibson et al. Angew. Chem. 1968, 80, 986, Smith and March, ADVANCED ORGANIC CHEMISTRY: REACTIONS, MECHANISMS AND STRUCTURES, 5th Ed.; Wiley: New York, 2001; p 513, and references cited herein). For an improved Gabriel synthesis, see also Sheehan et al. J. Amer. Chem. Soc. 1950, 72, 2786-2788. In a typical procedure, bromide VI (X=Br) and potassium phthalimide in DMF are kept at room temperature or heated to 90° C. for 0.5 to 4 hours, and extracted in a solvent or precipitated by addition of water, and recrystallized. The phthalimide of formula VIII thus obtained is treated in methanol with an 85% aqueous solution of hydrazine hydrate for 15 min to one hour. Addition of water and removal of the methanol is followed by addition of HCl and heating under reflux for 1 hour, removal of crystalline phthalhydrazide by cooling to 0° C., then workup of the amine V from the filtrate. In an alternative procedure potassium phthalimide and potassium carbonate in the presence of catalytic amounts of benzyltriethylammonium chloride in acetone are refluxed for 40 min, then a representative bromide of formula VI (X=Br) is added dropwise for 4 hr at reflux. When the reaction is complete, the mixture is subjected to separation and purification by known methods, such as chromatography or recrystallization. As a reference see Sasse et al. J. Med. Chem. 2001, 44, 694-702 and Khan J. Org. Chem. 1996, 61, 8063-8068. The reactions described above are all monitored by an analytical method such as HPLC, tlc or NMR. N-Alkylphthalimides of formula VIII are also prepared from an alcohol and phthalimide using Mitsunobu conditions (Mitsunobu et al. J. Amer. Chem. Soc. 1972, 94, 679-680). In a typical procedure, an alcohol of formula VI (X═OH) is treated with phthalimide in the presence of triphenylphosphine and diethyl azodicarboxylate (DEAD) in dry THF at 0° C., then the mixture is stirred overnight at room temperature. After evaporation of the solvent, the phthalimide is separated and purified in the usual manner. Subsequently, the phthalimide in ethanol is treated with hydrazine hydrate at reflux for 15 min, then the suspension is cooled, acidified and filtered. The amine of formula V is recovered from the filtrate either as a hydrochloride salt or as a free base by usual separation methods.

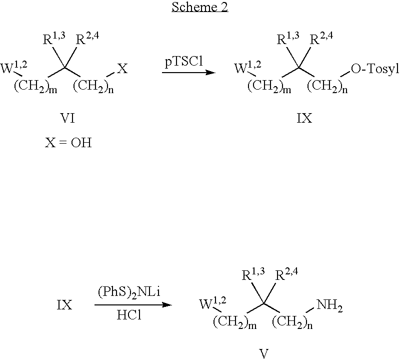

Amines V are also prepared from p-toluenesulfonic acid esters of formula IX, as illustrated in Scheme 2. Esters IX can be obtained by various means, including treating the corresponding alcohols with p-toluenesulfonyl chloride as described in International Patent Application No. 01/31873 to Dasseux, et al. and by other methods well-known in the field (for a review, see Sandler and Karo, Organic Functional Group Preparations, 2d ed., vol. 3, Academic Press: New York, 1989, pp. 129-151), or via commercial sources (e.g., Aldrich Chemical Co., Milwaukee, Wis.). Derivatives of formula IX afford amines V when subsequently treated with lithium bisarylsulfenimide (PhS)₂NLi in THF, and aqueous HCl in diethyl ether, as described in Mukaiyama et al. Bull. Soc. Chem, Jpn. 1971, 44, 2797-2800.

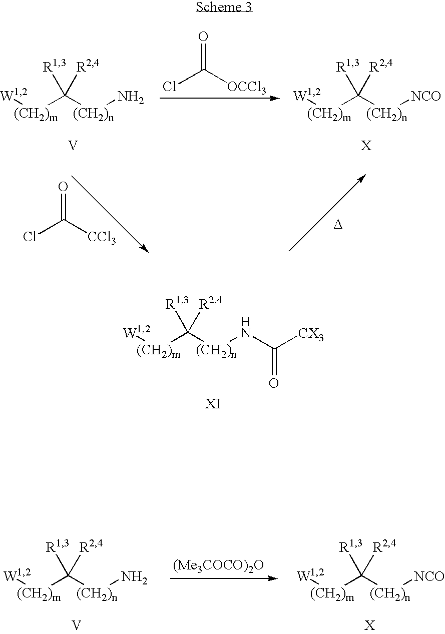

Scheme 3 illustrates the synthesis of isocyanates X via treatment of the corresponding amines V with phosgene or a phosgene substitute, such as trichloromethyl chloroformate, as shown in Scheme 3. Syntheses of isocyanates from amines V and phosgene and other phosgene substitutes are well-known and have been described in detail in the literature (for general references see Ozaki et al. Chem. Rev. 1972, 72, 457, Findeisen et al. in Houben-Weyl, Methoden der Organische Chemie, 4^{th} Ed., Georg Thieme Verlag Stuttgart 1952, vol. 4, p. 738, Smith and March, ADVANCED ORGANIC CHEMISTRY: REACTIONS, MECHANISMS AND STRUCTURES, 5^{th} Ed.; Wiley: New York, 2001; p 507, and references cited herein). In a typical procedure, the amine of formula V in a solvent such as dichloromethane, chloroform, dichloroethane, preferably dichloromethane, is treated with phosgene or a phosgene substitute at temperatures from −40° C. to ambient until disappearance of, or no more change in the starting material is observed by an analytical method such as but not limited to HPLC or tlc. The isocyanate is subsequently isolated and purified by usual chromatographic methods or recrystallization. In an alternative procedure trichloromethyl chloroformate and a catalytic amount of activated charcoal in dry ethyl acetate is added rapidly to amine V hydrochloride as a solid or to the free amine V in dry ethyl acetate at room temperature or at reflux (Sasse et al. J. Med. Chem. 2001, 44, 694-702). Other methods of converting amines to isocyanates rely on the generation of the isocyanate from an intermediate trichloro- or tribromoacetamide of formula XI (Braverman et al. Tet. Lett. 1999, 40, 3235-3238), obtained by treating amines with trichloro- or tribromoacetyl chloride, respectively. In a typical procedure, a trihalo-acetamide XI in dry DMSO or acetonitrile in the presence of equimolar amounts of DBU is heated at 60-80° C. for 0.5-4 hours. Similarly, isocyanates of formula X are also obtained from amines and di-tert-butyldicarbonate in the presence of dimethylaminopyridine (Knölker et al. Angew. Chem. Int. Ed. Engl. 1995, 34, 2497-2500). In a typical procedure, di-tert-butyldicarbonate is treated sequentially with equimolar amounts of 4-dimethylaminopyridine and amine in acetonitrile, then the mixture is stirred for 10 min to 1 hour at room temperature. The mixture is treated with a solvent, preferably dichloromethane, and sulfuric acid is added afterwards as a 40% solution in acetonitrile. Isocyanate X is extracted in an organic solvent, preferably a halogenated solvent, and afterwards is purified by methods well-known in the field.

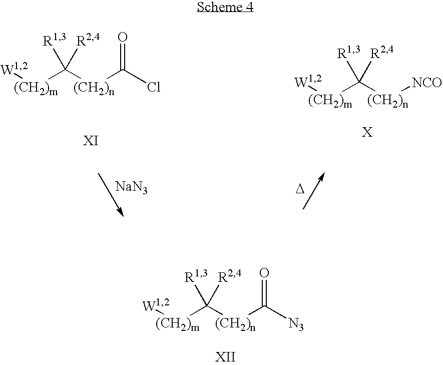

Isocyanates can also be prepared in situ from acyl azides XII (Y═COOH) via a Curtius rearrangement (Smith and March, ADVANCED ORGANIC CHEMISTRY: REACTIONS, MECHANISMS AND STRUCTURES, 5^{th} Ed.; Wiley: New York, 2001; p 1412, and references cited herein, White et al. J. Med. Chem. 1996, 39, 4382-4395) as depicted in Scheme 4. In a typical procedure, the acyl halide XI is treated with sodium azide in an organic solvent, preferably acetone, at temperatures from ambient to reflux, then the product is separated by methods such as column chromatography, distillation or recrystallization to yield acyl azide XII, which is then taken up in an appropriate organic solvent, preferably toluene, and heated to temperatures ranging from 40 to 120° C. to yield the isocyanate X.

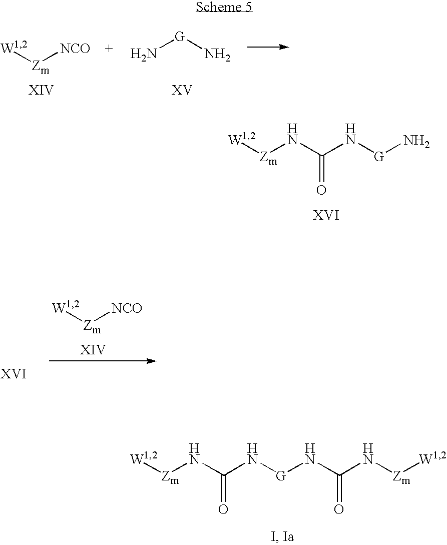

Scheme 5 illustrates methods of synthesis of both symmetric and asymmetric urea compounds of formulas I and Ia (Z=CH₂). In a typical experiment, an isocyanate derivative XIV is treated with a diamine XV in conditions similar to the ones described above, and then intermediate XVI as a crude or purified product is further treated with a second mole of isocyanate XIV to yield a urea of formula I or Ia.

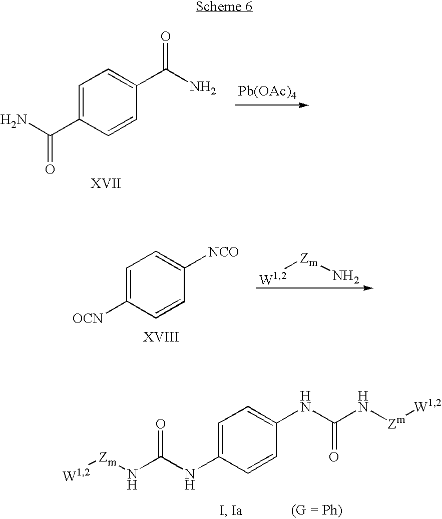

Scheme 6 illustrates methods of synthesis for both symmetric and asymmetric aryl ureas (G=phenyl) of formula I and Ia (when Z=CH₂) Aryl diamides of the general formula XVII may be transformed into their corresponding isocyanates XVIII via a Hofmann rearrangement effected by treatment with lead tetraacetate (see March, ADVANCED ORGANIC CHEMISTRY: REACTIONS, MECHANISMS AND STRUCTURES, 1091, 4^{th} Ed., 1992, pp. 1090-1091 and references cited herein). Isocyanates of formula XVIII may then be converted to aryl ureas of formulas I and Ia upon reaction with an appropriate amine as previously described.

Urea compounds of all general formulas (I, Ia-c, II and IIa) may also be prepared from the corresponding thioureas (see Schemes 12 and 13 for general methodologies used to prepare thiourea compounds of the invention) by oxidation in the presence of various oxidizing agents, as illustrated in Scheme 7. Typical oxidants employed for this transformation include manganese dioxide in dichloromethane, t-butyl hypochlorite in carbon tetrachloride, sodium nitrite in hydrochloric acid 4M, mercuric acetate in dichloromethane, and preferably hydrogen peroxide in sodium hydroxide (as reference see McKay et al. Can. J. Chem. 1958, 36, 147-150, Hegarty and Drennan, in COMPREHENSIVE ORGANIC FUNCTIONAL GROUP TRANSFORMATIONS, A. R. Katritzky, O. Meth-Cohn and C. W. Rees, Eds., Pergamon: Oxford 1995, Vol. 6, p. 499-525).

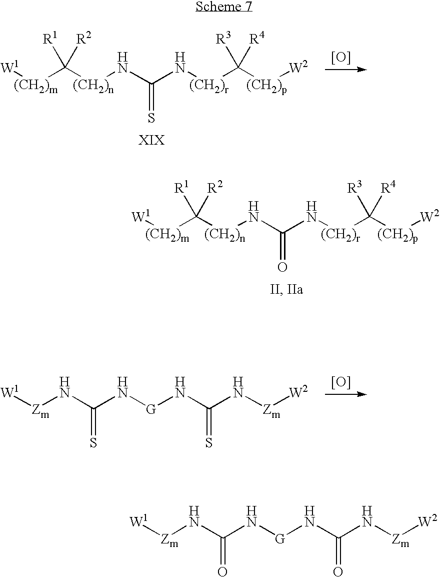

In a typical procedure, a thiourea (represented by structures XIX and XX) is dissolved in a mixture of an alcohol and aqueous sodium or potassium hydroxide, preferably ethanol and 30% sodium hydroxide. The solution is treated with a 50% solution of hydrogen peroxide at temperatures of −20 to 0° C., then the reaction is allowed to reach the ambient temperature or heated at up to 40° C., until no more change in the starting material is detected by an analytical method, such as HPLC or tlc. The reaction is then quenched by adding water, and the product is extracted or recrystallized. The crude urea is purified by known methods such as recrystallization, chromatography, etc.

Schemes 8-11 illustrate the syntheses of urea compounds of formula II and Ia. Their preparation rely on methods well-known in the field that have been described and reviewed previously (Hegarty and Drennan, in COMPREHENSIVE ORGANIC FUNCTIONAL GROUP TRANSFORMATIONS, A. R. Katritzky, O. Meth-Cohn and C. W. Rees, Eds., Pergamon: Oxford 1995, Vol. 6, p. 499-525, Tanaka et al. J. Med. Chem. 1998, 41, 2390-2410). As illustrated in Scheme 8, ureas of formula II and IIa are readily obtained by reacting an amine of formula V with an isocyanate of formula X. In a typical experiment, an amine is treated with an isocyanate in an appropriate solvent, at temperatures varying from 0° C. to room temperature or higher (reflux of the solvent) for 2 hours to 4 days, or until no more change in or disappearance of the starting materials is evidenced by an analytical method such as but not limited to NMR, GC, HPLC, or tlc. Suitable solvents are dichloromethane, diethyl ether, absolute ethanol, acetonitrile, chloroform, diglyme, toluene, anhydrous dioxane, anhydrous methanol and aqueous sodium hydroxide 2N, or mixtures of the above solvents, preferably dichloromethane. Examples are found in Tanaka et al. J. Med. Chem. 1998, 41, 2390-2410, Mounetou et al. J. Med. Chem. 2001, 44, 694-702, Hirst et al. J. Med. Chem. 1996, 39, 5236-5245, Scheerder et al. J. Org. Chem. 1995, 60, 6448-6454, Jensen et al. Acta Chem Scand. 1965, 19,438-442, Ryczek et al. Pol. J. Chem. 1996, 70, 1518-1521, Bamford et al. Bull Soc Chim. Belg. 1990, 99, 919-930, Artini et al. Arzneim. Forsch. 1971, 21, 30-36, Braverman et al. Tet. Lett. 1999, 40, 3235-3238, White et al. J. Med. Chem. 1996, 39, 4382-4395. Ureas of formulas II and IIa are also obtained from amines of formula V via either the trichloro- or tribromo-acetamide XXI as described above, via an elimination reaction with a second mole of amine V at temperatures from ambient to 80° C. (Braverman et al. Tet. Lett. 1999, 40, 3235-3238).

The synthesis of carbamates of formula XXII can be carried out as illustrated in Scheme 9 by treatment of an amine of formula V with a substituted chloroformate (ROCOCl), such as phenyl, p-nitrophenyl, 2,4,6-trichlorophenyl, benzyl, thioalkyl, and preferably p-nitrophenyl chloroformate, in the presence of an organic base, such as triethylamine or N,N-dimethylaniline, in a solvent such as toluene, 1,2-dichloroethane, chloroform, and preferably dichloromethane at 0-5° C. The reaction is brought to completion 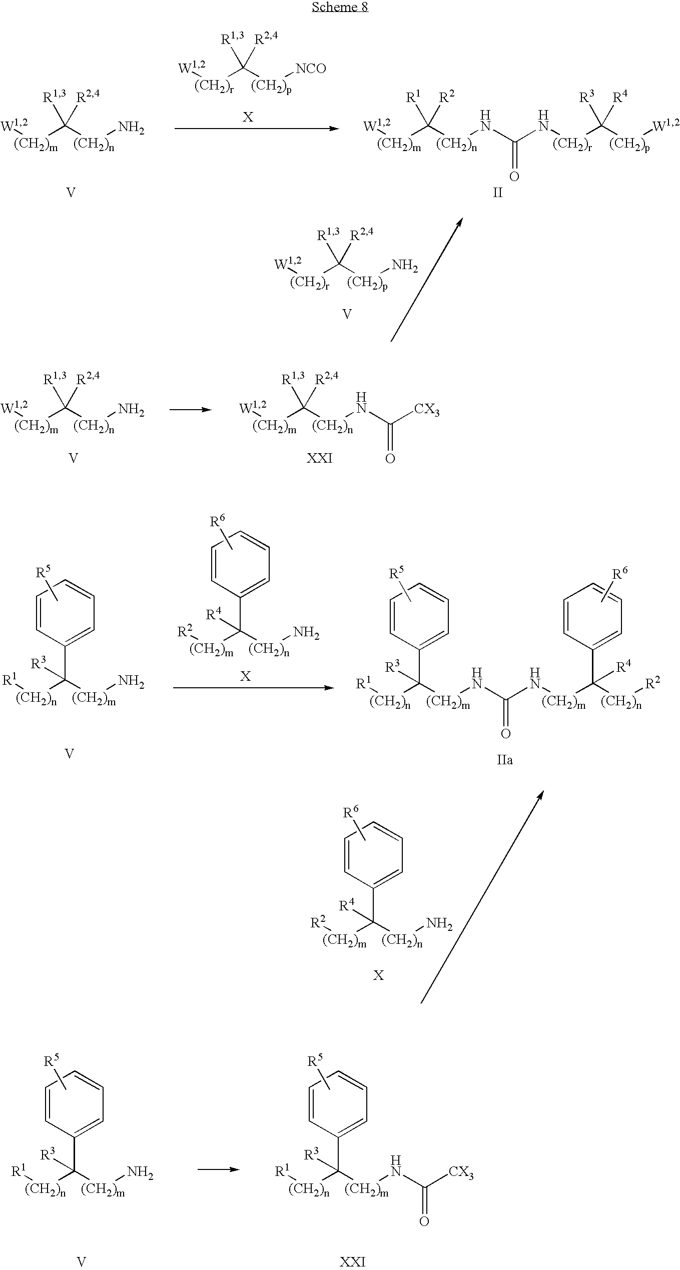
at ambient temperature or by heating under reflux for 3 to 24 hours (i.e., Tanaka et al. Bioorg. Med. Chem. 1988, 6, 15-30, Tanaka et al. J. Med. Chem. 1988, 41, 4408-4420). Chloroformates are commercially available reagents (e.g., Aldrich Chemical Co., Milwaukee, Wis.). Carbamates of the formula XXII may then be employed in the synthesis of ureas of formula II by treatment of an amine V with a phenyl, benzyl or thio N-substituted carbamate XXII most often in the presence of a base. Triethylamine, pyridine or N,N-dimethylaniline are typically utilized bases, preferably triethylamine. The reaction is performed in a solvent such as toluene, dimethylformamide, phenylmethanol, dioxane, chloroform or dichloromethane at temperatures between RT and reflux for 2 to 24 hours. Procedures are described in Tanaka et al. J. Med. Chem. 1998, 41, 2390-2410, Tanaka et al. J. Med. Chem. 1998, 41, 4408-4420, Crosby et al. J. Amer. Chem. Soc. 1954, 76, 4458, 4462, Shawali et al. J. Org. Chem. 1986, 51, 3498-3501, Loecsei et al. J. Prakt. Chem. 1982, 324, 816-826. In a typical procedure, a two- to ten-fold excess of amine V in a solvent, preferably dimethylformamide or dioxane, occasionally in the presence of equimolar amounts of a base such as triethylamine, is treated with carbamate at room temperature, and then heated at temperatures between 30 to 100° C. for 3-24 hours. The urea precipitates out as a solid or is extracted in an organic solvent, such as toluene, diethyl ether, t-butyl methyl ether or a low boiling point halogenated solvent, i.e. chloroform and dichloromethane. The crude product thus obtained is subjected to a suitable purification method, such as chromatography or recrystallization. Similarly, ureas of formula IIa may be prepared in this fashion by simply substituting the appropriate amine XX for amine V in Scheme 9.

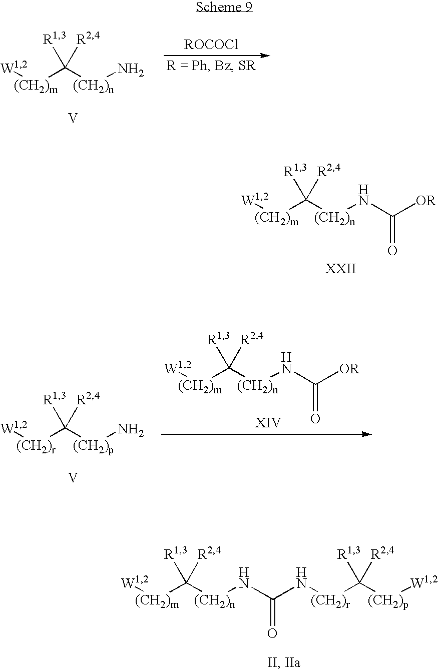

Scheme 10 illustrates the synthesis of ureas of formula II by treatment of an amine V with triphosgene (XVIIIa) at 0° C. to RT or at reflux in dichloromethane for 0.5 to 2 hours, then, after cooling, treatment of the trichloromethyl amido derivative XXIVa with an equivalent of the second amine in a solvent, preferably dichloromethane, at room temperature or higher for 2 to 24 hours (Kozikowski et al. J. Med. Chem. 2001, 44, 298-301, Tanaka et al. J. Med. Chem. 1998, 41, 2390-2410). Similarly, reaction of amine with bis(2H-imidazol-2-yl)-methanone or carbonyldiimidazole (XVIIIb) leads to intermediate XXIVb, which can then be further treated with a second mole of amine in equimolar amounts to afford asymmetric ureas of formula II. In a typical procedure, the amine, preferably in the presence of a base such as triethylamine, pyridine, or DMAP, dissolved in a solvent such as dichloromethane, chloroform, dimethylformamide, toluene, diglyme, preferably dichloromethane or dimethylformamide, is treated with carbonyldiimidazole for 24 to 48 h at 0° C. to room temperature. The solution is then evaporated in vacuo, and the residue is heated with a second equivalent of amine in dimethylformamide at temperatures in the range of 60 to 90° C. for 4 to 24 hours, or until no starting material is detected by a chromatographic method such as HPLC, GC or tlc, or other suitable analytical method (Keenan et al. Bioorg. Med. Chem. 1998, 6, 1309-1336, Hirst et al. J. Med. Chem. 1996, 39, 5236-5245, Zhang et al. J. Med. Chem. 1997, 40, 3498-3501). Ureas of formula IIa may be prepared by this methodology by simply replacing amine V in scheme 10 with amine X.

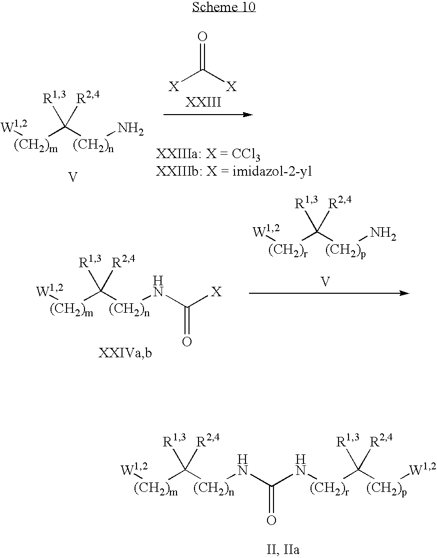

Amines V in the presence of DCC or other carbodiimides as condensing agents and triethylamine or pyridine as a base are treated with an excess amount of dry ice added gradually into a solution containing carbodiimide and triethylamine at −78° C. (i.e., Ogura et al. Synthesis 1978, 3498-3501). Finally, carbon dioxide is passed through a mixture of equimolar amounts of diphenyl phosphite and amine V in pyridine at 40° C. for 4 hours (i.e., Yamazaki et al. Tet. Lett. 1999, 40, 1191-1194). Also, by heating urea and an amine of formula V in water at reflux for 2-6 hours (Davis et al. ORGANIC SYNTHESES COLL. VOL. 1, Wiley: New York 1941, 453-455), or neat for 2 to 12 hours at 100-180° C. (Lavrov et al. J. Org. Chem. 1990, 26, 216-218) a symmetrically substituted urea of formula I is formed.

General methods utilized for the preparation of symmetrically substituted ureas of formula II are presented in Scheme 11. The synthesis of symmetric ureas of formula II and IIa by treatment of an amine of formula V with carbon dioxide (i.e., Kubota et al. J. Chem. Soc. Perkin Trans. 1 1993, 5-6, Ogura et al. Synthesis 1978, 3498-3501, Yamazaki et al. Tet. Lett. 1999, 40, 1191-1194). In a typical procedure, equimolar amounts of amine V, triphenylphosphine and triethylamine are taken up in an organic solvent, preferably acetonitrile, and the mixture is kept under carbon dioxide at atmospheric pressure in the presence of equivalent amounts of carbon tetrachloride at 0° C. to RT for 2 hours to 72 hours (i.e., Kubota et al. J. Chem. Soc. Perkin Trans.1 1993, 5-6). Alternatively, an amine of formula V can be treated with urea to yield a symmetrically substituted urea of formula II (i.e., Lavrov et al. J. Org. Chem. 1986, 51, 3498-3501, Davis et al. Organic Syntheses Coll. Vol. 1, Wiley: New York 1941, 453-455). Secondary amines of formula X will react with carbon monoxide in the presence of selenium or sulfur to provide symmetric ureas of formula x (see March, ADVANCED ORGANIC CHEMISTRY: REACTIONS, MECHANISMS AND STRUCTURES, 640, 4^{th} Ed., 1992 and references cited herein). In an additional procedure, symmetrically substituted ureas of formula II can also be obtained by treating a bromide or chloride of formula x (X=Br, Cl) with the sodium or potassium salt of cyanic acid in the presence of tetrabutylammonium bromide. See Scheme 9 for detailed descriptions of the procedures. Scheme 9 also illustrates the synthesis of symmetrical ureas of formula II and IIa from a bromide or chloride VI (X=Br, Cl) and cyanic acid (potassium or sodium salt) in the presence of tetrabutylammonium bromide (Dubosclard-Gottardi et al. Tetrahedron 1995, 51, 2561-2572). In a typical procedure the halide VI in acetonitrile is treated with sodium cyanate and a catalytic amount of tetrabutylammonium bromide (0.2 eq) in acetonitrile at room temperature, then water is added (1.2 to 1.5 equiv) with or without a catalyst, such as potassium iodide (0.2 eq). The mixture is heated at temperatures higher than 40° C., for up to 48 hours for bromide to up to 62 hours for chloride. After completion of the reaction, monitored by and analytical method, preferably GC or tlc, the mixture is cooled and the product is purified by standard purification methods, such as chromatography or recrystallization. Starting halo-derivatives VI are commercially available (e.g., Aldrich Chemical Co., Milwaukee, Wis.) or can be prepared as described in U.S. patent application Ser. No. 09/976,867, filed Oct. 11, 2001, which is incorporated herein by reference in its entirety.

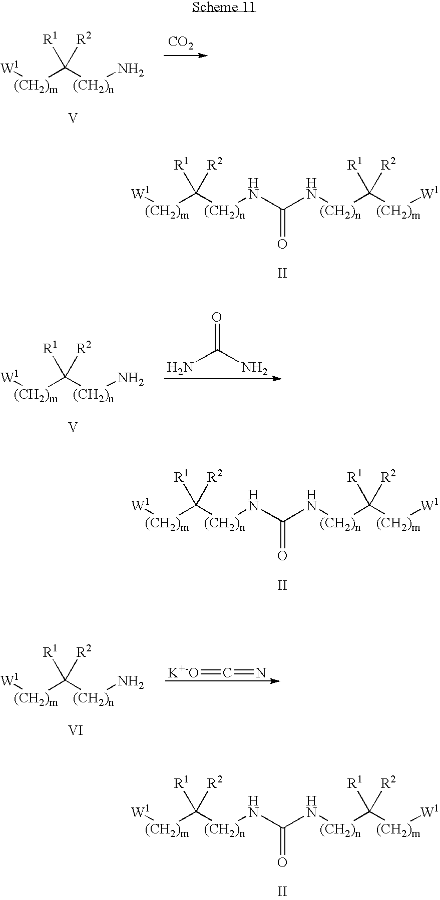

Scheme 12 illustrates various methodologies for the synthesis of symmetric thiourea compounds of the Invention. An amine of formula V can be reacted with carbon disulfide or potassium isothiocyanate to form symmetric thioureas of formula II.

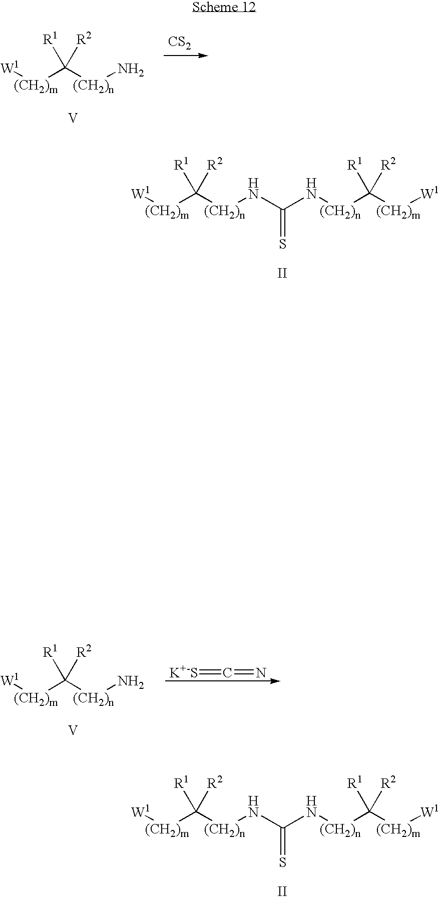

Shown in Scheme 13 is a method useful for the synthesis of symmetric and assymetric thioureas of formulas II and Ia, respectively. An amine of formula V can be reacted with thiocarbonyldiimidazole (XXV) to provide intermediate XXVI, which can then be reacted with a second amine of formula V in order to provide thioureas of formulas II and IIa.

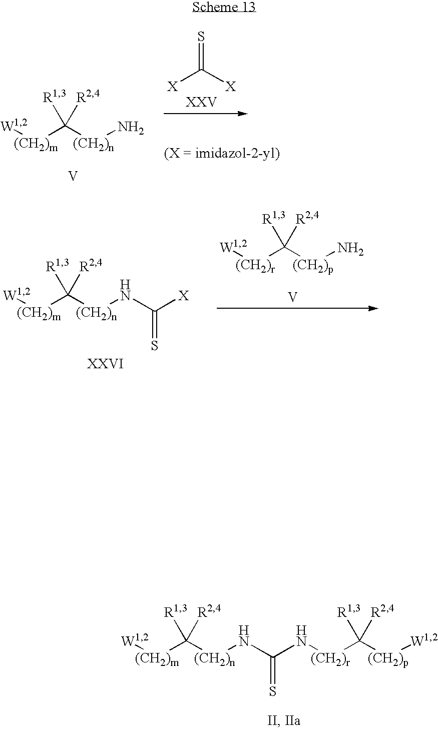

Scheme 14 illustrates the synthesis of urea compounds of the formula II, wherein m and n are each independently an integer from 2-5 and R¹ and R² are each independently phenyl, CO₂Me, CO₂Et, C(CH₃)₂CH₂OH or C(CH₃)(Ph)(CH₂OH).

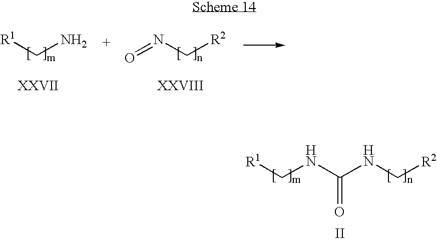

Urea compounds of formula II can be prepared via well-known methods (see, Larock, R. C. Comprehensive Organic Transformations; A Guide To Functional Group Preparations, 1989, pp 432, for a discussion of various methods see, March, J. Advanced Organic Chemistry; Reactions, Mechanisms, and Structure, 4th ed., 1992, pp 903, 904, 971, and 1091). For example, treatment of an amine with carbonmonoxide and a catalyst (e.g., selenium, see Sonoda, N; Yasuhara, T.; Kondo, K.; Ikeda, T.; Tsutsumi, S., J. Am. Chem. Soc., 1971, 93, 6344, or sulfur, see Franz, R. A., Applegath, F.; Morriss, F. V.; Baiocchi, F.; Bolze, C., J. Org. Chem., 1961, 26, 3309), treatment of an amine with carbondioxide and a catalyst (e.g., ruthenium complexes, see Fournier, J., Bruneau, C., Dixneuf, P. H., Lécolier, S., J. Org. Chem., 1991, 56, 4456), treatment of an alcohol with a cyanamide (e.g., see Anatol, J.; Berecoechea, J., Synthesis, 1975, 111), treatment of an amine with phosgene (e.g., see Petersen, U. in Methoden der Organischen Chemie, 1983, p 334), treatment of an amine with a chloroformate (e.g., see Abrahart, E. N., J. Chem. Soc., 1936, 1273), treatment of an amine with a carbonate (e.g., see Takeda, K.; Ogura, H., Synth. Commun., 1982, 12, 213), treatment of an amine with a formamide (e.g., see Kotachi, S.; Tsuji, Y.; Kondo, T.; Watanabe, Y., J. Chem. Soc., Chem. Commun., 1990, 549), treatment of an amine with a carbamate (e.g., see Petersen, U. in Methoden der Organischen Chemie, 1983, p 334 or Basha, A., Tetrahedron Lett., 1988, 29, 2525), or treatment of an amine with an isocyanate (e.g., see Petersen, U. in Methoden der Organischen Chemie, 1983, p 334 or Ozaki, S., Chem. Rev., 1972, 72, 457).

For example, a compound of formula II can be prepared by treatment of isocyanate XVIII with amine XVII at preferably, though not limited to, temperatures between 0° C. and room temperature. Preferably, though not limited, the reaction is run in an aprotic solvent, for example CH₂Cl₂. Isocyanate XVIII can for example be prepared from amine XVII by treatment with e.g. oxalyl chloride (Ozaki, S., Chem. Rev., 1972, 72, 457), phosgene (Saunders, J. H.; Slocombe, R. J., Chem. Rev., 1948, 43, 203, or a synthetic phosgene equivalent like diphoshene or preferably triphosgene.

### 4.3 Therapeutic Uses of Compounds or Compositions of the Invention

In accordance with the invention, a compound of the invention or a composition of the invention, comprising a compound of the invention and a pharmaceutically acceptable vehicle, is administered to a patient, preferably a human, with or at risk of aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. In one embodiment, “treatment” or “treating” refers to an amelioration of a disease or disorder, or at least one discernible symptom thereof. In another embodiment, “treatment” or “treating” refers to inhibiting the progression of a disease or disorder, either physically, e.g., stabilization of a discernible symptom, physiologically, e.g., stabilization of a physical parameter, or both.

In certain embodiments, the compounds of the invention or the compositions of the invention are administered to a patient, preferably a human, as a preventative measure against such diseases. As used herein, “prevention” or “preventing” refers to a reduction of the risk of acquiring a given disease or disorder. In a preferred mode of the embodiment, the compositions of the present invention are administered as a preventative measure to a patient, preferably a human having a genetic predisposition to a aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. Examples of such genetic predispositions include but are not limited to the ±4 allele of apolipoprotein E, which increases the likelihood of Alzheimer's Disease; a loss of function or null mutation in the lipoprotein lipase gene coding region or promoter (e.g., mutations in the coding regions resulting in the substitutions D9N and N291S; for a review of genetic mutations in the lipoprotein lipase gene that increase the risk of cardiovascular diseases, dyslipidemias and dyslipoproteinemias, see Hayden and Ma, 1992, Mol. Cell Biochem. 113:171-176); and familial combined hyperlipidemia and familial hypercholesterolemia.

In another preferred mode of the embodiment, the compounds of the invention or compositions of the invention are administered as a preventative measure to a patient having a non-genetic predisposition to a aging, Alzheimer's Disease, cancer, cardiovascular disease, diabetic nephropathy, diabetic retinopathy, a disorder of glucose metabolism, dyslipidemia, dyslipoproteinemia, enhancing bile production, enhancing reverse lipid transport, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. Examples of such non-genetic predispositions include but are not limited to cardiac bypass surgery and percutaneous transluminal coronary angioplasty, which often lead to restenosis, an accelerated form of atherosclerosis; diabetes in women, which often leads to polycystic ovarian disease; and cardiovascular disease, which often leads to impotence. Accordingly, the compositions of the invention may be used for the prevention of one disease or disorder and concurrently treating another (e.g., prevention of polycystic ovarian disease while treating diabetes; prevention of impotence while treating a cardiovascular disease).

### 4.3.1 Treatment of Cardiovascular Diseases

The present invention provides methods for the treatment or prevention of a cardiovascular disease, comprising administering to a patient a therapeutically effective amount of a compound or a composition comprising a compound of the invention and a pharmaceutically acceptable vehicle. As used herein, the term “cardiovascular diseases” refers to diseases of the heart and circulatory system. These diseases are often associated with dyslipoproteinemias and/or dyslipidemias. Cardiovascular diseases which the compositions of the present invention are useful for preventing or treating include but are not limited to arteriosclerosis; atherosclerosis; stroke; ischemia; endothelium dysfunctions, in particular those dysfunctions affecting blood vessel elasticity; peripheral vascular disease; coronary heart disease; myocardial infarcation; cerebral infarction and restenosis.

### 4.3.2 Treatment of Dyslipidemias

The present invention provides methods for the treatment or prevention of a dyslipidemia comprising administering to a patient a therapeutically effective amount of a compound or a composition comprising a compound of the invention and a pharmaceutically acceptable vehicle.

As used herein, the term “dyslipidemias” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (http://www.americanheart.org/cholesterol/ about_level.html and http://www.nhlbi.nih.gov/health/public/heart/chol/hbc_what.html, respectively). At the present time, the recommended level of HDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

Dyslipidemias which the compositions of the present invention are useful for preventing or treating include but are not limited to hyperlipidemia and low blood levels of high density lipoprotein (HDL) cholesterol. In certain embodiments, the hyperlipidemia for prevention or treatment by the compounds of the present invention is familial hypercholesterolemia; familial combined hyperlipidemia; reduced or deficient lipoprotein lipase levels or activity, including reductions or deficiencies resulting from lipoprotein lipase mutations; hypertriglyceridemia; hypercholesterolemia; high blood levels of urea bodies (e.g. β-OH butyric acid); high blood levels of Lp(a) cholesterol; high blood levels of low density lipoprotein (LDL) cholesterol; high blood levels of very low density lipoprotein (VLDL) cholesterol and high blood levels of non-esterified fatty acids.

The present invention further provides methods for altering lipid metabolism in a patient, e.g., reducing LDL in the blood of a patient, reducing free triglycerides in the blood of a patient, increasing the ratio of HDL to LDL in the blood of a patient, and inhibiting saponified and/or non-saponified fatty acid synthesis, said methods comprising administering to the patient a compound or a composition comprising a compound of the invention in an amount effective alter lipid metabolism.

### 4.3.3 Treatment of Dyslipoproteinemias

The present invention provides methods for the treatment or prevention of a dyslipoproteinemia comprising administering to a patient a therapeutically effective amount of a compound or a composition comprising a compound of the invention and a pharmaceutically acceptable vehicle.

As used herein, the term “dyslipoproteinemias” refers to disorders that lead to or are manifested by aberrant levels of circulating lipoproteins. To the extent that levels of lipoproteins in the blood are too high, the compositions of the invention are administered to a patient to restore normal levels. Conversely, to the extent that levels of lipoproteins in the blood are too low, the compositions of the invention are administered to a patient to restore normal levels. Normal levels of lipoproteins are reported in medical treatises known to those of skill in the art.

Dyslipoproteinemias which the compositions of the present invention are useful for preventing or treating include but are not limited to high blood levels of LDL; high blood levels of apolipoprotein B (apo B); high blood levels of Lp(a); high blood levels of apo(a); high blood levels of VLDL; low blood levels of HDL; reduced or deficient lipoprotein lipase levels or activity, including reductions or deficiencies resulting from lipoprotein lipase mutations; hypoalphalipoproteinemia; lipoprotein abnormalities associated with diabetes; lipoprotein abnormalities associated with obesity; lipoprotein abnormalities associated with Alzheimer's Disease; and familial combined hyperlipidemia.

The present invention further provides methods for reducing apo C-II levels in the blood of a patient; reducing apo C-III levels in the blood of a patient; elevating the levels of HDL associated proteins, including but not limited to apo A-I, apo A-II, apo A-IV and apo E in the blood of a patient; elevating the levels of apo E in the blood of a patient, and promoting clearance of triglycerides from the blood of a patient, said methods comprising administering to the patient a compound or a composition comprising a compound of the invention in an amount effective to bring about said reduction, elevation or promotion, respectively.

### 4.3.4 Treatment of Glucose Metabolism Disorders

The present invention provides methods for the treatment or prevention of a glucose metabolism disorder, comprising administering to a patient a therapeutically effective amount of a compound or a composition comprising a compound of the invention and a pharmaceutically acceptable vehicle. As used herein, the term “glucose metabolism disorders” refers to disorders that lead to or are manifested by aberrant glucose storage and/or utilization. To the extent that indicia of glucose metabolism (i.e., blood insulin, blood glucose) are too high, the compositions of the invention are administered to a patient to restore normal levels. Conversely, to the extent that indicia of glucose metabolism are too low, the compositions of the invention are administered to a patient to restore normal levels. Normal indicia of glucose metabolism are reported in medical treatises known to those of skill in the art.

Glucose metabolism disorders which the compositions of the present invention are useful for preventing or treating include but are not limited to impaired glucose tolerance; insulin resistance; insulin resistance related breast, colon or prostate cancer; diabetes, including but not limited to non-insulin dependent diabetes mellitus (NIDDM), insulin dependent diabetes mellitus (IDDM), gestational diabetes mellitus (GDM), and maturity onset diabetes of the young (MODY); pancreatitis; hypertension; polycystic ovarian disease; and high levels of blood insulin and/or glucose.

The present invention further provides methods for altering glucose metabolism in a patient, for example to increase insulin sensitivity and/or oxygen consumption of a patient, said methods comprising administering to the patient a compound or a composition comprising a compound of the invention in an amount effective to alter glucose metabolism.

### 4.3.5 Treatment of PPAR-Associated Disorders

The present invention provides methods for the treatment or prevention of a PPAR-associated disorder, comprising administering to a patient a therapeutically effective amount of a compound or a composition comprising a compound of the invention and a pharmaceutically acceptable vehicle. As used herein, “treatment or prevention of PPAR associated disorders” encompasses treatment or prevention of rheumatoid arthritis; multiple sclerosis; psoriasis; inflammatory bowel diseases; breast; colon or prostate cancer; low levels of blood HDL; low levels of blood, lymph and/or cerebrospinal fluid apo E; low blood, lymph and/or cerebrospinal fluid levels of apo A-I; high levels of blood VLDL; high levels of blood LDL; high levels of blood triglyceride; high levels of blood apo B; high levels of blood apo C-III and reduced ratio of post-heparin hepatic lipase to lipoprotein lipase activity. HDL may be elevated in lymph and/or cerebral fluid.

### 4.3.6 Treatment of Renal Diseases

The present invention provides methods for the treatment or prevention of a renal disease, comprising administering to a patient a therapeutically effective amount of a compound or a composition comprising a compound of the invention and a pharmaceutically acceptable vehicle. Renal diseases that can be treated by the compounds of the present invention include glomerular diseases (including but not limited to acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (including but not limited to acute tubular necrosis and acute renal failure, polycystic renal diseasemedullary sponge kidney, medullary cystic disease, nephrogenic diabetes, and renal tubular acidosis), tubulointerstitial diseases (including but not limited to pyelonephritis, drug and toxin induced tubulointerstitial nrephritis, hypercalcemic nephropathy, and hypokalemic nephropathy) acute and rapidly progressive renal failure, chronic renal failure, nephrolithiasis, or tumors (including but not limited to renal cell carcinoma and nephroblastoma). In a most preferred embodiment, renal diseases that are treated by the compounds of the present invention are vascular diseases, including but not limited to hypertension, nephrosclerosis, microangiopathic hemolytic anemia, atheroembolic renal disease, diffuse cortical necrosis, and renal infarcts.

### 4.3.7 Treatment of Cancer

The present invention provides methods for the treatment or prevention of cancer, comprising administering to a patient a therapeutically effective amount of a compound or a composition comprising a compound of the invention and a pharmaceutically acceptable vehicle. Types of cancer that can be treated using a Compound of the Invention include, but are not limited to, those listed in Table 2.

TABLE 2

Solid tumors, including but not limited to
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophogeal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
glioma
glioblastoma multiforme
astrocytoma
medulloblastoma
craniopharyngioma
ependymoma
pinealoma
hemangioblastoma
acoustic neuroma
oligodendroglioma
meningioma
skin cancer
melanoma
neuroblastoma
retinoblastoma
Blood-borne cancers, including but not limited to:
acute lymphoblastic B-cell leukemia
acute lymphoblastic T-cell leukemia
acute myeloblastic leukemia “AML”
acute promyelocytic leukemia “APL”
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia “CML”
chronic lymphocytic leukemia “CLL”
hairy cell leukemia
multiple myeloma
Acute and chronic leukemias
Lymphoblastic
myelogenous
lymphocytic
myelocytic leukemias
Lymphomas:
Hodgkin's disease
non-Hodgkin's Lymphoma
Multiple myeloma
Waldenström's macroglobulinemia
Heavy chain disease
Polycythemia vera

Cancer, including, but not limited to, a tumor, metastasis, or any disease or disorder characterized by uncontrolled cell growth, can be treated or prevented by administration of a Compound of the Invention.

### 4.3.8 Treatment of Other Diseases

The present invention provides methods for the treatment or prevention of Alzheimer's Disease, Syndrome X, septicemia, thrombotic disorders, obesity, pancreatitis, hypertension, inflammation, and impotence, comprising administering to a patient a therapeutically effective amount of a compound or a composition comprising a compound of the invention and a pharmaceutically acceptable vehicle.

As used herein, “treatment or prevention of Alzheimer's Disease” encompasses treatment or prevention of lipoprotein abnormalities associated with Alzheimer's Disease.

As used herein, “treatment or prevention of Syndrome X or Metabolic Syndrome” encompasses treatment or prevention of a symptom thereof, including but not limited to impaired glucose tolerance, hypertension and dyslipidemia/dyslipoproteinemia.

As used herein, “treatment or prevention of septicemia” encompasses treatment or prevention of septic shock.

As used herein, “treatment or prevention of thrombotic disorders” encompasses treatment or prevention of high blood levels of fibrinogen and promotion of fibrinolysis.

In addition to treating or preventing obesity, the compositions of the invention can be administered to an individual to promote weight reduction of the individual.

As used herein, “treatment or prevention of diabetic nephropathy” encompasses treating or preventing kidney disease that develops as a result of diabetes mellitus (DM). Diabetes mellitus is a disorder in which the body is unable to metabolize carbohydrates (e.g., food starches, sugars, cellulose) properly. The disease is characterized by excessive amounts of sugar in the blood (hyperglycemia) and urine; inadequate production and/or utilization of insulin; and by thirst, hunger, and loss of weight. Thus, the compounds of the invention can also be used to treat or prevent diabetes mellitus.

As used herein, “treatment or prevention of diabetic retinopathy” encompasses treating or preventing complications of diabetes that lead to or cause blindness. Diabetic retinopathy occurs when diabetes damages the tiny blood vessels inside the retina, the light-sensitive tissue at the back of the eye.

As used herein, “treatment or prevention of impotence” includes treating or preventing erectile dysfunction, which encompasses the repeated inability to get or keep an erection firm enough for sexual intercourse. The word “impotence” may also be used to describe other problems that interfere with sexual intercourse and reproduction, such as lack of sexual desire and problems with ejaculation or orgasm. The term “treatment or prevention of impotence includes, but is not limited to impotence that results as a result of damage to nerves, arteries, smooth muscles, and fibrous tissues, or as a result of disease, such as, but not limited to, diabetes, kidney disease, chronic alcoholism, multiple sclerosis, atherosclerosis, vascular disease, and neurologic disease.

As used herein, “treatment or prevention of hypertension” encompasses treating or preventing blood flow through the vessels at a greater than normal force, which strains the heart; harms the arteries; and increases the risk of heart attack, stroke, and kidney problems. The term hypertension includes, but is not limited to, cardiovascular disease, essential hypertension, hyperpiesia, hyperpiesis, malignant hypertension, secondary hypertension, or white-coat hypertension.

As used herein, “treatment or prevention of inflammation” encompasses treating or preventing inflammation diseases including, but not limited to, chronic inflammatory disorders of the joints including arthritis, e.g., rheumatoid arthritis and osteoarthritis; respiratory distress syndrome, inflammatory bowel diseases such as ileitis, ulcerative colitis and Crohn's disease; and inflammatory lung disorders such as asthma and chronic obstructive airway disease, inflammatory disorders of the eye such as corneal dystrophy, trachoma, onchocerciasis, uveitis, sympathetic ophthalmitis, and endophthalmitis; inflammatory disorders of the gum, e.g., periodontitis and gingivitis; tuberculosis; leprosy; inflammatory diseases of the kidney including glomerulonephritis and nephrosis; inflammatory disorders of the skin including acne, sclerodermatitis, psoriasis, eczema, photoaging and wrinkles; inflammatory diseases of the central nervous system, including AIDS-related neurodegeneration, stroke, neurotrauma, Alzheimer's disease, encephalomyelitis and viral or autoimmune encephalitis; autoimmune diseases including immune-complex vasculitis, systemic lupus and erythematodes; systemic lupus erythematosus (SLE); and inflammatory diseases of the heart such as cardiomyopathy.

### 4.4 Combination Therapy

In certain embodiments of the present invention, the compounds and compositions of the invention can be used in combination therapy with at least one other therapeutic agent. The compound of the invention and the therapeutic agent can act additively or, more preferably, synergistically. In a preferred embodiment, a compound or a composition comprising a compound of the invention is administered concurrently with the administration of another therapeutic agent, which can be part of the same composition as the compound of the invention or a different composition. In another embodiment, a compound or a composition comprising a compound of the invention is administered prior or subsequent to administration of another therapeutic agent. As many of the disorders for which the compounds and compositions of the invention are useful in treating are chronic disorders, in one embodiment combination therapy involves alternating between administering a compound or a composition comprising a compound of the invention and a composition comprising another therapeutic agent, e.g., to minimize the toxicity associated with a particular drug. The duration of administration of each drug or therapeutic agent can be, e.g., one month, three months, six months, or a year. In certain embodiments, when a composition of the invention is administered concurrently with another therapeutic agent that potentially produces adverse side effects including but not limited to toxicity, the therapeutic agent can advantageously be administered at a dose that falls below the threshold at which the adverse side is elicited.

The present compositions can be administered together with a statin. Statins for use in combination with the compounds and compositions of the invention include but are not limited to atorvastatin, pravastatin, fluvastatin, lovastatin, simvastatin, and cerivastatin.

The present compositions can also be administered together with a PPAR agonist, for example a thiazolidinedione or a fibrate. Thiazolidinediones for use in combination with the compounds and compositions of the invention include but are not limited to 5-((4-(2-(methyl-2-pyridinylamino)ethoxy)phenyl)methyl)-2,4-thiazolidinedione, troglitazone, pioglitazone, ciglitazone, WAY-120,744, englitazone, AD 5075, darglitazone, and rosiglitazone. Fibrates for use in combination with the compounds and compositions of the invention include but are not limited to gemfibrozil, fenofibrate, clofibrate, or ciprofibrate. As mentioned previously, a therapeutically effective amount of a fibrate or thiazolidinedione often has toxic side effects. Accordingly, in a preferred embodiment of the present invention, when a composition of the invention is administered in combination with a PPAR agonist, the dosage of the PPAR agonist is below that which is accompanied by toxic side effects.

The present compositions can also be administered together with a bile-acid-binding resin. Bile-acid-binding resins for use in combination with the compounds and compositions of the invention include but are not limited to cholestyramine and colestipol hydrochloride. The present compositions can also be administered together with niacin or nicotinic acid. The present compositions can also be administered together with a RXR agonist. RXR agonists for use in combination with the compounds of the invention include but are not limited to LG 100268, LGD 1069, 9-cis retinoic acid, 2-(1-(3,5,5,8,8-pentamethyl-5,6,7,8-tetrahydro-2-naphthyl)-cyclopropyl)-pyridine-5-carboxylic acid, or 4-((3,5,5,8,8-pentamethyl-5,6,7,8-tetrahydro-2-naphthyl)2-carbonyl)-benzoic acid. The present compositions can also be administered together with an anti-obesity drug. Anti-obesity drugs for use in combination with the compounds of the invention include but are not limited to β-adrenergic receptor agonists, preferably β-3 receptor agonists, fenfluramine, dexfenfluramine, sibutramine, bupropion, fluoxetine, and phentermine. The present compositions can also be administered together with a hormone. Hormones for use in combination with the compounds of the invention include but are not limited to thyroid hormone, estrogen and insulin. Preferred insulins include but are not limited to injectable insulin, transdermal insulin, inhaled insulin, or any combination thereof. As an alternative to insulin, an insulin derivative, secretagogue, sensitizer or mimetic may be used. Insulin secretagogues for use in combination with the compounds of the invention include but are not limited to forskolin, dibutryl cAMP or isobutylmethylxanthine (IBMX).

The present compositions can also be administered together with a phosphodiesterase type 5 (“PDE5”) inhibitor to treat or prevent disorders, such as but not limited to, impotence. In a particular, embodiment the combination is a synergistic combination of a composition of the invention and a PDE5 inhibitor.

The present compositions can also be administered together with a tyrophostine or an analog thereof. Tyrophostines for use in combination with the compounds of the invention include but are not limited to tryophostine 51.

The present compositions can also be administered together with sulfonylurea-based drugs. Sulfonylurea-based drugs for use in combination with the compounds of the invention include, but are not limited to, glisoxepid, glyburide, acetohexamide, chlorpropamide, glibornuride, tolbutamide, tolazamide, glipizide, gliclazide, gliquidone, glyhexamide, phenbutamide, and tolcyclamide. The present compositions can also be administered together with a biguanide. Biguanides for use in combination with the compounds of the invention include but are not limited to metformin, phenformin and buformin.

The present compositions can also be administered together with an α-glucosidase inhibitor. α-glucosidase inhibitors for use in combination with the compounds of the invention include but are not limited to acarbose and miglitol.

The present compositions can also be administered together with an apo A-I agonist. In one embodiment, the apo A-I agonist is the Milano form of apo A-I (apo A-IM). In a preferred mode of the embodiment, the apo A-IM for administration in conjunction with the compounds of the invention is produced by the method of U.S. Pat. No. 5,721,114 to Abrahamsen. In a more preferred embodiment, the apo A-I agonist is a peptide agonist. In a preferred mode of the embodiment, the apo A-I peptide agonist for administration in conjunction with the compounds of the invention is a peptide of U.S. Pat. No. 6,004,925 or 6,037,323 to Dasseux.

The present compositions can also be administered together with apolipoprotein E (apo E). In a preferred mode of the embodiment, the apoE for administration in conjunction with the compounds of the invention is produced by the method of U.S. Pat. No. 5,834,596 to Ageland.

In yet other embodiments, the present compositions can be administered together with an HDL-raising drug; an HDL enhancer; or a regulator of the apolipoprotein A-I, apolipoprotein A-IV and/or apolipoprotein genes.

In one embodiment, the other therapeutic agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron.

In another embodiment, the other therapeutic agent can be an hematopoietic colony stimulating factor. Suitable hematopoietic colony stimulating factors include, but are not limited to, filgrastim, sargramostim, molgramostim and erythropoietin alfa.

In still another embodiment, the other therapeutic agent can be an opioid or non-opioid analgesic agent. Suitable opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

### 4.4.1 Combination Therapy of Cardiovascular Diseases

The present compositions can be administered together with a known cardiovascular drug. Cardiovascular drugs for use in combination with the compounds of the invention to prevent or treat cardiovascular diseases include but are not limited to peripheral antiadrenergic drugs, centrally acting antihypertensive drugs (e.g., methyldopa, methyldopa HCl), antihypertensive direct vasodilators (e.g., diazoxide, hydralazine HCl), drugs affecting renin-angiotensin system, peripheral vasodilators, phentolamine, antianginal drugs, cardiac glycosides, inodilators (e.g., amrinone, milrinone, enoximone, fenoximone, imazodan, sulmazole), antidysrhythmic drugs, calcium entry blockers, ranitine, bosentan, and rezulin.

### 4.4.2 Combination Therapy of Cancer

The present invention includes methods for treating cancer, comprising administering to an animal in need thereof an effective amount of a Compound of the Invention and another therapeutic agent that is an anti-cancer agent. Suitable anticancer agents include, but are not limited to, those listed in Table 3.

TABLE 3

| | |
|---|---|
| Alkylating agents | |
| Nitrogen mustards: | Cyclophosphamide |
| | Ifosfamide |
| | trofosfamide |
| | Chlorambucil |
| | Treos |
| Nitrosoureas: | carbustine (BCNU) |
| | Lomustine (CCNU) |
| Alkylsulphonates | Busulfan |
| | Treosulfan |
| Triazenes: | Dacarbazine |
| Platinum containing compounds: | Cisplatin |
| | carboplatin |
| Plant Alkaloids | |
| Vinca alkaloids: | Vicristine |
| | Vinblastine |
| | Vindesine |
| | Vinorelbine |
| Taxoids: | paclitaxel |
| | Docetaxol |
| DNA Topoisomerase Inhibitors | |
| Epipodophyllins: | Etoposide |
| | Teniposide |
| | Topotecan |
| | 9-aminocamptothecin |
| | camptothecin |
| | crisnatol |
| mitomycins: | Mitomycin C |
| Anti-metabolites | |
| Anti-folates: | |
| DHFR inhibitors: | METHOTREXATE |
| | Trimetrexate |
| IMP dehydrogenase Inhibitors: | Mycophenolic acid |
| | Tiazofurin |
| | Ribavirin |
| | EICAR |
| Ribonuclotide reductase Inhibitors: | Hydroxyurea |
| | deferoxamine |
| Pyrimidine analogs: | |
| Uracil analogs | 5-Fluorouracil |
| | Floxuridine |
| | Doxifluridine |
| | Ratitrexed |
| Cytosine analogs | cytarabine (ara C) |
| | Cytosine arabinoside |
| | fludarabine |
| Purine analogs: | mercaptopurine |
| | Thioguanine |
| Hormonal therapies: | |
| Receptor antagonists: | |
| Anti-estrogen | Tamoxifen |
| | Raloxifene |
| | megestrol |
| | goscrclin |
| | Leuprolide acetate |
| LHRH agonists: | flutamide |
| | bicalutamide |
| Retinoids/Deltoids | |
| Vitamin D3 analogs: | EB 1089 |
| | CB 1093 |
| | KH 1060 |
| Photodynamic therapies: | vertoporfin (BPD-MA) |
| | Phthalocyanine |
| | photosensitizer Pc4 |
| | Demethoxy-hypocrellin A |
| | (2BA-2-DMHA) |
| Cytokines: | Interferon-α |
| | Interferon-γ |
| | Tumor necrosis factor |
| Others: | |
| Isoprenylation inhibitors: | Lovastatin |
| Dopaminergic neurotoxins: | 1-methyl-4-phenylpyridinium ion |
| Cell cycle inhibitors: | staurosporine |
| Actinomycines: | Actinomycin D |
| | Dactinomycin |
| Bleomycins: | bleomycin A2 |
| | Bleomycin B2 |
| | Peplomycin |
| Anthracyclines: | daunorubicin |
| | Doxorubicin (adriamycin) |
| | Idarubicin |
| | Epirubicin |
| | Pirarubicin |
| | Zorubicin |
| | Mitoxantrone |
| MDR inhibitors | verapamil |
| Ca²⁺ATPase inhibitors: | thapsigargin |

In a specific embodiment, a composition of the invention further comprises one or more chemotherapeutic agents and/or is administered concurrently with radiation therapy. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition of the invention.

In other embodiments, the invention provides methods for treating or preventing cancer, comprising administering to an animal in need thereof an effective amount of a Compound of the Invention and a chemotherapeutic agent. In one embodiment the chemotherapeutic agent is that with which treatment of the cancer has not been found to be refractory. In another embodiment, the chemotherapeutic agent is that with which the treatment of cancer has been found to be refractory. The Compounds of the Invention can be administered to an animal that has also undergone surgery as treatment for the cancer.

In one embodiment, the additional method of treatment is radiation therapy.

In a specific embodiment, the Compound of the Invention is administered concurrently with the chemotherapeutic agent or with radiation therapy. In another specific embodiment, the chemotherapeutic agent or radiation therapy is administered prior or subsequent to administration of a Compound of the Invention, preferably at least an hour, five hours, 12 hours, a day, a week, a month, more preferably several months (e.g., up to three months), prior or subsequent to administration of a Compound of the Invention.

A chemotherapeutic agent can be administered over a series of sessions, any one or a combination of the chemotherapeutic agents listed in Table 3 can be administered. With respect to radiation, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

Additionally, the invention provides methods of treatment of cancer with a Compound of the Invention as an alternative to chemotherapy or radiation therapy where the chemotherapy or the radiation therapy has proven or can prove too toxic, e.g., results in unacceptable or unbearable side effects, for the subject being treated. The animal being treated can, optionally, be treated with another cancer treatment such as surgery, radiation therapy or chemotherapy, depending on which treatment is found to be acceptable or bearable.

The Compounds of the Invention can also be used in an in vitro or ex vivo fashion, such as for the treatment of certain cancers, including, but not limited to leukemias and lymphomas, such treatment involving autologous stem cell transplants. This can involve a multi-step process in which the animal's autologous hematopoietic stem cells are harvested and purged of all cancer cells, the patient's remaining bone-marrow cell population is then eradicated via the administration of a high dose of a Compound of the Invention with or without accompanying high dose radiation therapy, and the stem cell graft is infused back into the animal. Supportive care is then provided while bone marrow function is restored and the animal recovers.

### 4.5 Surgical Uses

Cardiovascular diseases such as atherosclerosis often require surgical procedures such as angioplasty. Angioplasty is often accompanied by the placement of a reinforcing a metallic tube-shaped structure known as a “stent” into a damaged coronary artery. For more serious conditions, open heart surgery such as coronary bypass surgery may be required. These surgical procedures entail using invasive surgical devices and/or implants, and are associated with a high risk of restenosis and thrombosis. Accordingly, the compounds and compositions of the invention may be used as coatings on surgical devices (e.g., catheters) and implants (e.g., stents) to reduce the risk of restenosis and thrombosis associated with invasive procedures used in the treatment of cardiovascular diseases.

### 4.6 Veterinary and Livestock Uses

A composition of the invention can be administered to a non-human animal for a veterinary use for treating or preventing a disease or disorder disclosed herein.

In a specific embodiment, the non-human animal is a household pet. In another specific embodiment, the non-human animal is a livestock animal. In a preferred embodiment, the non-human animal is a mammal, most preferably a cow, horse, sheep, pig, cat, dog, mouse, rat, rabbit, or guinea pig. In another preferred embodiment, the non-human animal is a fowl species, most preferably a chicken, turkey, duck, goose, or quail.

In addition to veterinary uses, the compounds and compositions of the invention can be used to reduce the fat content of livestock to produce leaner meats. Alternatively, the compounds and compositions of the invention can be used to reduce the cholesterol content of eggs by administering the compounds to a chicken, quail, or duck hen. For non-human animal uses, the compounds and compositions of the invention can be administered via the animals' feed or orally as a drench composition.

### 4.7 Therapeutic/Prophylactic Administration and Compositions

Due to the activity of the compounds and compositions of the invention, they are useful in veterinary and human medicine. As described above, the compounds and compositions of the invention are useful for the treatment or prevention of aging, Alzheimer's Disease, cancer, cardiovascular disease, diabetic nephropathy, diabetic retinopathy, a disorder of glucose metabolism, dyslipidemia, dyslipoproteinemia, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, enhancing bile production, enhancing reverse lipid transport, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism.

The invention provides methods of treatment and prophylaxis by administration to a patient of a therapeutically effective amount of a compound or a composition comprising a compound of the invention. The patient is an animal, including, but not limited, to an animal such a cow, horse, sheep, pig, chicken, turkey, quail, cat, dog, mouse, rat, rabbit, guinea pig, etc., and is more preferably a mammal, and most preferably a human.

The compounds and compositions of the invention, are preferably administered orally. The compounds and compositions of the invention may also be administered by any other convenient route, for example, by intravenous infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds of the invention into the bloodstream.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In certain embodiments, for example, for the treatment of Alzheimer's Disease, it may be desirable to introduce one or more compounds of the invention into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds and compositions of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compounds and compositions of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target area to be treated, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions will contain a therapeutically effective amount of a compound of the invention, optionally more than one compound of the invention, preferably in purified form, together with a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In a preferred embodiment, the compounds and compositions of the invention are formulated in accordance with routine procedures as a pharmaceutical composition adapted for intravenous administration to human beings. Typically, compounds and compositions of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by intravenous infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compounds and compositions of the invention for oral delivery may be in the form of tablets, lozenges, aqueous or oily suspensions, granules, powders, emulsions, capsules, syrups, or elixirs. Compounds and compositions of the invention for oral delivery can also be formulated in foods and food mixes. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds and compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 2000 milligrams of a compound of the invention per kilogram body weight. In specific preferred embodiments of the invention, the oral dose is 0.01 milligram to 1000 milligrams per kilogram body weight, more preferably 0.1 milligram to 100 milligrams per kilogram body weight, more preferably 0.5 milligram to 25 milligrams per kilogram body weight, and yet more preferably 1 milligram to 10 milligrams per kilogram body weight. In a most preferred embodiment, the oral dose is 5 milligrams of a compound of the invention per kilogram body weight. The dosage amounts described herein refer to total amounts administered; that is, if more than one compound of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 milligram to 1000 milligrams per kilogram body weight, 0.1 milligram to 350 milligrams per kilogram body weight, and 1 milligram to 100 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a compound of the invention and another lipid-mediating compound, including but not limited to a statin, a thiazolidinedione, or a fibrate.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred for lowering fatty acid synthesis. The compounds and compositions of the invention may also be demonstrated to be effective and safe using animal model systems.

Other methods will be known to the skilled artisan and are within the scope of the invention.

The following examples are provided by way of illustration and not limitation.

### 5. SYNTHETIC EXAMPLES

### 5.1 Synthesis of Illustrative Compounds of the Invention

### 5.1.1 Preparation of Compound A

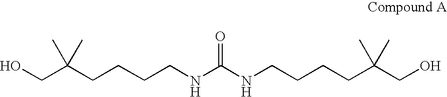

Under N₂ atmosphere, potassium phthalimide (49.1 g, 265.2 mmol) was added to a stirred solution of 2-(6-bromo-2,2-dimethylhexyloxy)-tetrahydropyran (70.7 g, 241.1 mmol) in DMF (150 mL, dried over 4 A molecular sieves) at rt. The suspension was heated to 80-95° C. for 3 h. The reaction mixture was cooled to rt, diluted with water (500 mL), and extracted with diethyl ether (2×250 mL, 1×100 mL). The combined organic layers were washed with saturated NaCl solution (100 mL), dried over MgSO₄, concentrated in vacuo, and dried under high vacuum to provide 2-(2,2-Dimethyl-6-phthalimido-hexyloxy)-tetrahydropyran as a yellow oil (78.4 g, 90%). ¹H NMR (300 MHZ, CDCl₃/TMS): δ (ppm): 7.84 (dd, 2H, J=5.4, 3.1 Hz), 7.71 (dd, 2H, J=5.4, 3.1 Hz), 4.53 (t, 1H, J=2.9 Hz), 3.81 (m, 1H), 3.68 (t, 2H, J=7.3 Hz), 3.48 (m, 1H), 3.46 (d, 1H, J=9.2 Hz), 2.97 (d, 1H, J=9.2 Hz), 2.97 (d, 1H, J=9.2 Hz), 1.90-1.42 (m, 9H), 1.31 (m, 3H), 0.89 (s, 3H), 0.88 (s, 3H). ¹³C NMR (75 MHZ, CDCl₃/TMS): δ (ppm): 168.42, 133.89, 132.26, 123.18, 99.09, 76.45, 61.88, 38.95, 38.13, 34.25, 30.70, 29.59, 25.65, 24.60, 21.44, 19.48. HRMS (LSIMS, POS, gly): Calcd. for C₁₆H₂₂NO₃ (MH⁺-DHP): 276.1600, found: 276.1597.

A solution of hydrazine hydrate in water (85% w/w, 17.3 g, 294.3 mmol) was added dropwise to a solution of 2-(2,2-dimethyl-6-phthalimido-hexyloxy)-tetrahydropyran (78.0 g, 216.98 mmol) in ethanol (400 mL) at rt. The reaction mixture was heated to reflux for 1 h, then cooled to rt. The precipitate was removed by filtration and washed with ethanol (2×100 mL). The filtrate was concentrated to a volume of ca. 100 mL. Further precipitate was filtered off and washed with diethyl ether (4×100 mL). The combined organic layers were washed with saturated NaCl solution (3×75 mL), dried over MgSO₄, concentrated in vacuo, and dried under high vacuum to provide 5,5-Dimethyl-6-(tetrahydro-pyran-2-yloxy)-hexylamine as a yellow oil (29.0 g, 58%). ¹H NMR (300 MHZ, CDCl₃/TMS), δ (ppm): 4.58 (s, 2H, OH), 4.54 (t, 1H, J=3.5 Hz), 3.82 (m, 1H), 3.49 (m, 4H), 3.46 (d, 1H, J=9.1 Hz), 2.98 (d, 1H, J=9.1 Hz), 2.78 (t, 2H, J=7.3 Hz), 1.94-1.44 (m, 8H), 1.40-1.20 (m, 4H), 0.89 (s, 6H). ¹³C NMR (75 MHZ, CDCl₃/TMS), δ (ppm): 99.08, 76.34, 61.89, 41.18, 38.91, 34.15, 32.29, 30.63, 25.53, 24.49, 21.17, 19.44. FRMS (LSIMS, nba): Calcd. for C₁₃H₂₈NO₂ (MH⁺): 230.2120, found: 230.2123.

To a solution of 5,5-dimethyl-6-(tetrahydropyran-2-yloxy)-hexylamine (14.9 g, 64.96 mmol) in pyridine (200 mL) was added triphenyl phosphite (10.1 g, 32.48 mmol). The solution was heated to 40-50° C. and CO₂ gas was introduced through a gas immersion tube (CO₂ was developed by evaporation of dry-ice and dried with Drierite. The duration of CO₂ introduction was estimated at ca. 8 h). The reaction mixture was cooled to rt after 18 h and a sample (2 mL of the solution) was taken for monitoring of the reaction progress by NMR spectroscopy (ratio product: starting material≈1:2). Additional triphenyl phosphite (10.2 g, 32.80 mmol) was added, CO₂ was introduced (for approximately 18 h), and the reaction mixture was heated to 55° C. for 23 h (NMR analysis indicated a ratio product: starting material≈2:1). The solvent was removed in vacuo and the reaction mixture concentrated under high vacuum to give the THP-protected product (53 g) as a yellow oil. The crude, protected urea (53 g) was dissolved in methanol (200 mL), then concd. HCl (20 mL) was added, and the mixture was heated to reflux for 3 h. The solution was cooled to rt, diluted with water (200 mL), and concentrated in vacuum to a volume of ca. 300 mL. The solution was extracted with CH₂Cl₂ (3×100 mL). The combined organic layers were washed with 10% NaOH solution (2×100 mL) and saturated NaCl solution (2×100 mL), dried over MgSO₄, concentrated in vacuo, and dried under high vacuum. The residual oil (5.30 g) was purified by flash chromatography (silica; hexanes/ethyl acetate=40/60, then 20/80, followed by chloroform/methanol=95/5) to provide Compound A as a yellow oil (2.24 g, 22% over two steps). ¹H NMR (300 MHZ, CD3OD/TMS), δ (ppm): 3.26 (s, 4H), 3.11 (t, 4H, J=6.8 Hz), 1.51-1.38 (m, 4H), 1.36-1.18 (m, 8H), 0.85 (s, 12H). ¹³C NMR (75 MHZ, CD3OD/TMS), δ (ppm): 161.50, 71.97, 41.08, 39.67, 36.07, 32.49, 24.59, 22.33. HRMS (LSIMS, gly): Calcd. for C₁₇H₃₇N₂O₃ (MH⁺): 317.2804, found: 317.2793.

### 5.1.2 Alternative Preparation of Compound A

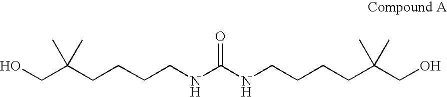

Using an alternative method, Compound A may be synthesized via the oxidation of the analogous thiourea [Compound A1]as described below.

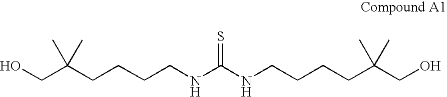

Under anhydrous conditions using a CaCl₂ drying tube, a solution of potassium phthalimide (261.6 g, 1.15 mol) and 6-bromo-2,2-dimethyl-1-hexanol (253.9 g, 1.38 mol) in DMF (1.7 L) was heated to 80-90° C. for two hours. The solvent was evaporated under vacuum. The residue was suspended in water (2 L) and extracted with ethyl acetate (3×1 L). The combined organic layers were washed with distilled water (2×2 L), dried over MgSO₄, and treated with activated carbon. The solution was concentrated in vacuo to give the product as a beige wax (265.95 g, 84% yield). A small portion (14.87 g) was re-crystallized from hexanes (400 mL) to provide 6-Phthalimido-2,2-dimethyl-1-hexanol as a crystalline solid (5.25 g, 37% recovery). Mp.: 74.8-75.0° C. ¹H NMR (300 MHZ, CDCl₃/TMS): δ (ppm): 7.82 (m, 2H), 7.72 (m, 2H), 3.71 (t, 2H, J=7.0 Hz), 3.30 (s, 2H), 1.65 (m, 2H), 1.31 (m, 4H), 0.87 (s, 12H). ¹³C NMR (75 MHZ, CDCl₃/TMS): δ (ppm): 168.7, 134.1, 132.3, 123.3, 71.6, 38.0, 35.2, 29.5, 24.1, 21.1.

To a solution of 6-phthalimido-2,2-dimethyl-1-hexanol (250.8 g, 893 mmol) in ethanol (3 L) was added hydrazine monohydrate (90 g, 1.52 mol). The mixture was refluxed for 45 min under formation of phthalhydrazide as a voluminous, white solid. After cooling to rt, the solid mass was suspended in methylene chloride (6 L) and filtered. The solution was concentrated and the residue was suspended in hexanes and filtered in order to remove residual phthalhydrazide. The filtrate was concentrated in vacuo to provide 6-amino-2,2-dimethyl-1-hexanol (131.8 g, 77% yield), which was used without further purification. ¹H NMR (300 MHZ, CDCl₃/TMS): δ (ppm): 3.22 (s, 2H), 2.75 (t, 2H, J=7.0 Hz), 1.45 (m, 2H), 1.32-1.12 (m, 4H), 0.80 (s, 6H). ¹³C NMR (75 MHZ, CDCl₃/TMS): δ (ppm): 70.8, 41.3, 38.2, 35.1, 32.8, 24.2, 20.9.

To a solution of 6-amino-2,2-dimethyl-1-hexanol (131.8 g, 690 mmol) in ethanol (1 L) was added carbon disulfide (25.3 g, 333 mol) and the mixture was heated to reflux for 22 h. On cooling, the solution was treated with activated carbon (10 g) for two hours, then filtered and concentrated. The residue was dissolved in ethyl acetate (1 L) and washed with 1 M HCl (3×500 mL) and saturated NaHCO₃ solution (2×500 mL). The organic phase was dried over MgSO₄ and concentrated in vacuo to give a crude product (108.3 g), which was further purified by column chromatography (silica, 330 g, eluting with ethyl acetate). The product-containing fractions were combined and concentrated in vacuo under azeotropic removal of the respective solvent residues with methanol and then with dichloromethane. The obtained oil was dried at 60° C./1 torr for 1 h to provide 1,3-bis-(6-hydroxy-5,5-dimethylhexyl)-thiourea [Compound A1]as a brown, highly viscous oil (79.9 g, 70% yield). ¹H NMR (300 MHZ, DMSO-d₆/TMS): δ (ppm): 7.28 (m, 2H), 4.41 (t, 2H, J=4.8 Hz), 3.32 (m, 4H), 3.08 (d, 4H, J=4.8 Hz), 1.42 (m, 4H), 1.30-1.10 (m, 8H), 0.78 (s, 12H). ¹³C NMR (75 MHZ, DMSO-d₆/TMS): δ (ppm): ˜180 (broad), 69.8, 43.5, 38.3, 34.7, 29.9, 24.1, 20.9. HRMS (LSIMS, gly): Calcd. for C₁₇H₃₇N₂O₂S (MH⁺): 333.2576, found: 333.2551. HPLC: 91.8% purity.

1,3-Bis-(6-hydroxy-5,5-dimethyl-hexyl)-thiourea (48.9 g, 140 mmol) was dissolved in a warm solution of sodium hydroxide (11.2 g, 280 mmol) in ethanol (500 mL). This solution was chilled to 0° C. in an ice bath and 50% hydrogen peroxide (29 mL) was added dropwise over 20 min. The reaction mixture was allowed to warm to rt and stirring was continued overnight. The solution was diluted with water (1.3 L) and evaporated to dryness. The crude material was purified by flash chromatography (silica, ethyl acetate/hexanes=80/20, then methanol) to provide Compound A as a clear, faint yellow oil (22.17 g, 44% yield). ¹H NMR (300 MHZ, DMSO-d₆/TMS): δ (ppm): 5.74 (t, 2H, J=5.6 Hz), 4.43 (t, 2H, J=5.1 Hz), 3.06 (d, 4H, J=4.8 Hz), 2.95 (m, 4H), 1.38-1.24 (m, 4H) 1.24-1.08 (m, 8H), 0.77 (s, 12H). ¹³C NMR (75 MHZ, DMSO-d₆/TMS): δ (ppm): 158.1, 69.7, 48.6, 38.2, 34.7, 31.2, 24.0, 20.7. HRMS (LSIMS, POS, gly): Calcd. for C₁₇H₃₇N₂O₃ (MH⁺): 317.2804, found: 317.2803. HPLC: 84.1% purity.

### 5.1.3 Preparation of Compound B

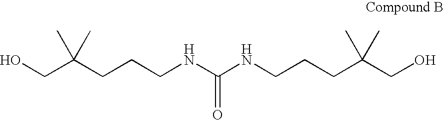

Under argon atmosphere, 2-(5-bromo-2,2-dimethylpentyloxy)-tetrahydropyran (67 g, 216 mmol, Dasseux et al., U.S. patent application Ser. No. 09/540,740, filed Mar. 31, 2000) was dissolved in DMF (140 mL) and potassium phthalimide (45 g, 238 mmol) was added. The solution was heated to 90° C. in an oil bath for 2.5 h, then cooled to rt. The DMF solution was diluted with water (3 L) and extracted with ethyl acetate (3×1 L). The combined organic phases were washed with water (3×1 L), dried over MgSO₄, and concentrated in vacuo to yield 2-(2,2-Dimethyl-5-phthalimido-pentyloxy)-tetrahydropyran as a light yellow oil (74.41 g, 95% yield). ¹H NMR (CDCl₃), δ (ppm): 7.84 (dd, 2H, J=3.1, 5.2), 7.71 (dd, 2H, J=3.1, 5.2), 4.52 (m, 1H), 3.80 (m, 1H), 3.65 (t, 2H, J=7.3), 3.50 (m, 1H), 3.43 (d, 1H, J=9.1), 2.96 (d, 1H, J=9.1), 1.92-1.40 (m, 8H), 1.34 (m, 2H), 0.89 (s, 3H), 0.88 (s, 3H). ¹³C NMR (CDCl₃), δ (ppm): 168.4, 133.9, 132.2, 123.1, 98.9, 76.0, 61.8, 38.8, 36.1, 34.1, 30.6, 25.6, 24.64, 24.56, 23.4, 19.3. HRMS (LSIMS, nba): Calcd. for C₂₀H₂₈NO₄ (MH⁺): 346.1966, found: 346.1992.

A solution of 2-(2,2-dimethyl-5-phthalimido-pentyloxy)-tetrahydropyran (74.2 g, 204 mmol) in ethanol (350 mL) was stirred as hydrazine hydrate (20 g, 85%, 340 mmol) was added slowly. Under argon atmosphere, the solution was heated to reflux for 40 minutes until the solution solidified with precipitate. After cooling to rt, the reaction mixture was broken up in methylene chloride (1.5 L) and filtered. The precipitate was washed with additional dichloromethane (1 L). The filtrate portions were combined and evaporated to near dryness and re-dissolved in hexanes (300 mL). After a second filtration, the filtrate was extracted with water (300 mL) and then dried over Na₂SO₄. Concentration of the solvent in vacuo and drying in high vacuo provided 4,4-dimethyl-5-(tetrahydropyran-2-yloxy)-pentylamine as a yellow oil (29.1 g, 60% yield). ¹H NMR (DMSO-d₆), δ (ppm): 4.50 (m, 1H), 3.72 (m, 1H), 3.43 (m, 1H), 3.37 (d, 1H, J=9.1 Hz), 2.95 (d, 1H, J=9.1 Hz), 2.50 (t, 2H, J=6.6 Hz), 1.66-1.38 (m, 6H), 1.38-1.12 (m, 4H), 0.86 (s, 6H). ¹³C NMR DMSO-d₆), δ (ppm): 98.0, 75.5, 60.8, 42.7, 36.2, 33.6, 30.2, 28.0, 25.1, 24.3, 19.0. HRMS (LSIMS, nba): Calcd. for C₁₂H₂₆NO₂ (MH⁺): 216.1964, found: 216.1957.

Under argon atmosphere, 4,4-dimethyl-5-(tetrahydropyran-2-yloxy)-pentylamine (18.54 g, 77.5 mmol) was dissolved in pyridine (80 g, 1 mol) and triphenylphosphite (26.53 g, 82.9 mmol) was added. Dry CO₂ gas was bubbled through the reaction mixture as it was heated to 40-60° C. for six hours. The gas flow was stopped and the mixture cooled to rt. After concentration in vacuo, the residue was dissolved in diethyl ether (300 mL) and washed with cold 2 N HCl (3×200 mL), then cold 10% KOH (3×200 mL). The organic layer was dried over MgSO₄ and concentrated in vacuo to yield the crude product as an orange oil. The crude product (31.3 g, 40% purity by NMR) was taken in methanol (100 mL) and concd. HCl (10 mL) was added. After refluxing for 3 hours, the mixture was cooled to rt and diluted with water (200 mL). The mixture was then extracted with methylene chloride (3×200 mL). The combined organic layers were washed with cold 10% KOH solution (3×300 mL), then sat. KCl solution (200 mL). After drying over Na₂SO₄, evaporation of the solvent gave the crude compound (3.89 g), which was purified by flash chromatography (silica, 0-12% MeOH/CHCl₃) to provide Compound B (620 mg, 7.8% yield) as a viscous, orange oil. 1H NMR (300 MHZ, DMSO-d₆/TMS): δ (ppm): 5.74 (t, 2H, J=5.7 Hz), 4.43 (t, 2H, J=5.3 Hz) 3.07 (d, 4H, J=5.2 Hz), 2.92 (m, 4H), 1.35-1.22 (m, 4H), 1.16-1.08 (m, 4H), 0.77 (s, 12H). ¹³C NMR (75 MHZ, DMSO-d₆/TMS): δ (ppm): 158.1, 69.8, 40.2, 35.7, 34.6, 24.7, 24.1. HRMS (LSIMS, nba): Calcd. for C₁₅H₃₃N₂O₃ (MH⁺): 289.2491, 289.2496.

### 5.1.4 Alternative Preparation of Compound B

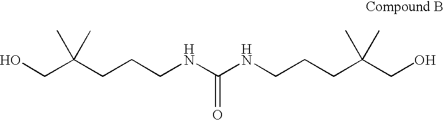

Using an alternative method, Compound B may be synthesized via the oxidation of the analogous thiourea as described below.

A solution of 5-amino-2,2,-dimethylpentan-1-ol (50.0 g, 381 mmol) and carbon disulfide (14.0 g, 184 mmol) in ethanol (200 mL) was refluxed for 20 h under argon atmosphere, then cooled and treated with activated carbon. Evaporation of the solvent and vacuum drying to provide 1,3-Bis-(5-hydroxy-4,4-dimethylpentyl)-thiourea as a yellow oil (42.04 g, 75% yield). ¹H NMR (300 MHZ, DMSO-d₆): δ (ppm): 7.27 (m, 2H), 4.42 (t, 2H, J=5.4 Hz), 3.3 (m, 4H), 3.08 (d, 4H, J=5.4 Hz), 1.52-1.35 (m, 4H), 1.21-1.10 (m, 4H), 0.78 (s, 12H). ¹³C NMR (75 MHZ, DMSO-d,): δ (ppm): ˜182 (broad), 69.8, ˜44 (broad), 35.6, 34.6, 24.0, 23.5. HRMS (LSIMS, gly): Calcd. for C₁₅H₃₂N₂O₃ (MH⁺): 305.2263, found: 305.2281. HPLC: 97.9% purity.

1,3-Bis-(5-hydroxy-4,4-dimethylpentyl)-thiourea (4.99 g, 13.8 mmol) was dissolved in ethanol (100 mL) and a solution of NaOH (1.10 g, 27.5 mmol) in water (3.3 mL) was added. To this mixture was added slowly 50% hydrogen peroxide solution (3.7 mL, 54 mmol), setting off a vigorous reaction. The mixture was stirred for 3 h, then diluted with water (700 mL) and extracted with chloroform (3×500 mL). The organic phases were combined and dried over MgSO₄, then evaporated to give the crude product (1.62 g), which was then re-crystallized from hot ethyl acetate (50 mL) to provide Compound B as white plates (1.0 g, 25% yield). Mp.: 81-82° C. HPLC: 93.2% purity.

### 5.1.5 Preparation of Compound C

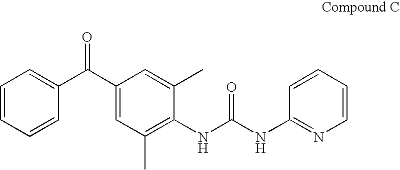

To a cooled solution of anhydrous AlCl₃ (48 mmol) in 1,2-dichloroethane (15 mL), benzotrichloride (8 mmol) was added dropwise in 5 min under efficient stirring. The mixture was stirred further for 15 min at 0-5° C. N-2,6-Dimethylphenyl-N′-2-pyridinylurea (prepared by reacting 2,6-dimethylphenyl isocyanate with 2-aminopyridine in THF as described in Pavia, M., Lobbestael, S. J., Taylor, C. P., Hershenson, F. M., and Miskell, D. L. J. Med. Chem. 1990, 33, 854) (7 mmol) was dissolved in 1,2-dichloroethane (5 mL) and added over 15 min, after which the cooling bath was removed and the mixture was stirred for additional 5 h. The completion of the conversion was checked by TLC (hexanes:ethyl acetate; 8:2). The dark brown mixture was poured into crushed ice (50 g) and the resulting mixture was stirred at 70° C. for 0.5 h. After cooling, the organic layer was separated, washed with water (3×25 mL), and dried. The solvent was removed under reduced pressure and the residue was purified by column chromatography on silica gel (using a gradient of 25% ethyl acetate/hexanes to 50% ethyl acetate/hexanes to 100% methanol). The resulting oil was triturated with diethyl ether to provide Compound 1 as a lightly colored powder (1.46 g, 4.2 mmol, 60%). Mp.: 148-153° C. (decomposition). ¹H NMR (300 MHZ, CDCl₃/TMS): δ (ppm): 9.75 (br., 1H), 8.2-6.8 (br. m., 11H), 2.4 (br. s, 3H), 2.2 (br. s, 3H). ¹³C NMR (75 MHZ, CDCl₃/TMS): δ (ppm): 197.4, 153.5, 149.5 (br.), 145.0 (br.), 138.3, 137.6, 136.5, 134.0, 133.1, 129.5, 128.1, 127.3, 126.7, 117.6 (br.), 115.5 (br.), 18.6, 15.5. HRMS (LSIMS, nba): Calcd. for C₂₁H₂₀N₃O₂ (MH⁺): 346.1579, found: 346.1556. HPLC: 96% purity.

### 5.1.6 Preparation of Compound D

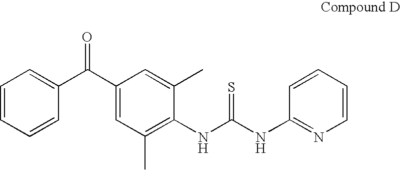

A solution of 4-benzoyl-2,6-dimethyl-nitrobenzene (prepared according to Goldstein, S. L.; McNellis, E. J. Org. Chem. 1984, 49, 1613) (6.06 g, 19 mmol, 80%) in THF (120 mL) was added to a mixture of iron (4.14 g, 72 mmol) and ammonium chloride (6.14 g, 120 mmol) in water (75 mL). Methanol (15 mL) was added to keep the nitro compound soluble. The reaction mixture was stirred under reflux for 8 h (TLC monitoring). The reaction mixture was cooled and filtered. The residue was washed with ethyl acetate (3×100 mL) and water (100 mL). The filtrate was separated and the organic layer was washed with brine (50 mL), dried over Na₂SO₄ and concentrated in vacuo to give the crude amino compound as a solid (4.57 g). Re-crystallization from iso-propanol provoided 4-Benzoyl-2,6-dimethylaniline as a pale powder (2.3 g, 54%). Mp.: 132-136° C. ¹H NMR (300 MHZ, CDCl₃/TMS): δ (ppm): 13.11 (br., 1H), 8.72 (br., 1H), 8.23 (d, J=3.8 Hz, 1H), 7.84 (d, J=7.3 Hz, 2H), 7.73 (t, J=8.5 Hz, 1H), 7.66-7.45 (m, 5H), 7.05 (t, J=6.3 Hz, 1H), 6.83 (d, J=8.5 Hz, 1H), 2.4 (s, 6H). ¹³C NMR (75 MHZ, CDCl₃/DMSO-d₆/TMS): δ (ppm): 195.7, 147.8, 138.9, 131.3, 131.1, 129.3, 127.8, 126.2, 120.1, 17.3.

A solution of 4-amino-3,5-dimethyl-benzophenone (3.02 g, 97%, 13 mmol) in anhydrous acetonitrile (25 mL) was mixed with a suspension of 1,1′-thiocarbonyldiimidazole (2.97 g, 90%, 15 mmol) in acetonitrile (50 mL) under stirring at rt. After 20 min, the mixture became clear. The mixture was stirred further for 12 h at 65° C. The solvent was removed under reduced pressure to give an oily residue, which was dissolved in anhydrous DMF (50 mL). 2-Aminopyridine (0.93 g, 9.75 mmol) was added and the mixture was heated for 10 h at 100° C. The reaction mixture was diluted with CH2Cl2 (500 mL) and washed successively with HCl (1 N, 2×50 mL) and water (50 mL). The organic phase was dried over MgSO₄, filtered, and concentrated under reduced pressure. The residue was crystallized from iso-propanol (10 mL) to provide Compound 2 as a light yellow solid (1.1 g, 23%). Mp.: 198-202° C. Washing with hot acetone (5 mL) increased the melting point to 203-206° C. ¹H NMR (300 MHZ, CDCl₃/TMS): δ (ppm): 13.11 (br., 1H), 8.72 (br., 1H), 8.23 (d, J=3.8 Hz, 1H), 7.84 (d, J=7.3 Hz, 2H), 7.73 (t, J=8.5 Hz, 1H), 7.66-7.45 (m, 5H), 7.05 (t, J=6.3 Hz, 1H), 6.83 (d, J=8.5 Hz, 1H), 2.4 (s, 6H). ¹³C NMR (75 MHZ, CDCl₃/DMSO-d₆/TMS): δ (ppm): 195.6, 179.3, 153.4, 144.7, 140.2, 138.2, 137.0, 135.7, 135.4, 131.7, 129.2, 127.6, 117.3, 112.5, 18.1. HRMS (LSIMS, nba): Calcd. for C₂₁H₂₀N₃OS(MH⁺): 362.1327, found 362.1313.

### 5.1.7 Preparation of Compound E

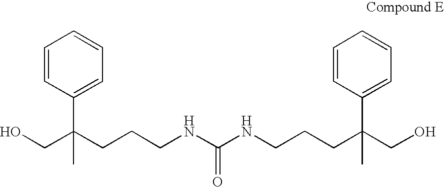

Under nitrogen atmosphere, LiBH₄ (3.0 g, 138 mmol) was added carefully to anhydrous methylene chloride (100 mL) and heated to 30° C. for 2 h, then cooled to rt. Anhydrous methanol (4.4 mL, 101 mmol) was added dropwise to this solution, followed by dropwise addition of ethyl 5-bromo-2-methyl-2-phenyl-pentanoate (20.0 g, 67 mmol) in anhydrous methylene chloride (50 mL). The mixture was heated to 40° C. overnight, then cooled with an ice bath, and hydrolized by careful addition of deionized water (35 mL) and 2 N HCl (80 mL). The layers were separated and the aqueous layer was extracted with methylene chloride (2×50 mL). The combined organic layers were washed with 2 N HCl (30 mL) and saturated NaHCO₃ solution (30 mL), dried over MgSO₄, and evaporated to provide 5-Bromo-2-methyl-2-phenyl-1-pentanol as a yellow oil (16.0 g, 93% yield). ¹H NMR (300 MHZ, CDCl₃/TMS): δ (ppm): 7.52-7.17 (m, 5H), 3.78-3.65 (m, 1H), 3.65-3.45 (m, 1H), 3.32 (t, J=6.3 Hz, 2H), 2.05-1.40 (m, 5H), 1.36 (s, 3H). ¹³C NMR (75 MHZ, CDCl₃/TMS): δ (ppm): 144.0, 128.5, 126.6, 126.3, 72.4, 43.0, 36.9, 34.4, 27.4, 21.4. HRMS (LSIMS, nba): Calcd. for C₁₂H₁₆Br (MH⁺—H₂O): 239.0435, found 239.0429.

Under anhydrous conditions, 5-bromo-2-methyl-2-phenyl-1-pentanol (15.8 g, 61 mmol) was combined with potassium phthalimide (9.6 g, 51 mmol) in DMF (200 mL). This solution was heated to 80-90° C. for 2 h and left overnight at rt under nitrogen atmosphere. The solvent was evaporated under reduced pressure and the residue was suspended in deionized water (80 mL). The mixture was extracted with ethyl acetate (4×50 mL). The combined organic layers were washed with water (50 mL), dried over MgSO₄, and concentrated to furnish the crude product as a beige wax (17.8 g). Purification by column chromatography (silica, hexanes/ethyl acetate=3/1, then 2/1) provided 2-methyl-2-phenyl-5-phthalimido-1-pentanol as a yellow oil (9.3 g, 56% yield). ¹H NMR (300 MHZ, CDCl₃/TMS): δ (ppm): 7.83-7.78 (m, 2H), 7.71-7.66 (m, 2H), 7.32-7.13 (m, 5H), 3.72-3.49 (m, 4H), 1.92-1.76 (m, 1H), 1.72-1.48 (m, 2H), 1.48-1.32 (m, 1H), 1.34 (s, 3H). ¹³C NMR (75 MHZ, CDCl₃/TMS): δ (ppm): 168.3, 144.1, 133.8, 131.9, 128.4, 126.5, 126.2, 123.1, 72.2, 43.1, 38.4, 35.3, 23.2, 21.4. HRMS (LSIMS, nba): Calcd. for C₂₀H₂₂NO₃ (MH⁺): 324.1600, found: 324.1593.

To a solution of 2-methyl-2-phenyl-5-phthalimido-1-pentanol (9.25 g, 29 mmol) in ethanol (100 mL) was added hydrazine monohydrate (2.9 g, 57 mmol) and the mixture was refluxed for 1 h, then kept overnight at rt. The reaction mixture solidified overnight. The solid mass was suspended in methylene chloride (350 mL) and filtered. The filtrate was concentrated in vacuo, the residue was re-dissolved in hexanes and filtered to remove residual phthalhydrazide. The filtrate was concentrated in vacuo and dried in high vacuo to afford crude 5-amino-2-methyl-2-phenyl-1-pentanol (4.7 g, 85% yield) as a yellow oil, which was used without further purification. ¹H NMR (300 MHZ, CDCl₃/TMS): δ (ppm): 7.40-7.10 (m, 5H), 3.64 (d, J=10.8 Hz, 1H), 3.56 (d, J=10.8 Hz, 1H), 2.85-2.65 (m, 3H), 2.58 (t, J=7.0 Hz, 2H), 1.85-1.65 (m, 1H), 1.65-1.45 (m, 1H), 1.45-1.05 (m, 2H), 1.31 (s, 3H). ¹³C NMR (75 MHZ, CDCl₃/TMS): δ (ppm): 145.1, 128.3, 126.6, 126.0, 71.7, 43.1, 42.6, 35.4, 27.7, 22.0. HRMS (LSIMS, nba): Calcd. for C₁₂H₂₀NO (MH⁺): 194.1545, found 194.1534.

To a solution of 5-amino-2-methyl-2-phenyl-1-pentanol (4.0 g, 20 mmol) in anhydrous methylene chloride (50 mL) was added 1,1′-carbonyldiimidazole (1.5 g, 9.4 mmol) and the mixture was stirred overnight at rt under a nitrogen atmosphere. The reaction progress was monitored by TLC. After 20 h, the reaction mixture was extracted with water (4×20 mL). The organic layer was separated, dried over MgSO₄, and concentrated under reduced pressure to give the crude product (3.4 g, 88% yield) as a yellow foam. The crude product was dissolved in ethyl acetate (100 mL), saturated NaHCO₃ (10 mL) was added and the mixture was stirred for 1 h. The layers were separated and the organic layer was washed with 3 N HCl (2×10 mL) and brine (10 mL), dried over MgSO₄, and concentrated under reduced pressure to provide Compound 3 as a light yellow, solid foam (1.5 g, 39% yield). Mp.: 35-37° C. ¹H NMR (300 MHZ, CDCl₃/TMS): δ (ppm): 7.45-7.05 (m, 10H), 3.70-3.45 (m, 4H), 3.05-2.79 (m, 4H), 1.80-1.40 (m, 4H), 1.40-0.95 (m, 4H), 1.24 (s, 6H). ¹³C NMR (75 MHZ, CDCl₃/TMS): δ (ppm): 159.2, 145.2, 128.2, 126.3, 125.9, 70.9, 42.6, 40.7, 34.9, 24.5, 22.1. HRMS (LSIMS, nba): Calcd. for C₂₅H₃₇N₂O₃ (MH⁺): 413.2804, found: 413.2764. HPLC: 69.8% purity.

### 5.1.8 Preparation of Compound F

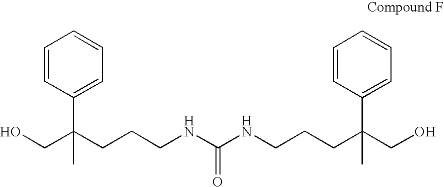

To a solution of 5-amino-2-methyl-2-phenyl-pentan-1-ol (4.6 g, 23.7 mmol) in ethanol (50 mL) was added carbon disulfide (0.65 ml, 10.8 mmol) and the mixture was heated to reflux overnight. After cooling to rt, the mixture was treated with activated carbon (1 g) for 2 h, then filtered, and concentrated. The residue was dissolved in ethyl acetate (50 mL) and washed with 1 N HCl (3×20 mL). The organic layer was dried over MgSO₄ and concentrated to give the crude product (5.0 g). Purification by column chromatography (silica, hexanes/ethyl acetate=3/1) provided Compound 4 as a viscous oil (1.9 g, 42%). Mp.: 44-45° C. ¹H NMR (300 MHZ, CDCl₃/TMS): δ (ppm): 7.39-7.10 (m, 10H), 6.18-5.90 (br. s, 2H), 3.70-3.50 (m, 4H), 3.39-3.10 (br. s, 4H), 2.50-2.35 (m, 2H), 1.82-1.68 (m, 2H), 1.68-1.50 (m, 2H), 1.50-1.10 (m, 4H), 1.27 (s, 6H). ³C NMR (75 MHZ, CDCl₃/TMS): δ (ppm): 181.0, 144.6, 128.4, 126.4, 126.2, 71.2, 44.7, 42.8, 35.0, 23.6, 22.0. HRMS (LSIMS, nba): Calcd. for C₂₅H₃₇N₂O₂S (MH⁺): 429.2576, found: 429.2589. HPLC: 92.7%.

### 5.1.9 Preparation of Compound G

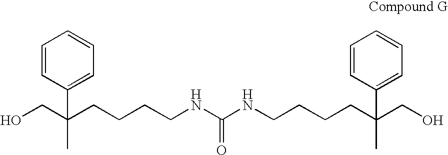

Under nitrogen atmosphere, to a solution of LiBH₄ (5.4 g, 250 mmol) in methylene chloride (180 mL) was added methanol (8.2 g, 256 mmol) dropwise at room temperature over 30 min. The reaction mixture was heated to reflux and ethyl 6-bromo-2-methyl-2-phenyl-hexanoate (52 g, 166 mmol) was added. After heating to reflux overnight, the reaction mixture was cooled to room temperature and hydrolized with saturated ammonium chloride solution (100 mL). The organic layer was separated and the aqueous layer was extracted with methylene chloride (3×60 mL). The combined organic layers were washed with 2 N hydrochloric acid (100 mL) and saturated sodium chloride solution (100 mL), dried over sodium sulfate, and concentrated in vacuo to provide 6-bromo-2-methyl-2-phenyl-hexanol (38 g 84.4%).

¹H NMR (300 MHZ, CDCl₃/TMS): δ (ppm): 7.7-7.3 (m, 5H), 3.88 (d, J=10.8 Hz, 1H), 3.73 (d, J=10.8 Hz, 1H), 3.54 (t, J=7.0 Hz, 2H), 2.3-1.2 (m, 10H). ¹³C NMR (75 MHZ, CDCl₃/TMS): δ (ppm): 144.4, 128.4, 126.5, 126.1, 72.4, 43.2, 37.4, 33.5, 33.3, 22.5, 21.4.

Under argon atmosphere, to a solution of 6-bromo-2-methyl-2-phenyl-hexanol (37 g, 136.5 mmol) in DMF (90 mL) was added potassium phthalimide (28 g, 151.3 mmol) and the reaction mixture was heated to 90° C. for 4 h, then cooled to rt. The solution was diluted with water (2 L) and the mixture was extracted with chloroform (3×600 mL). The combined organic layers were washed with water (3×600 mL), dried over sodium sulfate, and concentrated in vacuo to provide 6-phthalimido-2-methyl-2-phenyl-hexan-1-ol as a white solid (46 g, 100%). ¹H NMR (300 MHZ, CDCl₃/TMS): δ (ppm): 7.82 (m, 2H), 7.69 (m, 2H), 7.38-7.12 (m, 5H), 3.76-3.50 (m, 4H), 1.9-0.9 (m, 7H), 1.34 (s, 3H). ¹³C NMR (75 MHZ, CDCl₃/TMS): δ (ppm): 168.3, 144.5, 133.8, 132.0, 128.4, 126.6, 126.1, 123.1, 72.1, 43.3, 37.8, 37.6, 29.1, 21.6, 21.0. HRMS (LSIMS, nba): Calcd. for C₂₁H₂₄NO₃ (MH⁺): 338.1756, found: 338.1791. HPLC: 89%.

Under nitrogen atmosphere, a solution of 6-phthalimido-2-methyl-2-phenyl-hexan-1-ol (46 g, 136.5 mmol) in ethanol (230 mL) was stirred as hydrazine hydrate (13.4 g, 230 mmol, 85%) was added slowly. The reaction mixture was heated to -reflux and became homogeneous. After 10 minutes, a precipitate formed and reflux was continued for 1 h. After cooling to rt, the reaction mixture was acidified to pH 1 with concd. HCl (100 mL) and filtered. The solid was washed with dichloromethane (1 L). The organic layer was separated and the aqueous layer was extracted with dichloromethane (3×100 mL). The combined organic layers were dried over sodium sulfate, concentrated in vacuo, and dried in high vacuo to provide 6-amino-2-methyl-2-phenyl-hexanol as a yellow oil (22.3 g, 78.9%). ¹H NMR (300 MHZ, CDCl₃/TMS): δ (ppm): 7.40-7.00 (m, 5H), 3.70-3.45 (m, 2H), 2.53 (t, J=7.0 Hz, 2H), 2.14 (br., 3H), 1.82-0.87 (m, 6H), 1.32 (s, 3H). ¹³C NMR (75 MHZ, CDCl₃/TMS): δ (ppm): 145.3, 128.2, 126.5, 125.8, 71.8, 43.2, 41.5, 37.9, 33.7, 21.7, 20.9. HRMS (LSIMS, nba): Calcd. for C₁₃H₂₂NO (MH⁺): 208.1701, found: 208.1708.

To solution of N,N′-carbonyldiimidazole (CDI, 3.0 g, 18.4 mmol) in anhydrous methylene chloride (160 mL), was added 6-amino-2-methyl-2-phenyl-hexanol (8.0 g, 38.6 mmol). The reaction mixture was stirred for 36 h at room temperature under a nitrogen atmosphere. The reaction mixture was poured into ice-water (200 mL) and the organic layer was separated from the aqueous layer. The aqueous layer was extracted with CH₂Cl₂ (2×100 mL). The combined organic layers were washed with water (2×100 mL), dried, and concentrated in vacuo to give the crude product (8.5 g). The crude product was purified by flash chromatography (silica gel, ethyl acetate) to provide Compound 5 as a solid foam (4.9 g, 60.5%). ¹H NMR (300 MHZ, CDCl₃/TMS): δ (ppm): 7.35-7.12 (m, 10H), 4.74-4.58 (m, 2H), 3.72-3.46 (m, 4H), 3.10-2.92 (m, 4H), 2.34 (br., 2H), 1.82-0.90 (m, 18H). ¹³C NMR (75 MHZ, CDCl₃/TMS): δ (ppm): 159.2, 145.6, 128.8, 127.0, 126.5, 72.0, 43.6, 40.3, 38.1, 31.2, 22.6, 21.4. HPLC: 79.3% purity.

### 5.1.10 Preparation of Compound H

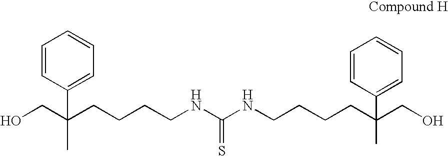

A solution of 6-amino-2-methyl-2-phenyl-hexanol (9.0 g, 43.5 mmol) and CS₂ (1.6 g, 21.1 mmol) in ethanol (50 ml) was refluxed for 20 h under a nitrogen atmosphere. The solvent was distilled off and the residue was purified twice by chromatography (silica gel, first: hexanes:ethyl acetate=1:1, second: chloroform:ethanol=1:1) to provide Compound 6 as a solid foam (3.1 g, yield: 31.3%). ¹H NMR (300 MHZ, CDCl₃/TMS): δ (ppm): 7.40-7.00 (m, 10H), 6.16 (br., 2H), 3.59-3.40 (m, 4H), 3.23 (br., 4H), 2.47 (br., 2H), 1.80-1.32 (m, 8H), 1.26 (s, 6H), 1.22-0.82 (m, 4H). ¹³C NMR (75 MHZ, CDCl₃/TMS): δ (ppm): 144.7, 128.1, 126.2, 125.8, 71.3, 44.0, 42.8, 37.5, 29.2, 21.7, 20.9. HRMS (LSIMS, nba): Calcd. for C₂₇H₄₁N₂O₂S (MH⁺): 457.2889, found 457.2884. HPLC: 94.5% purity.

### 5.1.11 Preparation of Compound I

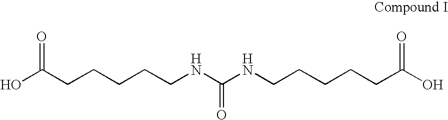

A solution of ethyl 6-bromo-2,2-dimethylhexanoate (Miyake, A. et al., Chem. Pharm. Bull. 1997, 45, 1447-1457; 50 g, 180 mmol) and potassium phthalimide (51 g, 270 mmol) in anhydrous DMF (200 mL) was heated to 100° C. for 17 h. After dilution with water (3.5 L), the aqueous mixture was extracted with ethyl acetate (3×1 L). The combined organic phases were washed with water (2×2 L) and brine (2 L), then dried over Na₂SO₄. Evaporation gave crude material (64.5 g) that was purified by Kugelrohr distillation (206-216° C./0.03 Torr), to provide 2,2-Dimethyl-6-phthalimido-hexanoic acid ethyl ester as a colorless oil (42.72 g, 90% yield). ¹H NMR (300 MHz, CDCl₃/TMS): δ (ppm): 7.87-7.81 (m, 2H), 7.75-7.68 (m, 2H), 4.10 (q, 2H, J=7.1 Hz), 3.66 (t, 2H, J=7.4 Hz), 1.72-1.51 (m, 4H), 1.34-1.24 (m, 2H), 1.22 (t, 3H, =7.1 Hz), 1.15 (s, 6H). ¹³C NMR (75 MHz, CDCl₃/TMS): δ (ppm): 178.0, 168.5, 134.0, 132.4, 123.3, 60.4, 42.3, 40.3, 38.0, 29.2, 25.3, 22.4, 14.4. HRMS (LSIMS, nba): Calcd. for C₁₈H₂₃NO₄ (MH⁺): 317.1643, found 317.1635. HPLC: 99.95%.

A solution of 2,2-dimethyl-6-phthalimido-hexanoic acid ethyl ester (16.0 g, 47.9 mmol) and hydrazine monohydrate (5.7 g, 97 mmol) in ethanol (500 mL) was heated to reflux for 1.5 h. The hot mixture was poured into a beaker and allowed to cool. The precipitated phthalhydrazide was filtered and the solids washed with cold ethanol (1 L). After condensation, the crude material was redissolved in hexanes (1 L) and filtered again. The filtrate was concentrated and purified by column chromatography (silica, 1% of 30% NH₄OH, 10% MeOH, 89% CH₂Cl₂) to provide 6-Amino-dimethylhexanoic acid ethyl ester as a colorless oil (4.68 g, 64% yield). ¹H NMR (300 MHz, CDCl₃/TMS): δ (ppm): 4.11 (q, 2H, J=7.1 Hz), 2.68 (t, 2H, J=7.0 Hz), 1.60-1.34 (m, 6H), 1.24 (t, 3H, J=7.1 Hz), 1.16 (s, 6H). ¹³C NMR (75 MHz, CDCl₃/TMS): δ (ppm): 178.0, 60.2, 42.2, 42.0, 40.5, 34.2, 25.1, 22.3, 14.3.

Under argon atmosphere, ethyl 6-amino-2,2-dimethylhexanoate (5.10 g, 26 mmol) was heated to reflux with 1,1′-carbonyldiimidazole (2.1 g, 13 mmol) in dichloromethane (120 mL) for five days. The solution was poured into ice water (200 mL) and extracted with dichloromethane (3×100 mL). The combined organic layers were washed with 1 N HCl (3×100 mL) and saturated NH₄Cl solution (2×100 mL) and dried over MgSO₄. Evaporation to provide 1,3-Bis(5-ethoxycarbonyl-5-methylhexyl)urea as a colorless oil (4.18 g, 39%). ¹H NMR (300 MHz, CDCl₃/TMS): δ (ppm): 5.05 (t, 2H, J=5.4 Hz), 4.10 (q, 4H, J=7.1 Hz), 3.13 (q, 4H, J=6.6 Hz), 1.60-1.39 (m, 4H), 1.32-1.20 (m, 2H), 1.24 (t, 3H, J=7.1 Hz), 1.15 (s, 6H).

A solution of 1,3-bis(5-ethoxycarbonyl-5-methylhexyl)urea (4.18 g, 10.2 mmol) in ethanol (80 mL) and 5% KOH solution (40 mL) was heated to reflux for 20 h. The ethanol was removed under reduced pressure and the aqueous solution was extracted with dichloromethane (2×100 mL). The aqueous layer was acidified to pH 1 with 3 N HCl and extracted with ethyl acetate (4×50 mL). The combined ethyl acetate layers were washed with saturated NH₄Cl solution (3×100 mL), dried over MgSO₄, and concentrated in vacuo to provide Compound 7 as a colorless oil (4.04 g, 92%). ¹H NMR (300 MHz, CDCl₃/TMS): δ (ppm): 3.09 (t, 4H, J=6.5 Hz), 1.57-1.20 (m, 12H), 1.15 (s, 12H). ¹³C NMR (75 MHz, CDCl₃/TMS): δ (ppm): 182.0, 161.4, 43.1, 41.7, 41.0, 32.0, 25.8, 23.6. HRMS (LSIMS, gly): Calcd. for C₁₇H₃₃N₂O₅ (MH⁺): 345.2389, found: 345.2390.

### 5.1.12 Preparation of N,N′-bis(4,4-Dimethyl-5-hydroxypentyl)urea

2,2-Dimethyl-5-phthalimido-1-(tetrahydropyranyloxy)pentane. Under argon, 5-bromo-2,2-dimethyl-1-(tetrahydropyranyloxy)pentane (67 g, 216 mmol, Dasseux et al. U.S. patent application Ser. No. 09/540,740 filed Mar. 31, 2000) was dissolved in DMF (140 mL). To this was added potassium phthalimide (45 g, 238 mmol). The solution was heated to 90° C. in an oil bath for 2.5 h, then cooled to rt. The DMF solution was then poured into a separatory funnel containing H₂O (3 L); this was extracted with ethyl acetate (3×1 L). The combined organics were washed with H₂O (3×1 L), then dried over MgSO₄. Concentration yielded the product as a light yellow oil (74.41 g, 95% yield). ¹H NMR (CDCl₃), δ (ppm): 7.84 (dd, 2H, J=3.1, 5.2); 7.71 (dd, 2H, J=3.1, 5.2); 4.52 (m, 1H); 3.80 (m, 1H); 3.65 (t, 2H, J=7.3); 3.50 (m, 1H); 3.43 (d, 1H, J=9.1); 2.96 (d, 1H, J=9.1); 1.92-1.40 (m, 8H); 1.34 (m, 2H); 0.89 (s, 3H); 0.88 (s, 2H). ¹³C NMR (CDCl₃), δ (ppm): 168.4, 133.9, 132.2, 123.1, 98.9, 76.0, 61.8, 38.8, 36.1, 34.1, 30.6, 25.6, 24.64, 24.56, 23.4, 19.3.

5-Amino-2,2-dimethyl-1-(tetrahydropyranyloxy)-pentane. A solution of 2,2-dimethyl-5-phthalimido-1-(tetrahydropyranyloxy)pentane (74.2 g, 204 mmol) in ethanol (350 mL) was stirred as hydrazine hydrate (20 g, 85%, 340 mmol) was added slowly. Under argon, the solution was heated to reflux for 40 minutes, until the solution solidified with precipitate. After cooling to rt, the reaction mixture was broken up in methylene chloride (1.5 L) and filtered. The precipiate was washed with an additional CH₂Cl₂ (1 L). The filtrate portions were combined and evaporated to near dryness and redissolved in hexane (300 mL). After a second filtration, the hexanes were extracted with H₂O (300 mL), and then dried over Na₂SO₄. Concentration and drying gave the product as a yellow oil (29.1 g, 60% yield). ¹H NMR (DMSO-d₆), δ (ppm): 4.50 (m, 1H); 3.72 (m, 1H); 3.43 (m, 1H); 3.37 (d, 1H, J=9.1); 2.95 (d, 1H, J=9.1); 2.50 (t, 2H, J=6.6); 1.66-1.38 (m, 6H); 1.38-1.12 (m, 4H); 0.86 (s, 6H). ¹³C NMR (DMSO-d₆), δ (ppm): 98.0, 75.5, 60.8, 42.7, 36.2, 33.6, 30.2, 28.0, 25.1, 24.3, 19.0.

N,N′-bis(4,4-dimethyl-5-hydroxypentyl)urea. Under argon, 5-amino-2,2-dimethyl-1-(tetrahydropyranyloxy)pentane (18.54 g, 77.5 mmol) was dissolved in pyridine (80 g, 1 mol). To this was added triphenylphosphite (26.53 g, 82.9 mmol). Finally, dry CO₂ was bubbled through the reaction mixture as it was heated to 40-60° C. for six hours. Gas flow was stopped and the mixture cooled to rt. After evaporation of the pyridine, the residue was dissolved in diethyl ether (300 mL) and washed with cold 2 N HCl (3×200 mL), then cold 10% KOH (3×200 mL). The organic layer was dried over MgSO₄, the ether was condensed to yield crude product as an orange oil. The crude product (31.3 g, 40% purity by NMR, estimated 27.5 mmol) was taken in methanol (100 mL) and concd. HCl (10 mL) was added. After refluxing for 3 hours, the mixture was cooled to rt and diluted with H₂O (200 mL). The mixture was then extracted with methylene chloride (3×200 mL). The combined organics were washed with cold 10% KOH (3×300 mL), then sat. KCl (200 mL). After drying over Na₂SO4, evaporation of the solvent gave crude compound (3.89 g), which was purified by flash chromatography (silica, 0-12% MeOH/CHCl₃) to give the product (620 mg, 7.8% yield) as a viscous, orange oil. ¹H NMR (DMSO-d₆), δ (ppm): 5.40 (t, 2H); 4.42 (t, 2H); 3.06 (d, 4H); 2.90 (m, 4H); 1.4-1.2 (m, 4H); 1.2-1.05 (m, 4H); 0.77 (s, 12H). HRMS calcd. For C₁₅H₃₃N₂O₃ (MH⁺): 289.2500, 289.2496.

### 5.1.13 Preparation of 1,3-Bis(5,5-dimethyl-6-hydroxy-hexyl)-urea

2,2-Dimethyl-5-phthalimido-1-(tetrahydropyranyloxy)hexane. Under N₂ atmosphere, potassium phthalimide (49.1 g, 265.2 mmol) was added to a stirred solution of 2-(6-bromo-2,2-dimethylhexyloxy)-tetrahydropyran (70.7 g, 241.1 mmol) in DMF (150 ml, dried over 4 A molecular sieves) at rt. The suspension was heated to 80-95° C. for 3 h. The reaction mixture was cooled to rt, diluted with water (500 ml), and extracted with diethyl ether (2×250 ml, 1×100 ml). The combined organic layers were washed with saturated NaCl solution (100 ml), dried over MgSO₄, concentrated in vacuo, and dried in high vacuo to furnish the expected product (78.4 g, 90%) as a yellow oil. ¹H NMR (300 MHz, CDCl₃): δ (ppm) 7.84 (dd, 2H, J=5.4, 3.1); 7.71 (dd, 2H, J=5.4, 3.1); 4.53 (t, 1H, J=2.9); 3.81 (m, 1H); 3.68 (t, 2H, J=7.3); 3.48 (m, 1H); 3.46 (d, 1H, J=9.2); 2.97 (d, 1H, J=9.2); 2.97 (d, 1H, J=9.2); 1.90-1.42 (m, 9H); 1.31 (m, 3H); 0.89 (s, 3H); 0.88 (s, 3H). ¹³C NMR (75 MHz, CDCl₃): δ (ppm) 168.42, 133.89, 132.26; 123.18; 99.09; 76.45, 61.88, 38.95, 38.13, 34.25, 30.70, 29.59, 25.65, 24.60, 21.44, 19.48. HRMS (LSIMS, POS, gly) calcd. for C₁₆H₂₂NO₃ (MH⁺-DHP): 276.1600, found: 276.1597.

5,5-Dimethyl-6-(tetrahydro-pyran-2-yloxy)-hexylamine. A solution of hydrazine hydrate in water (85% w/w, 17.3 g, 294.3 mmol) was added dropwise to a solution of 2,2-dimethyl-5-phthalimido-1-(tetrahydropyranyloxy)hexane (78.0 g, 216.98 mmol) in ethanol (400 ml) at rt. The reaction mixture was heated to reflux for 1 h, then cooled to rt. The precipitate was removed by filtration and washed with ethanol (2×100 ml). The filtrate was concentrated to a volume of ca. 100 ml. Further precipitate was filtered off and washed with diethyl ether (4×100 ml). The combined organic layers were washed with saturated NaCl solution (3×75 ml), dried over MgSO₄, concentrated in vacuo, and dried in high vacuo to give the expected product (29.0 g, 58%) as a yellowish oil. ¹H NMR (CDCl₃), δ (ppm): 4.58 (s, 2H, OH); 4.54 (t, 1H, J=3.5); 3.82 (m, 1H); 3.49 (m, 4H); 3.46 (d, 1H, J=9.1); 2.98 (d, 1H, J=9.1); 2.78 (t, 2H, J=7.3); 1.94-1.44 (m, 8H); 1.40-1.20 (m, 4H); 0.89 (s, 6H). ¹³C NMR (CDCl₃), δ (ppm): 99.08; 76.34; 61.89; 41.18; 38.91; 34.15; 32.29; 30.63; 25.53; 24.49; 21.17; 19.44. calcd. for C₁₃H₂₈NO₂ (MH⁺): 230.2120; found: 230.2123.

1,3-Bis(5,5-dimethyl-6-hydroxy-hexyl)-urea. To a solution of 5,5-dimethyl-6-(tetrahydro-pyran-2-yloxy)-hexylamine (14.9 g, 64.96 mmol) in pyridine (200 ml) was added triphenyl phosphite (10.1 g, 32.48 mmol). The solution was heated to 40-50° C. and CO₂ gas was introduced through a gas immersion tube (CO₂ was developed by evaporation of dry-ice and dried with Drierite. The duration of CO₂ introduction is estimated at ca. 8 h). The reaction mixture was cooled to rt after 18 h and a sample (2 ml 1-5 of the solution) was taken for monitoring of the reaction progress by NMR spectroscopy (ratio P/SM≈1/2). Additional triphenyl phosphite (10.2 g, 32.80 mmol) was added, CO₂ was introduced (for approximately 18 h), and the reaction mixture was heated to 55° C. for 23 h (NMR analysis indicated a ratio P/SM=2/1). The solvent was removed in vacuo and the reaction mixture concentrated in high vacuo to give the THP-protected product (53 g) as a yellow oil. The crude, protected urea (53 g) was dissolved in methanol (200 ml) and concd. HCl (20 ml) and heated to reflux for 3 h. The solution was cooled to rt, diluted with water (200 ml), and concentrated in vacuo to a volume of ca. 300 ml. The solution was extracted with CH₂Cl₂ (3×100 ml). The combined organic layers were washed with 10% NaOH solution (2×100 ml) and saturated NaCl solution (2×100 ml), dried over MgSO₄, concentrated in vacuo, and dried in high vacuo. The residual oil (5.30 g) was purified by flash chromatography (silica; hexanes/ethyl acetate=40/60, then 20/80, followed by chloroform/methanol=95/5) to furnish the expected product (2.24 g, 22% over two steps) as a yellow oil. ¹H NMR (CDCl₃), δ (ppm): 3.26 (s, 4H); 3.11 (t, 4H, J=6.8); 1.51-1.38 (m, 4H); 1.36-1.18 (m, 8H); 0.85 (s, 12H). ¹³C NMR (CDCl₃), d (ppm): 161.50, 71.97, 41.08, 39.67, 36.07, 32.49, 24.59, 22.33. HRMS calcd. for C₁₇H₃₇N₂O₃ (MH⁺): 317.2804, found 317.2793.

### 5.1.14 Preparation of N,N′-bis(5-hydroxy-4,4-dimethylpentyl)urea

N,N′-bis(5-hydroxy-4,4-dimethylpentyl)thiourea. A solution of 5-amino-2,2,-dimethylpentan-1-ol (50.0 g, 381 mmol) and carbon disulfide (14.0 g, 184 mmol) in ethanol (200 mL) was refluxed for 20 h under argon, then cooled and treated with activated carbon. Evaporation of the solvent and vacuum drying gave the product as a yellow oil (42.04 g, 75% yield). ¹H NMR (DMSO-d₆), δ (ppm): 7.27 (m, 2H); 4.42 (t, 2H, J 5.4); 3.3 (m, 4H); 3.08 (d, 4H, J 5.4); 1.52-1.35 (m, 4H); 1.21-1.10 (m, 4H); 0.78 (s, 12H). ¹³C NMR (75 MHz, DMSO-d₆): δ (ppm): ˜182 (broad), 69.8, ˜44 (broad), 35.6, 34.6, 24.0, 23.5. The broadened carbon signals near the thiourea unit appear to be characteristic of the group.

N,N′-bis(5-hydroxy-4,4-dimethylpentyl)urea. The above thiourea (4.99 g, 13.8 mmol) was dissolved in ethanol (100 mL), and a solution of NaOH (1.10 g, 27.5 mmol) in H₂O (3.3 mL) was added. To this mixture was added slowly 50% hydrogen peroxide solution (3.7 mL, 54 mmol). A vigorous reaction began; this mixture was stirred for 3 h, then diluted with 700 mL H₂O and extracted with chloroform (3×500 mL). The organics were combined and dried over MgSO₄, then evaporated to give the crude product (1.62 g), which was then recrystallized from hot ethyl acetate (50 mL) to give the final compound as small white plates mp 81-82° C. (1.0 g, 25% yield). ¹H NMR (CDCl₃/TMS): δ (ppm): 5.74 (t, 2H, J 5.7); 4.43 (t, 2H, J 5.3); 3.07 (d, 4H, J 5.2); 2.92 (m, 4H); 1.35-1.22 (m, 4H); 1.16-1.08 (m, 4H); 0.77 (s, 12H). ¹³C NMR (CDCl₃/TMS): δ (ppm): 158.1, 69.8, 40.2, 35.7, 34.6, 24.7, 24.1.

1-(4-Benzoyl-2,6-dimethyl-phenyl)-3(pyridin-2-yl)-urea: To a cooled solution of anhydrous AlCl₃ (48 mmol) in 1,2-dichloroethane (15 mL), benzotrichloride (8 mmol) was added dropwise in 5 min under efficient stirring. The mixture was stirred further for 15 min at 0-5° C. N-2,6-Dimethylphenyl-N′-2-pyridinylurea (prepared by reacting 2,6-dimethylphenyl isocyanate with 2-aminopyridine in THF as described in Pavia, M., Lobbestael, S. J., Taylor, C. P., Hershenson, F. M., and Miskell, D. L. J. Med. Chem. 1990, 33, 854) (7 mmol) was dissolved in 5 mL 1,2-dichloroethane and added over 15 min, after which the cooling bath was removed and the mixture was stirred for 5 h. The completion of the conversion was checked by TLC (hexanes/EtOAc 8:2). The dark brown mixture was poored into crushed ice (50 g) and the resulting mixture was stirred at 70° C. for 0.5 h. After cooling, the organic layer was separated, washed with water (3×25 mL) and dried. The solvent was removed on a rotary evaporator and the residue was purified by column chromatography on silica gel using EtOAc/hexanes with gradient from 1:3 to 1:1 and finally MeOH. The resulted oil was triturated with Et₂O to give a light colored powder. Yield 1.46 g (4.2 mmol, 60%). Calcd. for C₂₁H₂₀N₃O₂ (MH+): 346.1579, found 346.1556.

General methodology for parallel urea synthesis for examples set forth in 5.1.15 to 5.1.36. The following general methodology was used to prepared the compounds set forth in 5.1.15 to 5.1.36.

Under a N₂ atmosphere, the appropriate amine (1-1.4 eq) was dissolved in CH₂Cl₂ and cooled to 0° C. A solution of the appropriate isocyanate in CH₂Cl₂ (1 eq.) was added dropwise. After approximately 1 h, the reaction mixture was passed through a Varian Chemelut CE1005 column absorbed with aqueous KHSO₄ (1-2M, 0.3-1.2 eq.) and eluted with CH₂Cl₂. The combined organic layers were evaporated in vacuo to yield the urea.

### 5.1.15 Preparation of N-(5-hydroxy-4,4-dimethylpentyl)-N-phenethylurea

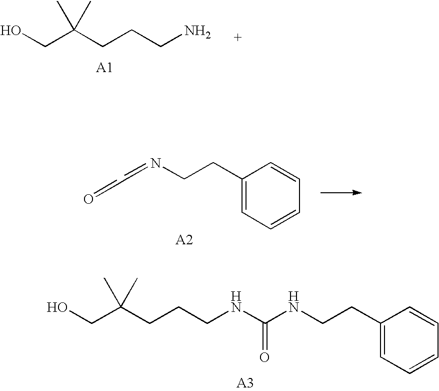

N-(5-hydroxy-4,4-dimethylpentyl)-N′-phenethylurea. Under a N₂ atmosphere, 5-amino-2,2-dimethyl-1-pentanol (0.50 g, 3.75 mmol) was dissolved in CH₂Cl₂ (1 mL) and cooled to 0° C. A solution of 1-(2-isocyanatoethyl)benzene in CH₂Cl₂ (1.36 M, 2.30 mL, 3.13 mmol) was added dropwise. The reaction mixture was passed through a Varian Chemelut CE1005 column absorbed with aqueous KHSO₄ (1M, 1.5 mL) and eluted with CH₂Cl₂ (2×5 mL). The combined organic layers were evaporated in vacuo to yield N-(5-hydroxy-4,4-dimethylpentyl)-N′-phenethylurea (1.101 g, 126%) as a thick yellow oil.

¹H NMR (CDCl₃): δ=7.30-7.14 (m, 5H), 5.20 (br s, 2H), 3.36 (t, J=7.1 Hz, 2H), 3.24 (s, 2H), 3.06 (t, J=6.8 Hz, 2H), 2.75 (t, J=7.2 Hz, 2H), 1.44-1.34 (m, 2H), 1.24-1.18 (m, 2H), 0.82 (s, 6H). ¹³C NMR (CDCl₃): δ=158.9, 139.2, 128.7 (2×), 128.4 (2×), 126.2, 70.7, 41.6, 41.0, 36.5, 35.2, 34.7, 24.7, 24.1 (2×).

### 5.1.16 Preparation of N-(5-hydroxy-4,4-dimethylpentyl)-N-(3-phenylpropyl)urea

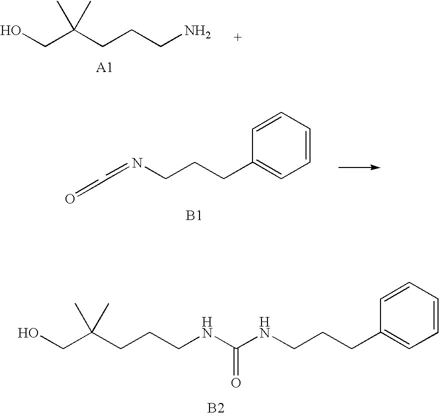

N-(5-hydroxy-4,4-dimethylpentyl)-N′-(3-phenylpropyl)urea. Under a N₂ atmosphere, 5-amino-2,2-dimethyl-1-pentanol (0.50 g, 3.75 mmol) was dissolved in CH₂Cl₂ (1 mL) and cooled to 0° C. A solution of 1-(2-isocyanatopropyl)benzene in CH₂Cl₂ (1.24 M, 2.52 mL, 3.13 mmol) was added dropwise. The reaction mixture was passed through a Varian Chemelut CE1005 column absorbed with aqueous KHSO₄ (1M, 1.5 mL) and eluted with CH₂Cl₂ (2×5 mL). The combined organic layers were evaporated in vacuo to yield N-(5-hydroxy-4,4-dimethylpentyl)-N′-(3-phenylpropyl)urea (1.087 g, 119%) as a thick yellow oil. ¹H NMR (CDCl₃): δ=7.27-7.12 (m, 5H), 5.24 (br s, 2H), 3.25 (s, 2H), 3.16 (t, J=7.1 Hz, 2H), 3.08 (t, J=6.7 Hz, 2H), 2.61 (t, J=7.8 Hz, 2H), 1.78 (quintet, J=7.4 Hz, 2H), 1.46-1.36 (m, 2H), 1.25-1.20 (m, 2H), 0.82 (s, 6H). ¹³C NMR (CDCl₃): δ=159.2, 141.6, 128.3 (2×), 128.3 (2×), 125.8, 70.8, 41.0, 39.9, 35.2, 34.8, 33.1, 31.9, 24.7, 24.1 (2×).

### 5.1.17 Preparation of N-(5-hydroxy-4,4-dimethylpentyl)-N′-(4-phenylbutyl)urea

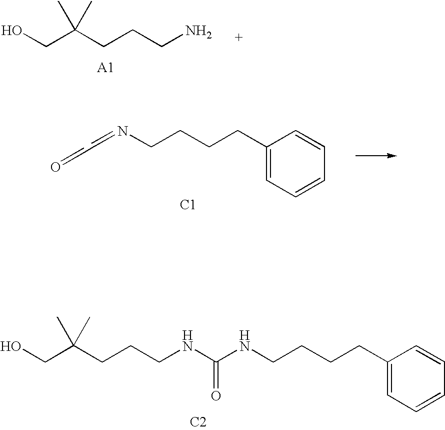

N-(5-hydroxy-4,4-dimethylpentyl)-N′-(4-phenylbutyl)urea. Under a N₂ atmosphere, 5-amino-2,2-dimethyl-1-pentanol (0.50 g, 3.75 mmol) was dissolved in CH₂Cl₂ (1 mL) and cooled to 0° C. A solution of 1-(4-isocyanatobutyl)benzene in CH₂Cl₂ (1.14 M, 2.74 mL, 3.13 mmol) was added dropwise. The reaction mixture was passed through a Varian Chemelut CE1005 column absorbed with aqueous KHSO₄ (1M, 1.5 mL) and eluted with CH₂Cl₂ (2×5 mL). The combined organic layers were evaporated in vacuo to yield N-(5-hydroxy-4,4-dimethylpentyl)-N′-(4-phenylbutyl)urea (1.152 g, 120%) as a thick yellow oil. ¹H NMR (CDCl₃): δ=7.28-7.22 (m, 2H), 7.18-7.12 (m, 3H), 3.25 (s, 2H), 3.13 (t, J=7.1 Hz, 2H), 3.08 (t, J=6.7 Hz, 2H), 2.59 (t, J=7.5 Hz), 1.67-1.36 (m, 6H), 1.26-1.20 (m, 2H), 0.82 (s, 6H). ¹³C NMR (CDCl₃): δ=159.1, 142.1, 128.3 (2×), 128.2 (2×), 125.7, 70.7, 41.0, 40.1, 35.5, 35.2, 34.8, 29.9, 28.6, 24.7, 24.1 (2×).

### 5.1.18 Preparation of N-(6-hydroxy-5,5-dimethylhexyl)-N′-(5-hydroxy-4,4-dimethylpentyl)urea

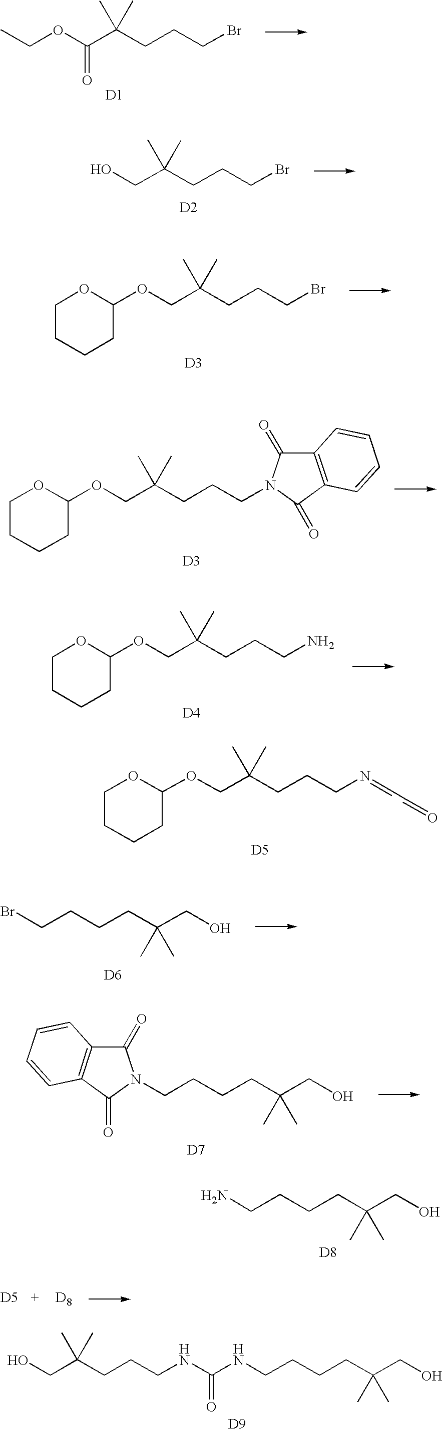

5-Bromo-2,2-dimethyl-1-pentanol. Under a N₂ atmosphere a solution of 5-bromo-2,2,-dimethylpentanoic acid ethyl ester (prepared according to Kuwahara, M.; Kawano, Y.; Kajino, M.; Ashida, Y.; Miyake, A., Chem. Pharm. Bull., 1997; 45, 1447-1457, 25.00 g, 100 mmol) in Et₂O (75 mL) was added slowly to a stirred suspension of LiAlH₄ (4.40 g, 110 mmol) in Et₂O (175 mL) in 25 min while keeping the internal temperature between −10° C. and −4° C. After 5 min, the reaction mixture was quenched with Na₂SO₄.10H₂O while stirring vigorously and keeping the internal temperature below −4° C. until the suspension turned white. The suspension was dried (Na₂SO₄) and filtered. The filtrate was evaporated in vacuo to yield 5-bromo-2,2-dimethyl-1-pentanol (19.97 g, 97%) as a colorless oil. ¹H NMR (CDCl₃): δ=3.39 (t, J=6.7 Hz, 2H), 3.32 (s, 2H), 1.90-1.77 (m, 2H), 1.43-1.31 (m, 2H), 0.88 (s, 6H). ¹³C NMR (CDCl₃): δ=69.7, 36.9, 35.7, 34.5, 27.4, 24.0 (2×).

2-[(5-Bromo-2,2-dimethylpentyl)oxy]tetrahydro-2H-pyran. At −10° C., 2,3-dihydro-4-H-pyran (11.9 mL, 126 mmol) was added to a solution of TsOH.H₂O (15 mg, 0.079 mmol) and 5-bromo-2,2-dimethyl-1-pentanol (19.97 g, 101 mmol) in CH₂Cl₂ (150 mL) in 10 min. After 5 min of stirring, the reaction mixture was allowed to warm to room temperature and stirring was continued for another 3 h after which saturated aqueous NaHCO₃ (50 mL) was added. The organic layer was separated and washed with saturated aqueous NaHCO₃ (50 mL) and brine (50 mL), dried (Na₂SO₄) and evaporated in vacuo. The residue was purified by column chromatography (silica, heptane:EtOAc=10:1) to yield 2-[(5-bromo-2,2-dimethylpentyl)oxy]tetrahydro-2H-pyran (22.89 g, 80%) as a colorless oil. ¹H NMR (CDCl₃): δ=4.53 (t, J=3.2 Hz, 1H), 3.82 (ddd, J=11.2, 8.2, 3.2 Hz, 1H), 3.53-3.43 (m, 1H), 3.46 (d, J=9.3 Hz, 1H), 3.37 (t, J=6.9 Hz, 2H), 2.98 (d, J=9.3 Hz, 1H), 1.92-1.45 (m, 8H), 1.44-1.34 (m, 2H), 0.91 (s, 3H), 0.90 (s, 3H). ¹³C NMR (CDCl₃): δ=98.9, 76.1, 61.9, 38.0, 34.8, 34.1, 30.7, 28.0, 25.7, 24.73, 24.65, 19.6. HRMS calcd for C₁₂H₂₄BrO₂ (MH⁺): 279.0950, found: 279.0955.

2-[4,4-Dimethyl-5-(tetrahydro-2H-2-pyranyloxy)pentyl]-1,3-isoindolinedione. Potassium phthalimide (8.96 g, 48.4 mmol) was added to a solution of 2-[(5-bromo-2,2-dimethylpentyl)oxy]tetrahydro-2H-pyran (12.15 g, 43.5 mmol) in DMF (100 mL) and the resulting mixture was stirred at 95° C. for 100 min and then allowed to cool to room temperature. Et₂O (100 mL) was added and the suspension was filtered. The residue was washed with Et₂O and the combined filtrates were washed with a solution of brine/H₂O (7:5, 120 mL) and brine (2×80 mL), dried (Na₂SO₄) and evaporated in vacuo to yield 2-[4,4-dimethyl-5-(tetrahydro-2H-2-pyranyloxy)pentyl]-1,3-isoindolinedione (15.03 g, 93%) as a colorless oil. ¹H NMR (CDCl₃): δ=0.80 (dd, J=5.6, 3.2 Hz, 2H), 7.67 (dd, J=5.4, 3.0 Hz, 2H), 4.49 (t, J=3.3 Hz, 1H), 3.80-3.70 (m, 1H), 3.62 (t, J=7.4 Hz, 2H), 3.47-3.36 (m, 1H), 3.39 (d, J=9.3 Hz, 1H), 2.92 (d, J=9.0 Hz, 1H), 1.82-1.36 (m, 8H), 1.33-1.24 (m, 2H), 0.85 (s, 3H), 0.84 (s, 3H). ¹³C NMR (CDCl₃): δ=168.2 (2×), 133.7 (2×), 132.1 (2×), 123.0 (2×), 98.8, 75.9, 61.6, 38.6, 36.0, 33.9, 30.4, 25.4, 24.5, 24.4, 23.3, 19.2. HRMS calcd for C₂₀H₂₈NO₄ (MH⁺): 346.1966, found: 346.1992.

4,4-Dimethyl-5-(tetrahydro-2H-2-pyranyloxy)-1-pentanamine. To a solution of 2-[4,4-dimethyl-5-(tetrahydro-2H-2-pyranyloxy)pentyl]-1,3-isoindolinedione (14.30 g, 41.4 mmol) in MeOH (50 mL) was added NH₂NH₂.H₂O (2.24 mL, 45.5 mmol) and the resulting mixture was stirred under reflux. After 2.3 h, EtOAc (50 mL) was added and the reaction mixture was cooled to room temperature in 5 min and then further cooled with an ice-bath without stirring. The formed suspension was filtered and the residue was washed with EtOAc (3×50 mL). The combined filtrates were washed with brine (3×100 mL), dried (Na₂SO₄), and evaporated in vacuo to yield 4,4-dimethyl-5-(tetrahydro-2H-2-pyranyloxy)-1-pentanamine (2.50 g, 28%) as a yellow oil. ¹H NMR (CDCl₃): δ=4.52 (t, J=3.3 Hz, 1H), 3.83 (ddd, J=11.3, 7.9, 3.5 Hz, 1H), 3.52-3.43 (m, 1H), 3.46 (d, J=9.0 Hz, 1H), 2.98 (d, J=9.0 Hz, 1H), 2.66 (t, J=6.9 Hz, 2H), 1.87-1.34 (m, 10H), 1.34-1.21 (m, 2H), 0.90 (s, 3H), 0.89 (s, 3H). ¹³C NMR (CDCl₃): δ=98.9, 76.3, 61.8, 43.1, 36.4, 34.0, 30.7, 28.3, 25.6, 24.6, 24.5, 19.5. HRMS calcd for C₁₂H₂₆NO₂ (MH⁺): 216.1964, found: 216.1957.

2-[(5-Isocyanato-2,2-dimethylpentyl)oxy]tetrahydro-2H-pyran. Under a N₂ atmosphere at 0° C., a solution of 4,4-dimethyl-5-(tetrahydro-2H-2-pyranyloxy)-1-pentanamine (1.195 g, 4.95 mmol) and Et₃N (1.54 mL, 11.0 mmol) in CH₂Cl₂ (40 mL) was added dropwise to a solution of triphosgene (549 mg, 1.85 mmol) in CH₂Cl₂ (40 mL) in 5 min. The reaction mixture was allowed to stir at room temperature for 15 min, after which the reaction mixture was washed with ice-cold aqueous HCl (0.1 M, 35 mL), ice-cold saturated aqueous NaHCO₃ (35 mL) and ice-cold brine (35 mL). After drying (Na₂SO₄), the organic layer was evaporated in vacuo to yield 2-[(5-isocyanato-2,2-dimethylpentyl)oxy]tetrahydro-2H-pyran (1.278 g, 95%) as a colorless oil/solid which was used as such.

2,2-Dimethyl-6-phthalimidohexanol. To a solution of 6-bromo-2,2-dimethylhexanol (prepared according to Ackerley, N.; Brewster, A. G.; Brown, G. R.; Clarke, D. S.; Foubister, A. J.; Griffin, S. J.; Hudson, J. A.; Smithers, M. J.; Whittamore, P. R. O., J. Med. Chem., 1995; 38; 1608-1628, 40.8 g, 194 mmol) in DMF (200 mL) was added potassium phthalimide (43.2 g, 233 mmol). The reaction mixture was stirred at 100° C. for 3.5 h, allowed to cool to room temperature and filtered. The residue was washed with Et₂O (200 mL) and the combined filtrates were poured out into Et₂O (500 mL). The resulting mixture was filtered again. The residue was washed with Et₂O (300 mL), and the combined filtrates were washed with aqueous NaCl (10%, 500 mL), brine (2×500 mL), dried (Na₂SO₄) and concentrated in vacuo to give 2,2-dimethyl-6-phtalimidohexanol (52.7 g, 97%) as a slightly off-white solid. ¹H NMR (CDCl₃): δ=7.89-7.81 (m, 2H), 7.74-7.68 (m, 2H), 3.70 (t, J=7.2 Hz, 2H), 3.30 (br s, 2H), 1.74-1.63 (m, 3H), 1.31-1.29 (m, 4H), 0.86 (s, 6H). ¹³C NMR (CDCl₃): δ=168.4 (2×), 133.8 (2×), 132.0 (2×), 123.1 (2×), 71.3, 37.7, 37.6, 34.9, 29.3, 23.8 (2×), 20.9.

6-Amino-2,2-dimethylhexanol. To a solution of 2,2-dimethyl-6-phthalimidohexanol (51.0 g, 182 mmol) in EtOH (250 mL) was added NH₂NH₂.H₂O (11.0 mL, 224 mmol). After stirring for 1 h at 90° C., a thick white precipitate had formed, and MeOH (100 mL) was added, in order to keep the mixture stirring. After another 1.5 h, concentrated HCl (20 mL) was added, causing a thick precipitate to form. The mixture was allowed to cool to room temperature and was filtered. The residue was washed with MeOH (3×100 mL), and the combined filtrates were concentrated in vacuo to a smaller volume, again causing precipitation. The mixture was filtered again, and the residue was washed with aqueous HCl (1 M, 3×100 mL). The filtrate (pH˜1) was washed with CH₂Cl₂ (3×100 mL) and brought to pH=11 with aqueous NaOH (6 M, 100 mL). The resulting mixture was extracted with CH₂Cl₂ (3×150 mL). The combined extracts were dried (Na₂SO₄), concentrated in vacuo and coevaporated in vacuo with CH₂Cl₂ (100 mL) to give 6-amino-2,2-dimethylhexanol (26.4 g, 95%) as a yellow oil. ¹H NMR (CDCl₃): δ=3.24 (s, 2H), 2.69 (t, J=6.9 Hz, 2H), 2.37 (br s, 3H), 1.47-1.38 (m, 2H), 1.38-1.19 (m, 4H), 0.84 (s, 6H). ¹³C NMR (CDCl₃): δ=70.5, 41.6, 38.0, 34.7, 33.9, 23.8 (2×), 20.7. HRMS calcd for C₈H₁₉NO₂ (M⁺): 145.1467, found: 145.1465.

N-(6-hydroxy-5,5-dimethylhexyl)-N′-(5-hydroxy-4,4-dimethylpentyl)urea. To a solution of 6-amino-2,2-dimethyl-1-hexanol (0.525 g, 3.61 mmol) in CH₂Cl₂ (1 mL), a solution of 2-[(5-isocyanato-2,2-dimethylpentyl)oxy]tetrahydro-2H-pyran (0.698 g, 2.89 mmol) in CH₂Cl₂ (1 mL) was added dropwise in 1 min. After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo. The residue was dissolved in MeOH (10 mL) and TsOH.H₂O was added until pH ˜3. After 20 h of stirring, K₂CO₃ was added until pH˜10. The reaction mixture was evaporated in vacuo and the residue was suspended in CH₂Cl₂ and a very small amount of MeOH, dried (Na₂SO₄) and evaporated in vacuo. The residue was coevaporated in vacuo with CH₂Cl₂ (5 mL) and vacuum dried to yield N-(6-hydroxy-5,5-dimethylhexyl)-N′-(5-hydroxy-4,4-dimethylpentyl)urea (1.069 g, 122%) as a turbid yellow oil. ¹H NMR (CDCl₃): δ=3.06 (s, 4H), 2.95 (t, J=6.9 Hz, 2H), 2.91 (t, J=6.9 Hz, 2H), 1.36-1.06 (m, 10H), 0.76 (s, 12H). ¹³C NMR (CDCl₃): δ=158.2, 69.7 (2×), 40.1, 39.1, 38.2, 35.7, 34.8, 34.6, 31.2, 24.7, 24.1 (4×), 20.8.

### 5.1.19 Preparation of N-(5-hydroxy-4,4-dimethylpentyl)-N′-(6-hydroxy-5-methyl-5-phenylhexyl)urea

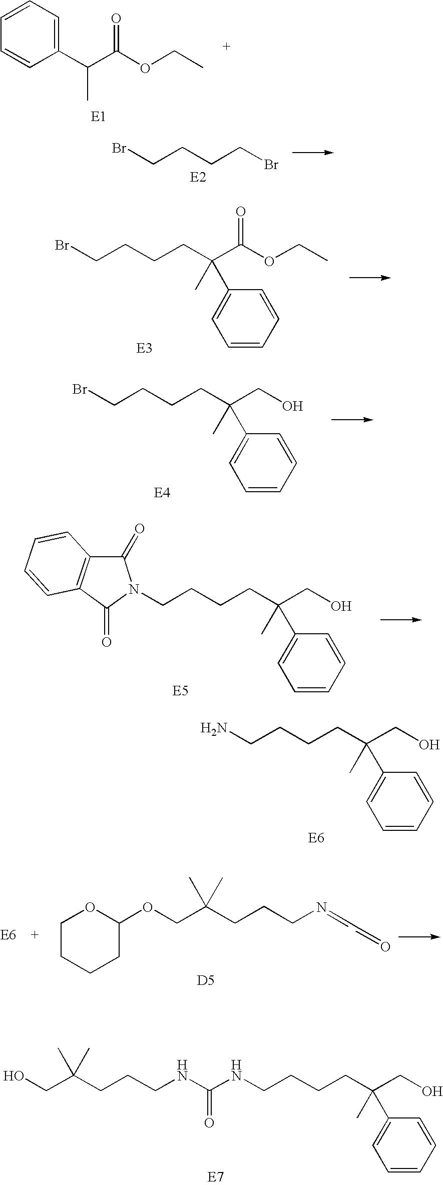

2-Phenyl-propionic acid ethyl ester. Under N₂ atmosphere, a solution of ethyl phenylacetate (50.0 g, 0.304 mol) in anhydrous THF (300 mL) was cooled to −40°αC and a solution of LDA (2.0 M in heptane, 152 mL, 304 mmol) was added dropwise over 30 min. The reaction mixture was stirred for 1 h, and MeI (60.51 g, 0.426 mmol) was added dropwise, followed by the addition of DMPU (20 mL). After 1 h, the reaction mixture was allowed to warm to room temperature and stirred overnight. The reaction mixture was poured into H₂O (400 mL) and extracted with EtOAc (3×100 mL). The combined organic layers were washed with saturated aqueous NH₄Cl (100 mL), aqueous HCl (1 M, 100 mL), saturated aqueous NaHCO₃ (100 mL), and brine (100 mL), dried (MgSO₄) and evaporated in vacuo. The residue was distilled in high vacuo to give 2-phenyl-propionic acid ethyl ester (46.13 g, 85%) as an oil. ¹H NMR (CDCl₃): δ=7.36-7.18 (m, 5H), 4.11 (m, 2H), 3.69 (q, J=7.1, 1H), 1.49 (d, J=7.1, 3H), 1.19 (t, J=7.1, 3H). ¹³C NMR (CDCl₃): δ=174.7, 140.9, 128.8 (2×), 127.6 (2×), 127.2, 60.9, 45.7, 18.8, 14.3.

Ethyl 6-bromo-2-methyl-2-phenylhexanoate. Under N₂ atmosphere, a solution of LDA (2.0 M in heptane, 14 mL, 28 mmol) was added dropwise to a stirred solution of 2-phenyl-propionic acid ethyl ester (5.0 g, 28.06 mmol) in anhydrous THF (50 mL) at −78° C. After 1 h, the reaction mixture was added to a −78° C. cold solution of 1,4-dibromobutane (10.06 g, 23.1 mmol) in THF. After the addition of DMPU (5 mL), the reaction mixture was stirred for 1 h, then warmed to room temperature, and stirred overnight. The mixture was poured into saturated aqueous NH₄Cl (500 mL) and extracted with EtOAc (4×100 mL). The combined organic phases were washed with brine (100 mL), HCl (1 M, 50 mL), saturated aqueous NaHCO₃ (50 mL), and brine (100 mL), dried (MgSO₄) and evaporated in vacuo. The residue was distilled to give ethyl 6-bromo-2-methyl-2-phenylhexanoate (7.16 g, 99%, bp. 130-131° C./0.2 mmHg; purity˜80%) as an oil. ¹H NMR (CDCl₃): δ=7.40-7.15 (m, 5H), 4.13 (q, J=6.7, 2H), 3.36 (t, J=6.7, 2H), 2.02 (m, 2H), 1.86 (m, 2H), 1.56 (s, 3H), 1.34 (m, 2H), 1.18 (t, J=6.7, 3H). ¹³C NMR (CDCl₃): δ=176.1, 143.9, 128.5 (2×), 126.8 (2×), 126.0, 60.9, 50.2, 38.5, 33.6, 33.3, 23.6, 22.8, 14.2. HRMS calcd for C₁₅H₂₂BrO₂ (MH⁺): 313.0803, found: 313.0836.

6-Bromo-2-methyl-2-phenyl-1-hexanol. Under a N₂ atmosphere, a solution of ethyl 6-bromo-2-methyl-2-phenylhexanoate (25.0 g, 75 mmol) in Et₂O (75 mL) was added slowly to a stirred suspension of LiAlH₄ (3.30 g, 82.5 mmol) in Et₂O (175 mL) in the course of 20 min while keeping the internal temperature between −10° C. and −4° C. After 5 min, the reaction mixture was quenched with Na₂SO₄.10H₂O while stirring vigorously and keeping the internal temperature below −4° C. until the suspension turned white. The suspension was dried (Na₂SO₄), filtered and the filtrate was evaporated in vacuo to yield 6-bromo-2-methyl-2-phenyl-1-hexanol (21.25 g, 99%) as a colorless oil.

¹H NMR (CDCl₃): δ=7.36-7.14 (m, 5H), 3.68 (d, J=10.8 Hz, 1H), 3.53 (d, J=10.8 Hz, 1H), 3.32 (t, J=6.8 Hz, 2H), 1.85-1.72 (m, 3H), 1.54 (dt, J=8.0, 4.8 Hz, 1H), 1.45 (s, 10H), 1.43-1.05 (m, 2H), 1.36 (s, 3H). ¹³C NMR (CDCl₃): δ=144.2, 128.3 (2×), 126.4 (2×), 126.1, 72.5, 43.4, 37.6, 33.6, 33.5, 22.7, 21.7. HRMS calcd for C₁₃H₁₉BrO (M)⁺: 270.0619, found: 270.0612.

2-(6-Hydroxy-5-methyl-5-phenylhexyl)-1,3-isoindolinedione. To a solution of 2-methyl-2-phenyl-6-bromohexanol (55.3 g, 204 mmol) in DMF (dry, 400 mL) was added potassium phthalimide (45.0 g, 243 mmol) and the resulting mixture was stirred at 100° C. After 2 h, the reaction mixture was allowed to cool to room temperature and Et₂O (400 mL) was added. The resulting mixture was filtered and the residue was washed with Et₂O (100 mL) and aqueous HCl (1M, 200 mL). To the combined filtrates was added aqueous HCl (1M, 100 mL) causing a precipitation. The resulting suspension was filtered and the residue was washed with H₂O (300 mL) to yield 2-(6-hydroxy-5-methyl-5-phenylhexyl)-1,3-isoindolinedione (8.72 g, 14%, mp: 148.8-150.1° C.) as a white powder. The combined filtrates were separated and the organic layer was evaporated in vacuo to yield 2-(6-hydroxy-5-methyl-5-phenylhexyl)-1,3-isoindolinedione (41.80 g, 68%, mp: 148.8-150.1° C.) as a white powder. An analytical sample (mp: 150.4-151.1° C.) was obtained by crystallization from EtOAc. ¹H NMR (CDCl₃): δ=7.79 (dd, J=5.4, 3.0 Hz, 2H), 7.67 (dd, J=5.4, 3.0 Hz, 2H), 7.32-7.24 (m, 4H), (sextet, J=4.2 Hz, 1H), 3.69 (d, J=11.1 Hz, 1H), 3.59 (t, J=7.5 Hz, 2H), 3.54 (d, J=11.1 Hz, 1H), 1.81 (dt, J=8.4, 4.4 Hz, 1H), 1.69-1.40 (m, 4H), 1.34 (s, 3H), 1.30-0.97 (m, 2H). ¹³C NMR (CDCl₃): δ=168.0 (2×), 144.3, 133.6 (2×), 131.9 (2×), 128.2 (2×), 126.4 (2×), 125.9, 122.9 (2×), 72.2, 43.4, 38.0, 37.8, 29.3, 21.8, 21.2. HRMS calcd for C₂₁H₂₄NO₃ (MH⁺): 338.1756, found: 338.1791.

6-Amino-2-methyl-2-phenyl-1-hexanol. To a solution of 2-(6-hydroxy-5-methyl-5-phenylhexyl)-1,3-isoindolinedione (50.12 g, 132 mmol) in MeOH (250 mL) was added NH₂NH₂.H₂O (7.95 mL, 164 mmol). The resulting mixture was stirred at reflux temperature for 2.5 h after which HCl (conc, 12 mL) was added. Stirring was continued for 1 h, after which the reaction mixture was allowed to cool to room temperature. The formed suspension was filtered and the residue was washed with MeOH (1×200 mL, 2×100 mL). To the combined filtrates was added HCl (conc, 2 mL) and this solution was concentrated in vacuo to approximately 100 mL. The resulting suspension was filtered and the residue was washed with aqueous HCl (1M, 1×200 mL, 2×50 mL). The combined filtrates were washed with CH₂Cl₂ (2×100 mL) and basified to pH˜11 with aqueous NaOH (6M). The resulting mixture was extracted with CH₂Cl₂ (2×250 mL). The combined extracts were concentrated in vacuo and coevaporated with toluene (2×100 mL) and CH₂Cl₂ (100 mL) to yield 6-amino-2-methyl-2-phenyl-1-hexanol (22.98 g, 84%) as a colorless oil. ¹H NMR (CDCl₃): δ=7.29-7.18 (m, 4H), 7.16-7.05 (m, 1H), 3.55 (d, J=10.8 Hz, 1H), 3.45 (d, J=10.8 Hz, 1H), 2.43 (t, J=7.1 Hz, 2H), 2.12 (br s, 3H), 1.72 (dt, J=8.7, 4.5 Hz, 1H), 1.49 (dt, J=8.7, 4.5 Hz, 1H), 1.37-1.20 (m, 2H), 1.29 (s, 3H), 1.20-1.3 (m, 1H), 1.20-0.85 (m, 1H). ¹³C NMR (CDCl₃): δ=145.1, 127.4 (2×), 126.0 (2×). 125.0, 71.2, 42.9, 41.2, 37.7, 33.6, 21.4, 20.7. HRMS calcd for C₁₃H₂₂NO for (MH⁺): 208.1701, found 208.1701.

N-(5-hydroxy-4,4-dimethylpentyl)-N′-(6-hydroxy-5-methyl-5-phenylhexyl)urea. To a solution of 6-amino-2-methyl-2-phenyl-1-hexanol (0.643 g, 3.10 mmol) in CH₂Cl₂ (1 mL), a solution of 2-[(5-isocyanato-2,2-dimethylpentyl)oxy]tetrahydro-2H-pyran (0.572 g, 2.37 mmol) in CH₂Cl₂ (1 mL) was added dropwise in 1 min. After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo. The remaining residue was dissolved in MeOH (10 mL) and TsOH.H₂O was added until pH=3. After 20 h of stirring, K₂CO₃ was added until pH=10 and the resulting mixture was concentrated in vacuo. The residue was suspended in CH₂Cl₂ and a very small amount of MeOH, dried (Na₂SO₄) and evaporated in vacuo. The residue was coevaporated with CH₂Cl₂ (5 mL) and vacuum dried to yield N-(5-hydroxy-4,4-dimethylpentyl)-N′-(6-hydroxy-5-methyl-5-phenylhexyl)urea (0.940 g, 102%) as a thick yellow oil. ¹H NMR (DMSO-d₆): δ=7.33-7.23 (m, 4H), 7.20-7.10 (m, 1H), 3.43 (s, 2H), 3.05 (s, 2H), 2.88 (t, J=6.9 Hz, 2H), 2.86 (t, J=7.1 Hz, 2H), 1.67 (dt, J=12.8, 4.4 Hz, 1H), 1.51 (dt, J=12.8, 4.6 Hz, 1H), 1.33-1.18 (m, 4H), 1.21 (s, 3H), 1.14-0.81 (m, 4H), 0.75 (s, 6H). ¹³C NMR (DMSO-d₆): δ=158.1, 146.5, 127.8 (2×), 126.5 (2×), 125.3, 70.1, 69.7, 42.7, 40.1, 39.0, 37.8, 35.6, 34.6, 30.9, 24.7, 24.1 (2×), 22.2, 21.0.

### 5.1.20 Preparation of N-(6-hydroxy-5,5-dimethylhexyl)-N′-phenethylurea

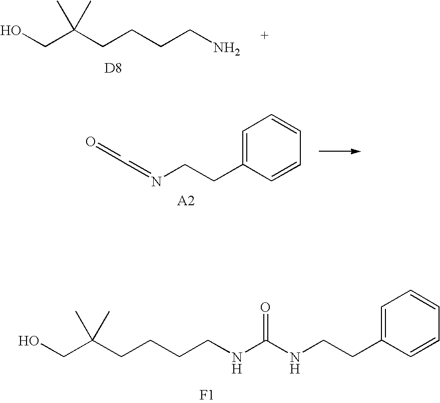

N-(6-hydroxy-5,5-dimethylhexyl)-N′-phenethylurea. Under a N₂ atmosphere at 0° C., a solution of 1-(2-isocyanatoethyl)benzene in CH₂Cl₂ (1.36 M, 2.09 mL, 2.84 mmol) was added dropwise to a solution of 6-amino-2,2-dimethyl-1-hexanol (0.50 g, 3.41 mmol) in CH₂Cl₂ (1 mL). The reaction mixture was passed through a Varian Chemelut CE1005 column absorbed with aqueous KHSO₄ (1 M, 1.5 mL) and eluted with CH₂Cl₂ (2×5 mL). The combined organic layers were evaporated in vacuo and vacuum dried to yield N-(6-hydroxy-5,5-dimethylhexyl)-N′-phenethylurea (1.024 g, 123%) as a thick yellow oil. ¹H NMR (CDCl₃): δ=7.29-7.23 (m, 2H), 7.20-7.15 (m, 3H), 5.20 (br s, 2H), 3.36 (t, J=6.9 Hz, 2H), 3.23 (s, 2H), 3.11 (t, J=6.4 Hz, 2H), 2.91 (br s, 1H), 2.76 (t, J=7.2 Hz), 1.45-1.35 (m, 2H), 1.25-1.20 (m, 4H), 0.83 (s, 6H). ¹³C NMR (CDCl₃): δ=159.0, 139.2, 128.7 (2×), 128.4 (2×), 126.2, 70.7, 41.6, 39.6, 37.5, 36.5, 34.9, 30.9, 24.2 (2×), 20.6.

### 5.1.21 Preparation of N-(6-hydroxy-5,5-dimethylhexyl)-N-(3-phenylpropyl)urea

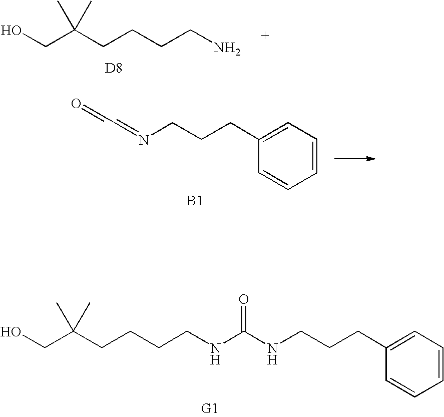

N-(6-hydroxy-5,5-dimethylhexyl)-N′-(3-phenylpropyl)urea. Under a N₂ atmosphere at 0° C., a solution of 1-(3-isocyanatopropyl)benzene in CH₂Cl₂ (1.24 M, 2.29 mL, 2.84 mmol) was added dropwise to a solution of 6-amino-2,2-dimethyl-1-hexanol (0.50 g, 3.41 mmol) in CH₂Cl₂ (1 mL). The reaction mixture was passed through a Varian Chemelut CE1005 column absorbed with aqueous KHSO₄ (1 M, 1.5 mL) and eluted with CH₂Cl₂ (2×5 mL). The combined organic layers were evaporated in vacuo and vacuum dried to yield N-(6-hydroxy-5,5-dimethylhexyl)-N′-(3-phenylpropyl)urea (1.036 g, 119%) as a thick yellow oil. ¹H NMR (CDCl₃): δ=7.26-7.21 (m, 2H), 7.17-7.12 (m, 3H), 5.37 (br s, 2H), 3.24 (s, 2H), 3.15 (t, J=7.1 Hz, 2H), 3.13 (t, J=7.1 Hz, 2H), 2.60 (t, J=7.8 Hz, 2H), 1.77 (quintet, J=7.4 Hz, 2H), 1.47-1.35 (m, 2H), 1.30-1.19 (m, 4H), 0.82 (s, 6H). ³C NMR (CDCl₃): δ=159.3, 141.6, 128.3 (2×), 128.2 (2×), 125.7, 70.7, 39.8, 39.6, 37.6, 34.9, 33.1, 31.9, 31.0, 24.2 (2×), 20.6.

### 5.1.22 Preparation of N-(6-hydroxy-5,5-dimethylhexyl)-N-(4-phenylbutyl)urea

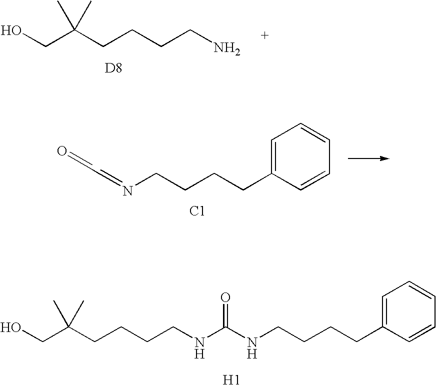

N-(6-hydroxy-5,5-dimethylhexyl)-N′-(4-phenylbutyl)urea. Under a N₂ atmosphere at 0° C., a solution of 1-(4-isocyanatobutyl)benzene in CH₂Cl₂ (1.14 M, 2.49 mL, 2.84 mmol) was added dropwise to a solution of 6-amino-2,2-dimethyl-1-hexanol (0.50 g, 3.41 mmol) in CH₂Cl₂ (1 mL). The reaction mixture was passed through a Varian Chemelut CE1005 column absorbed with aqueous KHSO₄ (1 M, 1.5 mL) and eluted with CH₂Cl₂ (2×5 mL). The combined organic layers were evaporated in vacuo and vacuum dried to yield N-(6-hydroxy-5,5-dimethylhexyl)-N′-(4-phenylbutyl)urea (1.025 g, 113%) as a thick yellow oil. ¹H NMR (CDCl₃): δ=7.27-7.22 (m, 2H), 7.17-7.12 (m, 3H), 3.24 (s, 2H), 3.13 (t, J=6.9 Hz, 4H), 2.59 (t, J=7.5 Hz, 2H), 1.67-1.36 (m, 6H), 1.26-1.21 (m, 4H), 0.82 (s, 6H). ¹³C NMR (CDCl₃): δ=159.2, 142.1, 128.3 (2×), 128.2 (2×), 125.7, 70.7, 40.1, 39.5, 37.5, 35.5, 34.9, 31.0, 29.9, 28.6, 24.2 (2×), 20.6.

### 5.1.23 Preparation of Methyl 4-([(6-hydroxy-5,5-dimethylhexyl)amino]carbonylamino)butanoate

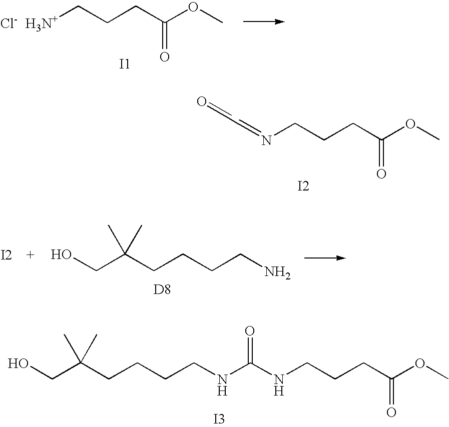

4-Isocyanato-butyric acid methyl ester. To a solution of triphosgene (3.87 g, 13.0 mmol) in CH₂Cl₂ (100 mL) was added portion wise 4-amino-butyric acid methyl ester hydrochloric acid salt (6.00 g, 39.1 mmol) at 0° C. in 5 min. Then, a solution of Et₃N (16.3 mL, 11.7 mmol) in CH₂Cl₂ (100 mL) was added dropwise to the reaction mixture in 45 min at 0° C. under N₂ atmosphere. After 5 min, the resultant mixture was washed with ice cold aqueous HCl (1M, 100 mL) and brine (100 mL), dried (Na₂SO₄), and concentrated in vacuo to give 4-isocyanato-butyric acid methyl ester (5.26 g, 94%) as a slightly yellow liquid, in which some crystalline material appeared. This compound was used as such in the following reactions. ¹H NMR (CDCl₃): δ=3.70 (s, 3H), 3.41 (t, J=6.5 Hz, 2H), 2.44 (t, J=7.2 Hz, 2H), 1.93 (quintet, J=6.8 Hz, 2H). ¹³C NMR (CDCl₃): δ=172.9, 122.0, 51.7, 42.1, 30.7, 26.2.

Methyl 4-([(6-hydroxy-5,5-dimethylhexyl)amino]carbonylamino)butanoate. Under a N₂ atmosphere at 0° C., a solution of methyl 4-isocyanatobutanoate (1.46 g, 10.22 mmol) in CH₂Cl₂ (7.0 mL) was added dropwise to a solution of 6-amino-2,2-dimethyl-1-hexanol (1.50 g, 10.22 mmol) in CH₂Cl₂ (5 mL) in 3 min. After 10 minutes of stirring, the reaction mixture was washed with aqueous HCl (1 M, 12 mL). To the organic layer were added a small amount of NaHCO₃ and Na₂SO₄. After filtration of the solids, the filtrate was evaporated in vacuo to yield methyl 4-([(6-hydroxy-5,5-dimethylhexyl)amino]carbonylamino)butanoate (2.828 g, 96%) as a thick yellow oil. ¹H NMR (CDCl₃): δ=3.68 (s, 3H), 3.29 (s, 2H), 3.20 (t, J=6.9 Hz, 2H), 3.18 (t, J=6.8 Hz, 2H), 2.38 (t, J=7.2 Hz, 2H), 1.82 (quintet, J=7.1 Hz, 2H), 1.52-1.39 (m, 2H), 1.35-1.21 (m, 4H), 0.85 (s, 6H). ¹³C NMR (CDCl₃): δ=174.2, 158.9, 70.9, 51.7, 39.8, 39.7, 37.6, 35.0, 31.3, 30.9, 25.4, 24.2 (2×), 20.7.

### 5.1.24 Preparation of Ethyl 6-([(6-hydroxy-5,5-dimethylhexyl)amino]carbonylamino)hexanoate

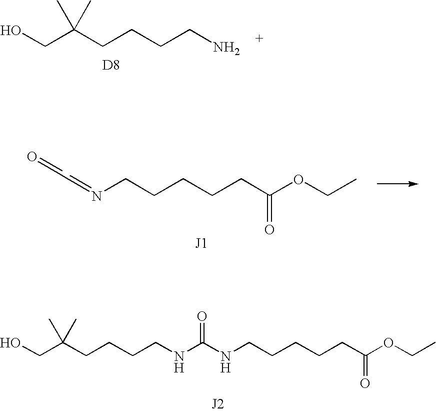

Ethyl 6-([(6-hydroxy-5,5-dimethylhexyl)amino]carbonylamino)hexanoate. Under a N₂ atmosphere at 0° C., a solution of ethyl 6-isocyanatohexanoate in CH₂Cl₂ (1.08 M, 2.63 mL, 2.84 mmol) was added dropwise to a solution of 6-amino-2,2-dimethyl-1-hexanol (0.50 g, 3.41 mmol) in CH₂Cl₂ (1 mL). The reaction mixture was passed through a Varian Chemelut CE1005 column absorbed with aqueous KHSO₄ (1 M, 1.5 mL) and eluted with CH₂Cl₂ (2×5 mL). The combined organic layers were evaporated in vacuo and vacuum dried to yield ethyl 6-([(6-hydroxy-5,5-dimethylhexyl)amino]carbonylamino)hexanoate (1.047 g, 112%) as a thick yellow oil.

¹H NMR (CDCl₃): δ=5.28 (br s, 2H), 4.12 (q, J=7.2 Hz, 2H), 3.27 (s, 2H), 3.20-3.10 (m, 5H), 2.29 (t, J=7.5 Hz, 2H), 1.63 (quintet, J=7.5 Hz, 2H), 1.49-1.30 (m, 6H), 1.30-1.22 (m, 7H), 0.85 (s, 6H). ¹³C NMR (CDCl₃): δ=173.8, 159.1, 70.7, 60.2, 40.0, 39.6, 37.6, 34.9, 34.1, 31.0, 29.9, 26.3, 24.5, 24.1 (2×), 20.6, 14.1.

### 5.1.25 Preparation of N-(6-hydroxy-5,5-dimethylhexyl)-N′-(6-hydroxy-5-methyl-5-phenylhexyl)urea

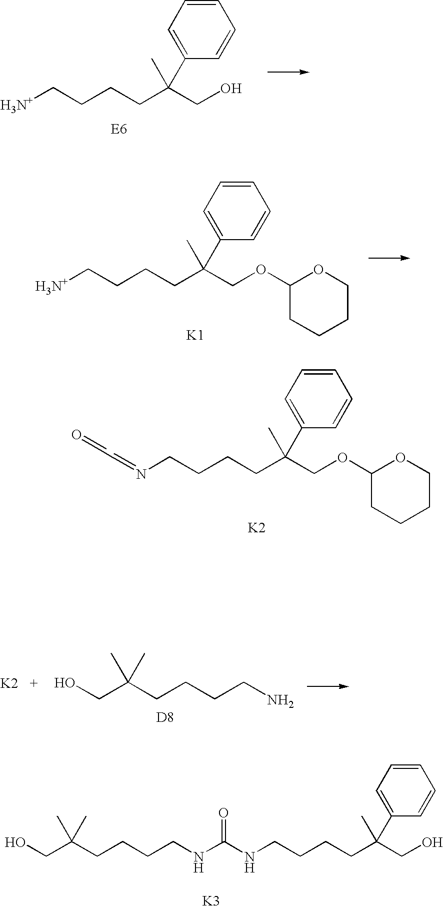

5-methyl-5-phenyl-6-(tetrahydro-2H-2-pyranyloxy)-1-hexanamine. To a solution of 6-amino-2-methyl-2-phenyl-1-hexanol (5.41 g, 26.1 mmol) in toluene (40 mL) was added TsOH.H₂O (4.95 g, 26.1 mmol). After 1 h of stirring, the reaction mixture was concentrated in vacuo and dissolved in CH₂Cl₂ (50 mL, it is necessary that pH˜2), after which 2,3-dihydro-4-H-pyran (2.98 mL, 32.6 mmol) was added. The reaction mixture was stirred for 1.5 h, after which K₂CO₃ (6.5 g, 47 mmol) was added and stirring was continued for another 15 min. The suspension was filtered and the residue was washed with CH₂Cl₂ (2×50 mL). The combined filtrates were washed with saturated aqueous NaHCO₃ (2×50 mL) and brine (75 mL), dried (Na₂SO₄), and concentrated in vacuo. The residue was purified by column chromatography (silica, CH₂Cl₂:MeOH:Et₃N, 1:0:025:0.020) to give 5-methyl-5-phenyl-6-(tetrahydro-2H-2-pyranyloxy)-1-hexanamine as a turbid oil. This oil was suspended in CH₂Cl₂ (150 mL), washed with brine (75 mL), dried (Na₂SO₄), and concentrated in vacuo to yield 5-methyl-5-phenyl-6-(tetrahydro-2H-2-pyranyloxy)-1-hexanamine (5.95 g, 78%) as a thick, colorless oil. ¹H NMR (CDCl₃): Mixture of diastereomers (1:1). 8=7.34-7.20 (m, 4H), 7.18-7.09 (m, 1H), 4.50 (t, J=3.3 Hz, 0.5H), 4.46 (t, J=3.2 Hz, 0.5H), 3.79 (d, J=9.3 Hz, 1H), 3.73-3.56 (m, 1H), 3.46-3.29 (m, 1H), 3.35 (d, J=9.3 Hz, 0.5H), 3.33 (d, J=9.3 Hz, 0.5H), 2.57 (t, J=6.9 Hz, 2H), 1.88-1.30 (m, 12H), 1.35 (s, 3H), 1.26-0.95 (m, 2H). ¹³C NMR (CDCl₃): Mixture of diastereomers (1:1). 8=145.46 (0.5×), 145.44 (0.5×), 127.5, 127.45, 126.025, 126.015, 125.25, 98.6 (0.5×), 98.5 (0.5×), 76.05, 61.6 (0.5×), 61.4 (0.5×), 41.87 (0.5×), 41.79 (0.5×), 41.685, 38.6 (0.5×), 38.4 (0.5×), 34.05, 30.45, 25.45, 22.7 (0.5×) 22.6 (0.5×), 21.20 (0.5×), 21.17 (0.5×), 19.2 (0.5×), 19.2 (0.5×). HRMS calcd for C₁₈H₂₉NO₂ (M)⁺: 291.2198, found: 291.2185.

2-[(6-Isocyanato-2-methyl-2-phenylhexyl)oxy]tetrahydro-2H-pyran. To a solution of triphosgene (261 mg, 0.88 mmol) in CH₂Cl₂ (15 mL) was added dropwise a solution of 5-methyl-5-phenyl-6-(tetrahydro-2H-2-pyranyloxy)-1-hexanamine (0.701 g, 2.21 mmol) and Et₃N (0.669 mL, 4.81 mmol) in CH₂Cl₂ (15 mL) at 0° C. under a N₂ atmosphere in 5 min. The reaction mixture was allowed to stir at room temperature for 15 min, after which the reaction mixture was washed with ice-cold aqueous HCl (0.1 M, 15 mL), ice-cold saturated aqueous NaHCO₃ (20 mL), and ice-cold brine (20 mL). After drying (Na₂SO₄), the organic layer was evaporated in vacuo to yield 2-[(6-isocyanato-2-methyl-2-phenylhexyl)oxy]tetrahydro-2H-pyran (0.761 g, 100%) as a colorless oil/solid which was used as such. ¹H NMR (CDCl₃): Mixture of diastereomers (1:1) δ=7.36-7.24 (m, 4H), 7.22-7.15 (m, 1H), 4.53 (t, J=3 Hz, 0.5H), 4.49 (t, J=3 Hz, 0.5H), 3.83 (d, J=6 Hz, 0.5H), 3.80 (d, J=6 Hz, 0.5H), 3.76-3.61 (m, 1H), 3.50-3.39 (m, 1H), 3.37 (d, J=1 Hz, 0.5H), 3.34 (d, J=1 Hz, 0.5H), 3.213 (t, J=7 Hz, 1H), 3.209 (t, J=7 Hz, 1H), 1.90-1.40 (m, 10H), 1.37 (s, 3H), 1.31-1.01 (m, 2H). ¹³C NMR (CDCl₃): Mixture of diastereomers (1:1) δ=145.70 (0.5×), 145.67 (0.5×), 128.3, 128.00, 127.99, 126.42, 126.40, 125.8, 99.1 (0.5×), 98.9 (0.5×), 76.2 (0.5×), 76.1 (0.5×), 61.9 (0.5×), 61.8 (0.5×), 42.7, 41.9 (0.5×), 41.8 (0.5×), 38.2 (0.5), 38.0 (0.5×), 31.9, 30.5, 25.5, 22.8 (0.5×), 22.6 (0.5×), 21.07 (0.5×), 21.06 (0.5×), 19.3 (0.5×), 19.2 (0.5×).

N-(6-hydroxy-5,5-dimethylhexyl)-N′-(6-hydroxy-5-methyl-5-phenylhexyl)urea. Under a N₂ atmosphere at 0° C., a solution of 2-[(6-isocyanato-2-methyl-2-phenylhexyl)oxy]tetrahydro-2H-pyran (0.763 g, 2.40 mmol) in CH₂Cl₂ (1 mL) was added dropwise to a solution of 6-amino-2,2-dimethyl-1-hexanol (0.437 g, 3.01 mmol) in CH₂Cl₂ (1 mL). After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo. The remaining residue was dissolved in MeOH (10 mL) and TsOH.H₂O was added until pH=3. After 20 h of stirring, K₂CO₃ was added until pH=10. The reaction mixture was concentrated in vacuo and suspended in CH₂Cl₂ and a very small amount of MeOH, dried (Na₂SO₄) and evaporated in vacuo. The residue was coevaporated with CH₂Cl₂ (5 mL) and vacuum dried to yield N-(6-hydroxy-5,5-dimethylhexyl)-N′-(6-hydroxy-5-methyl-5-phenylhexyl)urea (0.628 g, 69%) as a white yellowish foam. ¹H NMR (DMSO-d₆): δ=7.32-7.21 (m, 4H), 7.17-7.08 (m, 1H), 3.45 (s, 2H), 3.07 (s, 2H), 2.93 (t, J=6.6 Hz, 2H), 2.86 (t, J=6.9 Hz, 2H), 1.67 (dt, J=12.7, 4.4 Hz, 1H), 1.53 (dt, J=12.6, 4.4 Hz, 1H), 1.35-0.81 (m, 10H), 1.20 (s, 3H), 0.76 (s, 6H). ¹³C NMR (DMSO-d₆): δ=158.4, 146.9, 127.8 (2×), 126.5 (2×), 125.2, 70.3, 69.7, 42.8, 38.9, 38.1, 37.7, 34.8, 31.1, 30.9, 24.2 (2×), 22.5, 21.0, 20.7 (most probably one carbon atom overlaps with the DMSO-d₆ signals).

### 5.1.26 Preparation of N-(6-hydroxy-5,5-dimethylhexyl)-N-(7-hydroxy-6-methyl-6-phenylheptyl)urea

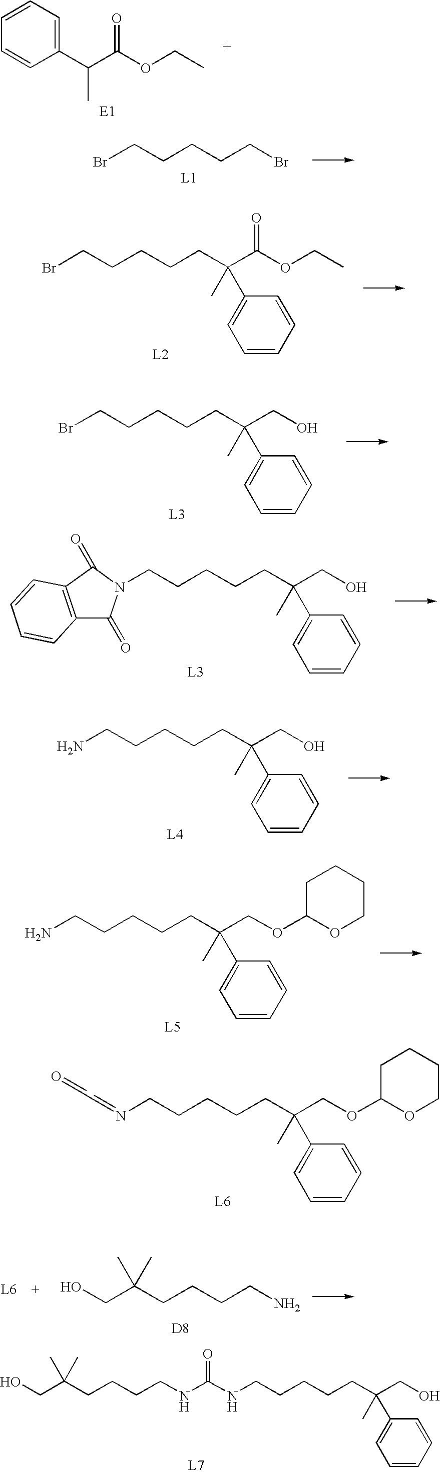

7-Bromo-2-methyl-2-phenyl-heptanoic acid ethyl ester. Under N₂ atmosphere, a solution of LDA (2.0 M in heptane, 1848 mL, 3.75 mol) was added dropwise to a stirred solution of 2-phenyl-propionic acid ethyl ester (660 g, 3.75 mol) in anhydrous THF (6600 mL) over 30 min at −78° C. After 1 h, 1,5-dibromopentane (1390 g, 6.04 mol) followed by DMPU (660 mL) was added. The reaction mixture was stirred for 1 h, warmed to room temperature and stirred overnight. The mixture was poured into saturated aqueous NH₄Cl solution (24 L) and extracted with EtOAc (4×6650 mL). The combined organic layers were washed with brine (9 L), aqueous HCl (1M, 6 L), saturated aqueous NaHCO₃ (6 L) and brine (6 L), dried (MgSO₄), and concentrated in vacuo. The residue was distilled to yield 7-bromo-2-methyl-2-phenyl-heptanoic acid ethyl ester (700 g, 57%, purity 91%) as a yellowish oil. Bp: 140-145° C./0.3 mmHg. ¹H NMR (CDCl₃): δ=7.30-7.20 (m, 5H), 4.09 (m, 2H), 3.34 (t, J=6.9 Hz, 2H), 2.05-1.80 (m, 4H), 1.53 (s, 3H), 1.43-1.14 (m, 4H), 1.16 (t, J=6.6 Hz, 3H). ¹³C NMR (CDCl₃): δ=176.0, 144.0, 128.3 (2×), 126.6 (2×), 125.9, 60.7, 50.1, 39.0, 33.8, 32.5, 28.6, 23.9, 22.8, 14.1. HRMS calcd. for C₁₆H₂₄BrO₂ (MH⁺): 327.0960, found: 327.0952.

7-Bromo-2-methyl-2-phenyl-1-heptanol. Under a N₂ atmosphere a solution of ethyl 7-bromo-2-methyl-2-phenyl-1-heptanoate (55.03 g, 91% pure, 153 mmol) in Et₂O (125 mL) was added to a stirred suspension of LiAlH₄ (7.4 g, 185 mmol) in Et₂O (300 mL) in the course of 35 min while keeping the internal temperature between −10° C. and −4° C. After 10 min, the reaction mixture was quenched with Na₂SO₄.10H₂O while stirring vigorously and keeping the internal temperature below −4° C. until the suspension turned white. The suspension was dried (Na₂SO₄) and filtered and the filtrate was concentrated in vacuo to yield 7-bromo-2-methyl-2-phenyl-1-heptanol (47.53 g, 103%) as a colorless oil. ¹H NMR (CDCl₃): δ=7.37-7.30 (m, 4H), 7.26-7.18 (m, 1H), 3.69 (dd, J=10.8, 5.4 Hz, 1H), 3.52 (dd, J=10.8, 7.8 Hz, 1H), 3.33 (t, J=6.9 Hz, 2H), 1.83-1.72 (m, 3H), 1.52 (ddd, J=13.5, 12.3, 4.8 Hz, 1H), 1.35 (s, 3H), 1.42-1.14 (m, 4H), 1.08-0.96 (m, 1H). ¹³C NMR (CDCl₃): δ=144.6, 128.5 (2×), 126.7 (2×), 126.2, 72.6, 43.4, 38.3, 33.8, 32.6, 28.8, 23.0, 21.5. HRMS calcd. for C₁₄H₂₁BrO (M)⁺: 284.0776, found: 284.0772.

2-(7-Hydroxy-6-methyl-6-phenylheptyl)-1,3-isoindolinedione. To a solution of 2-methyl-2-phenyl-7-bromoheptanol (46.81 g, 164 mmol) in DMF (350 mL) was added potassium phthalimide (36.8 g, 199 mmol) and the reaction mixture was stirred at 100° C. After 2.25 h, the reaction mixture was allowed to cool to room temperature and Et₂O (350 mL) was added. The resulting mixture was filtered and the residue was washed with Et₂O (100 mL). The combined filtrates were washed with HCl (1M, 175 ml and 150 mL) and brine (200 mL), dried (Na₂SO₄) and concentrated in vacuo to yield 2-(7-hydroxy-6-methyl-6-phenylheptyl)-1,3-isoindolinedione (52.42 g, 88%) as a thick, yellow oil. ¹H NMR (CDCl₃): δ=7.83 (dd, J=5.4, 3.2 Hz, 2H), 7.70 (dd, J=5.6, 3.2 Hz, 2H), 7.36-7.15 (m, 5H), 3.74-3.42 (m, 4H), 1.83-1.43 (m, 4H), 1.36-0.96 (m, 4H), 1.34 (s, 3H). ¹³C NMR (CDCl₃): δ=168.4 (2×), 144.7, 133.8 (2×), 132.1 (2×), 128.4 (2×), 126.6 (2×), 126.1, 123.1 (2×), 72.5, 43.3, 38.3, 37.9, 28.3, 27.5, 23.3, 21.5. HRMS calcd. for C₂₂H₂₆NO₃ (MH⁺): 352.1913, found: 352.1907.

7-Amino-2-methyl-2-phenyl-1-heptanol. To a solution of 2-(7-hydroxy-6-methyl-6-phenylheptyl)-1,3-isoindolinedione (51.73 g, 147 mmol) in EtOH (250 mL) was added NH₂NH₂.H₂O (8.67 mL, 178 mmol). The resulting mixture was stirred at 100° C. for 2 h after which HCl (conc, 12 mL) was added. Stirring was continued for 1 h, after which the reaction mixture was allowed to cool to room temperature. The formed suspension was filtered and the residue was washed with MeOH (3×100 mL). The combined filtrates were concentrated to approximately 100 mL. The formed suspension was filtered and the residue was washed with HCl (1M, 3×100 mL). The combined aqueous filtrates were washed with CH₂Cl₂ (3×100 mL) and the remaining aqueous suspension was filtered, the residue was washed with HCl (1M, 2×50 mL) and the combined aqueous filtrates were washed with CH₂Cl₂ (3×150 mL). The combined aqueous layers were brought to pH˜11 with aqueous NaOH (6M) and extracted with a mixture of CH₂Cl₂ and EtOH (9:1, 3×300 mL). The combined organic layers were dried (Na₂SO₄) and concentrated to yield 7-amino-2-methyl-2-phenyl-1-heptanol (32.58 g, 100%) as a colorless oil. ¹H NMR (CDCl₃): δ=7.32-7.22 (m, 4H), 7.20-7.11 (m, 1H), 3.62 (d, J=10.8 Hz, 1H), 3.49 (d, J=10.8 Hz, 1H), 2.84 (s, 3H), 2.54 (t, J=7.1 Hz, 2H), 1.80-1.66 (m, 1H), 1.51 (dt, J=8.9, 3.9 Hz, 1H), 1.42-0.89 (m, 6H), 1.31 (s, 3H). ¹³C NMR (CDCl₃): 3=145.0, 128.0 (2×), 126.4 (2×), 125.6, 72.0, 43.3, 41.5, 38.4, 32.3, 27.5, 23.6, 21.8. HRMS calcd. for C₁₄H₂₄NO (MH⁺): 222.1858, found: 222.1850.

6-Methyl-6-phenyl-7-(tetrahydro-2H-2-pyranyloxy)-1-heptanamine. To a solution of 7-amino-2-methyl-2-phenyl-1-heptanol (7.527 g, 33.9 mmol) in toluene (50 mL) was added TsOH.H₂O (6.46 g, 33.9 mmol, pH 6). After 15 min of stirring, the reaction mixture was concentrated in vacuo. The residue was dissolved in CH₂Cl₂ and cooled to about 15° C. 2,3-Dihydro-4-H-pyran (6.21 mL, 67.8 mmol) was added and the reaction mixture was stirred for 1 h, after which K₂CO₃ (9.85 g, 67.8 mmol) was added. The resulting mixture was filtered and the filtrate was washed with saturated aqueous NaHCO₃ (2×75 mL) and brine (100 mL), dried (Na₂SO₄) and evaporated in vacuo. The residue was purified by column chromatography (silica, CH₂Cl₂:MeOH:Et₃N=50:1.25:1) to yield 6-methyl-6-phenyl-7-(tetrahydro-2H-2-pyranyloxy)-1-heptanamine (7.03 g, 68%) as a yellow oil. ¹H NMR (CDCl₃): (mixture of diastereomers) δ=7.37-7.24 (m, 4H), 7.20-7.12 (m, 1H), 4.50 (dt, J=12.9, 3.3 Hz, 1H), 3.80 (dd, J=9.5, 2.0 Hz, 1H), 3.75-3.37 (m, 2H), 3.35 (t, J=9.0 Hz, 1H), 2.60 (t, J=7.2 Hz, 2H), 1.93-0.93 (m, 16H), 1.35 (s, 3H). ¹³C NMR (CDCl₃): (mixture of diastereomers) δ=145.9, 127.7, 127.6, 126.3 (2×), 125.4, 98.7, 98.6, 76.1, 61.5, 61.4, 41.8 (2×), 41.7, 38.7, 38.5, 33.2, 30.3, 27.4, 25.3, 23.6 (2×), 22.6, 22.5, 19.1, 19.0. HRMS calcd. for C₁₉H₃₁NO₂ (M)⁺: 305.2355, found: 305.2364.

2-[(7-isocyanato-2-methyl-2-phenylheptyl)oxy]tetrahydro-2H-pyran. Under a N₂ atmosphere at 0° C., a solution of 6-methyl-6-phenyl-7-(tetrahydro-2H-2-pyranyloxy)-1-heptanamine (0.587 g, 1.92 mmol) and Et₃N (0.534 mL, 3.84 mmol) in CH₂Cl₂ (10 mL) was added dropwise to a solution of triphosgene (190 mg, 0.64 mmol) in CH₂Cl₂ (10 mL) in 5 min. The reaction mixture was allowed to stir at room temperature for 15 min, after which the reaction mixture was washed with ice-cold aqueous HCl (0.1 M, 20 mL), ice-cold saturated aqueous NaHCO₃ (15 mL) and ice-cold brine (15 mL). After drying (Na₂SO₄), the organic layer was evaporated in vacuo to yield 2-[(7-isocyanato-2-methyl-2-phenylheptyl)oxy]tetrahydro-2H-pyran (0.600 g, 94%) as a yellow oil which was used as such. ¹H NMR (CDCl₃): (mixture of diastereomers) δ=7.37-7.25 (m, 4H), 7.20-7.13 (m, 1H), 4.50 (dt, J=11.7, 3.2 Hz, 1H), 3.81 (dd, J=9.3, 2.4 Hz, 1H), 3.75-3.60 (m, 1H), 3.48-3.38 (m, 1H), 3.35 (dd, J=9.5, 4.7 Hz, 1H), 3.19 (t, J=6.8, 2H), 1.88-0.94 (m, 14H), 1.36 (s, 3H). ¹³C NMR (CDCl₃): (mixture of diastereomers) δ=145.9, 145.8, 127.9 (2×), 126.4 (2×), 125.7, 99.0, 98.8, 61.8, 61.6, 42.8, 41.9, 41.8, 38.7, 38.5, 31.0, 30.5, 27.1 (2×), 25.4, 23.3 (2×), 22.7, 22.6, 19.3, 19.2.

N-(6-hydroxy-5,5-dimethylhexyl)-N′-(7-hydroxy-6-methyl-6-phenylheptyl)urea. Under a N₂ atmosphere at 0° C., a solution of 2-[(7-isocyanato-2-methyl-2-phenylheptyl)oxy]tetrahydro-2H-pyran (0.713 g, 2.15 mmol) in CH₂Cl₂ (1 mL) was added dropwise to a solution of 6-amino-2,2-dimethyl-1-hexanol (0.422 g, 2.91 mmol) in CH₂Cl₂ (1 mL) in 1 min. After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo. The remaining residue was dissolved in MeOH (10 mL) and TsOH.H₂O was added until pH=3. After 20 h of stirring, K₂CO₃ was added until pH=10. The reaction mixture was concentrated in vacuo and suspended in CH₂Cl₂ and a very small amount of MeOH, dried (Na₂SO₄) and evaporated in vacuo. The residue was coevaporated with CH₂Cl₂ (5 mL) and vacuum dried to yield N-(6-hydroxy-5,5-dimethylhexyl)-N′-(7-hydroxy-6-methyl-6-phenylheptyl)urea (0.992 g, 118%) as a turbid white oil. ¹H NMR (DMSO-d₆): δ=7.35-7.22 (m, 4H), 7.18-7.09 (m, 1H), 3.44 (s, 2H), 3.06 (s, 2H), 2.94 (t, J=6.6 Hz, 2H), 2.87 (t, J=6.6 Hz, 2H), 1.73-1.37 (m, 2H), 1.36-0.70 (m, 12H), 1.22 (s, 3H), 0.76 (s, 6H). ¹³C NMR (DMSO-d₆): δ=128.3, 146.6, 127.8 (2×), 126.5 (2×), 125.2, 70.3, 69.7, 42.7, 39.1 (2×), 38.2, 38.1, 34.8, 31.2, 30.0, 27.3, 24.2 (2×), 23.4, 22.2, 20.8.

### 5.1.27 Preparation of N-(6-hydroxy-5-methyl-5-phenylhexyl)-N′-phenethylurea

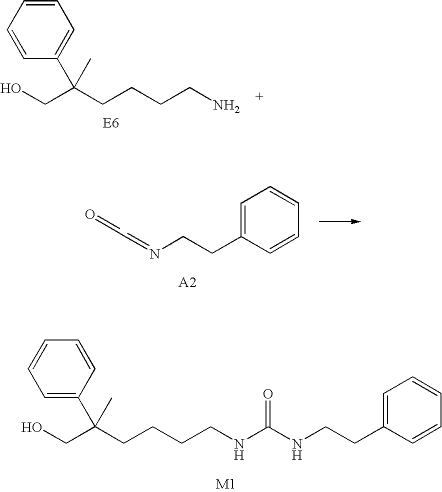

N-(6-hydroxy-5-methyl-5-phenylhexyl)-N′-phenethylurea. Under a N₂ atmosphere at 0° C., a solution of 1-(2-isocyanatoethyl)benzene in CH₂Cl₂ (1.36 M, 1.42 mL, 1.93 mmol) was added dropwise to a solution of 6-amino-2-methyl-2-phenyl-1-hexanol (0.50 g, 2.32 mmol) in CH₂Cl₂ (1 mL). The reaction mixture was passed through a Varian Chemelut CE1005 column absorbed with aqueous KHSO₄ (1 M, 1.5 mL) and eluted with CH₂Cl₂ (2×5 mL). The combined organic layers were evaporated in vacuo and vacuum dried to yield N-(6-hydroxy-5-methyl-5-phenylhexyl)-N′-phenethylurea (0.85 g, 124%) as a thick yellow oil. ¹H NMR (CDCl₃): δ=7.27-7.09 (m, 10H), 5.19 (t, J=5.7 Hz, 1H), 5.14 (t, J=5.7 Hz, 1H), 3.54 (q, J=11.0 Hz), 3.28 (q, J=6.7 Hz, 2H), 2.96 (q, J=6.5 Hz, 2H), 2.68 (t, J=7.2 Hz, 2H), 2.59 (br s, 1H), 1.60 (ddt, J=53.7, 12.6, 4.5 Hz, 2H), 1.31 (quintet, 7.2 Hz, 2H), 1.26 (s, 3H), 1.20-0.88 (m, 2H). ¹³C NMR (CDCl₃): δ=158.8, 145.2, 139.1, 128.6 (2×), 128.3 (2×), 128.2 (2×), 126.4 (2×), 126.1, 125.8, 71.2, 42.9, 41.4, 39.5, 37.5, 36.4, 30.7, 21.9, 20.8.

### 5.1.28 Preparation of N-(6-hydroxy-5-methyl-5-phenylhexyl)-N′-(3-phenylpropyl)urea

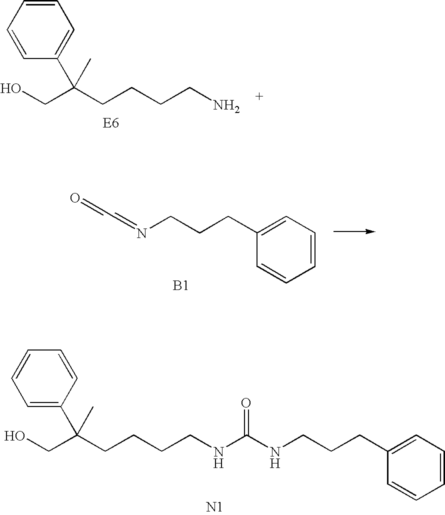

N-(6-hydroxy-5-methyl-5-phenylhexyl)-N′-(3-phenylpropyl)urea. Under a N₂ atmosphere at 0° C., a solution of 1-(3-isocyanatopropyl)benzene in CH₂Cl₂ (1.24 M, 1.56 mL, 1.93 mmol) was added dropwise to a solution of 6-amino-2-methyl-2-phenyl-1-hexanol (0.50 g, 2.32 mmol) in CH₂Cl₂ (1 mL). The reaction mixture was passed through a Varian Chemelut CE1005 column absorbed with aqueous KHSO₄ (1 M, 1.5 mL) and eluted with CH₂Cl₂ (2×5 mL). The combined organic layers were evaporated in vacuo and vacuum dried to yield N-(6-hydroxy-5-methyl-5-phenylhexyl)-N′-(3-phenylpropyl)urea (0.847 g, 119%) as a thick yellow oil. ¹H NMR (CDCl₃): δ=7.30-7.07 (m, 10H), 5.24-5.04 (m, 2H), 3.56 (q, J=10.8 Hz, 2H), 3.14-2.94 (m, 2H), 2.56 (t, J=7.7 Hz, 2H), 2.48 (br s, 1H), 1.79-1.65 (m, 3H), 1.51 (dt, J=12.8, 5.2 Hz, 1H), 1.34 (quintet, 7.2 Hz, 2H), 1.26 (s, 3H), 1.22-0.89 (m, 2H). ¹³C NMR (CDCl₃): δ=159.0, 145.2, 141.5, 128.2 (6×), 126.4 (2×), 125.9, 125.7, 71.3, 43.0, 39.7, 39.6, 37.6, 33.0, 31.8, 30.8, 22.0, 20.8.

### 5.1.29 Preparation of N-(6-hydroxy-5-methyl-5-phenylhexyl)-N′-(4-phenylbutyl)urea

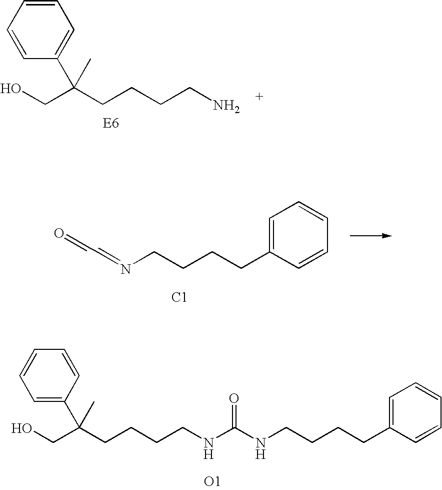

N-(6-hydroxy-5-methyl-5-phenylhexyl)-N′-(4-phenylbutyl)urea. Under a N₂ atmosphere at 0° C., a solution of 1-(4-isocyanatobutyl)benzene in CH₂Cl₂ (1.14 M, 1.69 mL, 1.93 mmol) was added dropwise to a solution of 6-amino-2-methyl-2-phenyl-1-hexanol (0.50 g, 2.32 mmol) in CH₂Cl₂ (1 mL). The reaction mixture was passed through a Varian Chemelut CE1005 column absorbed with aqueous KHSO₄ (1 M, 1.5 mL) and eluted with CH₂Cl₂ (2×5 mL). The combined organic layers were evaporated in vacuo and vacuum dried to yield N-(6-hydroxy-5-methyl-5-phenylhexyl)-N′-(4-phenylbutyl)urea (0.853 g, 115%) as a thick yellow oil. ¹H NMR (CDCl₃): δ=7.30-7.09 (m, 10H), 5.09-5.00 (m, 2H), 3.57 (q, J=10.7 Hz, 2H), 3.11-2.96 (m, 4H), 2.56 (t, J=7.5 Hz, 2H), 2.46 (br s, 1H), 1.72 (dt, J=12.8, 4.5 Hz, 1H), 1.64-1.29 (m, 7H), 1.27 (s, 3H), 1.20-0.90 (m, 2H). ¹³C NMR (CDCl₃): δ=158.9, 145.2, 142.1, 128.2 (6×), 126.4 (2×), 125.9, 125.6, 71.3, 43.0, 40.0, 39.6, 37.5, 35.4, 30.8, 29.8, 28.5, 22.0, 20.8.

### 5.1.30 Preparation of Ethyl 6-([(6-hydroxy-5-methyl-5-phenylhexyl)amino]carbonylamino)hexanoate

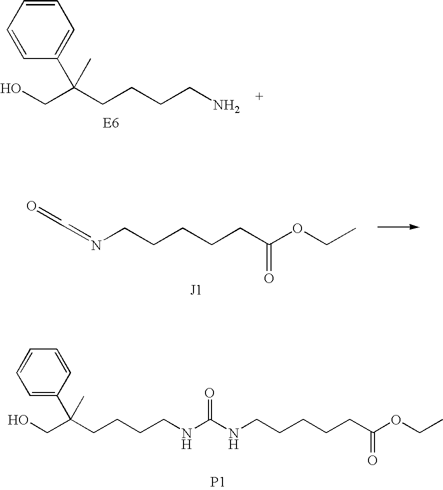

Ethyl 6-([(6-hydroxy-5-methyl-5-phenylhexyl)amino]carbonylamino)-hexanoate. Under a N₂ atmosphere at 0° C., a solution of ethyl 6-isocyanatohexanoate CH₂Cl₂ (1.08 M, 1.79 mL, 1.93 mmol) was added dropwise to a solution of 6-amino-2-methyl-2-phenyl-1-hexanol (0.50 g, 2.32 mmol) in CH₂Cl₂ (1 mL). The reaction mixture was passed through a Varian Chemelut CE1005 column absorbed with aqueous KHSO₄ (1 M, 1.5 mL) and eluted with CH₂Cl₂ (2×5 mL). The combined organic layers were evaporated in vacuo and vacuum dried to yield ethyl 6-([(6-hydroxy-5-methyl-5-phenylhexyl)amino]carbonylamino)hexanoate (0.922 g, 122%) as a thick yellow oil. ¹H NMR (CDCl₃): δ=7.32-7.26 (m, 4H), 7.19 (sextet, J=4.2 Hz, 1H), 4.97 (t, J=5.7, 1H), 4.93 (t, J=5.7 Hz), 4.10 (q, J=7.2 Hz, 2H), 3.70-3.52 (m, 2H), 3.14-2.99 (m, 4H), 2.44 (br s, 1H), 2.27 (t, J=7.4 Hz), 1.76 (dt, J=12.9, 4.5 Hz, 1H), 1.66-0.93 (m, 13H), 1.31 (s, 3H), 1.24 (t, J=7.2 Hz, 2H). ³C NMR (CDCl₃): δ=173.7, 158.8, 145.2, 128.2 (2×), 126.4 (2×), 125.9, 71.4, 60.2, 43.0, 39.9, 39.7, 37.6, 34.0, 30.8, 29.8, 26.2, 24.5, 22.0, 20.8, 14.1.

### 5.1.31 Preparation of N-(7-hydroxy-6-methyl-6-phenylheptyl)-N′-(6-hydroxy-5-methyl-5-phenylhexyl)urea

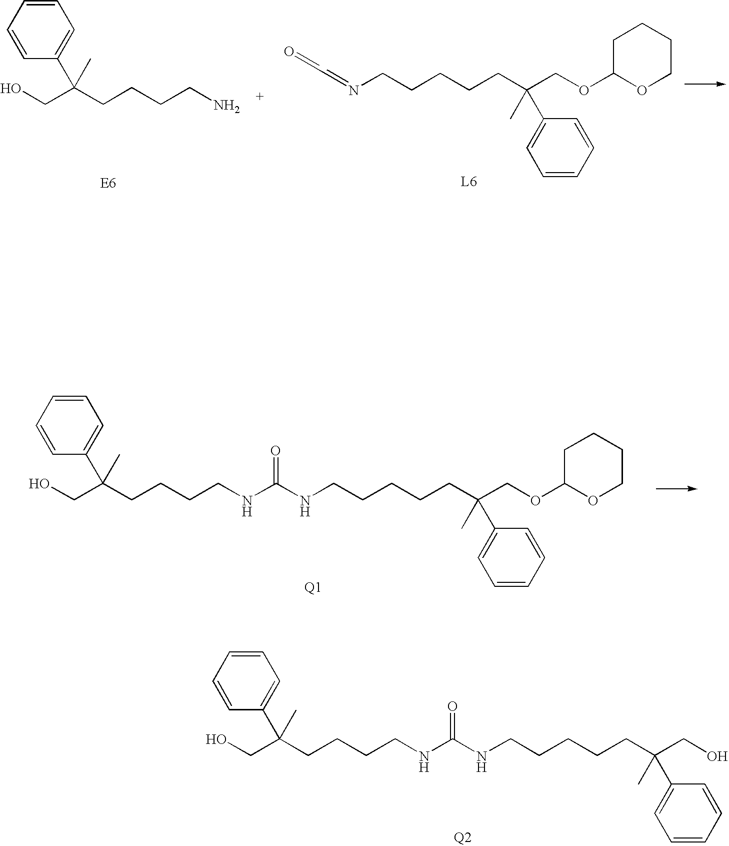

N-(6-hydroxy-5-methyl-5-phenylhexyl)-N′-[6-methyl-6-phenyl-7-(tetrahydro-2H-2-pyranyloxy)heptyl]urea. A solution of 2-[(7-isocyanato-2-methyl-2-phenylheptyl)oxy]tetrahydro-2H-pyran (0.600 g, 1.81 mmol) in CH₂Cl₂ (1 mL) was added dropwise to a solution of 6-amino-2-methyl-2-phenyl-1-hexanol (0.450 g, 2.17 mmol) in CH₂Cl₂ (1 mL). After 0.5 h, the reaction mixture was passed through a Varian Chemelut CE1005 column absorbed with aqueous KHSO₄ (1 M, 1.5 mL) and eluted with CH₂Cl₂ (2×10 mL). The combined organic layers were evaporated in vacuo and vacuum dried to yield N-(6-hydroxy-5-methyl-5-phenylhexyl)-N′-[6-methyl-6-phenyl-7-(tetrahydro-2H-2-pyranyloxy)heptyl]urea (0.952 g, 98%) as a yellow oil. ¹H NMR (CDCl₃): (mixture of diastereomers) δ=7.36-7.23 (m, 8H), 7.23-7.12 (m, 2H), 4.67-4.60 (m, 2H), 4.47 (dt, J=13.5, 3.2 Hz, 1H), 3.79 (d, J=9.3 Hz, 1H), 3.75-3.52 (m, 3H), 3.46-3.36 (m, 1H), 3.33 (t, J=9.6 Hz, 1H), 3.08-2.95 (m, 4H), 2.20 (br s, 1H), 1.84-0.90 (m, 20H), 1.34 (s, 3H), 1.30 (s, 3H). ³C NMR (CDCl₃): (mixture of diastereomers) δ=158.6, 146.0, 145.9, 145.2, 128.3 (2×), 127.9, 127.8, 126.5 (2×), 125.9, 125.6, 99.2, 99.0, 76.5, 76.4, 71.5, 62.0, 61.8, 43.1, 41.9, 41.8, 40.3, 39.7, 38.8, 38.7, 37.6, 30.8, 30.5, 30.1, 30.0, 27.6, 25.4, 23.7 (2×), 22.6 (2×), 22.0, 20.8, 19.4, 19.2.

N-(7-hydroxy-6-methyl-6-phenylheptyl)-N′-(6-hydroxy-5-methyl-5-phenylhexyl)urea. N-(6-hydroxy-5-methyl-5-phenylhexyl)-N′-[6-methyl-6-phenyl-7-(tetrahydro-2H-2-pyranyloxy)heptyl]urea (0.942 g, 1.75 mmol) was dissolved in MeOH (10 mL) and TsOH.H₂O was added until pH=3. After 20 h of stirring, K₂CO₃ was added until pH=10. The reaction mixture was concentrated in vacuo and suspended in CH₂Cl₂ and a very small amount of MeOH, dried (Na₂SO₄) and evaporated in vacuo. The residue was coevaporated with CH₂Cl₂ (10 mL) and vacuum dried to yield N-(7-hydroxy-6-methyl-6-phenylheptyl)-N′-(6-hydroxy-5-methyl-5-phenylhexyl)urea (0.792 g, 100%) as a foam/oil. ¹H NMR (CDCl₃): δ=7.34-7.25 (m, 8H), 7.23-7.13 (m, 2H), 3.66-3.50 (m, 4H), 3.02 (q, J=6.7 Hz, 4H), 1.81-1.66 (m, 2H), 1.62-1.46 (m, 3H), 1.44-0.94 (11H), 1.31 (s, 3H), 1.30 (s, 3H). ¹³C NMR (CDCl₃): δ=158.5, 145.2, 145.0, 128.0 (4×), 126.3 (2×), 126.2 (2×), 125.6, 76.4, 71.4, 70.9, 42.8 (2×), 39.7, 39.2, 37.9, 37.4, 30.6, 29.7, 27.2, 23.2, 21.8, 21.6, 20.6.

### 5.1.32 Preparation of N-(7-hydroxy-6-methyl-6-phenylheptyl)-N′-phenethylurea

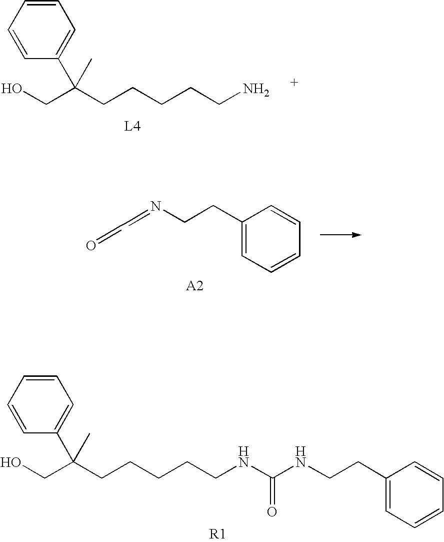

N-(7-hydroxy-6-methyl-6-phenylheptyl)-N′-phenethylurea. A solution of 1-(2-isocyanatoethyl)benzene (0.329 g, 2.24 mmol) in CH₂Cl₂ (1 mL) was added dropwise to a solution of 7-amino-2-methyl-2-phenyl-1-heptanol (0.596 g, 2.69 mmol) in CH₂Cl₂ (1 mL) in 1 min. After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo and vacuum dried to yield N-(7-hydroxy-6-methyl-6-phenylheptyl)-N′-phenethylurea (0.818 g, 102%) as a colorless oil. ¹H NMR (DMSO-d₆): δ=7.34-7.24 (m, 6H), 7.22-7.11 (m, 4H), 5.81 (t, J=5.6 Hz, 1H), 5.76 (t, J=5.8 Hz, 1H), 4.61 (t, J=5.4 Hz, 1H), 3.46 (dd, J=5.4, 1.8 Hz, 2H), 3.22 (q, J=6.7 Hz, 2H), 2.91 (q, J=6.0 Hz, 2H), 2.67 (t, J=7.3 Hz, 2H), 1.74-1.46 (m, 2H), 1.33-0.83 (m, 6H), 1.24 (s, 3H). ¹³C NMR (DMSO-d₆): δ=158.0, 146.4, 139.8, 128.6, (2×) 128.2 (2×), 127.8 (2×), 126.5 (2×), 125.9, 125.3, 70.2, 42.7, 40.9, 39.2, 38.1, 36.2, 30.0, 27.2, 23.4, 22.1.

### 5.1.33 Preparation of N-(7-hydroxy-6-methyl-6-phenylheptyl)-N′-(3-phenylpropyl)urea

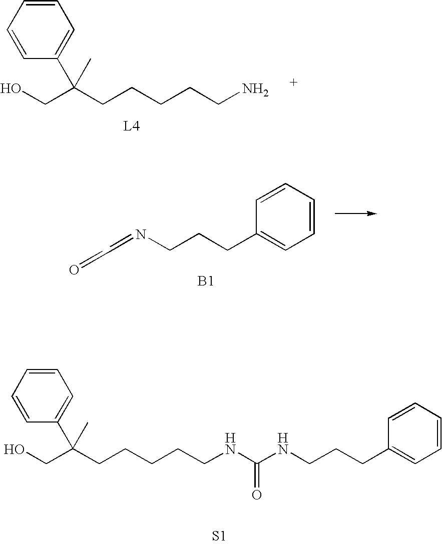

N-(7-hydroxy-6-methyl-6-phenylheptyl)-N′-(3-phenylpropyl)urea. A solution of 1-(3-isocyanatopropyl)benzene (0.345 g, 2.14 mmol) in CH₂Cl₂ (1 mL) was added dropwise to a solution of 7-amino-2-methyl-2-phenyl-1-heptanol (0.562 g, 2.54 mmol) in CH₂Cl₂ (1 mL) in 1 min. After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo and vacuum dried to yield N-(7-hydroxy-6-methyl-6-phenylheptyl)-N′-(3-phenylpropyl)urea (0.770 g, 94%) as a colorless oil. ¹H NMR (DMSO-d₆): δ=7.33-7.22 (m, 6H), 7.21-7.11 (m, 4H), 5.77 (t, J=5.8 Hz, 1H), 5.68 (t, J=5.6 Hz, 1H), 4.55 (t, J=5.4 Hz, 1H), 3.45 (dd, J=5.4, 2.1 Hz, 2H), 2.98 (q, J=6.5 Hz, 2H), 2.90 (q, J=6.1 Hz, 2H), 2.55 (t, J=7.8 Hz, 2H), 1.73-1.42 (m, 4H), 1.34-0.81 (m, 6H), 1.18 (s, 3H). ¹³C NMR (DMSO-d₆): δ=158.1, 146.4, 141.8, 128.2 (4×), 127.8 (2×), 126.5 (2×), 125.6, 125.3, 70.2, 42.7, 39.3, 38.8, 38.1, 32.5, 31.9, 30.0, 27.2, 23.4, 22.1.

### 5.1.34 Preparation of N-(7-hydroxy-6-methyl-6-phenylheptyl)-N′-(4-phenylbutyl)urea

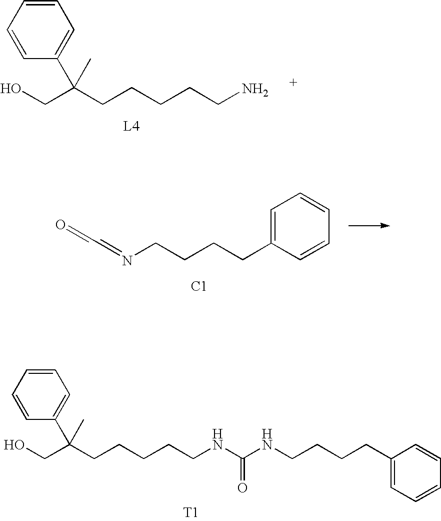

N-(7-hydroxy-6-methyl-6-phenylheptyl)-N′-(4-phenylbutyl)urea. A solution of 1-(4-isocyanatobutyl)benzene (0.356 g, 2;03 mmol) in CH₂Cl₂ (1 mL) was added dropwise to a solution of 7-amino-2-methyl-2-phenyl-1-heptanol (0.543 g, 2.45 mmol) in CH₂Cl₂ (1 mL) in 1 min. After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo and vacuum dried to yield N-(7-hydroxy-6-methyl-6-phenylheptyl)-N′-(4-phenylbutyl)urea (0.819 g, 102%) as a colorless oil. ¹H NMR (DMSO-d₆): δ=7.33-7.22 (m, 6H), 7.20-7.11 (m, 4H), 5.74 (t, J=5.8 Hz, 1H), 5.69 (t, J=5.8 Hz, 1H), 4.60 (t, J=5.2 Hz, 1H), 3.45 (dd, J=5.2, 2.2 Hz, 2H), 2.99 (q, J=6.5 Hz, 2H), 2.89 (q, J=6.3 Hz, 2H), 2.55 (t, J=7.5 Hz, 2H), 1.74-0.81 (m, 12H), 1.22 (s, 3H). ¹³C NMR (DMSO-d₆): δ=158.1, 146.4, 142.2, 128.2 (4×), 127.8 (2×), 126.5 (2×), 125.6, 125.3, 70.2, 42.7, 39.3, 39.0, 38.1, 34.9, 30.0, 29.7, 28.4, 27.2, 23.4, 22.1.

### 5.1.35 Preparation of Methyl 4-([(7-hydroxy-6-methyl-6-phenylheptyl)amino]carbonylamino)butanoate

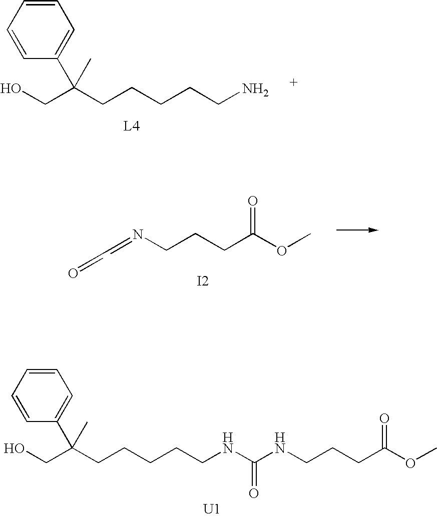

Methyl 4-([(7-hydroxy-6-methyl-6-phenylheptyl)amino]carbonylamino)-butanoate. A solution of methyl 4-isocyanatobutanoate (0.314 g, 2.19 mmol) in CH₂Cl₂ (1 mL) was added dropwise to a solution of 7-amino-2-methyl-2-phenyl-1-heptanol (0.593 g, 2.45 mmol) in CH₂Cl₂ (1 mL) in 1 min. After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo and vacuum dried to yield methyl 4-([(7-hydroxy-6-methyl-6-phenylheptyl)amino]carbonylamino)butanoate (0.818 g, 102%) as a colorless oil. ¹H NMR (DMSO-d₆): δ=7.34-7.23 (m, 4H), 7.18-7.10 (m, 1H), 5.80 (br s, 1H), 5.72 (br s, 1H), 3.57 (s, 3H), 3.45 (d, J=1.9 Hz, 2H), 2.97 (m, 2H), 2.89 (m, 2H). 2.27 (t, J=7.5 Hz, 2H), 1.73-1.43 (m, 4H), 1.34-0.81 (m, 6H), 1.23 (s, 3H). ¹³C NMR (DMSO-d₆): δ=173.2, 158.1, 146.4, 127.8 (2×), 126.5 (2×), 125.3, 70.2, 51.2, 42.7, 39.3, 38.5, 38.1, 30.8, 30.0, 27.2, 25.5, 23.4, 22.1.

### 5.1.36 Preparation of N,N′-di(7-hydroxy-6-methyl-6-phenylheptyl)urea

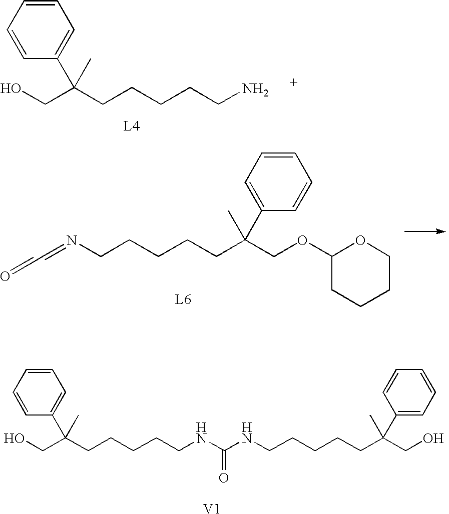

N,N′-di(7-hydroxy-6-methyl-6-phenylheptyl)urea. A solution of 2-((7-isocyanato-2-methyl-2-phenylheptyl)oxy)tetrahydro-2H-pyran (0.591 g, 1.78 mmol) in CH₂Cl₂ (1 mL) was added dropwise to a solution of 7-amino-2-methyl-2-phenyl-1-heptanol (0.509 g, 2.30 mmol) in CH₂Cl₂ (1 mL) in 1 min. After 1 h, the reaction mixture was passed through a Varian Chemelut CE1005 column absorbed with aqueous KHSO₄ (1 M, 2.0 mL) and eluted with CH₂Cl₂ (2×20 mL). The combined organic layers were evaporated in vacuo. The residue was dissolved in MeOH (10 mL) and TsOH.H₂O was added until pH=3. After 20 h of stirring, K₂CO₃ was added until pH=10. The reaction mixture was concentrated in vacuo and suspended in CH₂Cl₂ and a very small amount of MeOH, dried (Na₂SO₄) and evaporated in vacuo. The residue was coevaporated with CH₂Cl₂ (5 mL) and vacuum dried to yield N,N′-di(7-hydroxy-6-methyl-6-phenylheptyl)urea (1.054 g, 126%) as a turbid white oil. ¹H NMR (DMSO-d₆): δ=7.33-7.22 (m, 8H), 7.20-7.10 (m, 2H), 3.43 (s, 4H), 2.85 (t, J=6.4 Hz, 4H), 1.73-1.39 (m, 4H), 1.32-0.79 (m, 12H), 1.22 (s, 6H). ¹³C NMR (DMSO-d₆): δ=158.2, 146.6 (2×), 127.8 (4×), 126.5 (4×), 125.3 (2×), 70.2 (2×), 48.6 (2×), 39.1 (2×), 38.1 (2×), 27.3 (2×), 23.4 (2×), 22.6 (2×), 22.2 (2×).

### 6. Biological Assays

### 6.1 Effects of Illustrative Compounds A and A1 on NonHDL-Cholesterol, HDL-Cholesterol, Triglyceride Levels, Glycemic Control Indicators and Body Weight Control in Obese Female Zucker Rats

In a number of different experiments, illustrative compounds A and A1 of the invention were administered daily at a dose of up to 100 mg/kg to chow-fed obese female Zucker rats for fourteen days in the morning by oral gavage in 1.5% carboxymethylcellulose/0.2% Tween-20 or 20% ethanol/80% polyethylene glycol (dosing vehicles). Animals were weighed daily. Animals were allowed free access to rodent chow and water throughout the study except on days of blood sampling where food was restricted for six hours prior to blood sampling. Blood glucose was determined after the 6-hour fast in the afternoon without anesthesia from a tail vein. Serum was also prepared from pretreatment blood samples subsequently obtained from the orbital venous plexus (with O₂/CO₂ anesthesia) and following the fourteenth dose at sacrifice from the heart following O₂/CO₂ anesthesia. Serums were assayed for lipoprotein cholesterol profiles, triglycerides, total cholesterol, NonHDL cholesterol, HDL cholesterol, the ratio of HDL cholesterol to that of Non-HDL cholesterol, insulin, non-esterified fatty acids, and beta hydroxy butyric acid. The percent body weight gain and the ratio of liver to body weight was also determined. These are shown as absolute values or as a percent change of the pretreatment values in Table 4.

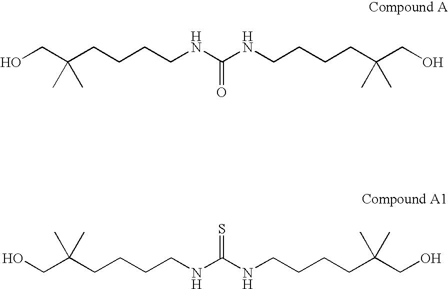

TABLE 4
Examples of effects of oral daily treatment of obese female Zucker rats
with compounds of the invention for fourteen days (n is number of animals per
experiment)

| | | | | | | Percent Change from Pre-treatment | | | | | | | |
|---|---|---|---|---|---|---|---|---|---|---|---|---|---|
| | | | Dose | % wt. | HDL-C/ | | | Non | | | | | |
| Compd | Expt. # | n | (mg/kg/day) | gain | nonHDL-C | TG | TC | HDL-C | HDL-C | Glucose | Insulin | NEFA | BHA |
| Vehicle | LR56 | 5 | 0 | 13 | 1.6 | 26 | 1 | 43 | −17 | 5 | −21 | 7 | 56 |
| A | LR56 | 4 | 100 | 4 | 2.7 | −31 | 50 | −5 | 92 | 2 | 60 | 8 | 172 |
| Vehicle | LR60 | 3 | 0 | 7 | 1.1 | 45 | −9 | 21 | −26 | −17 | −20 | 35 | 108 |
| A1 | LR60 | 4 | 30 | 10 | 2.0 | 7 | −9 | 32 | −11 | −4 | −29 | 11 | 283 |
| A1 | LR60 | 4 | 100 | 7 | 1.3 | −15 | −7 | −29 | 2 | −15 | 10 | 34 | 305 |

Generally, illustrative compounds improved the ratio of non-HDL cholesterol to HDL cholesterol content relative to control, acted as insulin sensitizers, and generally illustrative compounds reduced serum triglyceride content. Illustrative compounds reduced serum levels of harmful triglycerides, reduced serum levels of harmful non-esterified fatty acids, and elevated levels of the beneficial β-hydroxy butyrate.

Accordingly, the compounds of the present invention or pharmaceutically acceptable salts, solvates, hydrates, clathrates, or prodrugs thereof, are useful for improving the ratio of HDL:non-HDL cholesterol in the blood, reducing serum triglycerides, acting as insulin sensitizers, and/or elevating HDL-cholesterol, without the adverse side effect of promoting weight gain in a patient to whom the compound is administered.

### 6.2 Effects of Illustrative Compounds of the Invention on the in Vitro Lipid Synthesis in Isolated Hepatocytes

Compounds were tested for inhibition of lipid synthesis in primary cultures of rat hepatocytes. Male Sprague-Dawley rats were anesthetized with intraperitoneal injection of sodium pentobarbital (80 mg/kg). Rat hepatocytes were isolated essentially as described by the method of Seglen (Seglen, P. O. Hepatocyte suspensions and cultures as tools in experimental carcinogenesis. J. Toxicol. Environ. Health 1979, 5, 551-560). Hepatocytes were suspended in Dulbecco's Modified Eagles Medium containing 25 mM D-glucose, 14 mM HEPES, 5 mM L-glutamine, 5 mM leucine, 5 mM alanine, 10 mM lactate, 1 mM pyruvate, 0.2% bovine serum albumin, 17.4 mM non-essential amino acids, 20% fetal bovine serum, 100 nM insulin and 20 μg/mL gentamycin) and plated at a density of 1.5×10⁵ cells/cm² on collagen-coated 96-well plates. Four hours after plating, media was replaced with the same media without serum. Cells were grown overnight to allow formation of monolayer cultures. Lipid synthesis incubation conditions were initially assessed to ensure the linearity of [1-¹⁴C]-acetate incorporation into hepatocyte lipids for up to 4 hours. Hepatocyte lipid synthesis inhibitory activity was assessed during incubations in the presence of 0.25 μCi [1-¹⁴C]-acetate/well (final radiospecific activity in assay is 1 Ci/mol) and 0, 1, 3, 10, 30, 100 or 300 μM of compounds for 4 hours. At the end of the 4-hour incubation period, medium was discarded and cells were washed twice with ice-cold phosphate buffered saline and stored frozen prior to analysis. To determine total lipid synthesis, 170 μl of MicroScint-E® and 50 μl water was added to each well to extract and partition the lipid soluble products to the upper organic phase containing the scintillant. Lipid radioactivity was assessed by scintillation spectroscopy in a Packard TopCount NXT. Lipid synthesis rates were used to determine the IC₅₀s of the compounds that are presented in Table 5.

TABLE 5
Effect of Keto-diacids and -Diols on Lipid Synthesis in Primary Rat Hepatocytes.

| | | 95% Confidence | | |
|---|---|---|---|---|
| | | Interval | | |
| Compound | IC₅₀ (μM) | Lower | Upper | r^{2a} |
| 213 | 34 | 9.7 | 122 | 0.907 |
| 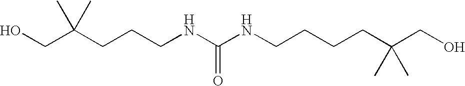 | | | | |
| 216 | 51 | 19.1 | 138 | 0.951 |
| 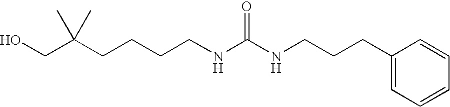 | | | | |
| 217 | 221 | 107 | 457 | 0.943 |
| 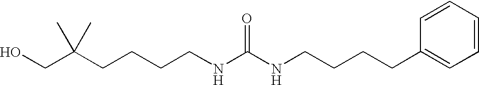 | | | | |
| 219 | 82 | 26.2 | 257 | 0.929 |
| 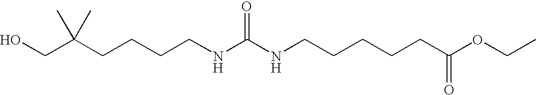 | | | | |
| 220 | 8.8 | 1.8 | 43.3 | 0.941 |
| 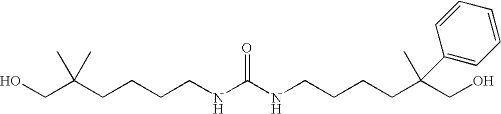 | | | | |
| 222 | 264 | | | 0.972 |
| 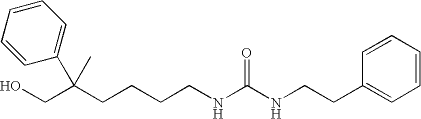 | | | | |
| 223 | 137 77 | 79.7 30.1 | 234 196 | 0.984 0.945 |
| 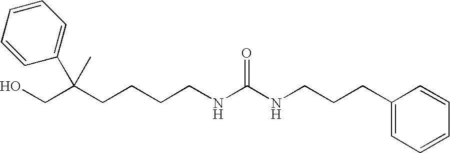 | | | | |
| 225 | 128 | 41.8 | 389 | 0.901 |
| 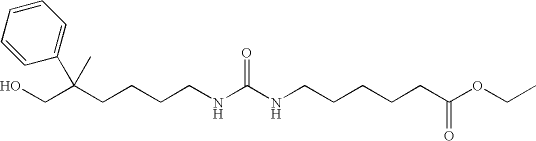 | | | | |
| 226 | 88 | | | 0.936 |
| 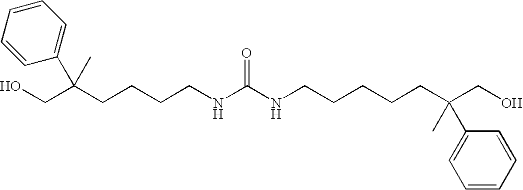 | | | | |
| 227 | 223 | | | 0.979 |
| 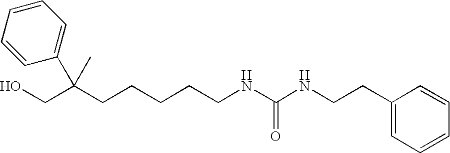 | | | | |
| 228 | 101 | 24.6 | 415 | 0.979 |
| 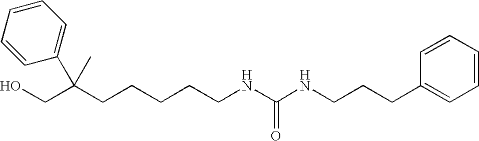 | | | | |
| 230 | 103 | 34.8 | 304 | 0.971 |
| 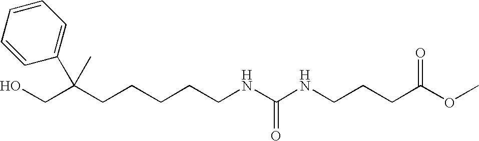 | | | | |
| 231 | 39 | 27.1 | 56.4 | 0.991 |
| 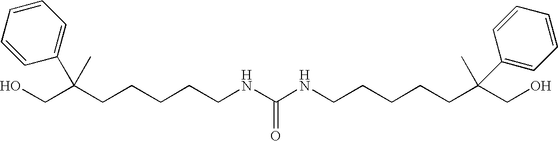 | | | | |
| 232 | 8.2 3.4 | 1.3 2.9 | 54.1 3.9 | 0.921 0.999 |
| 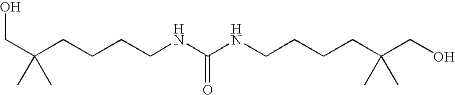 | | | | |

^{a}r² is the goodness of fit of the data to the non-linear sigmoidal model

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

A number of references have been cited, the entire disclosures of which are incorporated herein by reference.

1. A compound of a formula I: 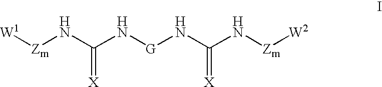
or a pharmaceutically acceptable salt, hydrate, solvate, or a mixture thereof wherein:
(a) X is O or S;
(b) each occurrence of Z is independently CH₂, CH═CH, or phenyl, wherein each occurrence of m is independently an integer ranging from 1 to 9, but when Z is phenyl then its associated m is 1;
(c) G is (CH₂)ᵣ, CH₂CH═CHCH₂, CH═CH, CH₂-phenyl-CH₂, or phenyl, wherein r is 2, 3, or 4;
(d) W¹ and W² are independently L, V, C(R¹)(R²)—(CH₂)_{c}—C(R³)(R⁴)—(CH₂)ₙ—Y, or C(R¹)(R²)—(CH₂)_{c}—V, wherein c is 1 or 2 and n is an independent integer ranging from 0 to 4;
(e) each occurrence of R¹ and R² is independently (C₁-C₆)alkyl, (C₂-C₆)alkenyl, (C₂-C₆)alkynyl, phenyl, or benzyl or when W¹ or W² is C(R¹)(R²)—(CH₂)_{c}—C(R³)(R⁴)—Y, then R¹ and R² can both be H; or R¹ and R² and the carbon to which they are both attached are taken together to form a (C₃-C₇)cycloakyl group;
(f) R³ is H, (C₁-C₆)alkyl, (C₂-C₆)alkenyl, (C₂-C₆)alkynyl, (C₁-C₆)alkoxy, phenyl, benzyl, Cl, Br, CN, NO₂, or CF₃;
(g) R⁴ is OH, (C₁-C₆)alkyl, (C₂-C₆)alkenyl, (C₂-C₆)alkynyl, (C₁-C₆)alkoxy, phenyl, benzyl, Cl, Br, CN, NO₂, or CF₃;
(h) L is C(R¹)(R²)—(CH₂)ₙ—Y; where n is an independent integer ranging from 0 to 4;
(i) V is 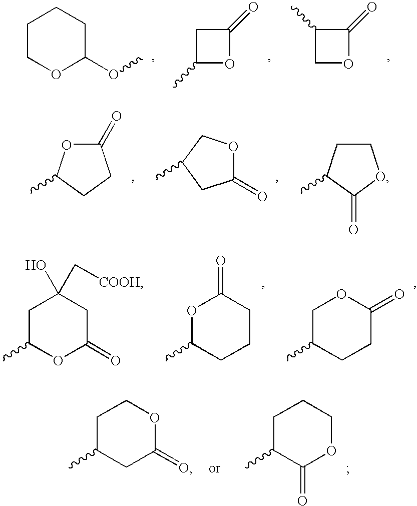
(j) each occurrence of Y is independently (C₁-C₆)alkyl, OH, COOH, CHO, COOR⁵, SO₃H, 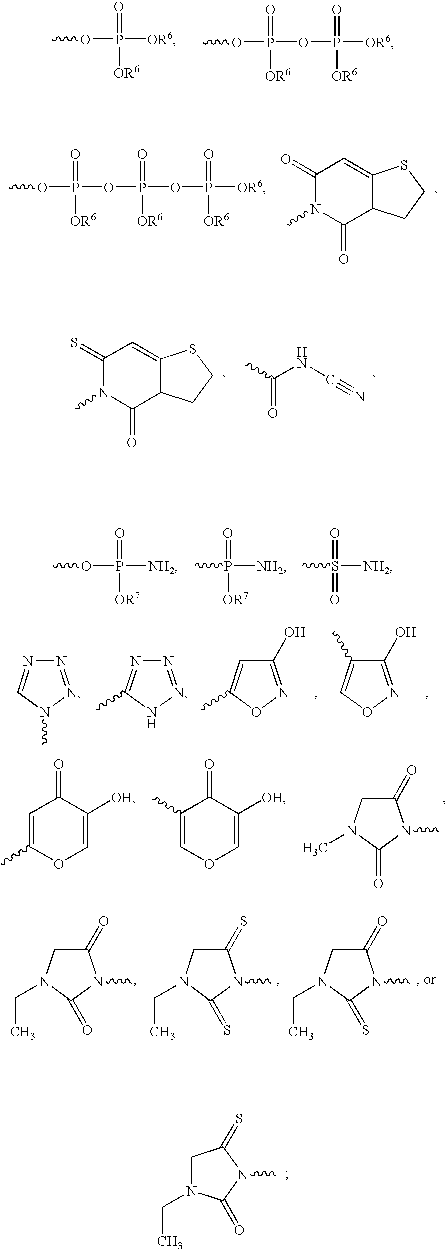
where:
(i) R⁵ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
(ii) each occurrence of R⁶ is independently H, (C₁-C₆)alkyl, (C₂-C₆)alkenyl, or (C₂-C₆)alkynyl and is unsubstituted or substituted with one or two halo, OH, C₁-C₆ alkoxy, or phenyl groups; and
(iii) each occurrence of R⁷ is independently H, (C₁-C₆)alkyl, (C₂-C₆)alkenyl, or (C₂-C₆)alkynyl.

2. The compound of claim 1, wherein:
(a) X is O or S;
(b) W¹ and W² are independently L, V, or C(R¹)(R²)—(CH₂)_{c}—V where c is 1 or 2; and
(c) R¹ or R² are independently (C₁-C₆)alkyl, (C₂-C₆)alkenyl, (C₂-C₆)alkynyl, phenyl, or benzyl.

3. The compound of claim 1, wherein W¹ is L.

4. The compound of claim 1, wherein W¹ is V.

5. The compound of claim 1, wherein W¹ is C(R¹)(R²)—(CH₂)_{c}—C(R³)(R⁴)—CH₂)ₙ—Y.

6. The compound of claim 1, wherein W¹ is C(R¹)(R²)—(CH₂)_{c}—V.

7. The compound of claim 1, wherein W¹ and W² are independent L groups.

8. The compound of claim 7, wherein each occurrence of Y is independently OH, COOR⁵, or COOH.

9. A compound of the formula Ia: 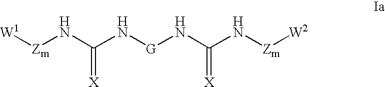
or a pharmaceutically acceptable salt, hydrate, solvate, or a mixture thereof wherein:
(a) X is O or S;
(b) each occurrence of Z is independently CH₂ or CH═CH, wherein each occurrence of m is independently an integer ranging from 1 to 9;
(b) G is (CH₂)ᵣ, CH₂CH═CHCH₂, or CH═CH, where r is 2, 3, or 4;
(c) W¹ and W² are independently L, V, or C(R¹)(R²)—(CH₂)_{c}—V, where c is 1 or 2;
(d) each occurrence of R¹ and R² is independently (C₁-C₆)alkyl, (C₂-C₆)alkenyl, (C₂-C₆)alkynyl, phenyl, or benzyl; or R¹ and R² and the carbon to which they are both attached are taken together to form a (C₃-C₇)cycloakyl group;
(e) L is C(R¹)(R²)—(CH₂)ₙ—Y, where n is an independent integer ranging from 0 to 4;
(f) V is 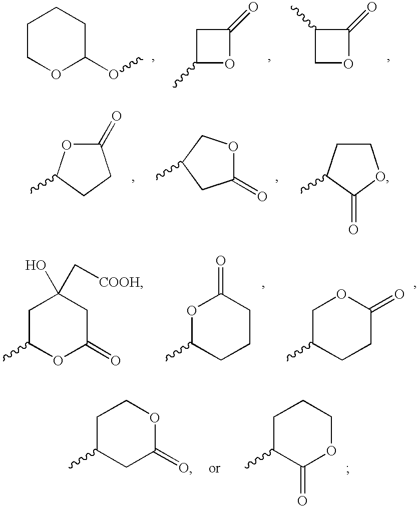
(g) each occurrence of Y is independently (C₁-C₆)alkyl, OH, COOH, CHO, COOR⁵, SO₃H, 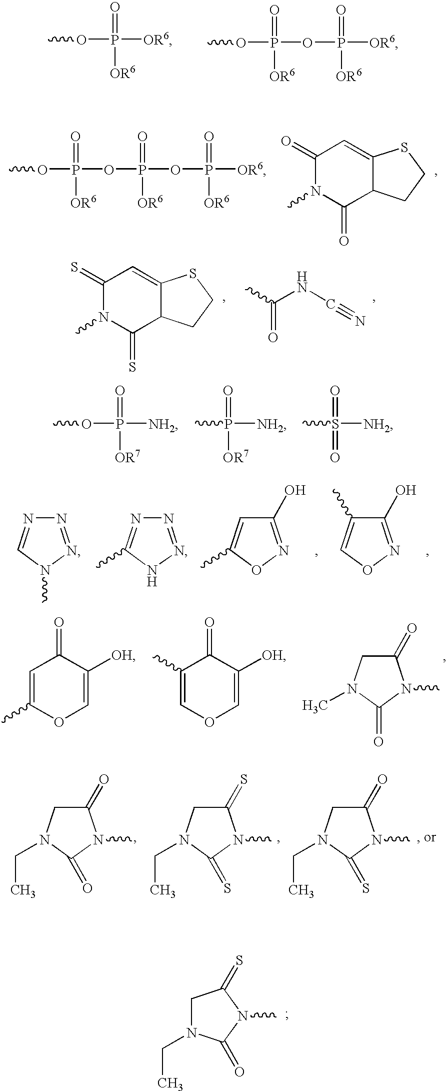
where:
(i) R³ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
(ii) each occurrence of R⁴ is independently H, (C₁-C₆)alkyl, (C₂-C₆)alkenyl, or (C₂-C₆)alkynyl and is unsubstituted or substituted with one or two halo, OH, C₁-C₆ alkoxy, or phenyl groups; and
(iii) each occurrence of R⁵ is independently H, (C₁-C₆)alkyl, (C₂-C₆)alkenyl, or (C₂-C₆)alkynyl.

10. The compound of claim 9, wherein W¹ is L.

11. The compound of claim 9, wherein W¹ is V.

12. The compound of claim 9, wherein W¹ is C(R¹)(R²)—(CH₂)_{c}—V.

13. The compound of claim 9, wherein W¹ and W² are independent L groups.

14. The compound of claim 13, wherein each occurrence of Y is independently OH, COOR³, or COOH.

15. A compound of the formula Ib: 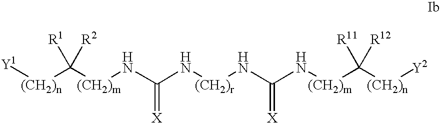
or a pharmaceutically acceptable salt, hydrate, solvate, clathrate, stereoisomer, geometric isomer or mixtures thereof wherein:
(a) X is O or S;
(b) each occurrence of m is independently an integer ranging from 1 to 9;
(c) r is 2, 3, or 4;
(d) each occurrence of n is an independent integer ranging from 0 to 4;
(e) each occurrence of R¹ and R² is independently (C₁-C₆)alkyl, (C₂-C₆)alkenyl, (C₂-C₆)alkynyl, phenyl, benzyl, or R¹, R¹, and the carbon to which they are both attached are taken together to form a (C₃-C₇)cycloalkyl group;
(f) each occurrence of R¹¹ and R¹² is independently H, (C₁-C₆)alkyl, (C₂-C₆)alkenyl, (C₂-C₆)alkynyl, phenyl, benzyl or R¹¹, R¹², and the carbon to which they are both attached are taken together to form a (C₃-C₇)cycloalkyl group; and
(g) each occurrence of Y is independently (C₁-C₆)alkyl, OH, COOH, CHO, COOR⁵, SO₃H, 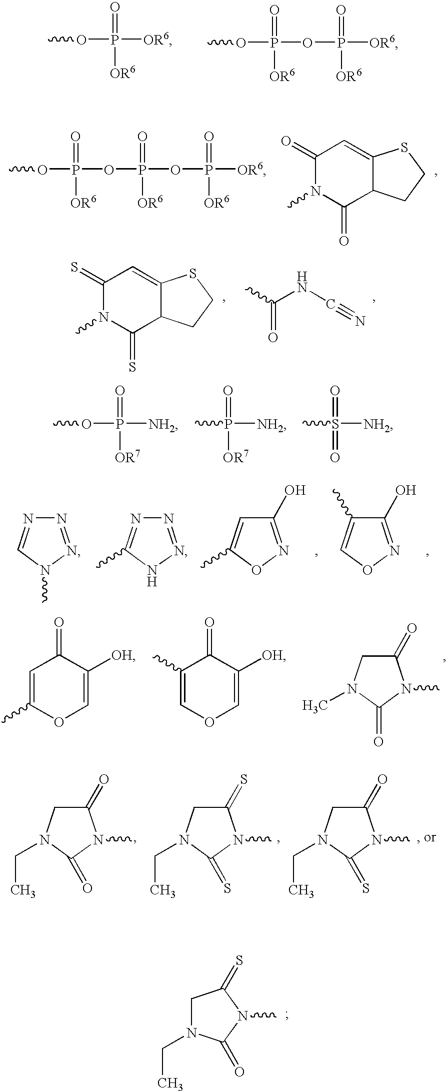
where:
(i) R³ is (C₁—C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
(ii) each occurrence of R⁴ is independently H, (C₁-C₆)alkyl, (C₂-C₆)alkenyl, or (C₂-C₆)alkynyl and is unsubstituted or substituted with one or two halo, OH, C₁-C₆ alkoxy, or phenyl groups; and
(iii) each occurrence of R⁵ is independently H, (C₁-C₆)alkyl, (C₂-C₆)alkenyl, or (C₂-C₆)alkynyl.

16. The compound of claim 15, wherein each occurrence of Y is independently OH, COOR³, or COOH.

17. The compound of claim 16, wherein each R¹ or R² is the same or different (C₁-C₆)alkyl group.

18. A compound of formula Ic: 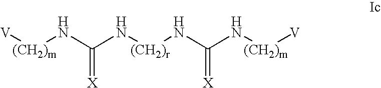
or a pharmaceutically acceptable salt, hydrate, solvate, or a mixture thereof wherein:
(a) X is O or S;
(b) each occurrence of m is an independent integer ranging from 1 to 9;
(c) r is 2, 3, or 4;
(d) V is

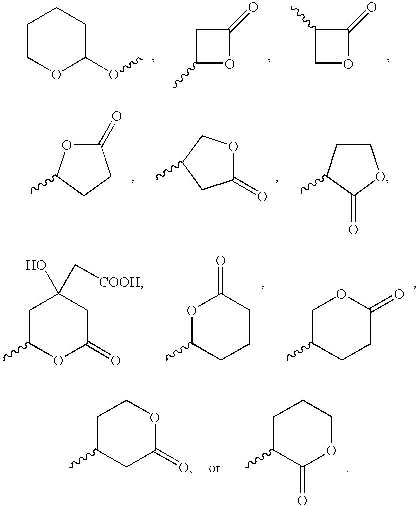

19. A compound according to claim 1, having the formula:
5-[2-(5-hydroxy-4,4-dimethyl-pentyloxy)-ethoxy]-2,2-dimethyl-pentan-1-ol or
4-[3-(3,3-Dimethyl-4-oxo-butoxy)-propoxy]-2,2-dimethyl-butyric acid.

20. A compound of the formula II: 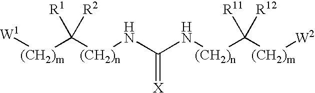
or a pharmaceutically acceptable salt, hydrate, solvate, or a mixture thereof wherein:
(a) X is O or S;
(b) R¹ and R² are independently (C₁-C₆)alkyl, (C₂-C₆)alkenyl, (C₂-C₆)alkynyl, phenyl, or benzyl; or R¹, R², and the carbon to which they are both attached are taken together to form a (C₃-C₇)cycloalkyl group;
(c) R¹¹ and R¹² are independently H, (C₁-C₆)alkyl, (C₂-C₆)alkenyl, (C₂-C₆)alkynyl, phenyl, benzyl; or R¹¹, R¹², and the carbon to which they are both attached are taken together to form a (C₃-C₇)cycloalkyl group;
(c) each occurrence of n is independently an integer ranging from 1 to 7;
(d) each occurrence of m is independently an integer ranging from 0 to 4;
(e) each occurrence of W¹ and W² is independently (C₁-C₆)alkyl, CH₂OH, COOH, CHO, OC(O)R³, C(O)OR³, SO₃H, 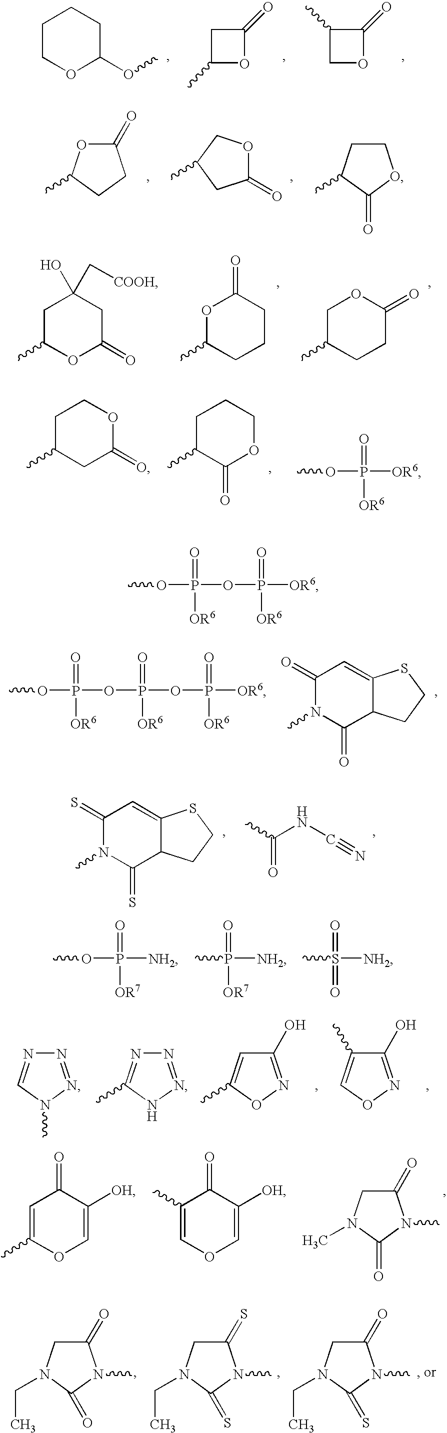 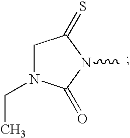
where:
(i) R³ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
(ii) each occurrence of R⁴ is independently H, (C₁-C₆)alkyl, (C₂-C₆)alkenyl, or (C₂-C₆)alkynyl and is unsubstituted or substituted with one or two halo, OH, C₁-C₆ alkoxy, or phenyl groups;
(iii) each occurrence of R⁵ is independently H, (C₁-C₆)alkyl, (C₂-C₆)alkenyl, or (C₂-C₆)alkynyl; and
(iv) each occurrence of n is independently an integer ranging from 0 to 4.

21. A compound of the formula IIa: 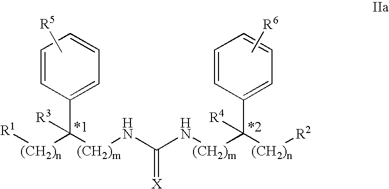
or a pharmaceutically acceptable salt, hydrate, solvate, clathrate, stereoisomer, geometric isomer or mixtures thereof wherein:
(a) X is O or S;
(b) R¹ and R² are (C₁-C₆)alkyl, CH₂OH, COOH, CHO, OC(O)R³, C(O)OR³, SO₃H, 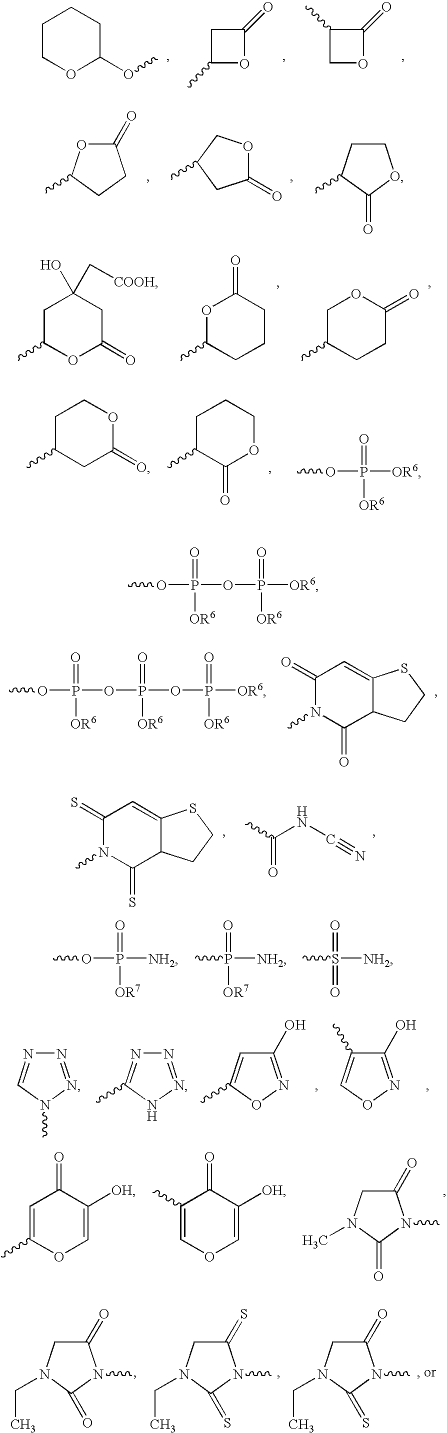 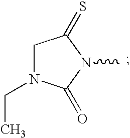
where:
(i) R⁷ is (C—C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
(ii) each occurrence of R⁸ is independently H, (C₁-C₆)alkyl, (C₂-C₆)alkenyl, or (C₂-C₆)alkynyl and is unsubstituted or substituted with one or two halo, OH, C₁—C₆ alkoxy, or phenyl groups, and
(iii) each occurrence of R⁹ is independently H, (C₁-C₆)alkyl, (C₂-C₆)alkenyl, or (C₂-C₆)alkynyl;
(c) R³ and R⁴ are (C₁-C₆)alkyl, (C₂-C₆)alkenyl, (C₂-C₆)alkynyl, phenyl, or benzyl;
(d) R⁵ and R⁶ are hydrogen, halogen, (C₁-C₄)alkyl, (C₁-C₄)alkoxy, (C₆)aryloxy, CN, or NO₂, N(R⁵)₂ where R⁵ is H, (C₁-C₄)alkyl, phenyl, or benzyl;
(e) each occurrence of m is independently an integer ranging from 1 to 5;
(f) each occurrence of n is independently an integer ranging from 0 to 4; and
(g) C*¹ and C*² represent independent chiral-carbon centers, wherein each center may independently be R or S.

22. The compound of claim 21 wherein C*¹ is a chiral-carbon center of the stereochemical configuration R or substantially R.

23. The compound of claim 21 wherein C*¹ is a chiral-center of the stereochemical configuration S or substantially S.

24. The compound of claim 21 wherein C*² is a chiral-carbon center of the stereochemical configuration R or substantially R.

25. The compound of claim 21 wherein C*² is a chiral-center of the stereochemical configuration S or substantially S.

26. A pharmaceutical composition comprising a compound of claim 1 and a pharmaceutically acceptable vehicle, excipient, or diluent.

27. A pharmaceutical composition comprising the following compound: 1,3-Bis-(6-hydroxy-5,5-dimethyl-hexyl)-urea or a pharmaceutically acceptable salt, hydrate, solvate, clathrate, enantiomer, diastereomer, racemate, or mixures of stereoisomers thereof and a pharmaceutically acceptable vehicle, excipient, or diluent.

28. A method for treating or preventing a cardiovascular disease in a patient, comprising administering to a patient in need of such treatment or prevention a therapeutically effective amount of a compound of claim 1.

29. A method for treating or preventing a dyslipidemia in a patient, comprising administering to a patient in need of such treatment or prevention a therapeutically effective amount of a compound of claim 1.

30. A method for treating or preventing a dyslipoproteinemia in a patient, comprising administering to a patient in need of such treatment or prevention a therapeutically effective amount of a compound of claim 1.

31. A method for treating or preventing a disorder of glucose metabolism in a patient, comprising administering to a patient in need of such treatment or prevention a therapeutically effective amount of a compound of claim 1.

32. A method for treating or preventing Alzheimer's Disease in a patient, comprising administering to a patient in need of such treatment or prevention a therapeutically effective amount of a compound of claim 1.

33. A method for treating or preventing Syndrome X in a patient, comprising administering to a patient in need of such treatment or prevention a therapeutically effective amount of a compound of claim 1.

34. A method for treating or preventing septicemia in a patient, comprising administering to a patient in need of such treatment or prevention a therapeutically effective amount of a compound of claim 1.

35. A method for treating or preventing a thrombotic disorder in a patient, comprising administering to a patient in need of such treatment or prevention a therapeutically effective amount of a compound of claim 1.

36. A method for treating or preventing a peroxisome proliferator activated receptor associated disorder in a patient, comprising administering to a patient in need of such treatment or prevention a therapeutically effective amount of a compound of claim 1.

37. A method for treating or preventing obesity in a patient, comprising administering to a patient in need of such treatment or prevention a therapeutically effective amount of a compound of claim 1.

38. A method for treating or preventing pancreatitis in a patient, comprising administering to a patient in need of such treatment or prevention a therapeutically effective amount of a compound of claim 1.

39. A method for treating or preventing hypertension in a patient, comprising administering to a patient in need of such treatment or prevention a therapeutically effective amount of a compound of claim 1.

40. A method for treating or preventing renal disease in a patient, comprising administering to a patient in need of such treatment or prevention a therapeutically effective amount of a compound of claim 1.

41. A method for treating or preventing cancer in a patient, comprising administering to a patient in claim 1.

42. A method for treating or preventing inflammation in a patient, comprising administering to a patient in need of such treatment or prevention a therapeutically effective amount of a compound of claim 1.

43. A method for treating or preventing impotence in a patient, comprising administering to a patient in need of such treatment or prevention a therapeutically effective amount of a compound of claim 1.

44. A method for treating or preventing a neurodegenerative disease or disorder in a patient, comprising administering to a patient in need of such treatment or prevention a therapeutically or prophylactically effective amount of a compound of claim 1.

45. A method of inhibiting hepatic fatty acid synthesis in a patient, comprising administering to a patient in need thereof a therapeutically or prophylactically effective amount of a compound of claim 1.

46. A method of inhibiting sterol synthesis in a patient, comprising administering to a patient in need thereof a therapeutically or prophylactically effective amount of a compound of claim 1.

47. A method of treating or preventing metabolic syndrome disorders in a patient, comprising administering to a patient in need of such treatment or prevention a therapeutically or prophylactically effective amount of a compound of claim 1.

48. A method of treating or preventing a disease or disorder that is capable of being treated or prevented by increasing HDL levels, which comprises administering to a patient in need of such treatment or prevention a therapeutically effective amount of a compound of claim 1.

49. A method of treating or preventing a disease or disorder that is capable of being treated or prevented by lowering LDL levels, which comprises administering to such patient in need of such treatment or prevention a therapeutically effective amount of a compound of claim 1.

50. A compound, or a pharmaceutically acceptable salt thereof, of the formula:
1) 1,3-Bis-(5-hydroxy-4,4-dimethylpentyl)thiourea;
2) 5-[3-(5-Hydroxy-4,4-dimethylpentyl)thioureido]-2,2-dimethyl pentanoic acid;
3) 5-[3-(4-Carboxy-4-methylpentyl)thioureido]-2,2-dimethyl pentanoic acid;
4) 1,3-Bis-(4-hydroxy-3,3-dimethylbutyl)thiourea;
5) 4-[3-(4-Hydroxy-3,3-dimethylbutyl)thioureido]-2,2-dimethyl butyric acid;
6) 4-[3-(3-Carboxy-3-methylbutyl)thioureido]-2,2-dimethyl butyric acid;
7) 1,3-Bis-(6-hydroxy-5,5-dimethylhexyl)thiourea;
8) 6-[3-(6-Hydroxy-5,5-dimethylhexyl)thioureido]-2,2-dimethyl hexanoic acid;
9) 6-[3-(5-Carboxy-5-methylhexyl)thioureido]-2,2-dimethyl hexanoic acid;
10) 1,3-Bis-(6-hydroxy-5-methyl-5-phenyl-hexyl)-thiourea;
11) 6-[3-(6-Hydroxy-5-methyl-5-phenylhexyl)thioureido]-2-methyl-2-phenyl hexanoic acid;
12) 6-[3-(5-Carboxy-5-phenylhexyl)thioureido]-2-methyl-2-phenyl hexanoic acid;
13) 1,3-Bis-(5-hydroxy-4-methyl-4-phenylpentyl)thiourea;
14) 5-[3-(5-Hydroxy-4-methyl-4-phenylpentyl)thioureido]-2-methyl-2-phenyl pentanoic acid;
15) 5-[3-(4-Carboxy-4-phenylpentyl)thioureido]-2-methyl-2-phenyl pentanoic acid;
16) 1,3-Bis-(4-hydroxy-3-methyl-3-phenylbutyl)thiourea;
17) 4-[3-(4-Hydroxy-3-methyl-3-phenylbutyl)thioureido]-2-methyl-2-phenyl butyric acid;
18) 4-[3-(3-Carboxy-3-phenylbutyl)thioureido]-2-methyl-2-phenyl butyric acid;
19) 1,3-Bis-(4,4-dimethyl-5-oxo-pentyl)thiourea;
20) 5-[3-(4-Methoxycarbonyl-4-methylpentyl)thioureido]-2,2-dimethyl pentanoic acid
methyl ester;
21) 2,2-Dimethyl-5-[3-(4-methyl-4-phenoxycarbonylpentyl)thioureido]pentanoic acid phenyl ester;
22) 5-[3-(4-Benzyloxycarbonyl-4-methylpentyl)thioureido]-2,2-dimethyl pentanoic acid benzyl ester;
23) 2-Methyl-5-[3-(4-methyl-4-sulfopentyl)thioureido]pentane-2-sulfonic acid;
24) Phosphoric acid mono-{1,1-dimethyl-4-[3-(4-methyl-4-phosphonooxypentyl)thioureido]butyl}ester;
25) 1,3-Bis-(5,5-dimethyl-6-oxohexyl)thiourea;
26) 6-[3-(5-Methoxycarbonyl-5-methylhexyl)thioureido]-2,2-dimethyl hexanoic acid methyl ester;
27) 2,2-Dimethyl-6-[3′-(5-methyl-5-phenoxycarbonylhexyl)thioureido]hexanoic acid phenyl ester;
28) 6-[3-(5-Benzyloxycarbonyl-5-methylhexyl)thioureido]-2,2-dimethyl hexanoic acid benzyl ester;
29) 2-Methyl-6-[3-(5-methyl-5-sulfohexyl)thioureido]hexane-2-sulfonic acid;
30) Phosphoric acid mono-(1,1-dimethyl-5-[3-(5-methyl-5-phosphonooxyhexyl)thioureido]pentyl}ester;
31) 1-(6-Hydroxy-5-methyl-5-phenylhexyl)-3-(5-methyl-6-oxo-5-phenylhexyl)thiourea;
32) 2-Methyl-6-[3-(5-methyl-6-methylperoxy-5-phenylhexyl)thioureido]-2-phenylhexanoic acid methyl ester;
33) 2-Methyl-6-[3-(5-phenoxycarbonyl-5-phenylhexyl)thioureido]-2-phenyl hexanoic acid phenyl ester;
34) 6-[3-(5-Benzyloxycarbonyl-5-phenylhexyl)thioureido]-2-methyl-2-phenyl hexanoic acid benzyl ester;
35) 6-[3-(5-Methyl-5-phenyl-hexyl)-thioureido]-2-phenyl-hexane-2-sulfonic acid;
36) Phosphoric acid mono-{1-methyl-1-phenyl-5-[3-(5-phenyl-5-phosphonooxyhexyl)thioureido]pentyl}ester;
37) 1,3-Bis-(6-hydroxy-4,4-dimethylhexyl)thiourea;
38) 6-[3-(6-Hydroxy-4,4-dimethylhexyl)thioureido]-3,3-dimethyl hexanoic acid;
39) 6-[3-(6-Hydroperoxy-4,4-dimethylhexyl)thioureido]-3,3-dimethyl hexanoic acid;
40) 1,3-Bis-(5-hydroxy-3,3-dimethylpentyl)thiourea;
41) 5-[3-(5-Hydroxy-3,3-dimethylpentyl)thioureido]-3,3-dimethyl pentanoic acid;
42) 5-[3-(4-Carboxy-3,3-dimethylbutyl)thioureido]-3,3-dimethyl pentanoic acid;
43) 1,3-Bis-(7-hydroxy-5,5-dimethylheptyl)thiourea;
44) 7-[3-(7-Hydroxy-5,5-dimethylheptyl)thioureido]-3,3-dimethyl heptanoic acid;
45) 7-[3-(6-Carboxy-5,5-dimethylhexyl)thioureido]-3,3-dimethyl heptanoic acid;
46) 1,3-Bis-(7-hydroxy-5-methyl-5-phenylheptyl)thiourea;
47) 7-[3-(7-Hydroxy-5-methyl-5-phenylheptyl)thioureido]-3-methyl-3-phenyl heptanoic acid;
48) 7-[3-(6-Carboxy-5-methyl-5-phenylhexyl)thioureido]-3-methyl-3-phenyl heptanoic acid;
49) 1,3-Bis-(6-hydroxy-4-methyl-4-phenylhexyl)thiourea;
50) 6-[3-(6-Hydroxy-4-methyl-4-phenylhexyl)thioureido]-3-methyl-3-phenyl hexanoic acid;
51) 6-[3-(5-Carboxy-4-methyl-4-phenylpentyl)thioureido]-3-methyl-3-phenyl hexanoic acid;
52) 1,3-Bis-(5-hydroxy-3-methyl-3-phenylpentyl)thiourea;
53) 5-[3-(5-Hydroxy-3-methyl-3-phenylpentyl)thioureido]-3-methyl-3-phenylpentanoic acid;
54) 5-[3-(4-Carboxy-3-methyl-3-phenylbutyl)thioureido]-3-methyl-3-phenyl pentanoic acid;
55) 1,3-Bis-(7-hydroxy-4,4-dimethylheptyl)thiourea;
56) 7-[3-(7-Hydroxy-4,4-dimethylheptyl)thioureido]-4,4-dimethyl heptanoic acid;
57) 7-[3-(6-Carboxy-4,4-dimethylhexyl)thioureido]-4,4-dimethyl heptanoic acid;
58) 1,3-Bis-(6-hydroxy-3,3-dimethylhexyl)thiourea;
59) 6-[3-(6-Hydroxy-3,3-dimethylhexyl)thioureido]-4,4-dimethyl hexanoic acid;
60) 5-[3-(5-Hydroxy-3-methyl-3-phenylpentyl)thioureido]-3-methyl-3-phenyl pentanoic acid;
61) 5-[3-(5-Hydroperoxy-3-methyl-3-phenylpentyl)thioureido]-3-methyl-3-phenyl pentanoic acid;
62) 1,3-Bis-(8-hydroxy-5,5-dimethyloctyl)thiourea;
63) 2-{5-[3-(8-Hydroxy-5,5-dimethyloctyl)thioureido]-1,1-dimethylpentyl}acrylic acid;
64) 2-{5-[3-(8-Hydroperoxy-5,5-dimethyloctyl)thioureido]-1,1-dimethylpentyl}acrylic acid;
65) 1-(6-Hydroxymethyl-5-methyl-5-phenyl-hept-6-enyl)-3-(8-hydroxy-5-methyl-5-phenyloctyl)thiourea;
66) 8-[3-(8-Hydroxy-5-methyl-5-phenyloctyl)thioureido]-4-methyl-4-phenyl octanoic acid;
67) 2-{5-[3-(8-Hydroperoxy-5-methyl-5-phenyloctyl)thioureido]-1-methyl-1-phenylpentyl}acrylic acid;
68) 1,3-Bis-(7-hydroxy-4-methyl-4-phenylheptyl)thiourea;
69) 7-[3-(7-Hydroxy-4-methyl-4-phenylheptyl)thioureido]-4-methyl-4-phenyl heptanoic acid;
70) 7-[3-(6-Carboxy-4-methyl-4-phenylhexyl)thioureido]-4-methyl-4-phenyl heptanoic acid;
71) 1,3-Bis-(6-hydroxy-3-methyl-3-phenylhexyl)thiourea;
72) 6-[3-(6-Hydroxy-3-methyl-3-phenylhexyl)thioureido]-4-methyl-4-phenyl hexanoic acid;
73) 6-[3-(5-Carboxy-3-methyl-3-phenylpentyl)thioureido]-4-methyl-4-phenyl hexanoic acid;
74) 1,3-Bis-[3-(4,6-dioxo-2,3,3a,6-tetrahydro-4H-thieno[3,2-c]pyridin-5-yl)-3-methylbutyl]thiourea
75) 1,3-Bis-[3-(4,6-dithioxo-2,3,3a,6-tetrahydro-4H-thieno[3,2-c]pyridin-5-yl)-3-methylbutyl]thiourea;
76) N,N′-Bis-[4-(N″-cyano-2,2-dimethylbutamide)]thiourea;
77) Phosphoramidic acid mono-(3-{3-[3-(amino-hydroxy-phosphoryloxy)-3-methylbutyl]thioureido}-1,1-dimethylpropyl)ester;
78) 1,3-Bis-[4-(4,6-dioxo-2,3,3a,6-tetrahydro-4H-thieno[3,2-c]pyridin-5-yl)-4-methylpentyl]thiourea;
79) 1,3-Bis-[4-(4,6-dithioxo-2,3,3a,6-tetrahydro-4H-thieno[3,2-c]pyridin-5-yl)-4-methylpentyl]-thiourea;
80) N,N′-Bis-[5-(N″-cyano-2,2-dimethylpentamide)]thiourea;
81) Phosphoramidic acid mono-(4-{3-[4-(amino-hydroxy-phosphoryloxy)-4-methylpentyl]thioureido}-1,1-dimethylbutyl)ester;
82) 1,3-Bis-[4-(4,6-dioxo-2,3,3a,6-tetrahydro-4H-thieno[3,2-c]pyridin-5-yl)-4-phenylpentyl]thiourea;
83) 1,3-Bis-[4-(4,6-dithioxo-2,3,3a,6-tetrahydro-4H-thieno[3,2-c]pyridin-5-yl)-4-phenylpentyl]thiourea;
84) N,N′-Bis-[5-(N″-cyano-2-methyl-2-phenylpentamide)]thiourea;
85) Phosphoramidic acid mono-(4-{3-[4-(amino-hydroxy-phosphoryloxy)-4-phenylpentyl]thioureido}-1-methyl-1-phenylbutyl)ester;
86) 1,3-Bis-(4-methyl-4-tetrazol-1-yl-pentyl)thiourea;
87) 1,3-Bis-[4-methyl-4-(1H-tetrazol-5-yl)pentyl]thiourea;
88) 1,3-Bis-[4-(3-hydroxy-isoxazol-5-yl)-4-methylpentyl]thiourea;
89) 1,3-Bis-[4-(3-hydroxy-isoxazol-4-yl)-4-methylpentyl]thiourea;
90) 1,3-Bis-[4-phenyl-4-(tetrazol-1-yl)penty]thiourea;
91) 1,3-Bis-[4-phenyl-4-(1H-tetrazol-5-yl)pentyl]thiourea;
92) 1,3-Bis-[4-(3-hydroxy-isoxazol-5-yl)-4-phenylpentyl]thiourea;
93) 1,3-Bis-[4-(3-hydroxy-isoxazol-4-yl)-4-phenylpentyl]thiourea;
94) 1,3-Bis-[4-methyl-4-(tetrahydro-pyran-2-yloxy)pentyl]thiourea;
95) 1,3-Bis-[4-methyl-4-(4-oxo-oxetan-2-yl)pentyl]thiourea;
96) 1,3-Bis-[4-methyl-4-(4-oxo-oxetan-2-yl)pentyl]thiourea;
97) 1,3-Bis-[4-methyl-4-(5-oxo-tetrahydro-furan-2-yl)pentyl]thiourea;
98) 1,3-Bis-[4-methyl-4-(5-oxo-tetrahydro-furan-3-yl)pentyl]thiourea;
99) 1,3-Bis-[4-methyl-4-(2-oxo-tetrahydro-furan-3-yl)pentyl]thiourea;
100) [2-(4-{3-[4-(4-Carboxymethyl-4-hydroxy-6-oxo-tetrahydro-pyran-2-yl)-4-methylpentyl]thioureido}-1,1-dimethylbutyl)-4-hydroxy-6-oxo-tetrahydro-pyran-4-yl]acetic acid;
101) 1,3-Bis-[4-phenyl-4-(tetrahydro-pyran-2-yloxy)pentyl]thiourea;
102) 1,3-Bis-[4-(4-oxo-oxetan-2-yl)-4-phenylpentyl]thiourea;
103) 1,3-Bis-[4-(2-oxo-oxetan-3-yl)-4-phenylpentyl]thiourea;
104) 1,3-Bis-[4-(5-oxo-tetrahydro-furan-2-yl)-4-phenylpentyl]thiourea;
105) 1,3-Bis-[4-(5-oxo-tetrahydro-furan-3-yl)-4-phenylpentyl]thiourea;
106) 1,3-Bis-[4-(2-oxo-tetrahydro-furan-3-yl)-4-phenylpentyl]thiourea;
107) [2-(4-{3-[4-(4-Carboxymethyl-4-hydroxy-6-oxo-tetrahydro-pyran-2-yl)-4-phenylpentyl]thioureido}-1-methyl-1-phenylbutyl)-4-hydroxy-6-oxo-tetrahydro-pyran-4-yl]acetic acid;
108) 1-(3-Hydroxy-3-methylbutyl)-3-{3-[3-(3-hydroxy-3-methylbutyl)thioureido]propyl}thiourea;
109) 1-(4-Hydroxy-3,3-dimethylbutyl)-3-{3-[3-(4-hydroxy-3,3-dimethylbutyl)thioureido]propyl}thiourea;
110) 4-(3-{3-[3-(3-Carboxy-3-methylbutyl)thioureido]propyl}thioureido)-2,2-dimethyl butyric acid;
111) 1-(3,3-Dimethyl-4-oxobutyl)-3-{3-[3-(3,3-dimethyl-4-oxobutyl)thioureido]propyl}thiourea;
112) 4-(3-{3-[3-(3-Methoxycarbonyl-3-methylbutyl)thioureido]propyl}thioureido)-2,2-dimethyl butyric acid methyl ester;
113) 2,2-Dimethyl-4-(3-{3-[3-(3-methyl-3-phenoxycarbonylbutyl)thioureido]propyl}thioureido)butyric acid phenyl ester;
114) 4-(3-{3-[3-(3-Benzyloxycarbonyl-3-methylbutyl)thioureido]propyl}thioureido)-2,2-dimethyl butyric acid benzyl ester;
115) 2-Methyl-4-(3-{3-[3-(3-methyl-3-sulfobutyl)thioureido]propyl}thioureido)butane-2-sulfonic acid;
116) Phosphoric acid mono-[1,1-dimethyl-3-(3-{3-[3-(3-methyl-3-phosphonooxybutyl)thioureido]propyl}thioureido)propyl]ester;
117) 1-[3-(4,6-Dioxo-2,3,3a,6-tetrahydro-4H-thieno[3,2-c]pyridin-5-yl)-3-methylbutyl]-3-(3-{3-[3-(4,6-dioxo-2,3,3a,6-tetrahydro-4H-thieno[3,2-c]pyridin-5-yl)-3-methylbutyl]thioureido}propyl)thiourea;
118) 1-[3-(4,6-Dithioxo-2,3,3a,6-tetrahydro-4H-thieno[3,2-c]pyridin-5-yl)-3-methylbutyl]-3-(3-{3-[3-(4,6-dithioxo-2,3,3a,6-tetrahydro-4H-thieno[3,2-c]pyridin-5-yl)-3-methylbutyl]thioureido}propyl)thiourea;
119) Phosphoramidic acid mono-[1,1-dimethyl-3-(3-{3-[3-(3-methyl-3-phosphonooxybutyl)thioureido]propyl}thioureido)propyl]ester;
120) N-cyano-4-[3-(3-{3-(N-cyano-carbamoyl)-3-methylbutyl]thioureido}propylthioureido]-2,2-dimethylbutyramide;
121) 1-[3-Methyl-3-(1H-tetrazol-5-yl)-butyl]-3-(3-{3-[3-methyl-3-(1H-tetrazol-5-yl)butyl]thioureido}propyl)thiourea;
122) 1-[3-(3-Hydroxy-isoxazol-5-yl)-3-methylbutyl]-3-(3-{3-[3-(3-hydroxy-isoxazol-5-yl)-3-methylbutyl]thioureido}propyl)thiourea;
123) 1-[3-(3-Hydroxy-isoxazol-4-yl)-3-methylbutyl]-3-(3-{3-[3-(3-hydroxy-isoxazol-4-yl)-3-methylbutyl]thioureido}propyl)thiourea;
124) 1-[3-(5-Hydroxy-4-oxo-4H-pyran-3-yl)-3-methylbutyl]-3-(3-{3-[3-(5-hydroxy-4-oxo-4H-pyran-3-yl)-3-methylbutyl]thioureido}propyl)thiourea;
125) 1-[3-(5-Hydroxy-4-oxo-4H-pyran-2-yl)-3-methylbutyl]-3-(3-{3-[3-(5-hydroxy-4-oxo-4H-pyran-2-yl)-3-methylbutyl]thioureido}propyl)thiourea;
126) 1-(3-{3-[3-(3-Ethyl-2,5-dioxo-imidazolidin-1-yl)-3-methylbutyl]thioureido}propyl)-3-[3-(3-ethyl-2,5-dithioxo-imidazolidin-1-yl)-3-methylbutyl]thiourea;
127) 1-[3-(3-Ethyl-2,5-dioxo-imidazolidin-1-yl)-3-methylbutyl]-3-(3-{3-[3-(3-ethyl-2,5-dioxo-imidazolidin-1-yl)-3-methylbutyl]thioureido}propyl)thiourea;
128) 1-[3-(3-Ethyl-5-oxo-2-thioxo-imidazolidin-1-yl)-3-methylbutyl]-3-(3-{3-[3-(3-ethyl-5-oxo-2-thioxo-imidazolidin-1-yl)-3-methylbutyl]thioureido}propyl)thiourea;
129) 1-[3-(3-Ethyl-2-oxo-5-thioxo-imidazolidin-1-yl)-3-methylbutyl]-3-(3-{3-[3-(3-ethyl-2-oxo-5-thioxo-imidazolidin-1-yl)-3-methylbutyl]thioureido}propyl)thiourea;
130) 1-[2-(Tetrahydro-pyran-2-yloxy)-ethyl]-3-(3-{3-[2-(tetrahydro-pyran-2-yloxy)ethyl]thioureido}propyl)thiourea;
131) 1-[2-(4-Oxo-oxetan-2-yl)ethyl]-3-(3-{3-[2-(4-oxo-oxetan-2-yl)ethyl]thioureido}propyl)thiourea;
132) 1-[2-(2-Oxo-oxetan-3-yl)ethyl]-3-(3-{3-[2-(2-oxo-oxetan-3-yl)ethyl]thioureido propyl)thiourea;
133) 1-[2-(5-Oxo-tetrahydro-furan-2-yl)ethyl]-3-(3-{3-[2-(5-oxo-tetrahydro-furan-2-yl)
ethyl]thioureido}propyl)thiourea;
134) 1-[2-(5-Oxo-tetrahydro-furan-3-yl)ethyl]-3-(3-{3-[2-(5-oxo-tetrahydro-furan-3-yl)ethyl]thioureido}propyl)thiourea;
135) 1-[2-(2-Oxo-tetrahydro-furan-3-yl)ethyl]-3-(3-{3-[2-(2-oxo-tetrahydro-furan-3-yl) ethyl]thioureido}propyl)thiourea;
136) (2-{2-[3-(3-{3-[2-(4-Carboxymethyl-4-hydroxy-6-oxo-tetrahydro-pyran-2-yl)ethyl]thioureido}propyl)thioureido]ethyl}-4-hydroxy-6-oxo-tetrahydro-pyran-4-yl)acetic acid;
137) 1-[2-(6-Oxo-tetrahydro-pyran-2-yl)ethyl]-3-(3-{3-[2-(6-oxo-tetrahydro-pyran-2-yl)ethyl]thioureido}propyl)thiourea;
138) 1-[2-(6-Oxo-tetrahydro-pyran-3-yl)ethyl]-3-(3-{3-[2-(6-oxo-tetrahydro-pyran-3-yl)ethyl]thioureido}propyl)thiourea;
139) 1-[2-(2-Oxo-tetrahydro-pyran-4-yl)ethyl]-3-(3-{3-[2-(2-oxo-tetrahydro-pyran-4-yl)ethyl]thioureido}propyl)thiourea;
140) 1-[2-(2-Oxo-tetrahydro-pyran-3-yl)ethyl]-3-(3-{3-[2-(2-oxo-tetrahydro-pyran-3-yl) ethyl]thioureido}propyl)thiourea;
141) 1-(5-Hydroxy-3,3-dimethylpentyl)-3-{3-[3-(5-hydroxy-3,3-dimethylpentyl)thioureido]propyl}thiourea;
142) 5-(3-{3-[3-(4-Carboxy-3,3-dimethylbutyl)thioureido]propyl}thioureido)-3,3-dimethyl pentanoic acid;
143) 1-(3,3-Dimethyl-5-oxo-pentyl)-3-{3-[3-(3,3-dimethyl-5-oxo-pentyl)thioureido]propylthiourea;
144) 5-(3-{3-[3-(4-Methoxycarbonyl-3,3-dimethylbutyl)thioureido]propyl}thioureido)-3,3-dimethyl pentanoic acid methyl ester;
145) 3,3-Dimethyl-5-(3-{3-[3-(3-methyl-3-phenoxycarbonylbutyl)thioureido]propyl}thioureido)pentanoic acid phenyl ester;
146) 5-(3-{3-[3-(3-Benzyloxycarbonyl-3-methylbutyl)thioureido]propyl}thioureido)-3,3-dimethyl pentanoic acid benzyl ester;
147) 4-(3-{3-[3-(3,3-Dimethyl-4-sulfobutyl)thioureido]propyl}thioureido)-2,2-dimethylbutane-1-sulfonic acid;
148) Phosphoricacidmono-[4-(3-{3-[3-(3,3-dimethyl-4-phosphonooxybutyl)thioureido]propyl}thioureido)-2,2-dimethylbutyl]ester;
149) 1-[4-(4,6-Dioxo-2,3,3a,6-tetrahydro-4H-thieno[3,2-c]pyridin-5-yl)-3,3-dimethylbutyl]-3-(3-{3-[4-(4,6-dioxo-2,3,3a,6-tetrahydro-4H-thieno[3,2-c]pyridin-5-yl)-3,3-dimethylbutyl]thioureido}propyl)thiourea;
150) 1-[4-(4,6-Dithioxo-2,3,3a,6-tetrahydro-4H-thieno[3,2-c]pyridin-5-yl)-3,3-dimethylbutyl]-3-(3-{3-[4-(4,6-dithioxo-2,3,3a,6-tetrahydro-4H-thieno[3,2-c]pyridin-5-yl)-3,3-dimethylbutyl]thioureido}propyl)thiourea;
151) 5-[3-(3-{3-[4-(N-cyano-carbamoyl)-3,3-dimethylbutyl]thioureido}propyl)thioureido]-3,3-dimethyl pentanoic acid cyanamide;
152) Phosphoramidicacidmono-{4-[3-(3-{3-[4-(amino-hydroxy-phosphoryloxy)-3,3-dimethylbutyl]thioureido}propyl)thioureido]-2,2-dimethylbutyl}ester;
153) 1-(3,3-Dimethyl-4-tetrazol-1-yl-butyl)-3-{3-[3-(3,3-dimethyl-4-tetrazol-1-yl-butyl)thioureido]propyl}thiourea;
154) 1-[3,3-Dimethyl-4-(1H-tetrazol-5-yl)butyl]-3-(3-{3-[3,3-dimethyl-4-(1H-tetrazol-5-yl)butyl]thioureido}propyl)thiourea;
155) 1-[4-(3-Hydroxy-isoxazol-5-yl)-3,3-dimethylbutyl]-3-(3-{3-[4-(3-hydroxy-isoxazol-5-yl)-3,3-dimethylbutyl]thioureido}propyl)thiourea;
156) 1-[4-(3-Hydroxy-isoxazol-4-yl)-3,3-dimethylbutyl]-3-(3-{3-[4-(3-hydroxy-isoxazol-4-yl)-3,3-dimethylbutyl]thioureido}propyl)thiourea;
157) 1-[4-(5-Hydroxy-4-oxo-4H-pyran-3-yl)-3,3-dimethylbutyl]-3-(3-{3-[4-(5-hydroxy-4-oxo-4H-pyran-2-yl)-3,3-dimethylbutyl]thioureido}propyl)thiourea;
158) 1-[4-(5-Hydroxy-4-oxo-4H-pyran-2-yl)-3,3-dimethylbutyl]-3-(3-{3-[4-(5-hydroxy-4-oxo-4H-pyran-2-yl)-3,3-dimethylbutyl]thioureido}propyl)thiourea;
159) 1-[4-(5-Hydroxy-4-oxo-4H-pyran-3-yl)-3,3-dimethylbutyl]-3-(3-{3-[4-(5-hydroxy-4-oxo-4H-pyran-3-yl)-3,3-dimethylbutyl]thioureido}propyl)thiourea;
160) 1-[4-(3-Ethyl-2,5-dioxo-imidazolidin-1-yl)-3,3-dimethylbutyl]-3-(3-{3-[4-(3-ethyl-2,5-dithioxo-imidazolidin-1-yl)-3,3-dimethylbutyl]thioureido}propyl)thiourea;
161) 1-[4-(3-Ethyl-2,5-dioxo-imidazolidin-1-yl)-3,3-dimethylbutyl]-3-(3-{3-[4-(3-ethyl-2,5-dioxo-imidazolidin-1-yl)-3,3-dimethylbutyl]thioureido}propyl)thiourea;
162) 1-[4-(3-Ethyl-2,5-dithioxo-imidazolidin-1-yl)-3,3-dimethylbutyl]-3-(3-{3-[4-(3-ethyl-2,5-dithioxo-imidazolidin-1-yl)-3,3-dimethylbutyl]thioureido}propyl)thiourea;
163) 1-[4-(3-Ethyl-5-oxo-2-thioxo-imidazolidin-1-yl)-3,3-dimethylbutyl]-3-(3-{3-[4-(3-ethyl-5-oxo-2-thioxo-imidazolidin-1-yl)-3,3-dimethylbutyl]thioureido}propyl)thiourea;
164) 1-[4-(3-Ethyl-2-oxo-5-thioxo-imidazolidin-1-yl)-3,3-dimethylbutyl]-3-(3-{3-[4-(3-ethyl-2-oxo-5-thioxo-imidazolidin-1-yl)-3,3-dimethylbutyl]thioureido}propyl)thiourea;
165) 1-(3-Hydroxy-3-methylbutyl)-3-{2-[3-(3-hydroxy-3-methylbutyl)thioureido]ethyl}thiourea;
166) 1-(4-Hydroxy-3,3-dimethylbutyl)-3-{2-[3-(4-hydroxy-3,3-dimethylbutyl)thioureido]ethyl}thiourea;
167) 4-(3-{2-[3-(3-Carboxy-3-methylbutyl)thioureido]ethyl}thioureido)-2,2-dimethyl butyric acid;
168) 2-Methyl-4-(3-{2-[3-(3-methyl-3-sulfobutyl)thioureido]ethyl}thioureido)butane-2-sulfonic acid;
169) 1-(3,3-Dimethyl-4-oxobutyl)-3-{2-[3-(3,3-dimethyl-4-oxobutyl)thioureido]ethyl}thiourea;
170) 4-(3-{2-[3-(3-Methoxycarbonyl-3-methylbutyl)thioureido]ethyl}thioureido)-2,2-dimethyl butyric acid methyl ester;
171) 2,2-Dimethyl-4-(3-{2-[3-(3-methyl-3-phenoxycarbonylbutyl)thioureido]ethyl}thioureido)butyric acid phenyl ester;
172) 4-(3-{2-[3-(3-Benzyloxycarbonyl-3-methylbutyl)thioureido]ethyl}thioureido)-2,2-dimethyl butyric acid benzyl ester;
173) Phosphoricacidmono-[1,1-dimethyl-3-(3-{2-[3-(3-methyl-3-phosphonooxybutyl)thioureido]ethyl}thioureido)propyl]ester;
174) 1-[4-(4,6-Dioxo-2,3,3a,6-tetrahydro-4H-thieno[3,2-c]pyridin-5-yl)-4-methylpentyl]-3-(3-{3-[4-(4,6-dioxo-2,3,3a,6-tetrahydro-4H-thieno[3,2-c]pyridin-5-yl)-4-methylpentyl]thioureido}propyl)thiourea;
175) 1-[4-(4,6-Dithioxo-2,3,3a,6-tetrahydro-4H-thieno[3,2-c]pyridin-5-yl)-4-methylpentyl]-3-(3-{3-[4-(4,6-dithioxo-2,3,3a,6-tetrahydro-4H-thieno[3,2-c]pyridin-5-yl)-4-methylpentyl]thioureido}propyl)thiourea;
176) 5-[3-(3-{3-[4-(N-cyano-carbamoyl)-4-methylpentyl]thioureido}propyl)thioureido]-2,2-dimethyl pentanoic acid cyanamide;
177) Phosphoramidicacidmono-{4-[3-(3-{3-[4-(amino-hydroxy-phosphoryloxy)-4-methylpentyl]thioureido}propyl)thioureido]-1,1-dimethylbutyl}ester;
178) 1-(4-Methyl-4-tetrazol-1-yl-pentyl)-3-{3-[3-(4-methyl-4-tetrazol-1-yl-pentyl)thioureido]propyl}thiourea;
179) 1-[4-Methyl-4-(1H-tetrazol-5-yl)pentyl]-3-(3-{3-[4-methyl-4-(1H-tetrazol-5-yl)pentyl]thioureido}propyl)thiourea;
180) Bis-[1-carboxyl-3,3-dimethylpentylthioureido]methane;
181) Bis-[1-aldo-3,3-dimethylpentylthioureido]methane;
182) Bis-[1-methoxycarbonyl-3,3-dimethylpentylthioureido]methane;
183) Bis-[1-hydroxy-3,3-dimethylpentylthioureido]methane;
184) Bis-[1-phenoxycarbonyl-3,3-dimethylpentylthioureido]methane;
185) Bis-[benzyloxycarbonyl-3,3-dimethylpentylthioureido]methane;
186) 1,6-Bis-[2′-(4″,6″-dioxo-2″,3″,3a″,6″-tetrahydro-4″H-thieno[3′″,2′″-c]pyridin-5″-yl)-2′-methylpropyl]bithiourea;
187) 1,6-Bis-[1′-(N-cyanamido)-2′,2′-dimethylpropyl]bithiourea;
188) 1,6-Bis-[2′-(4″,6″-dithioxo-2″,3″,3a″,6″-tetrahydro-4″H-thieno[3′″,2′″-c]pyridin-5″-yl)-2′-methylpropyl]bithiourea;
189) 1,6-Bis-[2′-(aminohydroxyphosphoryloxy)-2′-methylpropyl]bithiourea;
190) 1,6-Bis-[2′-methyl-2′-(tetrazol-1″-yl)propyl]bithiourea;
191) 1,6-Bis-[2′-methyl-2′-(1″H-tetrazol-5″-yl)propyl]bithiourea;
192) Bis-[1-sulfo-2,2-dimethylbutylthioureido]methane;
193) Bis-[1-phosphonooxy-2,2-dimethylbutylthioureido]methane;
194) Bis-[1-(4′,6′-dioxo-2′,3′,3a′,6′-tetrahydro-4′H-thieno[3″,2″-c]pyridin-5′-yl)-2,2-dimethylbutylthioureido]methane;
195) Bis-[1-(aminohydroxyphosphoryloxy)-2,2-dimethylbutylthioureido]methane;
196) Bis-[1-(N-cyanoamido)-3,3-dimethylpentylthioureido]methane;
197) Bis-[1-(4′,6′-dithioxo-2′,3′,3a′,6′-tetrahydro-4′H-thieno[3″,2″-c]pyridin-5′-yl)-2,2-dimethylbutylthioureido]methane;
198) Bis-[1-(tetrazol-1″-yl)-2,2-dimethylbutylthioureido]methane;
199) Bis-[1-(1″H-tetrazol-5″-yl)-2,2-dimethylbutylthioureido]methane;
200) Bis-[1-(3′-hydroxy-isoazol-5′-yl)-2,2-dimethylbutylthioureido]methane;
201) Bis-[1-(3′-hydorxy-isoxazol-4′-yl)-2,2-dimethylbutylthioureido]methane;
202) Bis-[1-(5′-hydroxy-4′-oxo-4′H-pyran-2′-yl)-2,2-dimethybutylthioureido]methane;
203) Bis-[1-(5′-hydroxy-4′-oxo-4′H-pyran-3′-yl)-2,2-dimethybutylthioureido]methane;
204) Bis-[1-(5′-hydroxy-4′-oxo-4′H-pyran-3′-yl)-2,2-dimethybutylthioureido]methane;
205) Bis-[1-(3′-ethyl-2′,5′-dithioxo-imidazolidin-1′-yl)-2,2-dimethylbutylthioureido]methane;
206) [{4-(3″-ethyl-2″,5″-dithioxo-imidazolidin-1″-yl)-3,3-dimethylbutylthioureido}-{4′-(3′″-ethyl-2′″,5′″-dioxo-imidazolidin-1′″-yl)-3′,3′-dimethylbutylthioureido}]methane;
207) Bis-[1-(3′-ethyl-2′,5′-dioxo-imidazolidin-1′-yl)-2,2-dimethylbutylthioureido]methane;
208) Bis-[1-(3′-ethyl-5′-oxo-2′-thioxo-imidazolidin-1′-yl)-2,2-dimethylbutylthioureido]methane;
209) Bis-[1-(3′-ethyl-2′-oxo-5′-thioxo-imidazolidin-1′-yl)-2,2-dimethylbutylthioureido]methane;
210) 1-(5-Hydroxy-4,4-dimethylpentyl)-3-phenethylurea;
211) 1-(5-Hydroxy-4,4-dimethylpentyl)-3-(3-phenylpropyl)urea;
212) 1-(5-Hydroxy-4,4-dimethylpentyl)-3-(4-phenylbutyl)urea;
213) 1-(5-Hydroxy-4,4-dimethylpentyl)-3-(6-hydroxy-5,5-dimethylhexyl)urea;
214) 1-(5-Hydroxy-4,4-dimethylpentyl)-3-(6-hydroxy-5-methyl-5-phenylhexyl)urea;
215) 1-(6-Hydroxy-5,5-dimethylhexyl)-3-phenethylurea;
216) 1-(6-Hydroxy-5,5-dimethylhexyl)-3-(3-phenylpropyl)urea;
217) 1-(6-Hydroxy-5,5-dimethylhexyl)-3-(4-phenylbutyl)urea;
218) methyl 4-(3-(6-hydroxy-5,5-dimethylhexyl)ureido)butanoate;
219) Ethyl 6-(3-(6-hydroxy-5,5-dimethylhexyl)ureido)hexanoate;
220) 1-(6-Hydroxy-5,5-dimethylhexyl)-3-(6-hydroxy-5-methyl-5-phenylhexyl)urea;
221) 1-(6-Hydroxy-5,5-dimethylhexyl)-3-(7-hydroxy-6-methyl-6-phenylheptyl)urea;
222) 1-(6-Hydroxy-5-methyl-5-phenylhexyl)-3-phenethylurea;
223) 1-(6-Hydroxy-5-methyl-5-phenylhexyl)-3-(3-phenylpropyl)urea;
224) 1-(6-Hydroxy-5-methyl-5-phenylhexyl)-3-(4-phenylbutyl)urea;
225) Ethyl 6-(3-(6-hydroxy-5-methyl-5-phenylhexyl)ureido)hexanoate;
226) 1-(6-Hydroxy-5-methyl-5-phenylhexyl)-3-(7-hydroxy-6-methyl-6-phenylheptyl)urea;
227) 1-(7-Hydroxy-6-methyl-6-phenylheptyl)-3-phenethylurea;
228) 1-(7-hydroxy-6-methyl-6-phenylheptyl)-3-(3-phenylpropyl)urea;
229) 1-(7-Hydroxy-6-methyl-6-phenylheptyl)-3-(4-phenylbutyl)urea;
230) Ethyl 7-(3-(7-hydroxy-6-methyl-6-phenylhexyl)ureido)butanoate;
231) 1,3-B is(7-hydroxy-6-methyl-6-phenylheptyl)urea;
232) 1,3-Bis(6-hydroxy-5,5-dimethylhexyl)urea; or
233) 1,3-Bis(5-hydroxy-4,4-dimethylpentyl)urea.
